(19) United States
(12) Patent Application Publication
(10) Pub. No.: US 2006/0276393 A1
(43) Pub. Date: Dec. 7, 2006

(54) Novel compositions for preventing and treating neurodegenerative and blood coagulation disorders

(75) Inventors: Michael Milburn, Cary, NC (US); Jill Milne, Brookline, MA (US); Christoph H. Westphal, Brookline, MA (US); Karl D. Normington, Acton, MA (US); Jennifer Fujii, Lexington, MA (US); Michelle Dipp, Cambridge, MA (US); Peter Elliott, Marlborough, MA (US)

Correspondence Address: FISH & NEAVE IP GROUP;ROPES & GRAY LLP, ONE INTERNATIONAL PLACE, BOSTON, MA, 02110-2624, US

(73) Assignee: Sirtris Pharmaceuticals, Inc., Cambridge, MA (US)

(21) Appl. No.: 11/332,056

(22) Filed: Jan. 13, 2006

Related U.S. Application Data
(60) Provisional application No. 60/643,921, filed on Jan. 13, 2005.
(60) Provisional application No. 60/667,179, filed on Mar. 30, 2005.
(60) Provisional application No. 60/692,785, filed on Jun. 22, 2005.
(60) Provisional application No. 60/736,528, filed on Nov. 14, 2005.
(60) Provisional application No. 60/753,606, filed on Dec. 23, 2005.

(51) Int. Cl. A61K 38/17; A61K 31/7048; A61K 31/353; A61K 31/05

(52) U.S. Cl. 514/12; 514/27; 514/456; 514/733

(57) ABSTRACT

Provided herein are methods and compositions for treating or preventing neurodegenerative disorders or blood coagulation disorders. Methods may comprise modulating the activity or level of a sirtuin, such as SIRT1 or Sir2. Exemplary methods comprise contacting a cell with a sirtuin activating compound, such as a flavone, stilbene, flavanone, isoflavone, catechin, chalcone, tannin or anthocyanidin; or an inhibitory compound, such as nicotinamide.

### RELATED APPLICATIONS

This application claims the benefit of priority to U.S. Provisional Patent Application No. 60/643,921, filed on Jan. 13, 2005, 60/692,785, filed on Jun. 22, 2005, 60/667179, filed Mar. 30, 2005, 60/736,528, filed Nov. 14, 2005, and 60/753,606, filed Dec. 23, 2005, which applications are hereby incorporated by reference in their entireties.

### BACKGROUND

Neurodegenerative disorders are progressively debilitating and most are ultimately fatal. Although proteins were recently identified to be involved in the overall pathogenic progression of certain neurodegenerative diseases (such as Parkinson's disease, Alzheimer's disease, Huntington's disease, Spinocerebellar Ataxia Type 1, Type 2, and Type 3, and dentatorubral pallidoluysian atrophy (DRLPA)), there is currently no cure for these neurodegenerative diseases. Further amplifying the problem of neurodegenerative diseases (especially Alzheimer's disease and Parkinson's disease) is that their prevalence continues to increase, thus creating a serious public health problem.

Blood coagulation disorders result from abnormal hemostatic reaction in the living body. Hemostatic reaction generally consists of primary hemostasis wherein platelets adhere and agglutinate to impaired portions of the blood vessel and secondary hemostasis wherein soluble fibrinogens are transformed into insoluble fibrins to plug the impaired portions. The process of secondary hemostasis is accomplished by successive reactions known as a blood coagulation cascade by a variety of blood coagulation factors and cofactors and has two courses (the intrinsic and extrinsic coagulation pathways). Thus, if any factor or cofactor in the blood coagulation cascade is deficient or does not work properly, blood coagulation is hindered which may lead to hemorrhage. For example, typical diseases caused by congenital disorders in blood coagulation factors are hemophilia A and B, deficient in Factor VIII and Factor IX, respectively.

With the number of individuals affected with neurodegenerative disorders and blood coagulation disorders on the increase, there is a dire need for medications that prevent and treat these conditions.

### SUMMARY

Provided herein are methods for treating or preventing neurodegenerative disorders in a subject. A method may comprise administering to a subject in need thereof a therapeutically effective amount of an agent that increases the activity or protein level of a sirtuin in a cell, such as SIRT1 or Sir2. The agent may be a sirtuin-activating compound, or a salt or prodrug thereof. The sirtuin-activating compound preferably stimulates human Sir2, i.e., SIRT1, protein activity. The method may comprise providing a sirtuin-activating compound having a formula selected from the group consisting of formulas 1-25, 30, 32-65, and 69-88, or a salt or prodrug thereof. Sirtuin-activating compounds may be flavones, stilbenes, flavanones, isoflavones, catechins, chalcones, tannins and anthocyanidins or analog or derivative thereof. Sirtuin-activating compounds may be selected from the group consisting of resveratrol, butein, piceatannol, isoliquiritgenin, fisetin, luteolin, 3,6,3′,4′-tetrahydroxyfalvone, quercetin, and analogs and derivatives thereof. In certain embodiments, the method may also comprise administering, e.g., conjointly administering, to a subject a therapeutically effective amount of another anti-neurodegeneration agent. A range of techniques for administering sirtuin-activating compounds and anti-neurodegeneration agents are contemplated. Sirtuin-activating compounds and anti-neurodegeneration agents do not need to be administered in the same way or at the same time, but they are preferably administered such that their effects overlap, are synergistic, complementary or additive. Exemplary neurodegenerative disorders include, but are not limited to, Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, Multiple Sclerosis (MS), Friedreich's ataxia, and chemotherapeutic induced neuropathy. In exemplary embodiments, the subject is a human. In an exemplary embodiment, an amount of a sirtuin activating compound that has a sirtuin activating effect equal to or greater than 18, 20, 25, 30, 35, 40, 50, 60, 75, 100, 150 mg/kg, or more, of resveratrol is administered to a human subject. In another embodiment, at least 18, 20, 25, 30, 35, 40, 50, 60, 75, 100, 150 mg/kg, or more, of resveratrol is administered to a human subject.

In yet another embodiment, methods for treating or preventing a polyglutamine disease are provided. The methods may comprise administering to a subject in need thereof a therapeutically effective amount of an agent that increases the activity or protein level of a sirtuin, e.g., SIRT1 or Sir2. The agent may be a sirtuin-activating compound, or a salt or prodrug thereof. The sirtuin-activating compound preferably stimulates human Sir2, i.e., SIRT1, protein activity. The method may comprise providing a sirtuin-activating compound having a formula selected from the group consisting of formulas 1-25, 30, 32-65, and 69-88, or a salt or prodrug thereof. Sirtuin-activating compounds may be flavones, stilbenes, flavanones, isoflavones, catechins, chalcones, tannins and anthocyanidins or analog or derivative thereof. Sirtuin-activating compounds may be selected from the group consisting of resveratrol, butein, piceatannol, isoliquiritgenin, fisetin, luteolin, 3,6,3′,4′-tetrahydroxyfalvone, quercetin, and analogs and derivatives thereof. In certain embodiments, the method may also comprise administering, e.g., conjointly administering, to a subject a therapeutically effective amount of an HDAC I/II inhibitor. In certain embodiments, the HDAC I/II inhibitor may be a hydroxamic acid, a cyclic peptie, a benzamide, a short-chain fatty acid, or depudecin. In other embodiments, the HDAC I/II inhibitor may be selected from the group consisting of: suberoylanilide hydroxamic acid (SAHA), butyrate, pyroxamide, depsipeptide, or MS-27-275. A range of techniques for administering sirtuin-activating compounds and HDAC I/II inhibitors are contemplated. Sirtuin-activating compounds and HDAC I/II inhibitors do not need to be administered in the same way or at the same time, but they are preferably administered such that their effects overlap, are synergistic, complementary or additive. Exemplary polyglutamine diseases include, but are not limited to, spinobulbar muscular atrophy (Kennedy disease), Huntington's disease, dentatorubralpallidoluysian atrophy (Haw River syndrome), spinocerebellar ataxia type 1, spinocerebellar ataxia type 2, spinocerebellar ataxia type 3 (Machado-Joseph disease), spinocerebellar ataxia type 6, spinocerebellar ataxia type 7, or spinocerebellar ataxia type 17. In exemplary embodiments, the subject is a human. In an exemplary embodiment, an amount of a sirtuin activating compound that has a sirtuin activating effect equal to or greater than 18, 20, 25, 30, 35, 40, 50, 60, 75, 100, 150 mg/kg, or more, of resveratrol is administered to a human subject. In another embodiment, at least 18, 20, 25, 30, 35, 40, 50, 60, 75, 100, 150 mg/kg, or more, of resveratrol is administered to a human subject.

In another embodiment, the invention provides a method for treating or preventing neuropathy associated with an ischemic event or disease comprising administering to a subject in need thereof a therapeutically effective amount of an agent that increases the activity or protein level of a sirtuin, e.g., SIRT1 or Sir2. The agent may be a sirtuin-activating compound, or a salt or prodrug thereof. The sirtuin-activating compound preferably stimulates human Sir2, i.e., SIRT1, protein activity. The method may comprise providing a sirtuin-activating compound having a formula selected from the group consisting of formulas 1-25, 30, 32-65, and 69-88, or a salt or prodrug thereof. Sirtuin-activating compounds may be flavones, stilbenes, flavanones, isoflavones, catechins, chalcones, tannins and anthocyanidins or analog or derivative thereof. Sirtuin-activating compounds may be selected from the group consisting of resveratrol, butein, piceatannol, isoliquiritgenin, fisetin, luteolin, 3,6,3′,4′-tetrahydroxyfalvone, quercetin, and analogs and derivatives thereof. Exemplary ischemic events or diseases include, for example, stroke, coronary heart disease, stroke, emphysema, hemorrhagic shock, arrhythmia (e.g. atrial fibrillation), peripheral vascular disease, transplant related injuries, congestive heart failure, or a myocardial infarction. In exemplary embodiments, the subject is a human. In an exemplary embodiment, an amount of a sirtuin activating compound that has a sirtuin activating effect equal to or greater than 18, 20, 25, 30, 35, 40, 50, 60, 75, 100, 150 mg/kg, or more, of resveratrol is administered to a human subject. In another embodiment, at least 18, 20, 25, 30, 35, 40, 50, 60, 75, 100, 150 mg/kg, or more, of resveratrol is administered to a human subject.

In yet another embodiment, the invention provides a method for treating or preventing chemotherapeutic induced neuropathy comprising administering to a subject in need thereof a therapeutically effective amount of an agent that increases the activity or protein level of a sirtuin, e.g., SIRT1 or Sir2. The agent may be a sirtuin-activating compound, or a salt or prodrug thereof. The sirtuin-activating compound preferably stimulates human Sir2, i.e., SIRT1, protein activity. The method may comprise providing a sirtuin-activating compound having a formula selected from the group consisting of formulas 1-25, 30, 32-65, and 69-88, or a salt or prodrug thereof. Sirtuin-activating compounds may be flavones, stilbenes, flavanones, isoflavones, catechins, chalcones, tannins and anthocyanidins or analog or derivative thereof. Sirtuin-activating compounds may be selected from the group consisting of resveratrol, butein, piceatannol, isoliquiritgenin, fisetin, luteolin, 3,6,3′,4′-tetrahydroxyfalvone, quercetin, and analogs and derivatives thereof. In certain embodiments, the chemotherapeutic comprises a vinka alkaloid (such as, for example, vinblastine, vincristine, or vindesine) or cisplatin. In exemplary embodiments, the subject is a human. In an exemplary embodiment, an amount of a sirtuin activating compound that has a sirtuin activating effect equal to or greater than 18, 20, 25, 30, 35, 40, 50, 60, 75, 100, 150 mg/kg, or more, of resveratrol is administered to a human subject. In another embodiment, at least 18, 20, 25, 30, 35, 40, 50, 60, 75, 100, 150 mg/kg, or more, of resveratrol is administered to a human subject.

In another embodiment, the invention provides a method for treating or preventing a neurodegenerative disease or disorder comprising administering to a subject in need thereof a therapetucially effective amount of a PPAR-delta agonist, such as, for example, GW0742 or GW501516.

In another embodiment, the invention provides a method for treating or preventing a neurodegenerative disease or disorder comprising administering to a subject in need thereof a therapetucially effective amount of at least one sirtuin-activating compound in combination with at least one PPAR agonist. The sirtuin-activating compound preferably stimulates human Sir2, i.e., SIRT1, protein activity. The method may comprise providing a sirtuin-activating compound having a formula selected from the group consisting of formulas 1-25, 30, 32-65, and 69-88, or a salt or prodrug thereof. Sirtuin-activating compounds may be flavones, stilbenes, flavanones, isoflavones, catechins, chalcones, tannins and anthocyanidins or analog or derivative thereof. Sirtuin-activating compounds may be selected from the group consisting of resveratrol, butein, piceatannol, isoliquiritgenin, fisetin, luteolin, 3,6,3′,4′-tetrahydroxyfalvone, quercetin, and analogs and derivatives thereof. In various embodiments, the PPAR agonist may be a PPAR-alpha agonist, a PPAR-gamma agonist, or a PPAR delta agonist. A range of techniques for administering sirtuin-activating compounds and PPAR agonists are contemplated. Sirtuin-activating compounds and PPAR agonists do not need to be administered in the same way or at the same time, but they are preferably administered such that their effects overlap, are synergistic, complementary or additive. Exemplary neurodegenerative disorders include, but are not limited to, Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, Multiple Sclerosis (MS), Friedreich's ataxia, and chemotherapeutic induced neuropathy. In exemplary embodiments, the subject is a human. In an exemplary embodiment, an amount of a sirtuin activating compound that has a sirtuin activating effect equal to or greater than 18, 20, 25, 30, 35, 40, 50, 60, 75, 100, 150 mg/kg, or more, of resveratrol is administered to a human subject. In another embodiment, at least 18, 20, 25, 30, 35, 40, 50, 60, 75, 100, 150 mg/kg, or more, of resveratrol is administered to a human subject.

In yet another embodiment, the invention provides a method for treating a neurodegenerative disease or disorder associated with inflammation comprising administering to a subject tin need thereof a therapeutically effective amount of a combination of an anti-inflammatory agent and a sirtuin-activating compound. The sirtuin-activating compound preferably stimulates human Sir2, i.e., SIRT1, protein activity. The method may comprise providing a sirtuin-activating compound having a formula selected from the group consisting of formulas 1-25, 30, 32-65, and 69-88, or a salt or prodrug thereof. Sirtuin-activating compounds may be flavones, stilbenes, flavanones, isoflavones, catechins, chalcones, tannins and anthocyanidins or analog or derivative thereof. Sirtuin-activating compounds may be selected from the group consisting of resveratrol, butein, piceatannol, isoliquiritgenin, fisetin, luteolin, 3,6,3′,4′-tetrahydroxyfalvone, quercetin, and analogs and derivatives thereof. Exemplary anti-inflammatory agents include, for example, steroidal anti-inflammatory agents, non-steroidal anti-inflammatory agents, and non-steroidal immunomodulatory agents. Exemplary neurodegenerative diseases associated with inflammation include, for example, Alzheimer's disease (AD), Huntington's Disease (HD) and other polyglutamine diseases, Parkinsons Disease (PD), amyotrophic lateral sclerosis (ALS; Lou Gehrig's disease), and multiple sclerosis (MS). A range of techniques for administering sirtuin-activating compounds and anti-inflammatory agents are contemplated. Sirtuin-activating compounds and anti-inflammatory agents do not need to be administered in the same way or at the same time, but they are preferably administered such that their effects overlap, are synergistic, complementary or additive. Exemplary neurodegenerative disorders include, but are not limited to, Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, Multiple Sclerosis (MS), Friedreich's ataxia, and chemotherapeutic induced neuropathy. In exemplary embodiments, the subject is a human. In an exemplary embodiment, an amount of a sirtuin activating compound that has a sirtuin activating effect equal to or greater than 18, 20, 25, 30, 35, 40, 50, 60, 75, 100, 150 mg/kg, or more, of resveratrol is administered to a human subject. In another embodiment, at least 18, 20, 25, 30, 35, 40, 50, 60, 75, 100, 150 mg/kg, or more, of resveratrol is administered to a human subject.

In another embodiment, the invention provides a method for preventing or treating a traumatic injury to a neuronal cell, comprising contacting a neuronal cell with an agent that increases the activity or protein level of a sirtuin, e.g., SIRT1 or Sir2. The agent may be a sirtuin-activating compound, or a salt or prodrug thereof. The sirtuin-activating compound preferably stimulates human Sir2, i.e., SIRT1, protein activity. The method may comprise providing a sirtuin-activating compound having a formula selected from the group consisting of formulas 1-25, 30, 32-65, and 69-88, or a salt or prodrug thereof. Sirtuin-activating compounds may be flavones, stilbenes, flavanones, isoflavones, catechins, chalcones, tannins and anthocyanidins or analog or derivative thereof. Sirtuin-activating compounds may be selected from the group consisting of resveratrol, butein, piceatannol, isoliquiritgenin, fisetin, luteolin, 3,6,3′,4′-tetrahydroxyfalvone, quercetin, and analogs and derivatives thereof. The traumatic injury may be caused by, for example, a surgical procedure or a physical insult. In exemplary embodiments, the subject is a human. In an exemplary embodiment, an amount of a sirtuin activating compound that has a sirtuin activating effect equal to or greater than 18, 20, 25, 30, 35, 40, 50, 60, 75, 100, 150 mg/kg, or more, of resveratrol is administered to a human subject. In another embodiment, at least 18, 20, 25, 30, 35, 40, 50, 60, 75, 100, 150 mg/kg, or more, of resveratrol is administered to a human subject.

In another embodiment, the invention provides a method for treating a neurodegenerative disease or disorder in a subject that would benefit from increased mitochondrial activity, comprising administering to a subject in need thereof a therapeutically effective amount of a sirtuin activating compound. The sirtuin activating compound may increases mitochondrial activity and/or mitochondrial mass. In certain embodiments, the method may further comprising administering to the subject one or more of the following: a vitamin, a cofactor, an antioxidant, coenzyme Q₁₀, L-carnitine, thiamine, riboflavin, niacinamide, folate, vitamin E, selenium, lipoic acid, or prednisone, in combination with the sirtuin activating compound. In certain embodiments, the method may comprise administering a combination of a sirtuin-activating compound in combination with one or more agents that alleviate a symptom of the neurodegenerative disease or disorder, such as, for example, an agent alleviates seizures, an agent that alleviates neuropathic pain, or anti-neurodegenerative agent. In exemplary embodiments, the subject is a human. In an exemplary embodiment, an amount of a sirtuin activating compound that has a sirtuin activating effect equal to or greater than 18, 20, 25, 30, 35, 40, 50, 60, 75, 100, 150 mg/kg, or more, of resveratrol is administered to a human subject. In another embodiment, at least 18, 20, 25, 30, 35, 40, 50, 60, 75, 100, 150 mg/kg, or more, of resveratrol is administered to a human subject.

Also provided are methods for preventing or treating blood coagulation disorders. A method may comprise administering to a subject in need thereof a therapeutically effective amount of an agent that increases the activity or protein level of a sirtuin, e.g., SIRT1 or Sir2. The agent may be a sirtuin-activating compound, or a salt or prodrug therof. The sirtuin-activating compound may stimulate human Sir2, i.e., SIRT1, protein activity. The method may comprise providing a sirtuin-activating compound having a formula selected from the group consisting of formulas 1-25, 30, 32-65, and 69-88, or a salt or prodrug thereof. Sirtuin-activating compounds may be flavones, stilbenes, flavanones, isoflavones, catechins, chalcones, tannins and anthocyanidins or analog or derivative thereof. Sirtuin-activating compounds may be selected from the group consisting of resveratrol, butein, piceatannol, isoliquiritgenin, fisetin, luteolin, 3,6,3′,4′-tetrahydroxyfalvone, quercetin, and analogs and derivatives thereof. In certain embodiments, the method for preventing or treating blood coagulation disorders may further comprise administering, e.g., conjointly administering, to a subject a therapeutically effective amount of another anti-coagulation, anti-thromboembolic agent or anti-thrombosis agent. A range of techniques for administering sirtuin-activating compounds and anti-coagulation/anti-thrombosis agents are contemplated. Sirtuin-activating compounds and anti-coagulation/anti-thrombosis agents do not need to be administered in the same way or at the same time, but they are preferably administered such that their effects overlap, are synergistic, complementary or additive. Exemplary blood coagulation disorders include, but are not limited to, thromboembolism, deep vein thrombosis, pulmonary embolism, stroke, myocardial infarction, arrhythmia (e.g. atrial fibrillation), miscarriage, thrombophilia associated with anti-thrombin III deficiency, protein C deficiency, protein S deficiency, resistance to activated protein C, dysfibrinogenemia, fibrinolytic disorders, homocystinuria, pregnancy, inflammatory disorders, myeloproliferative disorders, arteriosclerosis, angina, disseminated intravascular coagulation, thrombotic thrombocytopenic purpura, cancer metastasis, sickle cell disease, nephritides such as glomerular nephritis, drug induced thrombocytopenia, and re-occlusion during or after therapeutic clot lysis or procedures such as angioplasty or surgery. In exemplary embodiments, the subject is a human. In an exemplary embodiment, an amount of a sirtuin activating compound that has a sirtuin activating effect equal to or greater than 18, 20, 25, 30, 35, 40, 50, 60, 75, 100, 150 mg/kg, or more, of resveratrol is administered to a human subject. In another embodiment, at least 18, 20, 25, 30, 35, 40, 50, 60, 75, 100, 150 mg/kg, or more, of resveratrol is administered to a human subject.

Also provided herein are methods for preventing or treating a disorder associated with hypocoagulation in a subject. A method may comprise administering to a subject in need thereof a therapeutically effective amount of an agent that decreases the activity or protein level of a sirtuin, such as SIRT1 or Sir2. The agent may be a sirtuin-inhibiting compound, or a salt or prodrug thereof. The sirtuin-inhibiting compound may inhibit the activity of the human Sir2, i.e., SIRT1 protein. The method may comprise administering to the subject an effective amount of a sirtuin-inhibiting compound having a formula selected from the group of formulas 26-29, 31, and 66-68, or a salt or prodrug thereof. In certain embodiments, the sirtuin-inhibiting compound may be nicotinamide. The method may also further comprise administering, e.g., conjointly administering, to the subject a therapeutically effective amount of another pro-coagulation agent. A range of techniques for administering sirtuin-inhibiting compounds and pro-coagulation agents of the invention are contemplated. Sirtuin-inhibiting compounds and pro-coagulation agents do not need to be administered in the same way or at the same time, but they are preferably administered such that their effects overlap, are synergistic, complementary, or additive. Exemplary disorders associated with hypocoagulation include, but are not limited to, hemophilia A, hemophilia B, and von Willebrand disease. In exemplary embodiments, the subject is a human.

In another embodiment, the invention provides a method for inhibiting blood coagulation, comprising contacting a blood cell with an agent that increases the activity or protein level of a sirtuin, such as SIRT1 or Sir2. The agent may be a sirtuin-inhibiting compound, or a salt or prodrug thereof. The sirtuin-inhibiting compound may inhibit the activity of the human Sir2, i.e., SIRT1 protein. In exemplary embodiments, the subject is a human. In an exemplary embodiment, an amount of a sirtuin activating compound that has a sirtuin activating effect equal to or greater than 18, 20, 25, 30, 35, 40, 50, 60, 75, 100, 150 mg/kg, or more, of resveratrol is administered to a human subject. In another embodiment, at least 18, 20, 25, 30, 35, 40, 50, 60, 75, 100, 150 mg/kg, or more, of resveratrol is administered to a human subject.

In another embodiment, the invention provides a method for enhancing blood coagulation, comprising contacting a blood cell with an agent that decreases the activity or protein level of a sirtuin, such as SIRT1 or Sir2. The agent may be a sirtuin-inhibiting compound, or a salt or prodrug thereof. The sirtuin-inhibiting compound may inhibit the activity of the human Sir2, i.e., SIRT1 protein. In exemplary embodiments, the subject is a human. In an exemplary embodiment, an amount of a sirtuin activating compound that has a sirtuin activating effect equal to or greater than 18, 20, 25, 30, 35, 40, 50, 60, 75, 100, 150 mg/kg, or more, of resveratrol is administered to a human subject. In another embodiment, at least 18, 20, 25, 30, 35, 40, 50, 60, 75, 100, 150 mg/kg, or more, of resveratrol is administered to a human subject.

Also provided is the use of a sirtuin-activating compound for the manufacture of a medicament for treating or preventing neurodegenerative disorders or blood coagulation disorders; or use of a sirtuin-activating compound for the manufacture of a medicament for preventing or inhibiting a traumatic injury to a neuronal cell in a subject, or for inhibiting blood coagulation in a subject. In another embodiment, provided is use of a sirtuin-inhibiting compound for the manufacture of a medicament for promoting or inducing blood coagulation in a subject. In exemplary embodiments, the subject is a human.

### BRIEF DESCRIPTION OF THE DRAWINGS

FIG. 1 shows the effects of resveratrol on the kinetics of recombinant human SIRT1. a, Resveratrol dose-response of SIRT1 catalytic rate at 25 μM NAD⁺, 25 μM p53-382 acetylated peptide. Relative initial rates are the mean of two determinations, each derived from the slopes of fluorescence (arbitrary fluorescence units, AFU) vs. time plots with data obtained at 0, 5, 10 and 20 min. of deacetylation. b, SIRT1 initial rate at 3 mM NAD⁺, as a function of p53-382 acetylated peptide concentration in the presence (Δ) or absence (ν) of 100 μM resveratrol. Lines represent non-linear least-squares fits to the Michaelis-Menten equation. Kinetic constants: Kₘ(control, ν)=64 μM, Kₘ(+resveratrol, Δ)=1.8 μM; Vₘₐₓ(control, ν)=1107 AFU/min., Vₘₐₓ(+resveratrol, Δ)=926 AFU/min. c, SIRT1 initial rate at 1 mM p53-382 acetylated peptide, as a function of NAD⁺ concentration, in the presence (Δ) or absence (ν) of 100 μM resveratrol. Lines represent non-linear least-squares fits to the Michaelis-Menten equation. Kinetic constants: Kₘ(control, ν)=558 μM, Kₘ(+resveratrol, Δ)=101 μM; Vₘₐₓ(control, ν)=1863 AFU/min., Vₘₐₓ(+resveratrol, Δ)=1749 AFU/min. d, Effects of resveratrol on nicotinamide inhibition of SIRT1. Kinetic constants are shown relative to those of the control (no nicotinamide, no resveratrol) and represent the mean of two determinations. Error bars are standard errors of the mean. The variable substrate in each experiment (N=NAD⁺, P=p53 acetylated peptide), the presence/absence of nicotinamide (±) and the resveratrol concentration (μM) are indicated beneath each pair of Kₘ−Vₘₐₓ bars.

FIG. 2 shows the effects of polyphenols on Sir2 and S. cerevisiae lifespan. a, Initial deacetylation rate of recombinant GST-Sir2 as a function of resveratrol concentration. Rates were determined at the indicated resveratrol concentrations, either with 100 μM ‘Fluor de Lys’ acetylated lysine substrate (FdL) plus 3 mM NAD⁺ (A) or with 200 μM p53-382 acetylated peptide substrate plus 200 μM NAD⁺(ν). b, Lifespan analyses were determined by micro-manipulating individual yeast cells as described³⁷ on complete 2% glucose medium with 10 μM of each compound, unless otherwise stated. Average lifespan for wild type, 22.9 generations, quercetin, 23.4; piceatannol. 24.0. c, Average lifespan for wild type, 22.9 generations; fisetin, 30.0; butein, 35.5; resveratrol, 36.8. d, Average lifespan for wild type untreated, 21.0 generations; growth on resveratrol, 10 μM, 35.7; 100 μM, 29.4; 500 μM, 29.3.

FIG. 3 shows that resveratrol extends lifespan by mimicking CR and suppressing rDNA recombination. Yeast lifespans were determined as in FIG. 2. a, Average lifespan for wild type (wt) untreated, 19.0 generations; wild type+resveratrol (wt+R) 37.8; glucose-restricted+resveratrol (CR+R), 39.9. b, Average lifespans for wild type sir2Δ, 9.9; sir2Δ+resveratrol, 10.0; pnclΔ, 19.2; pnclΔ+resveratrol, 33.1. c, Resveratrol suppresses the frequency of ribosomal DNA recombination in the presence and absence of nicotinamide (NAM). Frequencies were determined by loss of the ADE2 marker gene from the rDNA locus (RDN1). d, Resveratrol does not suppress rDNA recombination in a sir2 strain. e, Resveratrol and other sirtuin activators do not significantly increase rDNA silencing compared to a 2×SIR2 strain. Pre-treated cells (RDN1::URA3) were harvested and spotted as 10-fold serial dilutions on either SC or SC with 5-fluororotic acid (5-FOA). In this assay, increased rDNA silencing results in increased survival on 5-FOA medium. f, Quantitation of the effect of resveratrol on rDNA silencing by counting numbers of surviving cells on FOA/total plated.

FIG. 4 shows that resveratrol and other polyphenols stimulate SIRT1 activity in human cells. a, Method for assaying intracellular deacetylase activity with a fluorogenic, cell-permeable substrate, FdL (‘Fluor de Lys’, BIOMOL). FdL (200 μM) is added to growth media and cells incubated for 1-3 hours to allow FdL to enter the cells and the lysine-deacetylated product (deAc-FdL) to accumulate intracellularly. Cells are lysed with detergent in the presence of 1 μM TSA, 1 mM nicotinamide. Addition of the non-cell-permeable Developer (BIOMOL) releases a fluorophor, specifically from deAc-FdL. b, SIRT1 activating polyphenols can stimulate TSA-insensitive, FdL deacetylation by HeLa S3 cells. Cells were grown adherently in DMEM/10% FCS and treated for 1 hour with 200 μM FdL, 1 μM TSA and either vehicle (0.5% final DMSO, Control) or 500 μM of the indicated compound. Intracellular accumulation of deAc-FdL was then determined as described briefly in a. The intracellular deAc-FdL level for each compound (mean of six replicates) are plotted against the ratios to the control rate obtained in the in vitro SIRT1 polyphenol screen (see Table 1, Supplementary Tables 1 and 3). c, U2OS osteosarcoma cells grown to ≧90% confluence in DMEM/10% FCS were exposed to 0 or 10 grays of gamma irradiation (IR). Whole cell lysates were prepared 4 hours post-irradiation and were probed by Western blotting with indicated antibodies. d, U2OS cells cultured as above were pre-treated with the indicated amounts of resveratrol or a 0.5% DMSO blank for 4 hours after which cells were exposed to 0 or 50 J/cm² of UV radiation. Lysates were prepared and analyzed by Western blot as in c. e, Human embryonic kidney cells (HEK 293) expressing wild type SIRT1 or dominant negative SIRT1-H363Y (SIRT1-HY) protein were cultured as above, pre-treated with the indicated amounts of resveratrol or a 0.5% DMSO blank for 4 hours and exposed to 50 J/cm² of UV radiation as above. Lysates were prepared and analyzed as above.

FIG. 5 shows that intracellular deacetylation activity may be measured with a cell-permeable, fluorogenic HDAC and sirtuin substrate. HeLa S3 cells were grown to confluence in DMEM/10% FCS and then incubated with fresh medium containing 200 μM FdL for the indicated times, 37° C. Intracellular and medium levels of deacetylated substrate (deAc-FdL) were determined according to the manufacturer's instructions (HDAC assay kit, BIOMOL). All data points represent the mean of two determinations. a, Concentration ratio of intracellular ([deAc-FdL]ᵢ) to medium ([deAc-FdL]ₒ) concentrations in the presence (Δ) or absence (ν) of 1 μM trichostatin A (TSA). b, Total accumulation of deacetylated substrate (deAc-FdL) in the presence (Δ) or absence (ν) of 1 μM TSA. c, Intracellular accumulation of deacetylated substrate (deAc-FdL) in the presence (Δ) or absence (ν) of 1 μM TSA.

FIG. 6 shows that deacetylation site preferences of recombinant SIRT1. Initial rates of deacetylation were determined for a series of fluorogenic acetylated peptide substrates based on short stretches of human histone H3, H4 and p53 sequence (see key to substrate name and single letter peptide sequence below the bar graph). Recombinant human SIRT1 (1 μg, BIOMOL), was incubated 10 min, 37° C., with 25 μM of the indicated fluorogenic acetylated peptide substrate and 500 μM NAD⁺. Reactions were stopped by the addition of 1 mM nicotinamide and the deacetylation-dependent. fluorescent signal was determined.

FIG. 7 is a graph representing SIRT2 activity as a function of resveratrol concentration.

FIG. 8 shows an alignment of the amino acid sequences of hSIRT2, hSIRT1 and S. cerevisiae Sir2.

FIG. 9A shows resveratrol and BML-230 dose responses of SIRT1 catalytic rate.

FIG. 9B shows the ratio of BML-230-activated to resveratrol-activated SIRT1 rates as a function of activator concentration (the ratios were calculated from data of FIG. 9A).

FIG. 10 shows the effect of polyphenolic STACs on metazoan sirtuins. a, Schematic of Sir2 polypeptides from human, yeast, C. elegans and D. melanogaster aligned to show conserved regions. Amino acids forming the NAD⁺-binding pocket (grey) and substrate binding groove (black) are indicated. Percentages refer to the homology to SIRT1. b, Effect of polyphenolic STACs (500 μM) on NAD⁺-dependent, trichostatin A (TSA)-insensitive deacetylase activity in Drosophila S2 cells. c, Fold stimulation of recombinant SIR-2.1 by STACs (10 μM). d, Fold stimulation of recombinant dSir2 by STACs (10 μM). Values are the mean of at least three determinations (±standard error). e, Dose-dependent activation of C. elegans SIR-2.1 by resveratrol. Rates were determined using a fluorigenic acetylated lysine substrate (Fluor de Lys). f, Dose-dependent activation of Drosophila dSir2 by resveratrol. g, SIR-2.1 initial rate at 10 μM Fluor de Lys as a function of NAD⁺ concentration, in the presence or absence of 100 μM resveratrol. AFU, arbitrary fluorescence units.

FIG. 11 shows the C. elegans survival on resveratrol. a, Survivorship of adult wild-type N2 C. elegans treated with 100 μM resveratrol fed with heat-killed OP50 E. coli. Mean lifespan relative to control (triangles, n=47) was increased by 14.5% (Log-Rank test, P<0.0001) by 100 μM resveratrol (squares, n=46). b, Survivorship of sir-2.1 mutants treated with resveratrol fed with heat-killed OP50. Adult lifespan of sir-2.1 animals does not differ significantly from N2 controls (Log-Rank, P=0.68) and the effect on lifespan of 100 μM resveratrol on sir-2.1 mutant animals was not statistically significant (5.2% extension, Log-Rank P=0.058; n=60 control, 58 treated). c, Survivorship of wild-type N2 C. elegans on 100 μM resveratrol fed with live OP50 (12.6% extension, P<0.0001; n=47 control, 67 treated). d, Survivorship of sir-2.1 mutants on 100 μM resveratrol fed with live OP50 (3.3% extension, P=0.81; n=57 control, 51 treated) e, Fecundity of adult hermaphrodites treated with 100 □M resveratrol. Controls: 106 eggs/5 worms/5 hours (s.d. 10.0); resveratrol-treated: 99 eggs/5 worms/5 hours (s.d. 13.0). f, Feeding rates of LA larval and adult hermaphrodites treated with 100 μM resveratrol. LA on live OP50: control 310±10.2 pumps/min, resveratrol 315±9.8; Adult on dead OP50: control 228±26.2, resveratrol 283±31.9; Adult on live OP50: control 383±16.0, resveratrol 383±2.7.

FIG. 12 shows wild-type female D. melanogaster survival with adults fed resveratrol or fisetin. a, Canton-S on 15% SY media. b, Canton-S on 5% SY media with resveratrol at two concentrations. c, Strain yw on 3% CSY media. d, Strain yw on 2% CSY media with resveratol at two concentrations. e, Strain yw on 3% CSY media with 100 μM resveratrol or fisetin. f, Strain yw on 2% CSY media with 100 μM resveratrol or fisetin. Life table statistics for this figure, for males and for additional trials are in Table 20. g, Mean daily fecundity per female (s.e.) estimated over 5-day intervals of Canton-S on 15% SY media with 0 or 10 μM resveratrol. h, Proportion (s.e.) of yw females feeding on diet with and without resveratrol in crop-filling assay. i, Mean (s.e.) body mass of Canton-S males and females feeding on diet without and with resveratrol (10 μM).

FIG. 13 shows the survivorship of D. melanogaster adults with mutant alleles of dSir2 when fed resveratrol (100μM). Females (a) and males (b) with loss-of-function genotype dSir2^{4.5}/dSir2^{5.26}. Females (c) and males (d) with strong hypomorphic genotype dSir2¹⁷/dSir2^{KG00871}.

FIG. 14 shows the mortality rates of control and resveratrol treated adults. Mortality was estimated as ln(-ln(pₓ)) where pₓ is the survival probability at day x to x+1. a, C. elegans wild-type N2 on heat-killed OP50 E. coli. b, C. elegans wild-type N2 on live OP50 E. coli. In a and b mortality is plotted only at days with observed mortality. c, D. melanogaster wildtype females of Trial 1 at effective doses of resveratrol on 15% SY diet. d, D. melanogaster wildtype males of Trial 1 at effective doses of resveratrol on 15% SY diet. In c and d mortality is smoothed from 3-day running average of pₓ.

FIG. 15 shows the stimulation of SIRT 1 catalytic rate by 100 μM plant polyphenols (Table 1).

FIG. 16 shows the effect of 100 μM stilbenes and chalcones on SIRT 1 catalytic rate (Supplementary Table 1).

FIG. 17 shows the effect of 100 μM flavones on SIRT 1 catalytic rate (Supplementary Table 2).

FIG. 18 shows the effect of 100 μM flavones on SIRT 1 catalytic rate (Supplementary Table 3).

FIG. 19 shows the effect of 100 μM isoflavones, flavanones and anthocyanidins on SIRT 1 catalytic rate (Supplementary Table 4).

FIG. 20 shows the effect of 100 μM catechins (Flavan-3-ols) on SIRT 1 catalytic rate (Supplementary Table 5).

FIG. 21 shows the effect of 100 μM free radical protective compounds on SIRT 1 catalytic rate (Supplementary Table 6).

FIG. 22 shows the effect of 100 μM miscellaneous compounds on SIRT 1 catalytic rate (Supplementary Table 7).

FIG. 23 shows the effect of 100 μM of various modulators on SIRT 1 catalytic rate (Supplementary Table 8).

FIG. 24 shows the effect of 100 μM of new resveratrol analogs on SIRT 1 catalytic rate (Table 9).

FIG. 25 shows the effect of 100 μM of new resveratrol analogs on SIRT 1 catalytic rate (Table 10).

FIG. 26 shows the effect of 100 μM of new resveratrol analogs on SIRT 1 catalytic rate (Table 11).

FIG. 27 shows the effect of 100 μM of new resveratrol analogs on SIRT 1 catalytic rate (Table 12).

FIG. 28 shows the effect of 100 μM of new resveratrol analogs on SIRT 1 catalytic rate (Table 13).

FIG. 29 shows synthetic intermediates of resveratrol analog synthesis (Table 14).

FIG. 30 shows synthetic intermediates of resveratrol analog synthesis (Table 15).

FIG. 31 shows synthetic intermediates of resveratrol analog synthesis (Table 16).

FIG. 32 shows synthetic intermediates of resveratrol analog synthesis (Table 17).

FIG. 33 shows synthetic intermediates of resveratrol analog synthesis (Table 18).

FIG. 34 shows the effect of resveratrol on Drosophila melanogaster (Table 20).

FIGS. 35A-G shows sirtuin activators and the fold activation of SIRT1 (Table 21).

FIG. 36 shows sirtuin inhibitors and the fold inhibition of SIRT1 (Table 22).

FIG. 37 shows plots of EAE scores over time. The four groups are animals in the vehicle control group (labeled as 318-319); 200 mg/kg resveratrol (320-321); 400 mg/kg resveratrol (322-323); and 5 mg/kg FK506 (324-325).

FIG. 38 shows plots of the degree of damage in the ventral/lateral (Top) and dorsal (Bottom) white matter of the thoracic spinal cords. The animals were treated with vehicle, 200 mg/kg resveratrol (Res low), 400 mg/kg resveratrol (Res high), or FK506.

FIG. 39 show representative sections from thoracic spinal cord from two mice treated with vehicle.

FIG. 40 shows representative sections from thoracic spinal cord from two mice treated with resveratrol (200 mg/kg).

FIG. 41 shows representative sections from thoracic spinal cord from two mice treated with resveratrol (400 mg/kg).

FIG. 42 shows representative sections from thoracic spinal cord from two mice treated with FK506 (5 mg/kg).

### DETAILED DESCRIPTION

### 1. DEFINITIONS

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

A “form that is naturally occurring” when referring to a compound means a compound that is in a form, e.g., a composition, in which it can be found naturally. For example, since resveratrol can be found in red wine, it is present in red wine in a form that is naturally occurring. A compound is not in a form that is naturally occurring if, e.g., the compound has been purified and separated from at least some of the other molecules that are found with the compound in nature. A “naturally occurring compound” refers to a compound that can be found in nature, i.e., a compound that has not been designed by man. A naturally occurring compound may have been made by man or by nature.

“Sirtuin modulator” refers to a compound that up regulates (e.g., activate or stimulate), down regulates (e.g., inhibit or suppress) or otherwise changes a functional property or biological activity of a sirtuin protein. Sirtuin modulators may act to modulate a sirtuin protein either directly or indirectly. In certain embodiments, a sirtuin modulator may be a sirtuin activator or a sirtuin inhibitor.

“Sirtuin activator” refers to a compound that increases the level of a sirtuin protein and/or increases at least one activity of a sirtuin protein. In an exemplary embodiment, a sirtuin activator may increase at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells. Exemplary sirtuin activating compounds include, for example, compounds having a formula selected from the group of formulas 1-25, 30, 32-65, and 69-88.

“Sirtuin inhibitor” refers to a compound that decreases the level of a sirtuin protein and/or decreases at least one activity of a sirtuin protein. In an exemplary embodiment, a sirtuin inhibitor may decrease at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells. Exemplay sirtuin inhibitors include, for example, compounds having a formula selected from the group of formulas 26-29, 31 and 66-68.

“Sirtuin protein” refers to a member of the sirtuin deacetylase protein family or preferably to the Sir2 family, which include yeast Sir2 (GenBank Accession No. P53685), C. elegans Sir-2.1 (GenBank Accession No. NP_{—}501912), and human SIRT1 (GenBank Accession No. NM_{—}012238 and NP_{—}036370 (or AF083106)) and SIRT2 (GenBank Accession No. NM_{—}030593 and AF083107) proteins. Other family members include the four additional yeast Sir2-like genes termed “HST genes” (homologues of Sir two) HST1, HST2, HST3 and HST4, and the five other human homologues hSIRT3, hSIRT4, hSIRT5, hSIRT6 and hSIRT7 (Brachmann et al. (1995) Genes Dev. 9:2888 and Frye et al. (1999) BBRC 260:273). Preferred sirtuins are those that share more similarities with SIRT1, i.e., hSIRT1, and/or Sir2 than with SIRT2, such as those members having at least part of the N-terminal sequence present in SIRT1 and absent in SIRT2 such as SIRT3 has.

“SIRT1 protein” refers to a member of the sir2 family of sirtuin deacetylases. In one embodiment, a SIRT1 protein includes yeast Sir2 (GenBank Accession No. P53685), C. elegans Sir-2.1 (GenBank Accession No. NP_{—}501912), human SIRT1 (GenBank Accession No. NM_{—}012238 and NP_{—}036370 (or AF083106)), human SIRT2 (GenBank Accession No. NM_{—}012237, NM_{—}030593, NP_{—}036369, NP_{—}085096, and AF083107) proteins, and equivalents and fragments thereof. In another embodiment, a SIRT1 protein includes a polypeptide comprising a sequence consisting of, or consisting essentially of, the amino acid sequence set forth in GenBank Accession Nos. NP_{—}036370, NP_{—}501912, NP_{—}085096, NP_{—}036369, and P53685. SIRT1 proteins include polypeptides comprising all or a portion of the amino acid sequence set forth in GenBank Accession Nos. NP_{—}036370, NP_{—}501912, NP_{—}085096, NP_{—}036369, and P53685; the amino acid sequence set forth in GenBank Accession Nos. NP_{—}036370, NP_{—}501912, NP_{—}085096, NP_{—}036369, and P53685 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. NP_{—}036370, NP_{—}501912, NP_{—}085096, NP_{—}036369, and P53685 and functional fragments thereof. Polypeptides of the invention also include homologs (e.g., orthologs and paralogs), variants, or fragments, of GenBank Accession Nos. NP_{—}036370, NP_{—}501912, NP_{—}085096, NP_{—}036369, and P53685.

“Biologically active portion of a sirtuin” refers to a portion of a sirtuin protein having a biological activity, such as the ability to deacetylate. Biologically active portions of sirtuins may comprise the core domain of sirtuins. For example, amino acids 62-293 of the SIRT1 protein sequence, which are encoded by nucleotides 237 to 932 of the SIRT1 nucleic acid sequence, encompass the NAD⁺ binding domain and the substrate binding domain. Therefore, this region is sometimes referred to as the core domain. Other biologically active portions of SIRT1, also sometimes referred to as core domains, include about amino acids 261 to 447 of the SIRT1 protein sequence, which are encoded by nucleotides 834 to 1394 of the SIRT1 nucleic acid sequence; about amino acids 242 to 493 of the SIRT1 protein sequence, which are encoded by nucleotides 777 to 1532 of the SIRT1 nucleic acid sequence; or about amino acids 254 to 495 of the SIRT1 protein sequence, which are encoded by nucleotides 813 to 1538 of the SIRT1 nucleic acid sequence.

A “direct activator” of a sirtuin is a molecule that activates a sirtuin by binding to it. A “direct inhibitor” of a sirtuin is a molecule that inhibits a sirtuin by binding to it.

The terms “comprise” and “comprising” are used in the inclusive, open sense, meaning that additional elements may be included.

The term “including” is used to mean “including but not limited to”. “Including” and “including but not limited to” are used interchangeably.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a nonnatural arrangement.

A “patient”, “subject” or “host” refers to either a human or a non-human animal.

The term “substantially homologous” when used in connection with amino acid sequences, refers to sequences which are substantially identical to or similar in sequence with each other, giving rise to a homology of conformation and thus to retention, to a useful degree, of one or more biological (including immunological) activities. The term is not intended to imply a common evolution of the sequences.

The term “modulation” is art-recognized and refers to up regulation (i.e., activation or stimulation), down regulation (i.e., inhibition or suppression) of a response, or the two in combination or apart.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “mammal” is known in the art, and exemplary mammals include humans, primates, bovines, porcines, canines, felines, and rodents (e.g., mice and rats).

The term “bioavailable” when referring to a compound is art-recognized and refers to a form of a compound that allows for it, or a portion of the amount of compound administered, to be absorbed by, incorporated to, or otherwise physiologically available to a subject or patient to whom it is administered.

The term “pharmaceutically-acceptable salts” is art-recognized and refers to the relatively non-toxic, inorganic and organic acid addition salts of compounds, including, for example, those contained in compositions described herein.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrastemal injection and infusion.

“Transcriptional regulatory sequence” is a generic term used throughout the specification to refer to DNA sequences, such as initiation signals, enhancers, and promoters, which induce or control transcription of protein coding sequences with which they are operable linked. In preferred embodiments, transcription of one of the recombinant genes is under the control of a promoter sequence (or other transcriptional regulatory sequence) which controls the expression of the recombinant gene in a cell-type which expression is intended. It will also be understood that the recombinant gene can be under the control of transcriptional regulatory sequences which are the same or which are different from those sequences which control transcription of the naturally-occurring forms of genes as described herein.

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

“Treating” a condition or disease refers to curing as well as ameliorating at least one symptom of the condition or disease.

The term “cis” is art-recognized and refers to the arrangement of two atoms or groups around a double bond such that the atoms or groups are on the same side of the double bond. Cis configurations are often labeled as (Z) configurations.

The term “trans” is art-recognized and refers to the arrangement of two atoms or groups around a double bond such that the atoms or groups are on the opposite sides of a double bond. Trans configurations are often labeled as (E) configurations.

The term “covalent bond” is art-recognized and refers to a bond between two atoms where electrons are attracted electrostatically to both nuclei of the two atoms, and the net effect of increased electron density between the nuclei counterbalances the internuclear repulsion. The term covalent bond includes coordinate bonds when the bond is with a metal ion.

The term “therapeutic agent” is art-recognized and refers to any chemical moiety that is a biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions described herein may be administered in a sufficient amount to produce a desired effect on neurodegenerative disorders or blood coagulation disorders or complications thereof, at a reasonable benefit/risk ratio applicable to such treatment.

The term “synthetic” is art-recognized and refers to production by in vitro chemical or enzymatic synthesis.

The term “meso compound” is art-recognized and refers to a chemical compound which has at least two chiral centers but is achiral due to a plane or point of symmetry.

The term “chiral” is art-recognized and refers to molecules which have the property of non-superimposability of the mirror image partner, while the term “achiral” refers to molecules which are superimposable on their mirror image partner. A “prochiral molecule” is a molecule which has the potential to be converted to a chiral molecule in a particular process.

The term “stereoisomers” is art-recognized and refers to compounds which have identical chemical constitution, but differ with regard to the arrangement of the atoms or groups in space. In particular, “enantiomers” refer to two stereoisomers of a compound which are non-superimposable mirror images of one another. “Diastereomers”, on the other hand, refers to stereoisomers with two or more centers of dissymmetry and whose molecules are not mirror images of one another.

Furthermore, a “stereoselective process” is one which produces a particular stereoisomer of a reaction product in preference to other possible stereoisomers of that product. An “enantioselective process” is one which favors production of one of the two possible enantiomers of a reaction product.

The term “regioisomers” is art-recognized and refers to compounds which have the same molecular formula but differ in the connectivity of the atoms. Accordingly, a “regioselective process” is one which favors the production of a particular regioisomer over others, e.g., the reaction produces a statistically significant increase in the yield of a certain regioisomer.

The term “epimers” is art-recognized and refers to molecules with identical chemical constitution and containing more than one stereocenter, but which differ in configuration at only one of these stereocenters.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The term “structure-activity relationship” or “(SAR)” is art-recognized and refers to the way in which altering the molecular structure of a drug or other compound alters its biological activity, e.g., its interaction with a receptor, enzyme, nucleic acid or other target and the like.

The term “aliphatic” is art-recognized and refers to a linear, branched, cyclic alkane, alkene, or alkyne. In certain embodiments, aliphatic groups in the present compounds are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure. The term “alkyl” is also defined to include halosubstituted alkyls.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “heteroatom” is art-recognized and refers to an atom of any element other than carbon or hydrogen. Illustrative heteroatoms include boron, nitrogen, oxygen, phosphorus, sulfur and selenium.

The term “aryl” is art-recognized and refers to 5-, 6- and 7-membered single-ring aromatic groups that may include from zero to four heteroatoms, for example, benzene, naphtalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas: 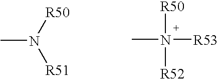
wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula: 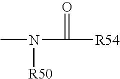
wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula: 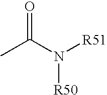
wherein R50 and R51 are as defined above. Certain embodiments of amides may not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art recognized and includes such moieties as may be represented by the general formulas: 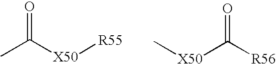
wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula: 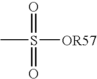
in which R57 is an electron pair, hydrogen, alkyl, cycloalkyl, or aryl.

The term “sulfate” is art recognized and includes a moiety that may be represented by the general formula: 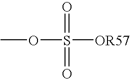
in which R57 is as defined above.

The term “sulfonamido” is art recognized and includes a moiety that may be represented by the general formula: 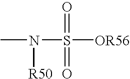
in which R50 and R56 are as defined above.

The term “sulfamoyl” is art-recognized and refers to a moiety that may be represented by the general formula: 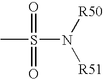
in which R50 and R51 are as defined above.

The term “sulfonyl” is art-recognized and refers to a moiety that may be represented by the general formula: 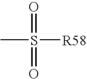
in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula: 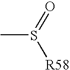
in which R58 is defined above.

The term “phosphoryl” is art-recognized and may in general be represented by the formula: 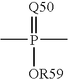
wherein Q50 represents S or O, and R59 represents hydrogen, a lower alkyl or an aryl. When used to substitute, e.g., an alkyl, the phosphoryl group of the phosphorylalkyl may be represented by the general formulas: 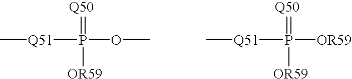
wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas: 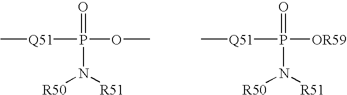
wherein Q51, R50, R51 and R59 are as defined above.

The term “phosphonarnidite” is art-recognized and may be represented in the general formulas: 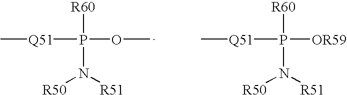
wherein Q51, R50, R5 1 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions described herein may exist in particular geometric or stereoisomeric forms. In addition, compounds may also be optically active. Contemplated herein are all such compounds, including cis- and trans-isomers, R- and S-enantiomers, diastereomers, (D)-isomers, (L)-isomers, the racemic mixtures thereof, and other mixtures thereof. Additional asymmetric carbon atoms may be present in a substituent such as an alkyl group. All such isomers, as well as mixtures thereof, are encompassed herein.

If, for instance, a particular enantiomer of a compound is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. Heteroatoms such as nitrogen may have hydrogen substituents and/or any permissible substituents of organic compounds described herein which satisfy the valences of the heteroatoms. Compounds are not intended to be limited in any manner by the permissible substituents of organic compounds.

The chemical elements are identified in accordance with the Periodic Table of the Elements, CAS version, Handbook of Chemistry and Physics, 67th Ed., 1986-87, inside cover.

The term “protecting group” is art-recognized and refers to temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed by Greene and Wuts in Protective Groups in Organic Synthesis (2^{nd} ed., Wiley: N.Y., 1991).

The term “hydroxyl-protecting group” is art-recognized and refers to those groups intended to protect a hydrozyl group against undesirable reactions during synthetic procedures and includes, for example, benzyl or other suitable esters or ethers groups known in the art.

The term “carboxyl-protecting group” is art-recognized and refers to those groups intended to protect a carboxylic acid group, such as the C-terminus of an amino acid or peptide or an acidic or hydroxyl azepine ring substituent, against undesirable reactions during synthetic procedures and includes. Examples for protecting groups for carboxyl groups involve, for example, benzyl ester, cyclohexyl ester, 4-nitrobenzyl ester, t-butyl ester, 4-pyridylmethyl ester, and the like.

The term “amino-blocking group” is art-recognized and refers to a group which will prevent an amino group from participating in a reaction carried out on some other functional group, but which can be removed from the amine when desired. Such groups are discussed by in Ch. 7 of Greene and Wuts, cited above, and by Barton, Protective Groups in Organic Chemistry ch. 2 (McOmie, ed., Plenum Press, New York, 1973). Examples of suitable groups include acyl protecting groups such as, to illustrate, formyl, dansyl, acetyl, benzoyl, trifluoroacetyl, succinyl, methoxysuccinyl, benzyl and substituted benzyl such as 3,4-dimethoxybenzyl, o-nitrobenzyl, and triphenylmethyl; those of the formula —COOR where R includes such groups as methyl, ethyl, propyl, isopropyl, 2,2,2-trichloroethyl, 1-methyl-1-phenylethyl, isobutyl, t-butyl, t-amyl, vinyl, allyl, phenyl, benzyl, p-nitrobenzyl, o-nitrobenzyl, and 2,4-dichlorobenzyl; acyl groups and substituted acyl such as formyl, acetyl, chloroacetyl, dichloroacetyl, trichloroacetyl, trifluoroacetyl, benzoyl, and p-methoxybenzoyl; and other groups such as methanesulfonyl, p-toluenesulfonyl, p-bromobenzenesulfonyl, p-nitrophenylethyl, and p-toluenesulfonyl-aminocarbonyl. Preferred amino-blocking groups are benzyl (—CH₂C₆H₅), acyl [C(O)R1] or SiR1₃ where R1 is C₁-C₄ alkyl, halomethyl, or 2-halo-substituted-(C₂-C₄ alkoxy), aromatic urethane protecting groups as, for example, carbonylbenzyloxy (Cbz); and aliphatic urethane protecting groups such as t-butyloxycarbonyl (Boc) or 9-fluorenylmethoxycarbonyl (FMOC).

The definition of each expression, e.g. lower alkyl, m, n, p and the like, when it occurs more than once in any structure, is intended to be independent of its definition elsewhere in the same structure.

The term “electron-withdrawing group” is art-recognized, and refers to the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, March, Advanced Organic Chemistry 251-59 (McGraw Hill Book Company: New York, 1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

2. Exemplary Sirtuin-Activating Compounds

In one embodiment, exemplary sirtuin-activating compounds are those described in Howitz et al. (2003) Nature 425: 191 and include, for example, resveratrol (3,5,4′-Trihydroxy-trans-stilbene), butein (3,4,2′,4′-Tetrahydroxychalcone), piceatannol (3,5,3′,4′-Tetrahydroxy-trans-stilbene), isoliquiritigenin (4,2′,4′-Trihydroxychalcone), fisetin (3,7,3′,4′-Tetrahyddroxyflavone), quercetin (3,5,7,3′,4′-Pentahydroxyflavone), Deoxyrhapontin (3,5-Dihydroxy-4′-methoxystilbene 3-O-β-D-glucoside); trans-Stilbene; Rhapontin (3,3′,5-Trihydroxy-4′-methoxystilbene 3-O-β-D-glucoside); cis-Stilbene; Butein (3,4,2′,4′-Tetrahydroxychalcone); 3,4,2′4′6′-Pentahydroxychalcone; Chalcone; 7,8,3′,4′-Tetrahydroxyflavone; 3,6,2′,3′-Tetrahydroxyflavone; 4′-Hydroxyflavone; 5,4′-Dihydroxyflavone; 5,7-Dihydroxyflavone; Morin (3,5,7,2′,4′- Pentahydroxyflavone); Flavone; 5-Hydroxyflavone; (−)-Epicatechin (Hydroxy Sites: 3,5,7,3′,4′); (−)-Catechin (Hydroxy Sites: 3,5,7,3′,4′); (−)-Gallocatechin (Hydroxy Sites: 3,5,7,3′,4′,5′) (+)-Catechin (Hydroxy Sites: 3,5,7,3′,4′); 5,7,3′,4′,5′-pentahydroxyflavone; Luteolin (5,7,3′,4′-Tetrahydroxyflavone); 3,6,3′,4′-Tetrahydroxyflavone; 7,3′,4′,5′-Tetrahydroxyflavone; Kaempferol (3,5,7,4′-Tetrahydroxyflavone); 6-Hydroxyapigenin (5,6,7,4′-Tetrahydoxyflavone); Scutellarein); Apigenin (5,7,4′-Trihydroxyflavone); 3,6,2′,4′-Tetrahydroxyflavone; 7,4′-Dihydroxyflavone; Daidzein (7,4′-Dihydroxyisoflavone); Genistein (5,7,4′-Trihydroxyflavanone); Naringenin (5,7,4′-Trihydroxyflavanone); 3,5,7,3′,4′-Pentahydroxyflavanone; Flavanone; Pelargonidin chloride (3,5,7,4′-Tetrahydroxyflavylium chloride); Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-imidazole4-ethanaminium inner salt); Caffeic Acid Phenyl Ester; MCI-186 (3-Methyl-1-phenyl-2-pyrazolin-5-one); HBED (N,N′-Di-(2-hydroxybenzyl)ethylenediamine-N,N′-diacetic acid●H2O); Ambroxol (trans-4-(2-Amino-3,5-dibromobenzylamino)cyclohexane-HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol●2HCl). Analogs and derivatives thereof can also be used.

Other sirtuin-activating compounds may have any of formulas 1-25, 30, 32-65, and 69-88 below. In one embodiment, a sirtuin-activating compound is a stilbene or chalcone compound of formula 1: 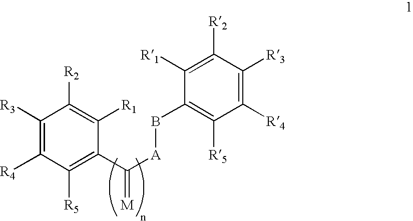
wherein, independently for each occurrence,

R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents O, NR, or S;

A-B represents a bivalent alkyl, alkenyl, alkynyl, amido, sulfonamido, diazo, ether, alkylamino, alkylsulfide, hydroxylamine, or hydrazine group; and

n is 0 or 1.

In a further embodiment, a sirtuin-activating compound is a compound of formula 1 and the attendant definitions, wherein n is 0. In a further embodiment, a sirtuin-activating compound is a compound of formula 1 and the attendant definitions, wherein n is 1. In a further embodiment, a sirtuin-activating compound is a compound of formula 1 and the attendant definitions, wherein A-B is ethenyl. In a further embodiment, a sirtuin-activating compound is a compound of formula 1 and the attendant definitions, wherein A-B is —CH₂CH(Me)CH(Me)CH₂—. In a further embodiment, a sirtuin-activating compound is a compound of formula 1 and the attendant definitions, wherein M is O. In a further embodiment, the methods comprises a compound of formula 1 and the attendant definitions, wherein R₁, R₂, R₃, R₄, R₅, R′₁, R′₂, R′₃, R′₄, and R′₅ are H. In a further embodiment, a sirtuin-activating compound is a compound of formula 1 and the attendant definitions, wherein R₂, R₄, and R′₃ are OH. In a further embodiment, a sirtuin-activating compound is a compound of formula 1 and the attendant definitions, wherein R₂, R₄, R′₂ and R′₃ are OH. In a further embodiment, a sirtuin-activating compound is a compound of formula 1 and the attendant definitions, wherein R₃, R₅, R′₂ and R′₃ are OH. In a further embodiment, a sirtuin-activating compound is a compound of formula 1 and the attendant definitions, wherein R₁, R₃, R₅, R′₂ and R′₃ are OH. In a further embodiment, a sirtuin-activating compound is a compound of formula 1 and the attendant definitions, wherein R₂ and R′₂ are OH; R₄ is O-β-D-glucoside; and R′₃ is OCH₃. In a further embodiment, a sirtuin-activating compound is a compound of formula 1 and the attendant definitions, wherein R₂ is OH; R₄ is O-β-D-glucoside; and R′₃ is OCH₃.

In a further embodiment, a sirtuin-activating compound is a compound of formula 1 and the attendant definitions, wherein n is 0; A-B is ethenyl; and R₁, R₂, R₃, R₄, R₅, R′₁, R′₂, R′₃, R′₄, and R′₅ are H (trans stilbene). In a further embodiment, a sirtuin-activating compound is a compound of formula 1 and the attendant definitions, wherein n is 1; A-B is ethenyl; M is O; and R₁, R₂, R₃, R₄, R₅, R′₁, R′₂, R′₃, R′₄, and R′₅ are H (chalcone). In a further embodiment, a sirtuin-activating compound is a compound of formula 1 and the attendant definitions, wherein n is 0; A-B is ethenyl; R₂, R₄, and R′₃ are OH; and R₁, R₃, R₅, R′₁, R′₂, R′₄, and R′₅ are H (resveratrol). In a further embodiment, a sirtuin-activating compound is a compound of formula 1 and the attendant definitions, wherein n is 0; A-B is ethenyl; R₂, R₄, R′₂ and R′₃ are OH; and R₁, R₃, R₅, R′₁, R′₄ and R′₅ are H (piceatannol). In a further embodiment, a sirtuin-activating compound is a compound of formula 1 and the attendant definitions, wherein n is 1; A-B is ethenyl; M is O; R₃, R₅, R′₂ and R′₃ are OH; and R₁, R₂, R₄, R′₁, R′₄, and R′₅ are H (butein). In a further embodiment, a sirtuin-activating compound is a compound of formula 1 and the attendant definitions, wherein n is 1; A-B is ethenyl; M is O; R₁, R₃, R₅, R′₂ and R′₃ are OH; and R₂, R₄, R′₁, R′₄, and R′₅ are H (3,4,2′,4′,6′-pentahydroxychalcone). In a further embodiment, a sirtuin-activating compound is a compound of formula 1 and the attendant definitions, wherein n is 0; A-B is ethenyl; R₂ and R′₂ are OH, R₄ is O-β-D-glucoside, R′₃ is OCH₃; and R₁, R₃, R₅, R′₁, R′₄, and R′₅ are H (rhapontin). In a further embodiment, a sirtuin-activating compound is a compound of formula 1 and the attendant definitions, wherein n is 0; A-B is ethenyl; R₂ is OH, R₄ is O-β-D-glucoside, R′₃ is OCH₃; and R₁, R₃, R₅, R′₁, R′₂, R′₄, and R′₅ are H (deoxyrhapontin). In a further embodiment, a sirtuin-activating compound is a compound of formula 1 and the attendant definitions, wherein n is 0; A-B is —CH₂CH(Me)CH(Me)CH₂—; R₂, R₃, R′₂, and R′₃ are OH; and R₁, R₄, R₅, R′₁, R′₄, and R′₅ are H (NDGA).

In another embodiment, a sirtuin-activating compound is a flavanone compound of formula 2:

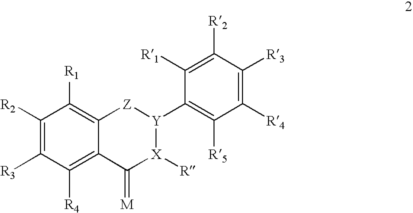

wherein, independently for each occurrence,

R₁, R₂, R₃, R₄, R′₁, R′₂, R′₃, R′₄, R′₅, and R″ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents CR, O, NR, or S;

X represents CR or N; and

Y represents CR or N.

In a further embodiment, a sirtuin-activating compound is a compound of formula 2 and the attendant definitions, wherein X and Y are both CH. In a further embodiment, a sirtuin-activating compound is a compound of formula 2 and the attendant definitions, wherein M is O. In a further embodiment, a sirtuin-activating compound is a compound of formula 2 and the attendant definitions, wherein M is H₂. In a further embodiment, a sirtuin-activating compound is a compound of formula 2 and the attendant definitions, wherein Z is O. In a further embodiment, a sirtuin-activating compound is a compound of formula 2 and the attendant definitions, wherein R″ is H. In a further embodiment, a sirtuin-activating compound is a compound of formula 2 and the attendant definitions, wherein R″ is OH. In a further embodiment, a sirtuin-activating compound is a compound of formula 2 and the attendant definitions, wherein R″ is an alkoxycarbonyl. In a further embodiment, a sirtuin-activating compound is a compound of formula 2 and the attendant definitions, wherein R₁ is 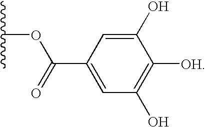
In a further embodiment, a sirtuin-activating compound is a compound of formula 2 and the attendant definitions, wherein R₁, R₂, R₃, R₄, R′₁, R′₂, R′₃, R′₄, R′₅ and R″ are H. In a further embodiment, a sirtuin-activating compound is a compound of formula 2 and the attendant definitions, wherein R₂, R₄, and R′₃ are OH. In a further embodiment, a sirtuin-activating compound is a compound of formula 2 and the attendant definitions, wherein R₄, R′₂, R′₃, and R″ are OH. In a further embodiment, a sirtuin-activating compound is a compound of formula 2 and the attendant definitions, wherein R₂, R₄, R′₂, R′₃, and R″ are OH. In a further embodiment, a sirtuin-activating compound is a compound of formula 2 and the attendant definitions, wherein R₂, R₄, R′₂, R′₃, R′₄, and R″ are OH.

In a further embodiment, a sirtuin-activating compound is a compound of formula 2 and the attendant definitions, wherein X and Y are CH; M is O; Z and O; R″ is H; and R₁, R₂, R₃, R₄, R′₁, R′₂, R′₃, R′₄, R′₅ and R″ are H (flavanone). In a further embodiment, a sirtuin-activating compound is a compound of formula 2 and the attendant definitions, wherein X and Y are CH; M is O; Z and O; R″ is H; R₂, R₄, and R′₃ are OH; and R₁, R₃, R′₁, R′₂, R′₄, and R′₅ are H (naringenin). In a further embodiment, a sirtuin-activating compound is a compound of formula 2 and the attendant definitions, wherein X and Y are CH; M is O; Z and O; R″ is OH; R₂, R₄, R′₂, and R′₃ are OH; and R_{I}, R₃, R′₁, R′₄, and R′₅ are H (3,5,7,3′,4′-pentahydroxyflavanone). In a further embodiment, a sirtuin-activating compound is a compound of formula 2 and the attendant definitions, wherein X and Y are CH; M is H₂; Z and O; R″ is OH; R₂, R₄, R′₂, and R′₃, are OH; and R₁, R₃, R′₁, R′₄ and R′₅ are H (epicatechin). In a further embodiment, a sirtuin-activating compound is a compound of formula 2 and the attendant definitions, wherein X and Y are CH; M is H₂; Z and O; R″ is OH; R₂, R₄, R′₂, R′₃, and R′₄ are OH; and R₁, R₃, R′₁, and R′₅ are H (gallocatechin). In a further embodiment, a sirtuin-activating compound is a compound of formula 2 and the attendant definitions, wherein X and Y are CH; M is H₂; Z and O; R″ is 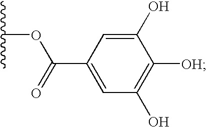
R₂, R₄, R′₂, R′₃, R′₄, and R″ are OH; and R₁, R₃, R′₁, and R′₅ are H (epigallocatechin gallate).

In another embodiment, a sirtuin-activating compound is an isoflavanone compound of formula 3:

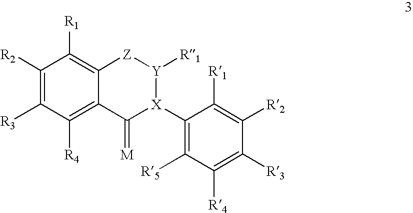

wherein, independently for each occurrence,

R₁, R₂, R₃, R₄, R′₁, R′₂, R′₃, R′₄, R′₅, and R″₁ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents C(R)₂, O, NR, or S;

X represents CR or N; and

Y represents CR or N.

In another embodiment, a sirtuin-activating compound is a flavone compound of formula 4:

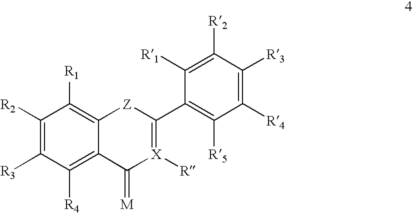

wherein, independently for each occurrence,

R₁, R₂, R₃, R₄, R′₁, R′₂, R′₃, R′₄, and R′₅, represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents CR, O, NR, or S; and

X represents CR″ or N, wherein

R″ is H, alkyl, aryl, heteroaryl, alkaryl, heteroaralkyl, halide, NO₂, SR, OR, N(R)₂, or carboxyl.

In a further embodiment, a sirtuin-activating compound is a compound of formula 4 and the attendant definitions, wherein X is C. In a further embodiment, a sirtuin-activating compound is a compound of formula 4 and the attendant definitions, wherein X is CR. In a further embodiment, a sirtuin-activating compound is a compound of formula 4 and the attendant definitions, wherein Z is O. In a further embodiment, a sirtuin-activating compound is a compound of formula 4 and the attendant definitions, wherein M is O. In a further embodiment, a sirtuin-activating compound is a compound of formula 4 and the attendant definitions, wherein R″ is H. In a further embodiment, a sirtuin-activating compound is a compound of formula 4 and the attendant definitions, wherein R″ is OH. In a further embodiment, a sirtuin-activating compound is a compound of formula 4 and the attendant definitions, wherein R₁, R₂, R₃, R₄, R′₁, R′₂, R′₃, R′₄, and R′₅ are H. In a further embodiment, a sirtuin-activating compound is a compound of formula 4 and the attendant definitions, wherein R₂, R′₂, and R′₃ are OH. In a further embodiment, a sirtuin-activating compound is a compound of formula 4 and the attendant definitions, wherein R₂, R₄, R′₂, R′₃, and R′₄ are OH. In a further embodiment, a sirtuin-activating compound is a compound of formula 4 and the attendant definitions, wherein R₂, R₄, R′₂, and R′₃ are OH. In a further embodiment, a sirtuin-activating compound is a compound of formula 4 and the attendant definitions, wherein R₃, R′₂, and R′₃ are OH. In a further embodiment, a sirtuin-activating compound is a compound of formula 4 and the attendant definitions, wherein R₂, R₄, R′₂, and R′₃ are OH. In a further embodiment, a sirtuin-activating compound is a compound of formula 4 and the attendant definitions, wherein R₂, R′₂, R′₃, and R′₄ are OH. In a further embodiment, a sirtuin-activating compound is a compound of formula 4 and the attendant definitions, wherein R₂, R₄, and R′₃ are OH. In a further embodiment, a sirtuin-activating compound is a compound of formula 4 and the attendant definitions, wherein R₂, R₃, R₄, and R′₃ are OH. In a further embodiment, a sirtuin-activating compound is a compound of formula 4 and the attendant definitions, wherein R₂, R₄, and R′₃ are OH. In a further embodiment, a sirtuin-activating compound is a compound of formula 4 and the attendant definitions, wherein R₃, R′₁, and R′₃ are OH. In a further embodiment, a sirtuin-activating compound is a compound of formula 4 and the attendant definitions, wherein R₂ and R′₃ are OH. In a further embodiment, a sirtuin-activating compound is a compound of formula 4 and the attendant definitions, wherein R₁, R₂, R′₂, and R′₃ are OH. In a further embodiment, a sirtuin-activating compound is a compound of formula 4 and the attendant definitions, wherein R₃, R′₁, and R′₂ are OH. In a further embodiment, a sirtuin-activating compound is a compound of formula 4 and the attendant definitions, wherein R′₃ is OH. In a further embodiment, a sirtuin-activating compound is a compound of formula 4 and the attendant definitions, wherein R4 and R′₃ are OH. In a further embodiment, a sirtuin-activating compound is a compound of formula 4 and the attendant definitions, wherein R₂ and R₄ are OH. In a further embodiment, a sirtuin-activating compound is a compound of formula 4 and the attendant definitions, wherein R₂, R₄, R′₁, and R′₃ are OH. In a further embodiment, a sirtuin-activating compound is a compound of formula 4 and the attendant definitions, wherein R₄ is OH. In a further embodiment, a sirtuin-activating compound is a compound of formula 4 and the attendant definitions, wherein R₂, R₄, R′₂, R′₃, and R′₄ are OH. In a further embodiment, a sirtuin-activating compound is a compound of formula 4 and the attendant definitions, wherein R₂, R′₂, R′₃, and R′₄ are OH. In a further embodiment, a sirtuin-activating compound is a compound of formula 4 and the attendant definitions, wherein R₁, R₂, R₄, R′₂, and R′₃ are OH.

In a further embodiment, a sirtuin-activating compound is a compound of formula 4 and the attendant definitions, wherein X is CH; Z is O; M is O; and R₁, R₂, R₃, R₄, R′₁, R′₂, R′₃, R′₄, and R′₅ are H (flavone). In a further embodiment, a sirtuin-activating compound is a compound of formula 4 and the attendant definitions, wherein X is COH; Z is O; M is O; R₂, R′₂, and R′₃ are OH; and R₁, R₃, R₄, R′₁, R′₄, and R′₅ are H (fisetin). In a further embodiment, a sirtuin-activating compound is a compound of formula 4 and the attendant definitions, wherein X is CH; Z is O; M is O; R₂, R₄, R′₂, R′₃, and R′₄ are OH; and R₁, R₃, R′₁, and R′₅ are H (5,7,3′,4′,5′-pentahydroxyflavone). In a further embodiment, a sirtuin-activating compound is a compound of formula 4 and the attendant definitions, wherein X is CH; Z is O; M is O; R₂, R₄, R′₂, and R′₃ are OH; and R₁, R₃, R′₁, R′₄, and R′₅ are H (luteolin). In a further embodiment, a sirtuin-activating compound is a compound of formula 4 and the attendant definitions, wherein X is COH; Z is O; M is O; R₃, R′₂, and R′₃ are OH; and R₁, R₂, R₄, R′₁, R′₄, and R′₅ are H (3,6,3′,4′-tetrahydroxyflavone). In a further embodiment, a sirtuin-activating compound is a compound of formula 4 and the attendant definitions, wherein X is COH; Z is O; M is O; R₂, R₄, R′₂, and R′₃ are OH; and R₁, R₃, R′₁, R′₄, and R′₅ are H (quercetin). In a further embodiment, a sirtuin-activating compound is a compound of formula 4 and the attendant definitions, wherein X is CH; Z is O; M is O; R₂, R′₂, R′₃, and R′₄ are OH; and R₁, R₃, R₄, R′₁, and R′₅ are H. In a further embodiment, a sirtuin-activating compound is a compound of formula 4 and the attendant definitions, wherein X is COH; Z is O; M is O; R₂, R₄, and R′₃ are OH; and R₁, R₃, R′₁, R′₂, R′₄, and R′₅ are H. In a further embodiment, a sirtuin-activating compound is a compound of formula 4 and the attendant definitions, wherein X is CH; Z is O; M is O; R₂, R₃, R₄, and R′₃ are OH; and R₁, R′₁, R′₂, R′₄, and R′₅ are H. In a further embodiment, a sirtuin-activating compound is a compound of formula 4 and the attendant definitions, wherein X is CH; Z is O; M is O; R₂, R₄, and R′₃ are OH; and R₁, R₃, R′₁, R′₂, R′₄, and R′₅ are H. In a further embodiment, a sirtuin-activating compound is a compound of formula 4 and the attendant definitions, wherein X is COH; Z is O; M is O; R₃, R′₁, and R′₃ are OH; and R₁, R₂, R₄, R′₂, R′₄, and R′₅ are H. In a further embodiment, a sirtuin-activating compound is a compound of formula 4 and the attendant definitions, wherein X is CH; Z is O; M is O; R₂ and R′₃ are OH; and R₁, R₃, R₄, R′₁, R′₂, R′₄, and R′₅ are H. In a further embodiment, a sirtuin-activating compound is a compound of formula 4 and the attendant definitions, wherein X is COH; Z is O; M is O; R₁, R₂, R′₂, and R′₃ are OH; and R₁, R₂, R₄, R′₃, R′₄, and R′₅ are H. In a further embodiment, a sirtuin-activating compound is a compound of formula 4 and the attendant definitions, wherein X is COH; Z is O; M is O; R₃, R′₁, and R′₂ are OH; and R₁, R₂, R₄; R′₃, R′₄, and R′₅ are H. In a further embodiment, a sirtuin-activating compound is a compound of formula 4 and the attendant definitions, wherein X is CH; Z is O; M is O; R′₃ is OH; and R₁, R₂, R₃, R₄, R′₁, R′₂, R′₄, and R′₅ are H. In a further embodiment, a sirtuin-activating compound is a compound of formula 4 and the attendant definitions, wherein X is CH; Z is O; M is O; R₄ and R′₃ are OH; and R₁, R₂, R₃, R′₁, R′₂, R′₄, and R′₅ are H. In a further embodiment, a sirtuin-activating compound is a compound of formula 4 and the attendant definitions, wherein X is CH; Z is O; M is O; R₂ and R₄ are OH; and R₁, R₃, R′₁, R′₂, R′₃, R′₄, and R′₅ are H. In a further embodiment, a sirtuin-activating compound is a compound of formula 4 and the attendant definitions, wherein X is COH; Z is O; M is O; R₂, R₄, R′₁, and R′₃ are OH; and R₁, R₃, R′₂, R′₄, and R′₅ are H. In a further embodiment, a sirtuin-activating compound is a compound of formula 4 and the attendant definitions, wherein X is CH; Z is O; M is O; R₄ is OH; and R₁, R₂, R₃, R′₁, R′₂, R′₃, R′₄, and R′₅ are H. In a further embodiment, a sirtuin-activating compound is a compound of formula 4 and the attendant definitions, wherein X is COH; Z is O; M is O; R₂, R₄, R′₂, R′₃, and R′₄ are OH; and R₁, R₃, R′₁, and R′₅ are H. In a further embodiment, a sirtuin-activating compound is a compound of formula 4 and the attendant definitions, wherein X is COH; Z is O; M is O; R₂, R′₂, R′₃, and R′₄ are OH; and R₁, R₃, R₄, R′₁, and R′₅ are H. In a further embodiment, a sirtuin-activating compound is a compound of formula 4 and the attendant definitions, wherein X is COH; Z is O; M is O; R₁, R₂, R₄, R′₂, and R′₃ are OH; and R₃, R′₁, R′₄, and R′₅ are H.

In another embodiment, a sirtuin-activating compound is an isoflavone compound of formula 5:

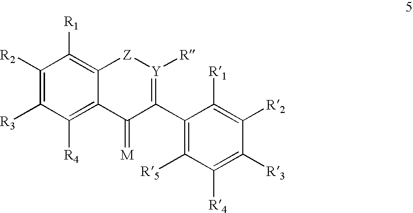

wherein, independently for each occurrence,

R₁, R₂, R₃, R₄, R′₁, R′₂, R′₃, R′₄, and R′₅, represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents C(R)₂, O, NR, or S; and

Y represents CR″ or N, wherein

R″ represents H, alkyl, aryl, heteroaryl, alkaryl, heteroaralkyl, halide, NO₂, SR, OR, N(R)₂, or carboxyl.

In a further embodiment, a sirtuin-activating compound is a compound of formula 5 and the attendant definitions, wherein Y is CR″. In a further embodiment, a sirtuin-activating compound is a compound of formula 5 and the attendant definitions, wherein Y is CH. In a further embodiment, a sirtuin-activating compound is a compound of formula 5 and the attendant definitions, wherein Z is O. In a further embodiment, a sirtuin-activating compound is a compound of formula 5 and the attendant definitions, wherein M is O. In a further embodiment, a sirtuin-activating compound is a compound of formula 5 and the attendant definitions, wherein R₂ and R′₃ are OH. In a further embodiment, a sirtuin-activating compound is a compound of formula 5 and the attendant definitions, wherein R₂, R₄, and R′₃ are OH.

In a further embodiment, a sirtuin-activating compound is a compound of formula 5 and the attendant definitions, wherein Y is CH; Z is O; M is O; R₂ and R′₃ are OH; and R₁, R₃, R₄, R′₁, R′₂, R′₄, and R′₅ are H. In a further embodiment, a sirtuin-activating compound is a compound of formula 5 and the attendant definitions, wherein Y is CH; Z is O; M is O; R₂, R₄, and R′₃ are OH; and R₁, R₃, R′₁, R′₂, R′₄, and R′₅ are H.

In another embodiment, a sirtuin-activating compound is an anthocyanidin compound of formula 6:

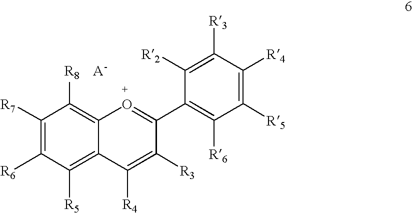

wherein, independently for each occurrence,

R₃, R₄, R₅, R₆, R₇, R₈, R′₂, R′₃, R′₄, R′₅, and R′₆ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl; and

A⁻ represents an anion selected from the following: Cl⁻, Br⁻, or I⁻.

In a further embodiment, a sirtuin-activating compound is a compound of formula 6 and the attendant definitions, wherein A⁻ is Cl⁻. In a further embodiment, a sirtuin-activating compound is a compound of formula 6 and the attendant definitions, wherein R₃, R₅, R₇, and R′₄ are OH. In a further embodiment, a sirtuin-activating compound is a compound of formula 6 and the attendant definitions, wherein R₃, R₅, R₇, R′₃, and R′₄ are OH. In a further embodiment, a sirtuin-activating compound is a compound of formula 6 and the attendant definitions, wherein R₃, R₅, R₇, R′₃, R′₄, and R′₅ are OH.

In a further embodiment, a sirtuin-activating compound is a compound of formula 6 and the attendant definitions, wherein A⁻ is Cl⁻; R₃, R₅, R₇, and R′₄ are OH; and R₄, R₆, R₈, R′₂, R′₃, R′₅, and R′₆ are H. In a further embodiment, a sirtuin-activating compound is a compound of formula 6 and the attendant definitions, wherein A⁻ is Cl⁻; R₃, R₅, R₇, R′₃, and R′₄ are OH; and R₄, R₆, R₈, R′₂, R′₅, and R′₆ are H. In a further embodiment, a sirtuin-activating compound is a compound of formula 6 and the attendant definitions, wherein A⁻ is Cl⁻; R₃, R₅, R₇, R′₃, R′₄, and R′₅ are OH; and R₄, R₆, R₈, R′₂, and R′₆ are H.

In a further embodiment, a sirtuin-activating compound is a stilbene, chalcone, or flavone compound represented by formula 7:

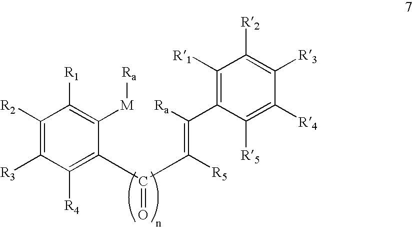

wherein, independently for each occurrence,

M is absent or O;

R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;

Rₐ represents H or the two instances of Rₐ form a bond;

R represents H, alkyl, aryl, heteroaryl, aralkyl; and

n is 0 or 1.

In a further embodiment, a sirtuin-activating compound is an activating compound represented by formula 7 and the attendant definitions, wherein n is 0. In a further embodiment, a sirtuin-activating compound is an activating compound represented by formula 7 and the attendant definitions, wherein n is 1. In a further embodiment, a sirtuin-activating compound is an activating compound represented by formula 7 and the attendant definitions, wherein M is absent. In a further embodiment, a sirtuin-activating compound is an activating compound represented by formula 7 and the attendant definitions, wherein M is O. In a further embodiment, a sirtuin-activating compound is an activating compound represented by formula 7 and the attendant definitions, wherein Rₐ is H. In a further embodiment, a sirtuin-activating compound is an activating compound represented by formula 7 and the attendant definitions, wherein M is O and the two Rₐ form a bond.

In a further embodiment, a sirtuin-activating compound is an activating compound represented by formula 7 and the attendant definitions, wherein R₅ is H. In a further embodiment, a sirtuin-activating compound is an activating compound represented by formula 7 and the attendant definitions, wherein R₅ is OH. In a further embodiment, a sirtuin-activating compound is an activating compound represented by formula 7 and the attendant definitions, wherein R₁, R₃, and R′₃ are OH. In a further embodiment, a sirtuin-activating compound is an activating compound represented by formula 7 and the attendant definitions, wherein R₂, R₄, R′₂, and R′₃ are OH. In a further embodiment, a sirtuin-activating compound is an activating compound represented by formula 7 and the attendant definitions, wherein R₂, R′₂, and R′₃ are OH. In a further embodiment, a sirtuin-activating compound is an activating compound represented by formula 7 and the attendant definitions, wherein R₂ and R₄ are OH.

In a further embodiment, a sirtuin-activating compound is a compound represented by formula 7 and the attendant definitions, wherein n is 0; M is absent; Rₐ is H; R₅ is H; R₁, R₃, and R′₃ are OH; and R₂, R₄, R′₁, R′₂, R′₄, and R′₅ are H. In a further embodiment, a sirtuin-activating compound is an activating compound represented by formula 7 and the attendant definitions, wherein n is 1; M is absent; Rₐ is H; R₅ is H; R₂, R₄, R′₂, and R′₃ are OH; and R₁, R₃, R′₁, R′₄, and R′₅ are H. In a further embodiment, a sirtuin-activating compound is an activating compound represented by formula 7 and the attendant definitions, wherein n is 1; M is O; the two Rₐ form a bond; R₅ is OH; R₂, R′₂, and R′₃ are OH; and R₁, R₃, R₄, R′₁, R′₄, and R′₅ are H.

Other sirtuin-activating compounds include compounds having a formula selected from the group consisting of formulas 8-25 and 30 set forth below.

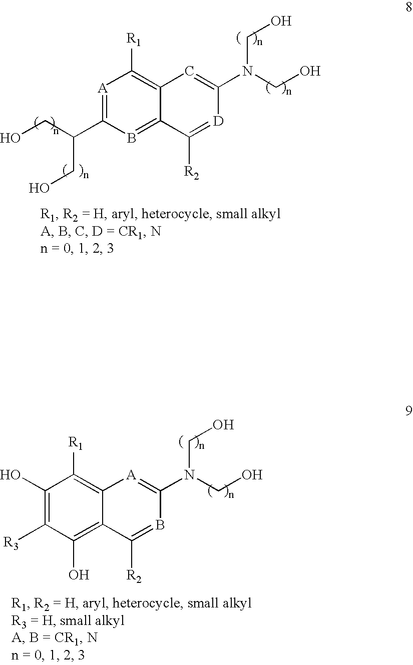

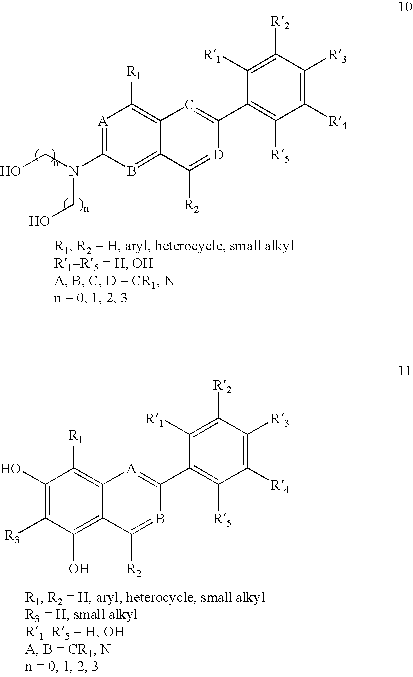

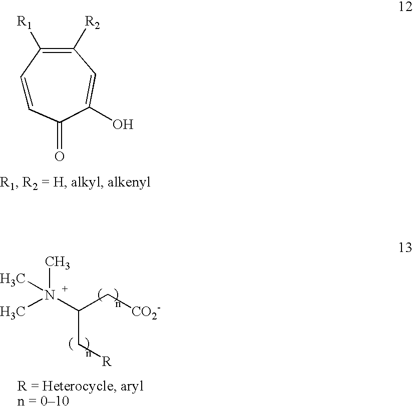

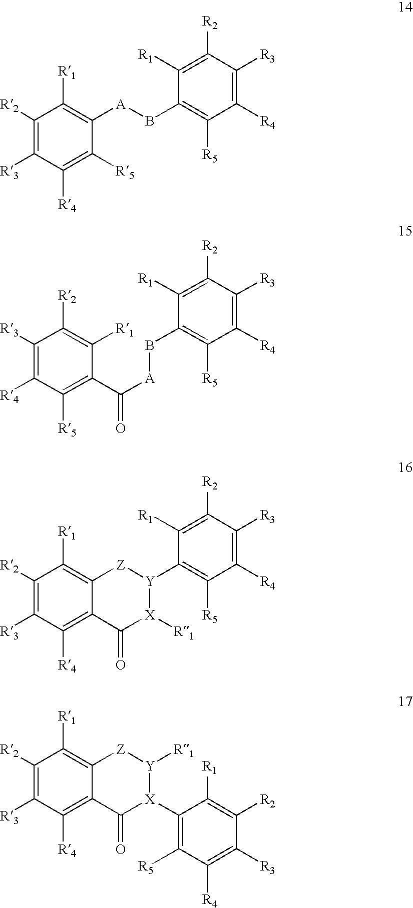

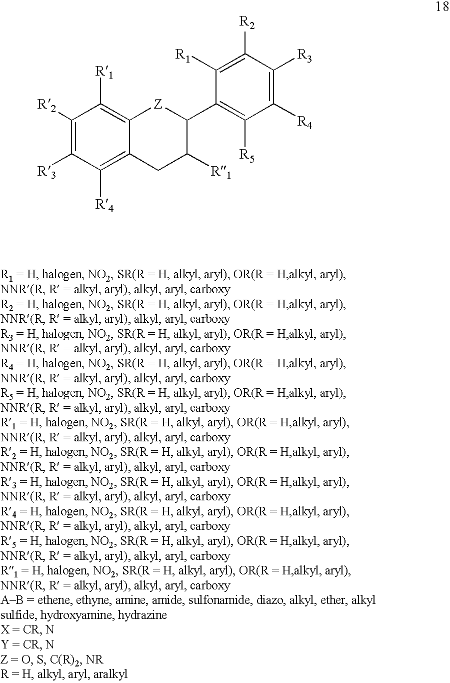

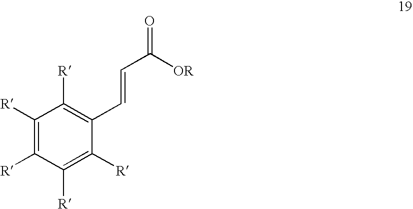

wherein, independently for each occurrence,

R=H, alkyl, aryl, heterocyclyl, heteroaryl, or aralkyl; and

R′=H, halogen, NO₂, SR, OR, NR₂, alkyl, aryl, or carboxy.

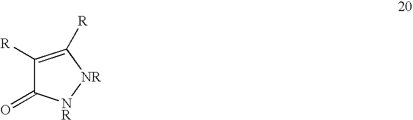

wherein, independently for each occurrence,

R═H, alkyl, aryl, heterocyclyl, heteroaryl, or aralkyl.

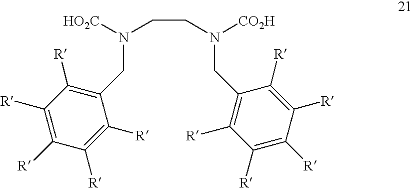

wherein, independently for each occurrence,

R′═H, halogen, NO₂, SR, OR, NR₂, alkyl, aryl, aralkyl, or carboxy; and

R═H, alkyl, aryl, heterocyclyl, heteroaryl, or aralkyl.

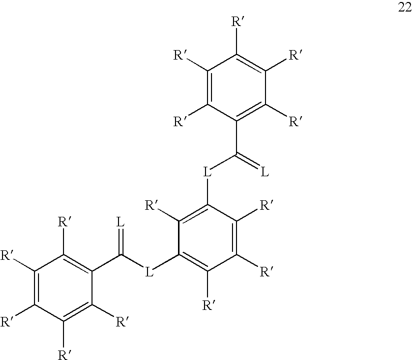

wherein, independently for each occurrence,

L represents CR₂, O, NR, or S;

R represents H, alkyl, aryl, aralkyl, or heteroaralkyl; and

R′ represents H, halogen, NO₂, SR, OR, NR₂, alkyl, aryl, aralkyl, or carboxy.

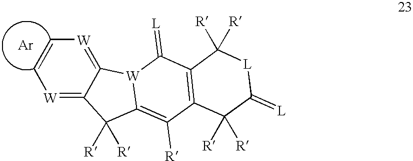

wherein, independently for each occurrence,

L represents CR₂, O, NR, or S;

W represents CR or N;

R represents H, alkyl, aryl, aralkyl, or heteroaralkyl;

Ar represents a fused aryl or heteroaryl ring; and

R′ represents H, halogen, NO₂, SR, OR, NR₂, alkyl, aryl, aralkyl, or carboxy.

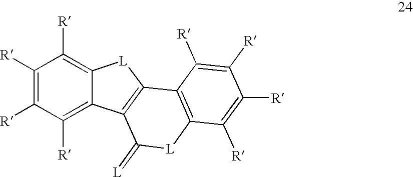

wherein, independently for each occurrence,

L represents CR₂, O, NR, or S;

R represents H, alkyl, aryl, aralkyl, or heteroaralkyl; and

R′ represents H, halogen, NO₂, SR, OR, NR₂, alkyl, aryl, aralkyl, or carboxy.

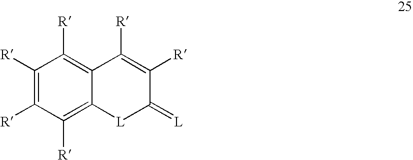

wherein, independently for each occurrence,

L represents CR₂, O, NR, or S;

R represents H, alkyl, aryl, aralkyl, or heteroaralkyl; and

R′ represents H, halogen, NO₂, SR, OR, NR₂, alkyl, aryl, aralkyl, or carboxy.

In a further embodiment, a sirtuin-activating compound is a stilbene, chalcone, or flavone compound represented by formula 30:

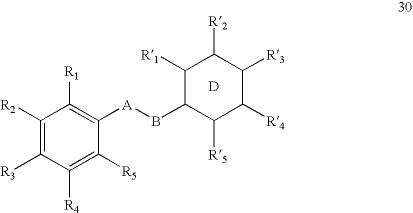

wherein, independently for each occurrence,

D is a phenyl or cyclohexyl group;

R₁, R₂, R₃, R₄, R₅, R′₁, R′₂, R′₃, R′₄, and R′₅ represent H, alkyl, aryl, heteroaryl, alkaryl, heteroaralkyl, halide, NO₂, SR, OR, N(R)₂, carboxyl, azide, ether; or any two adjacent R or R′ groups taken together form a fused benzene or cyclohexyl group;

R represents H, alkyl, aryl, or aralkyl; and

A-B represents an ethylene, ethenylene, or imine group;

provided that when A-B is ethenylene, D is phenyl, and R′₃ is H: R₃ is not OH when R₁, R₂, R₄, and R₅ are H; and R₂ and R₄ are not OMe when R₁, R₃, and R₅ are H; and R₃ is not OMe when R₁, R₂, R₄, and R₅ are H.

In a further embodiment, a sirtuin-activating compound is a compound represented by formula 30 and the attendant definitions, wherein D is a phenyl group.

In a further embodiment, a sirtuin-activating compound is a compound represented by formula 30 and the attendant definitions, wherein A-B is an ethenylene or imine group.

In a further embodiment, a sirtuin-activating compound is a compound represented by formula 30 and the attendant definitions, wherein A-B is an ethenylene group.

In a further embodiment, a sirtuin-activating compound is a compound represented by formula 30 and the attendant definitions, wherein R₂ is OH.

In a further embodiment, a sirtuin-activating compound is a compound represented by formula 30 and the attendant definitions, wherein R₄ is OH

In a further embodiment, a sirtuin-activating compound is a compound represented by formula 30 and the attendant definitions, wherein R₂ and R₄ are OH.

In a further embodiment, a sirtuin-activating compound is a compound represented by formula 30 and the attendant definitions, wherein D is a phenyl group; and A-B is an ethenylene group.

In a further embodiment, a sirtuin-activating compound is a compound represented by formula 30 and the attendant definitions, wherein D is a phenyl group; A-B is an ethenylene group; and R₂ and R₄ are OH.

In a further embodiment, a sirtuin-activating compound is a compound represented by formula 30 and the attendant definitions, wherein A-B is ethenylene; D is a phenyl ring; R₂ and R₄ are OH; and R′₃ is Cl.

In a further embodiment, a sirtuin-activating compound is a compound represented by formula 30 and the attendant definitions, wherein A-B is ethenylene; D is a phenyl ring; R₂ and R₄ are OH; and R′₃ is OH.

In a further embodiment, a sirtuin-activating compound is a compound represented by formula 30 and the attendant definitions, wherein A-B is ethenylene; D is a phenyl ring; R₂ and R₄ are OH; and R′₃ is H.

In a further embodiment, a sirtuin-activating compound is a compound represented by formula 30 and the attendant definitions, wherein A-B is ethenylene; D is a phenyl ring; R₂ and R₄ are OH; and R′₃ is CH₂CH₃.

In a further embodiment, a sirtuin-activating compound is a compound represented by formula 30 and the attendant definitions, wherein A-B is ethenylene; D is a phenyl ring; R₂ and R₄ are OH; and R′₃ is F.

In a further embodiment, a sirtuin-activating compound is a compound represented by formula 30 and the attendant definitions, wherein A-B is ethenylene; D is a phenyl ring; R₂ and R₄ are OH; and R′₃ is Me.

In a further embodiment, a sirtuin-activating compound is a compound represented by formula 30 and the attendant definitions, wherein A-B is ethenylene; D is a phenyl ring; R₂ and R₄ are OH; and R′₃ is an azide.

In a further embodiment, a sirtuin-activating compound is a compound represented by formula 30 and the attendant definitions, wherein A-B is ethenylene; D is a phenyl ring; R₂ and R₄ are OH; and R′₃ is SMe.

In a further embodiment, a sirtuin-activating compound is a compound represented by formula 30 and the attendant definitions, wherein A-B is ethenylene; D is a phenyl ring; R₂ and R₄ are OH; and R′₃ is NO₂.

In a further embodiment, a sirtuin-activating compound is a compound represented by formula 30 and the attendant definitions, wherein A-B is ethenylene; D is a phenyl ring; R₂ and R₄ are OH; and R′₃ is CH(CH₃)₂.

In a further embodiment, a sirtuin-activating compound is a compound represented by formula 30 and the attendant definitions, wherein A-B is ethenylene; D is a phenyl ring; R₂ and R₄ are OH; and R′₃ is OMe.

In a further embodiment, a sirtuin-activating compound is a compound represented by formula 30 and the attendant definitions, wherein A-B is ethenylene; D is a phenyl ring; R₂ and R₄ are OH; R′₂ is OH; and R′₃ is OMe.

In a further embodiment, a sirtuin-activating compound is a compound represented by formula 30 and the attendant definitions, wherein A-B is ethenylene; D is a phenyl ring; R₂ is OH; R₄ is carboxyl; and R′₃ is OH.

In a further embodiment, a sirtuin-activating compound is a compound represented by formula 30 and the attendant definitions, wherein A-B is ethenylene; D is a phenyl ring; R₂ and R₄ are OH; and R′₃ is carboxyl.

In a further embodiment, a sirtuin-activating compound is a compound represented by formula 30 and the attendant definitions, wherein A-B is ethenylene; D is a phenyl ring; R₂ and R₄ are OH; and R′₃ and R′₄ taken together form a fused benzene ring.

In a further embodiment, a sirtuin-activating compound is a compound represented by formula 30 and the attendant definitions, wherein A-B is ethenylene; D is a phenyl ring; and R₄ is OH.

In a further embodiment, a sirtuin-activating compound is a compound represented by formula 30 and the attendant definitions, wherein A-B is ethenylene; D is a phenyl ring; R₂ and R₄ are OCH₂OCH₃; and R′₃ is SMe.

In a further embodiment, a sirtuin-activating compound is a compound represented by formula 30 and the attendant definitions, wherein A-B is ethenylene; D is a phenyl ring; R₂ and R₄ are OH; and R′₃ is carboxyl.

In a further embodiment, a sirtuin-activating compound is a compound represented by formula 30 and the attendant definitions, wherein A-B is ethenylene; D is a cyclohexyl ring; and R₂ and R₄ are OH.

In a further embodiment, a sirtuin-activating compound is a compound represented by formula 30 and the attendant definitions, wherein A-B is ethenylene; D is a phenyl ring; and R₃ and R₄ are OMe.

In a further embodiment, a sirtuin-activating compound is a compound represented by formula 30 and the attendant definitions, wherein A-B is ethenylene; D is a phenyl ring; R₂ and R₄ are OH; and R′₃ is OH.

In another embodiment, a sirtuin-activating compound is a compound of formula 32: 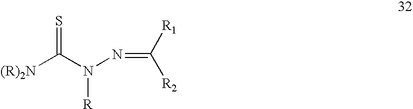
wherein, independently for each occurrence:

R is H, or a substituted or unsubstituted alkyl, aryl, aralkyl, heterocyclyl, heterocyclylalkyl, heteroaryl, or heteroaralkyl; and

R₁ and R₂ are a substituted or unsubstituted alkyl, aryl, aralkyl, heterocyclyl, heterocyclylalkyl, heteroaryl, or heteroaralkyl.

In a further embodiment, a sirtuin-activating compound is a compound of formula 32 and the attendant definitions wherein R is H.

In a further embodiment, a sirtuin-activating compound is a compound of formula 32 and the attendant definitions wherein R₁ is 3-hydroxyphenyl.

In a further embodiment, a sirtuin-activating compound is a compound of formula 32 and the attendant definitions wherein R₂ is methyl.

In a further embodiment, a sirtuin-activating compound is a compound of formula 32 and the attendant definitions wherein R is H and R₁ is 3-hydroxyphenyl.

In a further embodiment, a sirtuin-activating compound is a compound of formula 32 and the attendant definitions wherein R is H, R₁ is 3-hydroxyphenyl, and R₂ is methyl.

In another embodiment, a sirtuin-activating compound is a compound of formula 33: 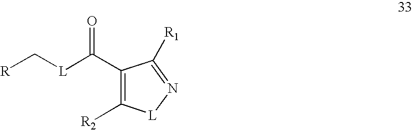
wherein, independently for each occurrence:

R is H, or a substituted or unsubstituted alkyl, alkenyl, or alkynyl;

R₁ and R₂ are a substituted or unsubstituted alkyl, aryl, aralkyl, heterocyclyl, heterocyclylalkyl, heteroaryl, or heteroaralkyl; and

L is O, S, or NR.

In a further embodiment, a sirtuin-activating compound is a compound of formula 33 and the attendant definitions wherein R is alkynyl.

In a further embodiment, a sirtuin-activating compound is a compound of formula 33 and the attendant definitions wherein R₁ is 2,6-dichlorophenyl.

In a further embodiment, a sirtuin-activating compound is a compound of formula 33 and the attendant definitions wherein R₂ is methyl.

In a further embodiment, a sirtuin-activating compound is a compound of formula 33 and the attendant definitions wherein L is O.

In a further embodiment, a sirtuin-activating compound is a compound of formula 33 and the attendant definitions wherein R is alkynyl and R₁ is 2,6-dichlorophenyl.

In a further embodiment, a sirtuin-activating compound is a compound of formula 33 and the attendant definitions wherein R is alkynyl, R₁ is 2,6-dichlorophenyl, and R₂ is methyl.

In a further embodiment, a sirtuin-activating compound is a compound of formula 33 and the attendant definitions wherein R is alkynyl, R₁ is 2,6-dichlorophenyl, R₂ is methyl, and L is O.

In another embodiment, a sirtuin-activating compound is a compound of formula 34: 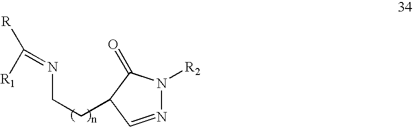
wherein, independently for each occurrence:

R, R₁, and R₂ are H, or a substituted or unsubstituted alkyl, aryl, aralkyl, heterocyclyl, heterocyclylalkyl, heteroaryl, or heteroaralkyl; and

n is an integer from 0 to 5 inclusive.

In a further embodiment, a sirtuin-activating compound is a compound of formula 34 and the attendant definitions wherein R is 3,5-dichloro-2-hydroxyphenyl.

In a further embodiment, a sirtuin-activating compound is a compound of formula 34 and the attendant definitions wherein R₁ is H.

In a further embodiment, a sirtuin-activating compound is a compound of formula 34 and the attendant definitions wherein R₂ is H.

In a further embodiment, a sirtuin-activating compound is a compound of formula 34 and the attendant definitions wherein n is 1.

In a further embodiment, a sirtuin-activating compound is a compound of formula 34 and the attendant definitions wherein R is 3,5-dichloro-2-hydroxyphenyl and R₁ is H.

In a further embodiment, a sirtuin-activating compound is a compound of formula 34 and the attendant definitions wherein R is 3,5-dichloro-2-hydroxyphenyl, R₁ is H, and R₂ is H.

In a further embodiment, a sirtuin-activating compound is a compound of formula 34 and the attendant definitions wherein R is 3,5-dichloro-2-hydroxyphenyl, R₁ is H, R₂ is H, and n is 1.

In another embodiment, a sirtuin-activating compound is a compound of formula 35: 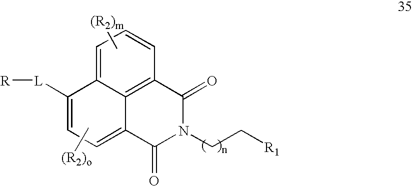
wherein, independently for each occurrence:

R is H or a substituted or unsubstituted alkyl, aryl, aralkyl, heterocyclyl, heterocyclylalkyl, heteroaryl, or heteroaralkyl;

R₁ is a substituted or unsubstituted alkyl, aryl, aralkyl, heterocyclyl, heterocyclylalkyl, heteroaryl, or heteroaralkyl;

R₂ is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, heteroaralkyl;

L is O, NR, or S;

m is an integer from 0 to 3 inclusive;

n is an integer from 0 to 5 inclusive; and

o is an integer from 0 to 2 inclusive.

In a further embodiment, a sirtuin-activating compound is a compound of formula 35 and the attendant definitions wherein R is phenyl.

In a further embodiment, a sirtuin-activating compound is a compound of formula 35 and the attendant definitions wherein R₁ is pyridine.

In a further embodiment, a sirtuin-activating compound is a compound of formula 35 and the attendant definitions wherein L is S.

In a further embodiment, a sirtuin-activating compound is a compound of formula 35 and the attendant definitions wherein m is 0.

In a further embodiment, a sirtuin-activating compound is a compound of formula 35 and the attendant definitions wherein n is 1.

In a further embodiment, a sirtuin-activating compound is a compound of formula 35 and the attendant definitions wherein o is 0.

In a further embodiment, a sirtuin-activating compound is a compound of formula 35 and the attendant definitions wherein R is phenyl and R₁ is pyridine.

In a further embodiment, a sirtuin-activating compound is a compound of formula 35 and the attendant definitions wherein R is phenyl, R₁ is pyridine, and L is S.

In a further embodiment, a sirtuin-activating compound is a compound of formula 35 and the attendant definitions wherein R is phenyl, R₁ is pyridine, L is S, and m is 0.

In a further embodiment, a sirtuin-activating compound is a compound of formula 35 and the attendant definitions wherein R is phenyl, R₁ is pyridine, L is S, m is 0, and n is 1.

In a further embodiment, a sirtuin-activating compound is a compound of formula 35 and the attendant definitions wherein R is phenyl, R₁ is pyridine, L is S, m is 0, n is 1, and o is 0.

In another embodiment, a sirtuin-activating compound is a compound of formula 36: 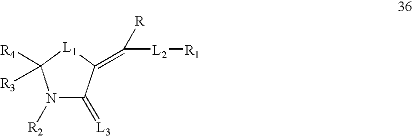
wherein, independently for each occurrence:

R, R₃, and R₄ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, heteroaralkyl;

R₁ and R₂ are H or a substituted or unsubstituted alkyl, aryl, aralkyl, heterocyclyl, heterocyclylalkyl, heteroaryl, heteroaralkyl;

L₁ is O, NR₁, S, C(R)₂, or SO₂; and

L₂ and L₃ are O, NR₁, S, or C(R)₂.

In a further embodiment, a sirtuin-activating compound is a compound of formula 36 and the attendant definitions wherein R is H.

In a further embodiment, a sirtuin-activating compound is a compound of formula 36 and the attendant definitions wherein R₁ is 4-chlorophenyl.

In a further embodiment, a sirtuin-activating compound is a compound of formula 36 and the attendant definitions wherein R₂ is 4-chlorophenyl.

In a further embodiment, a sirtuin-activating compound is a compound of formula 36 and the attendant definitions wherein R₃ is H.

In a further embodiment, a sirtuin-activating compound is a compound of formula 36 and the attendant definitions wherein R₄ is H.

In a further embodiment, a sirtuin-activating compound is a compound of formula 36 and the attendant definitions wherein L₁ is SO₂.

In a further embodiment, a sirtuin-activating compound is a compound of formula 36 and the attendant definitions wherein L₂ is NH.

In a further embodiment, a sirtuin-activating compound is a compound of formula 36 and the attendant definitions wherein L₃ is O.

In a further embodiment, a sirtuin-activating compound is a compound of formula 36 and the attendant definitions wherein R is H and R₁ is 4-chlorophenyl.

In a further embodiment, a sirtuin-activating compound is a compound of formula 36 and the attendant definitions wherein R is H, R₁ is 4-chlorophenyl, and R₂ is 4-chlorophenyl.

In a further embodiment, a sirtuin-activating compound is a compound of formula 36 and the attendant definitions wherein R is H, R₁ is 4-chlorophenyl, R₂ is 4-chlorophenyl, and R₃ is H.

In a further embodiment, a sirtuin-activating compound is a compound of formula 36 and the attendant definitions wherein R is H, R₁ is 4-chlorophenyl, R₂ is 4-chlorophenyl, R₃ is H, and R₄ is H.

In a further embodiment, a sirtuin-activating compound is a compound of formula 36 and the attendant definitions wherein R is H, R₁ is 4-chlorophenyl, R₂ is 4-chlorophenyl, R₃ is H, R₄ is H, and L₁ is SO₂.

In a further embodiment, a sirtuin-activating compound is a compound of formula 36 and the attendant definitions wherein R is H, R₁ is 4-chlorophenyl, R₂ is 4-chlorophenyl, R₃ is H, R₄ is H, L₁ is SO₂, and L₂ is NH.

In a further embodiment, a sirtuin-activating compound is a compound of formula 36 and the attendant definitions wherein R is H, R₁ is 4-chlorophenyl, R₂ is 4-chlorophenyl, R₃ is H, R₄ is H, L₁ is SO₂, L₂ is NH, and L₃ is O.

In another embodiment, a sirtuin-activating compound is a compound of formula 37: 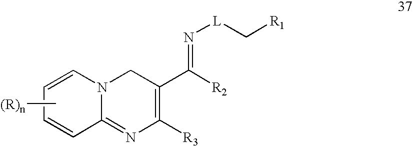
wherein, independently for each occurrence:

R is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, heteroaralkyl;

R₁ is H or a substituted or unsubstituted alkyl, aryl, aralkyl, heterocyclyl, heterocyclylalkyl, heteroaryl, heteroaralkyl;

R₂ and R₃ are H or a substituted or unsubstituted alkyl, aryl, aralkyl, heterocyclyl, heterocyclylalkyl, heteroaryl, heteroaralkyl;

L is O, NR₁, or S; and

n is an integer from 0 to 4 inclusive.

In a further embodiment, a sirtuin-activating compound is a compound of formula 37 and the attendant definitions wherein R is methyl.

In a further embodiment, a sirtuin-activating compound is a compound of formula 37 and the attendant definitions wherein n is 1.

In a further embodiment, a sirtuin-activating compound is a compound of formula 37 and the attendant definitions wherein R₁ is 3-fluorophenyl.

In a further embodiment, a sirtuin-activating compound is a compound of formula 37 and the attendant definitions wherein R₂ is H.

In a further embodiment, a sirtuin-activating compound is a compound of formula 37 and the attendant definitions wherein R₃ is 4-chlorophenyl.

In a further embodiment, a sirtuin-activating compound is a compound of formula 37 and the attendant definitions wherein L is O.

In a further embodiment, a sirtuin-activating compound is a compound of formula 37 and the attendant definitions wherein R is methyl and n is 1.

In a further embodiment, a sirtuin-activating compound is a compound of formula 37 and the attendant definitions wherein R is methyl, n is 1, and R₁ is 3-fluorophenyl.

In a further embodiment, a sirtuin-activating compound is a compound of formula 37 and the attendant definitions wherein R is methyl, n is 1, R₁ is 3-fluorophenyl, and R₂ is H.

In a further embodiment, a sirtuin-activating compound is a compound of formula 37 and the attendant definitions wherein R is methyl, n is 1, R₁ is 3-fluorophenyl, R₂ is H, and R₃ is 4-chlorophenyl.

In another embodiment, a sirtuin-activating compound is a compound of formula 38: 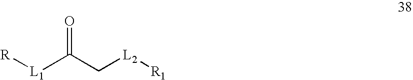
wherein, independently for each occurrence:

R and R₁ are H or a substituted or unsubstituted alkyl, aryl, aralkyl, heterocyclyl, heterocyclylalkyl, heteroaryl, or heteroaralkyl; and

L₁ and L₂ are O, NR, or S.

In a further embodiment, a sirtuin-activating compound is a compound of formula 38 and the attendant definitions wherein R is 3-methoxyphenyl.

In a further embodiment, a sirtuin-activating compound is a compound of formula 38 and the attendant definitions wherein R₁ is 4-t-butylphenyl.

In a further embodiment, a sirtuin-activating compound is a compound of formula 38 and the attendant definitions wherein L₁ is NH.

In a further embodiment, a sirtuin-activating compound is a compound of formula 38 and the attendant definitions wherein L₂ is O.

In a further embodiment, a sirtuin-activating compound is a compound of formula 38 and the attendant definitions wherein R is 3-methoxyphenyl and R₁ is 4-t-butylphenyl.

In a further embodiment, a sirtuin-activating compound is a compound of formula 38 and the attendant definitions wherein R is 3-methoxyphenyl, R₁ is 4-t-butylphenyl, and L₁ is NH.

In a further embodiment, a sirtuin-activating compound is a compound of formula 38 and the attendant definitions wherein R is 3-methoxyphenyl, R₁ is 4-t-butylphenyl, L₁ is NH, and L₂ is O.

In another embodiment, a sirtuin-activating compound is a compound of formula 39: 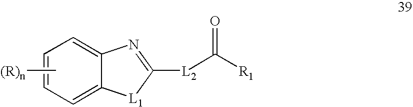
wherein, independently for each occurrence:

R is H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ is H or a substituted or unsubstituted alkyl, aryl, alkaryl, heterocyclyl, heterocyclylalkyl, heteroaryl, or heteroaralkyl;

L₁ and L₂ are O, NR, or S; and

n is an integer from 0 to 4 inclusive.

In a further embodiment, a sirtuin-activating compound is a compound of formula 39 and the attendant definitions wherein R is methyl.

In a further embodiment, a sirtuin-activating compound is a compound of formula 39 and the attendant definitions wherein n is 1.

In a further embodiment, a sirtuin-activating compound is a compound of formula 39 and the attendant definitions wherein R₁ is 3,4,5-trimethoxyphenyl.

In a further embodiment, a sirtuin-activating compound is a compound of formula 39 and the attendant definitions wherein L₁ is S.

In a further embodiment, a sirtuin-activating compound is a compound of formula 39 and the attendant definitions wherein L₂ is NH.

In a further embodiment, a sirtuin-activating compound is a compound of formula 39 and the attendant definitions wherein R is methyl and n is 1.

In a further embodiment, a sirtuin-activating compound is a compound of formula 39 and the attendant definitions wherein R is methyl, n is 1, and R₁ is 3,4,5-trimethoxyphenyl.

In a further embodiment, a sirtuin-activating compound is a compound of formula 39 and the attendant definitions wherein R is methyl, n is 1, R₁ is 3,4,5-trimethoxyphenyl, and L₁ is S.

In a further embodiment, a sirtuin-activating compound is a compound of formula 39 and the attendant definitions wherein R is methyl, n is 1, R₁ is 3,4,5-trimethoxyphenyl, L₁ is S, and L₂ is NH.

In another embodiment, a sirtuin-activating compound is a compound of formula 40: 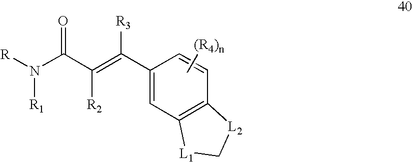
wherein, independently for each occurrence:

R, R₁, R₂, R₃ are H or a substituted or unsubstituted alkyl, aryl, alkaryl, heterocyclyl, heterocyclylalkyl, heteroaryl, or heteroaralkyl;

R₄ is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR, or S; and

n is an integer from 0 to 3 inclusive.

In a further embodiment, a sirtuin-activating compound is a compound of formula 40 and the attendant definitions wherein R is H.

In a further embodiment, a sirtuin-activating compound is a compound of formula 40 and the attendant definitions wherein R₁ is perfluorophenyl.

In a further embodiment, a sirtuin-activating compound is a compound of formula 40 and the attendant definitions wherein R₂ is H.

In a further embodiment, a sirtuin-activating compound is a compound of formula 40 and the attendant definitions wherein R₃ is H.

In a further embodiment, a sirtuin-activating compound is a compound of formula 40 and the attendant definitions wherein L₁ is O.

In a further embodiment, a sirtuin-activating compound is a compound of formula 40 and the attendant definitions wherein L₂ is O.

In a further embodiment, a sirtuin-activating compound is a compound of formula 40 and the attendant definitions wherein n is 0.

In a further embodiment, a sirtuin-activating compound is a compound of formula 40 and the attendant definitions wherein R is H and R₁ is perfluorophenyl.

In a further embodiment, a sirtuin-activating compound is a compound of formula 40 and the attendant definitions wherein R is H, R₁ is perfluorophenyl, and R₂ is H.

In a further embodiment, a sirtuin-activating compound is a compound of formula 40 and the attendant definitions R is H, R₁ is perfluorophenyl, R₂ is H, and R₃ is H.

In a further embodiment, a sirtuin-activating compound is a compound of formula 40 and the attendant definitions wherein R is H, R₁ is perfluorophenyl, R₂ is H, R₃ is H, and L₁ is O.

In a further embodiment, a sirtuin-activating compound is a compound of formula 40 and the attendant definitions wherein R is H, R₁ is perfluorophenyl, R₂ is H, R₃ is H, L₁ is O, and L₂ is O.

In a further embodiment, a sirtuin-activating compound is a compound of formula 40 and the attendant definitions wherein R is H, R₁ is perfluorophenyl, R₂ is H, R₃ is H, L₁ is O, L₂ is O, and n is 0.

In another embodiment, a sirtuin-activating compound is a compound of formula 41: 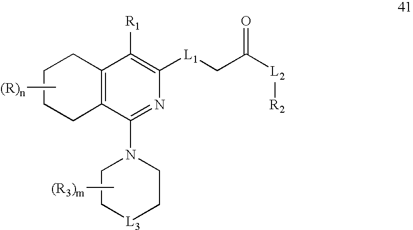
wherein, independently for each occurrence:

R, R₁, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is H or a substituted or unsubstituted alkyl, aryl, aralkyl, heterocyclyl, heterocyclylalkyl, heteroaryl, or heteroaralkyl;

L₁, L₂, and L₃ are O, NR₂, or S; and

m and n are integers from 0 to 8 inclusive.

In a further embodiment, a sirtuin-activating compound is a compound of formula 41 and the attendant definitions wherein n is 0.

In a further embodiment, a sirtuin-activating compound is a compound of formula 41 and the attendant definitions wherein R₁ is cyano.

In a further embodiment, a sirtuin-activating compound is a compound of formula 41 and the attendant definitions wherein R₂ is ethyl.

In a further embodiment, a sirtuin-activating compound is a compound of formula 41 and the attendant definitions wherein m is 0.

In a further embodiment, a sirtuin-activating compound is a compound of formula 41 and the attendant definitions wherein L₁ is S.

In a further embodiment, a sirtuin-activating compound is a compound of formula 41 and the attendant definitions wherein L₂ is O.

In a further embodiment, a sirtuin-activating compound is a compound of formula 41 and the attendant definitions wherein L₃ is O.

In a further embodiment, a sirtuin-activating compound is a compound of formula 41 and the attendant definitions wherein n is 0 and R₁ is cyano.

In a further embodiment, a sirtuin-activating compound is a compound of formula 41 and the attendant definitions wherein n is 0, R₁ is cyano, and R₂ is ethyl.

In a further embodiment, a sirtuin-activating compound is a compound of formula 41 and the attendant definitions wherein n is 0, R₁ is cyano, R₂ is ethyl, and m is 0.

In a further embodiment, a sirtuin-activating compound is a compound of formula 41 and the attendant definitions wherein n is 0, R₁ is cyano, R₂ is ethyl, m is 0, and L₁ is S.

In a further embodiment, a sirtuin-activating compound is a compound of formula 41 and the attendant definitions wherein n is 0, R₁ is cyano, R₂ is ethyl, m is 0, L₁ is S, and L₂ is O.

In a further embodiment, a sirtuin-activating compound is a compound of formula 41 and the attendant definitions wherein n is 0, R₁ is cyano, R₂ is ethyl, m is 0, L₁ is S, L₂ is O, and L₃ is O.

In another embodiment, a sirtuin-activating compound is a compound of formula 42: 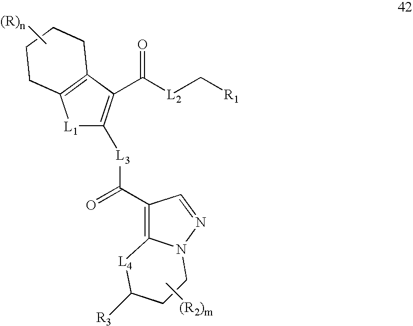
wherein, independently for each occurrence:

R and R₂ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ and R₃ are H or a substituted or unsubstituted alkyl, aryl, aralkyl, heterocyclyl, heterocyclylalkyl, heteroaryl, or heteroaralkyl;

L₁, L₂, L₃, and L₄ are O, NR₁, or S;

m is an integer from 0 to 6 inclusive; and

n is an integer from 0 to 8 inclusive.

In a further embodiment, a sirtuin-activating compound is a compound of formula 42 and the attendant definitions wherein n is 0.

In a further embodiment, a sirtuin-activating compound is a compound of formula 42 and the attendant definitions wherein R₁ is methyl.

In a further embodiment, a sirtuin-activating compound is a compound of formula 42 and the attendant definitions wherein R₂ is CF₃ and m is 1.

In a further embodiment, a sirtuin-activating compound is a compound of formula 42 and the attendant definitions wherein R₃ is 4-methylphenyl.

In a further embodiment, a sirtuin-activating compound is a compound of formula 42 and the attendant definitions wherein L₁ is S.

In a further embodiment, a sirtuin-activating compound is a compound of formula 42 and the attendant definitions wherein L₂ is O.

In a further embodiment, a sirtuin-activating compound is a compound of formula 42 and the attendant definitions wherein L₃ is NR₁.

In a further embodiment, a sirtuin-activating compound is a compound of formula 42 and the attendant definitions wherein L₄ is NR₁.

In a further embodiment, a sirtuin-activating compound is a compound of formula 42 and the attendant definitions wherein n is 0 and R₁ is methyl.

In a further embodiment, a sirtuin-activating compound is a compound of formula 42 and the attendant definitions wherein n is 0, R₁ is methyl, R₂ is CF₃, and m is 1.

In a further embodiment, a sirtuin-activating compound is a compound of formula 42 and the attendant definitions wherein n is 0, R₁ is methyl, R₂ is CF₃, m is 1; and R₃ is 4-methylphenyl.

In a further embodiment, a sirtuin-activating compound is a compound of formula 42 and the attendant definitions wherein n is 0, R₁ is methyl, R₂ is CF₃, m is 1; R₃ is 4-methylphenyl; and L₁ is S.

In a further embodiment, a sirtuin-activating compound is a compound of formula 42 and the attendant definitions wherein n is 0, R₁ is methyl, R₂ is CF₃, m is 1; R₃ is 4-methylphenyl; L₁ is S, and L₂ is O.

In a further embodiment, a sirtuin-activating compound is a compound of formula 42 and the attendant definitions wherein n is 0, R₁ is methyl, R₂ is CF₃, m is 1; R₃ is 4-methylphenyl; L₁ is S, L₂ is O; and L₃ is NR₁.

In a further embodiment, a sirtuin-activating compound is a compound of formula 42 and the attendant definitions wherein n is 0, R₁ is methyl, R₂ is CF₃, m is 1; R₃ is 4-methylphenyl; L₁ is S, L₂ is O; L₃ is NR₁, and L₄ is NR₁.

In another embodiment, a sirtuin-activating compound is a compound of formula 43: 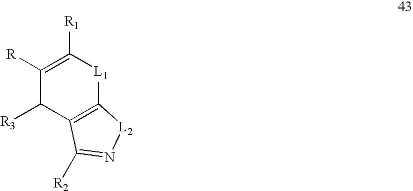
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ and R₃ are H or a substituted or unsubstituted alkyl, aryl, aralkyl, heterocyclyl, heterocyclylalkyl, heteroaryl, or heteroaralkyl; and

L₁ and L₂ are O, NR₂, or S.

In a further embodiment, a sirtuin-activating compound is a compound of formula 43 and the attendant definitions wherein R is cyano.

In a further embodiment, a sirtuin-activating compound is a compound of formula 43 and the attendant definitions wherein R₁ is NH₂.

In a further embodiment, a sirtuin-activating compound is a compound of formula 43 and the attendant definitions wherein R₂ is 4-bromophenyl.

In a further embodiment, a sirtuin-activating compound is a compound of formula 43 and the attendant definitions wherein R₃ is 3-hydroxy-4-methoxyphenyl.

In a further embodiment, a sirtuin-activating compound is a compound of formula 43 and the attendant definitions wherein L₁ is O.

In a further embodiment, a sirtuin-activating compound is a compound of formula 43 and the attendant definitions wherein L₂ is NR₂.

In a further embodiment, a sirtuin-activating compound is a compound of formula 43 and the attendant definitions wherein R is cyano and R₁ is NH₂.

In a further embodiment, a sirtuin-activating compound is a compound of formula 43 and the attendant definitions wherein R is cyano, R₁ is NH₂, and R₂ is 4-bromophenyl.

In a further embodiment, a sirtuin-activating compound is a compound of formula 43 and the attendant definitions wherein R is cyano, R₁ is NH₂, R₂ is 4-bromophenyl, and R₃ is 3-hydroxy-4-methoxyphenyl.

In a further embodiment, a sirtuin-activating compound is a compound of formula 43 and the attendant definitions wherein R is cyano, R₁ is NH₂, R₂ is 4-bromophenyl, R₃ is 3-hydroxy-4-methoxyphenyl, and L₁ is O.

In a further embodiment, a sirtuin-activating compound is a compound of formula 43 and the attendant definitions wherein R is cyano, R₁ is NH₂, R₂ is 4-bromophenyl, R₃ is 3-hydroxy-4-methoxyphenyl, L₁ is O, and L₂ is NR₂.

In another embodiment, a sirtuin-activating compound is a compound of formula 44: 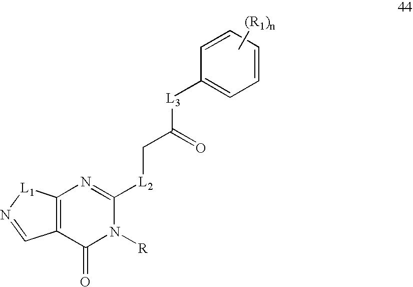
wherein, independently for each occurrence:

R is H or a substituted or unsubstituted alkyl, aryl, aralkyl, heterocyclyl, heterocyclylalkyl, heteroaryl, or heteroaralkyl;

R₁ is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR, or S; and

n is an integer from 0 to 5 inclusive.

In a further embodiment, a sirtuin-activating compound is a compound of formula 44 and the attendant definitions wherein R is 3-trifluoromethylphenyl.

In a further embodiment, a sirtuin-activating compound is a compound of formula 44 and the attendant definitions wherein R₁ is C(O)OCH₃.

In a further embodiment, a sirtuin-activating compound is a compound of formula 44 and the attendant definitions wherein L₁ is NR.

In a further embodiment, a sirtuin-activating compound is a compound of formula 44 and the attendant definitions wherein L₂ is S.

In a further embodiment, a sirtuin-activating compound is a compound of formula 44 and the attendant definitions wherein L₃ is NR.

In a further embodiment, a sirtuin-activating compound is a compound of formula 44 and the attendant definitions wherein n is 2.

In a further embodiment, a sirtuin-activating compound is a compound of formula 44 and the attendant definitions wherein R is 3-trifluoromethylphenyl and R₁ is C(O)OCH₃.

In a further embodiment, a sirtuin-activating compound is a compound of formula 44 and the attendant definitions wherein R is 3-trifluorormethylphenyl, R₁ is C(O)OCH₃, and L₁ is NR.

In a further embodiment, a sirtuin-activating compound is a compound of formula 44 and the attendant definitions wherein R is 3-trifluoromethylphenyl, R₁ is C(O)OCH₃, L₁ is NR, and L₂ is S.

In a further embodiment, a sirtuin-activating compound is a compound of formula 44 and the attendant definitions wherein R is 3-trifluoromethylphenyl, R₁ is C(O)OCH₃, L₁ is NR, L₂ is S, and L₃ is NR.

In a further embodiment, a sirtuin-activating compound is a compound of formula 44 and the attendant definitions wherein R is 3-trifluoromethylphenyl, R₁ is C(O)OCH₃, L₁ is NR, L₂ is S, L₃ is NR, and n is 2.

In another embodiment, a sirtuin-activating compound is a compound of formula 45: 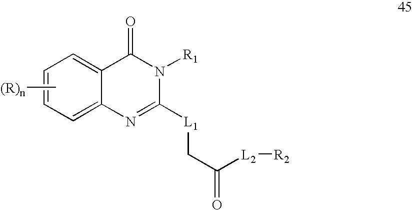
wherein, independently for each occurrence:

R is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ and R₂ are H or a substituted or unsubstituted alkyl, aryl, aralkyl, heterocyclyl, heterocyclylalkyl, heteroaryl, or heteroaralkyl;

L₁ and L₂ are O, NR₁, or S; and

n is an integer from 0 to 4 inclusive.

In a further embodiment, a sirtuin-activating compound is a compound of formula 45 and the attendant definitions wherein n is 0.

In a further embodiment, a sirtuin-activating compound is a compound of formula 45 and the attendant definitions wherein R₁ is 2-tetrahydrofuranylmethyl.

In a further embodiment, a sirtuin-activating compound is a compound of formula 45 and the attendant definitions wherein R₂ is —CH₂CH₂C₆H₄SO₂NH₂.

In a further embodiment, a sirtuin-activating compound is a compound of formula 45 and the attendant definitions wherein L₁ is S.

In a further embodiment, a sirtuin-activating compound is a compound of formula 45 and the attendant definitions wherein L₂ is NR₁.

In a further embodiment, a sirtuin-activating compound is a compound of formula 45 and the attendant definitions wherein n is 0 and R₁ is 2-tetrahydrofuranylmethyl.

In a further embodiment, a sirtuin-activating compound is a compound of formula 45 and the attendant definitions wherein n is 0, R₁ is 2-tetrahydrofuranylmethyl, and R₂ is —CH₂CH₂C₆H₄SO₂NH₂.

In a further embodiment, a sirtuin-activating compound is a compound of formula 45 and the attendant definitions wherein n is 0, R₁ is 2-tetrahydrofuranylmethyl, R₂ is —CH₂CH₂C₆H₄SO₂NH₂, and L₁ is S.

In a further embodiment, a sirtuin-activating compound is a compound of formula 45 and the attendant definitions wherein n is 0, R₁ is 2-tetrahydrofuranylmethyl, R₂ is —CH₂CH₂C₆H₄SO₂NH₂, L₁ is S, and L₂ is NR₁.

In another embodiment, a sirtuin-activating compound is a compound of formula 46: 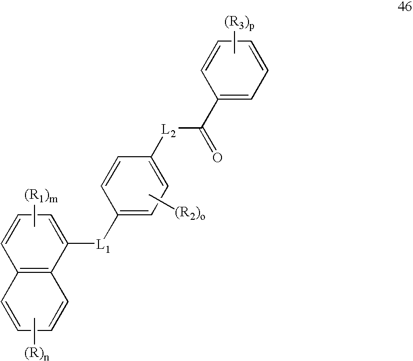
wherein, independently for each occurrence:

R, R₁, R₂, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR₄, or S;

R₄ is H or a substituted or unsubstituted alkyl, aryl, aralkyl, heterocyclyl, heterocyclylalkyl, heteroaryl, or heteroaralkyl;

n is an integer from 0 to 4 inclusive;

m is an integer from 0 to 3 inclusive;

o is an integer from 0 to 4 inclusive; and

p is an integer from 0 to 5 inclusive.

In a further embodiment, a sirtuin-activating compound is a compound of formula 46 and the attendant definitions wherein n is 0.

In a further embodiment, a sirtuin-activating compound is a compound of formula 46 and the attendant definitions wherein m is 1.

In a further embodiment, a sirtuin-activating compound is a compound of formula 46 and the attendant definitions wherein R₁ is Cl.

In a further embodiment, a sirtuin-activating compound is a compound of formula 46 and the attendant definitions wherein o is 1.

In a further embodiment, a sirtuin-activating compound is a compound of formula 46 and the attendant definitions wherein R₂ is Cl.

In a further embodiment, a sirtuin-activating compound is a compound of formula 46 and the attendant definitions wherein p is 3.

In a further embodiment, a sirtuin-activating compound is a compound of formula 46 and the attendant definitions wherein R₃ is OH or I.

In a further embodiment, a sirtuin-activating compound is a compound of formula 46 and the attendant definitions wherein n is 0 and m is 1.

In a further embodiment, a sirtuin-activating compound is a compound of formula 46 and the attendant definitions wherein n is 0, m is 1, and o is 1.

In a further embodiment, a sirtuin-activating compound is a compound of formula 46 and the attendant definitions wherein n is 0, m is 1, o is 1, and R₁ is Cl.

In a further embodiment, a sirtuin-activating compound is a compound of formula 46 and the attendant definitions wherein n is 0, m is 1, o is 1, R₁ is Cl, and p is 3.

In a further embodiment, a sirtuin-activating compound is a compound of formula 46 and the attendant definitions wherein n is 0, m is 1, o is 1, R₁ is Cl, p is 3, and R₂ is OH or I.

In another embodiment, a sirtuin-activating compound is a compound of formula 47: 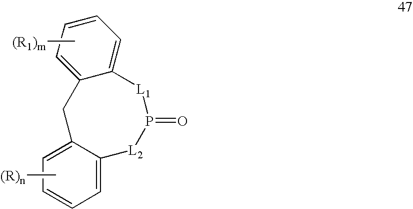
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR₄, or S;

R₄ is H or a substituted or unsubstituted alkyl, aryl, aralkyl, heterocyclyl, heterocyclylalkyl, heteroaryl, or heteroaralkyl; and

m and n are integers from 0 to 4 inclusive.

In a further embodiment, a sirtuin-activating compound is a compound of formula 47 and the attendant definitions wherein n is 2.

In a further embodiment, a sirtuin-activating compound is a compound of formula 47 and the attendant definitions wherein R is methyl or t-butyl.

In a further embodiment, a sirtuin-activating compound is a compound of formula 47 and the attendant definitions wherein m is 2.

In a further embodiment, a sirtuin-activating compound is a compound of formula 47 and the attendant definitions wherein R₁ is methyl or t-butyl.

In a further embodiment, a sirtuin-activating compound is a compound of formula 47 and the attendant definitions wherein L₁ is O.

In a further embodiment, a sirtuin-activating compound is a compound of formula 47 and the attendant definitions wherein L₂ is O.

In a further embodiment, a sirtuin-activating compound is a compound of formula 47 and the attendant definitions wherein n is 2 and R is methyl or t-butyl.

In a further embodiment, a sirtuin-activating compound is a compound of formula 47 and the attendant definitions wherein n is 2, R is methyl or t-butyl, and m is 2.

In a further embodiment, a sirtuin-activating compound is a compound of formula 47 and the attendant definitions wherein n is 2, R is methyl or t-butyl, m is 2, and R₁ is methyl or t-butyl.

In a further embodiment, a sirtuin-activating compound is a compound of formula 47 and the attendant definitions wherein n is 2, R is methyl or t-butyl, m is 2, R₁ is methyl or t-butyl, and L₁ is O.

In a further embodiment, a sirtuin-activating compound is a compound of formula 47 and the attendant definitions wherein n is 2, R is methyl or t-butyl, m is 2, R₁ is methyl or t-butyl, L₁ is O, and L₂ is O.

In another embodiment, a sirtuin-activating compound is a compound of formula 48: 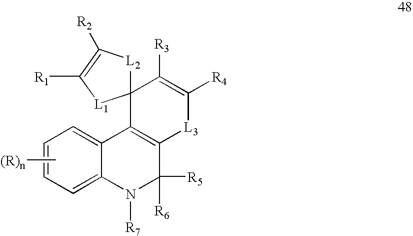
wherein, independently for each occurrence:

R, R₁, R₂, R₃, R₄, R₅, and R₆ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₇ is H or a substituted or unsubstituted alkyl, acyl, aryl, aralkyl, heterocyclyl, heterocyclylalkyl, heteroaryl, or heteroaralkyl;

L₁, L₂, and L₃ are O, NR₇, or S and

n is an integer from 0 to 4 inclusive.

In a further embodiment, a sirtuin-activating compound is a compound of formula 48 and the attendant definitions wherein n is 1.

In a further embodiment, a sirtuin-activating compound is a compound of formula 48 and the attendant definitions wherein R is methyl.

In a further embodiment, a sirtuin-activating compound is a compound of formula 48 and the attendant definitions wherein R₁ is C(O)OCH₃.

In a further embodiment, a sirtuin-activating compound is a compound of formula 48 and the attendant definitions wherein R₂ is C(O)OCH₃.

In a further embodiment, a sirtuin-activating compound is a compound of formula 48 and the attendant definitions wherein R₃ is C(O)OCH₃.

In a further embodiment, a sirtuin-activating compound is a compound of formula 48 and the attendant definitions wherein R₄ is C(O)OCH₃.

In a further embodiment, a sirtuin-activating compound is a compound of formula 48 and the attendant definitions wherein R₅ is methyl.

In a further embodiment, a sirtuin-activating compound is a compound of formula 48 and the attendant definitions wherein R₆ is methyl.

In a further embodiment, a sirtuin-activating compound is a compound of formula 48 and the attendant definitions wherein R₇ is C(O)CF₃.

In a further embodiment, a sirtuin-activating compound is a compound of formula 48 and the attendant definitions wherein L₁ is S.

In a further embodiment, a sirtuin-activating compound is a compound of formula 48 and the attendant definitions wherein L₂ is S.

In a further embodiment, a sirtuin-activating compound is a compound of formula 48 and the attendant definitions wherein L₃ is S.

In a further embodiment, a sirtuin-activating compound is a compound of formula 48 and the attendant definitions wherein n is 1 and R is methyl.

In a further embodiment, a sirtuin-activating compound is a compound of formula 48 and the attendant definitions wherein n is 1, R is methyl, and R₁ is C(O)OCH₃.

In a further embodiment, a sirtuin-activating compound is a compound of formula 48 and the attendant definitions wherein n is 1, R is methyl, R₁ is C(O)OCH₃, and R₂ is C(O)OCH₃.

In a further embodiment, a sirtuin-activating compound is a compound of formula 48 and the attendant definitions wherein n is 1, R is methyl, R₁ is C(O)OCH₃, R₂ is C(O)OCH₃, and R₃ is C(O)OCH₃.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, and R₄ is C(O)OCH₃.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, and R₅ is methyl.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, and R₆ is methyl.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, and R₇ is C(O)CF₃.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, and L₁ is S.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, L₁ is S, and L₂ is S.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, L₁ is S, L₂ is S, and L₃ is S.

In another embodiment, a sirtuin-activating compound is a compound of formula 49: 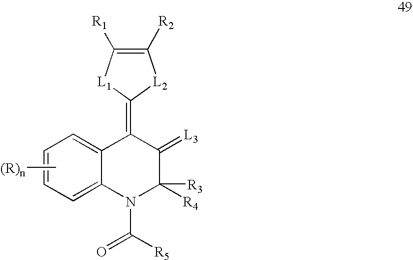
wherein, independently for each occurrence:

R, R₁, R₂, R₃, R₄, and R₅ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ , L₂, and L₃ are O, NR₆, or S;

R₆ is H or a substituted or unsubstituted alkyl, aryl, aralkyl, heterocyclyl, heterocyclylalkyl, heteroaryl, or heteroaralkyl; and

n is an integer from 0 to 4 inclusive.

In a further embodiment, a sirtuin-activating compound is a compound of formula 49 and the attendant definitions wherein n is 1.

In a further embodiment, a sirtuin-activating compound is a compound of formula 49 and the attendant definitions wherein R is methyl.

In a further embodiment, a sirtuin-activating compound is a compound of formula 49 and the attendant definitions wherein R₁ is C(O)OCH₃.

In a further embodiment, a sirtuin-activating compound is a compound of formula 49 and the attendant definitions wherein R₂ is C(O)OCH₃.

In a further embodiment, a sirtuin-activating compound is a compound of formula 49 and the attendant definitions wherein R₃ is methyl.

In a further embodiment, a sirtuin-activating compound is a compound of formula 49 and the attendant definitions wherein R₄ is methyl.

In a further embodiment, a sirtuin-activating compound is a compound of formula 49 and the attendant definitions wherein R₅ is CH₂CH(CH₃)₂.

In a further embodiment, a sirtuin-activating compound is a compound of formula 49 and the attendant definitions wherein L₁ is S.

In a further embodiment, a sirtuin-activating compound is a compound of formula 49 and the attendant definitions wherein L₂ is S.

In a further embodiment, a sirtuin-activating compound is a compound of formula 49 and the attendant definitions wherein L₃ is S.

In a further embodiment, a sirtuin-activating compound is a compound of formula 49 and the attendant definitions wherein n is 1 and R is methyl.

In a further embodiment, a sirtuin-activating compound is a compound of formula 49 and the attendant definitions wherein n is 1, R is methyl, and R₁ is C(O)OCH₃.

In a further embodiment, a sirtuin-activating compound is a compound of formula 49 and the attendant definitions wherein n is 1, R is methyl, R₁ is C(O)OCH₃, and R₂ is C(O)OCH₃.

In a further embodiment, a sirtuin-activating compound is a compound of formula 49 and the attendant definitions wherein n is 1, R is methyl, R₁ is C(O)OCH₃, R₂ is C(O)OCH₃, and R₃ is methyl.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, and R₄ is methyl.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, and R₅ is CH₂CH(CH₃)₂.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, and L₁ is S.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, and L₁ is S.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, and L₂ is S.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, and L₂ is S.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, L₂ is S, and L₃ is S.

In another embodiment, a sirtuin-activating compound is a compound of formula 50: 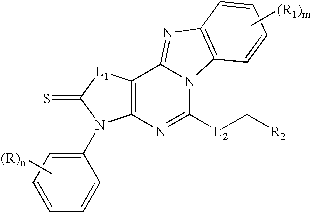
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR₃, or S;

R₃ is H or a substituted or unsubstituted alkyl, aryl, aralkyl, heterocyclyl, heterocyclylalkyl, heteroaryl, or heteroaralkyl;

n is an integer from 0 to 5 inclusive; and

m is an integer from 0 to 4 inclusive.

In a further embodiment, a sirtuin-activating compound is a compound of formula 50 and the attendant definitions wherein n is 1.

In a further embodiment, a sirtuin-activating compound is a compound of formula 50 and the attendant definitions wherein R is CO₂Et.

In a further embodiment, a sirtuin-activating compound is a compound of formula 50 and the attendant definitions wherein m is 0.

In a further embodiment, a sirtuin-activating compound is a compound of formula 50 and the attendant definitions wherein R₂ is cyano.

In a further embodiment, a sirtuin-activating compound is a compound of formula 50 and the attendant definitions wherein L₁ is S.

In a further embodiment, a sirtuin-activating compound is a compound of formula 50 and the attendant definitions wherein L₂ is S.

In a further embodiment, a sirtuin-activating compound is a compound of formula 50 and the attendant definitions wherein n is 1 and R is CO₂Et.

In a further embodiment, a sirtuin-activating compound is a compound of formula 50 and the attendant definitions wherein n is 1, R is CO₂Et, and m is 0.

In a further embodiment, a sirtuin-activating compound is a compound of formula 50 and the attendant definitions wherein n is 1, R is CO₂Et, m is 0, and R₂ is cyano.

In a further embodiment, a sirtuin-activating compound is a compound of formula 50 and the attendant definitions wherein n is 1, R is CO₂Et, m is 0, R₂ is cyano, and L₁ is S.

In a further embodiment, a sirtuin-activating compound is a compound of formula 50 and the attendant definitions wherein n is 1, R is CO₂Et, m is 0, R₂ is cyano, L₁ is S, and L₂ is S.

In another embodiment, a sirtuin-activating compound is a compound of formula 51: 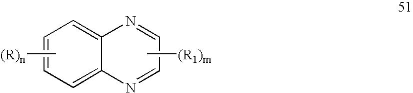
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

n is an integer from 0 to 4 inclusive; and

m is an integer from 0 to 2 inclusive.

In a further embodiment, a sirtuin-activating compound is a compound of formula 51 and the attendant definitions wherein n is 2.

In a further embodiment, a sirtuin-activating compound is a compound of formula 51 and the attendant definitions wherein R is Cl or trifluoromethyl.

In a further embodiment, a sirtuin-activating compound is a compound of formula 51 and the attendant definitions wherein m is 2.

In a further embodiment, a sirtuin-activating compound is a compound of formula 51 and the attendant definitions wherein R₁ is phenyl.

In a further embodiment, a sirtuin-activating compound is a compound of formula 51 and the attendant definitions wherein n is 2 and R is Cl or trifluoromethyl.

In a further embodiment, a sirtuin-activating compound is a compound of formula 51 and the attendant definitions wherein n is 2, R is Cl or trifluoromethyl, and m is 2.

In a further embodiment, a sirtuin-activating compound is a compound of formula 51 and the attendant definitions wherein n is 2, R is Cl or trifluoromethyl, m is 2, and R₁ is phenyl.

In a further embodiment, a sirtuin-activating compound is a compound of formula 51 and the attendant definitions wherein n is 1.

In a further embodiment, a sirtuin-activating compound is a compound of formula 51 and the attendant definitions wherein R is F.

In a further embodiment, a sirtuin-activating compound is a compound of formula 51 and the attendant definitions wherein R₁ is 4-methylphenyl.

In a further embodiment, a sirtuin-activating compound is a compound of formula 51 and the attendant definitions wherein n is 1 and R is F.

In a further embodiment, a sirtuin-activating compound is a compound of formula 51 and the attendant definitions wherein n is 1, R is F, and m is 2.

In a further embodiment, a sirtuin-activating compound is a compound of formula 51 and the attendant definitions wherein n is 1, R is F, m is 2, and R₁ is 4-methylphenyl.

In another embodiment, a sirtuin-activating compound is a compound of formula 52: 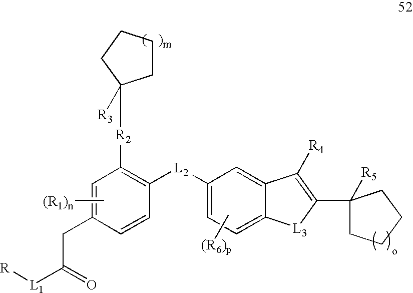
wherein, independently for each occurrence:

R is H or a substituted or unsubstituted alkyl, aryl, aralkyl, heterocyclyl, heterocyclylalkyl, heteroaryl, or heteroaralkyl;

R₁ and R₆ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is alkylene, alkenylene, or alkynylene;

R₃, R₄, and R₅ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR, or S;

n and p are integers from 0 to 3 inclusive; and

m and o are integers from 0 to 2 inclusive.

In a further embodiment, a sirtuin-activating compound is a compound of formula 52 and the attendant definitions wherein R is CH₂CH₂OH.

In a further embodiment, a sirtuin-activating compound is a compound of formula 52 and the attendant definitions wherein n is 1.

In a further embodiment, a sirtuin-activating compound is a compound of formula 52 and the attendant definitions wherein R₁ is I.

In a further embodiment, a sirtuin-activating compound is a compound of formula 52 and the attendant definitions wherein R₂ is alkynylene.

In a further embodiment, a sirtuin-activating compound is a compound of formula 52 and the attendant definitions wherein m is 1.

In a further embodiment, a sirtuin-activating compound is a compound of formula 52 and the attendant definitions wherein R₃ is OH.

In a further embodiment, a sirtuin-activating compound is a compound of formula 52 and the attendant definitions wherein R₄ is C(O)OEt.

In a further embodiment, a sirtuin-activating compound is a compound of formula 52 and the attendant definitions wherein o is 1.

In a further embodiment, a sirtuin-activating compound is a compound of formula 52 and the attendant definitions wherein R₅ is OH.

In a further embodiment, a sirtuin-activating compound is a compound of formula 52 and the attendant definitions wherein p is 0.

In a further embodiment, a sirtuin-activating compound is a compound of formula 52 and the attendant definitions wherein L₁ is NH.

In a further embodiment, a sirtuin-activating compound is a compound of formula 52 and the attendant definitions wherein L₂ is O.

In a further embodiment, a sirtuin-activating compound is a compound of formula 52 and the attendant definitions wherein L₃ is O.

In a further embodiment, a sirtuin-activating compound is a compound of formula 52 and the attendant definitions wherein R is CH₂CH₂OH and n is 1.

In a further embodiment, a sirtuin-activating compound is a compound of formula 52 and the attendant definitions wherein R is CH₂CH₂OH, n is 1, and R₁ is I.

In a further embodiment, a sirtuin-activating compound is a compound of formula 52 and the attendant definitions wherein R is CH₂CH₂OH, n is 1, R₁ is I, and R₂ is alkynylene.

In a further embodiment, a sirtuin-activating compound is a compound of formula 52 and the attendant definitions wherein R is CH₂CH₂OH, n is 1, R₁ is I, R₂ is alkynylene, and m is 1.

In a further embodiment, a sirtuin-activating compound is a compound of formula 52 and the attendant definitions wherein R is CH₂CH₂OH, n is 1, R₁ is I, R₂ is alkynylene, m is 1, and R₃ is OH.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, and R₄ is C(O)OEt.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, and o is 1.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, o is 1, and R₅ is OH.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, and p is 0.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, p is 0, and L₁ is NH.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, p is 0, L₁ is NH, and L₂ is O.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, p is 0, L₁ is NH, L₂ is O, and L₃ is O.

In another embodiment, a sirtuin-activating compound is a compound of formula 53: 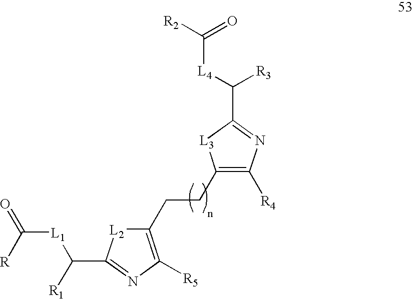
wherein, independently for each occurrence:

R, R₁, R₂, R₃, R₄, and R₅ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, L₃, and L₄ are O, NR₆, or S;

R₆ is and H, or a substituted or unsubstituted alkyl, aryl, aralkyl, heterocyclyl, heterocyclylalkyl, heteroaryl, or heteroaralkyl; and

n is an integer from 0 to 5 inclusive.

In a further embodiment, a sirtuin-activating compound is a compound of formula 53 and the attendant definitions wherein R is O-t-butyl.

In a further embodiment, a sirtuin-activating compound is a compound of formula 53 and the attendant definitions wherein R₁ is t-butyl.

In a further embodiment, a sirtuin-activating compound is a compound of formula 53 and the attendant definitions wherein R₂ is O-t-butyl.

In a further embodiment, a sirtuin-activating compound is a compound of formula 53 and the attendant definitions wherein R₃ is t-butyl.

In a further embodiment, a sirtuin-activating compound is a compound of formula 53 and the attendant definitions wherein R₄ is C(O)OMe.

In a further embodiment, a sirtuin-activating compound is a compound of formula 53 and the attendant definitions wherein R₅ is C(O)OMe.

In a further embodiment, a sirtuin-activating compound is a compound of formula 53 and the attendant definitions wherein L₁ is NH.

In a further embodiment, a sirtuin-activating compound is a compound of formula 53 and the attendant definitions wherein L₂ is O.

In a further embodiment, a sirtuin-activating compound is a compound of formula 53 and the attendant definitions wherein L₃ is O.

In a further embodiment, a sirtuin-activating compound is a compound of formula 53 and the attendant definitions wherein L₄ is NH.

In a further embodiment, a sirtuin-activating compound is a compound of formula 53 and the attendant definitions wherein n is 1.

In a further embodiment, a sirtuin-activating compound is a compound of formula 53 and the attendant definitions wherein R is O-t-butyl and R₁ is t-butyl.

In a further embodiment, a sirtuin-activating compound is a compound of formula 53 and the attendant definitions wherein R is O-t-butyl, R₁ is t-butyl, and R₂ is O-t-butyl.

In a further embodiment, a sirtuin-activating compound is a compound of formula 53 and the attendant definitions wherein R is O-t-butyl, R₁ is t-butyl, R₂ is O-t-butyl, and R₃ is t-butyl.

In a further embodiment, a sirtuin-activating compound is a compound of formula 53 and the attendant definitions wherein R is O-t-butyl, R₁ is t-butyl, R₂ is O-t-butyl, R₃ is t-butyl, and R₄ is C(O)OMe.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, and R₅ is C(O)OMe.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, and L₁ is NH.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, and L₂ is O.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, and L₃ is O.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, L₃ is O, and L₄ is NH.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, L₃ is O, L₄ is NH, and n is 1.

In another embodiment, a sirtuin-activating compound is a compound of formula 54: 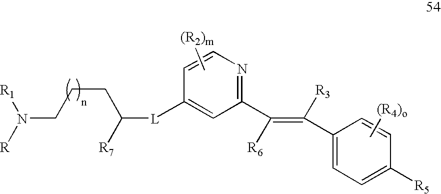
wherein, independently for each occurrence:

R and R₁ are H or a substituted or unsubstituted alkyl, aryl, aralkyl, heterocyclyl, heterocyclylalkyl, heteroaryl, or heteroaralkyl;

R₂, R₄, and R₅ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₃, R₆, and R₇ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L is O, NR, or S;

n and o are integers from 0 to 4 inclusive; and

m is an integer from 0 to 3 inclusive.

In a further embodiment, a sirtuin-activating compound is a compound of formula 54 and the attendant definitions wherein R is ethyl.

In a further embodiment, a sirtuin-activating compound is a compound of formula 54 and the attendant definitions wherein R₁ is ethyl.

In a further embodiment, a sirtuin-activating compound is a compound of formula 54 and the attendant definitions wherein m is 0.

In a further embodiment, a sirtuin-activating compound is a compound of formula 54 and the attendant definitions wherein R₃ is H.

In a further embodiment, a sirtuin-activating compound is a compound of formula 54 and the attendant definitions wherein o is 0.

In a further embodiment, a sirtuin-activating compound is a compound of formula 54 and the attendant definitions wherein R₅ is Cl.

In a further embodiment, a sirtuin-activating compound is a compound of formula 54 and the attendant definitions wherein R₆ is H.

In a further embodiment, a sirtuin-activating compound is a compound of formula 54 and the attendant definitions wherein R₇ is methyl.

In a further embodiment, a sirtuin-activating compound is a compound of formula 54 and the attendant definitions wherein L is NH.

In a further embodiment, a sirtuin-activating compound is a compound of formula 54 and the attendant definitions wherein n is 1.

In a further embodiment, a sirtuin-activating compound is a compound of formula 54 and the attendant definitions wherein R is ethyl and R₁ is ethyl.

In a further embodiment, a sirtuin-activating compound is a compound of formula 54 and the attendant definitions wherein R is ethyl, R₁ is ethyl, and m is 0.

In a further embodiment, a sirtuin-activating compound is a compound of formula 54 and the attendant definitions wherein R is ethyl, R₁ is ethyl, m is 0, and R₃ is H.

In a further embodiment, a sirtuin-activating compound is a compound of formula 54 and the attendant definitions wherein R is ethyl, R₁ is ethyl, m is 0, R₃ is H, and o is 0.

In a further embodiment, a sirtuin-activating compound is a compound of formula 54 and the attendant definitions wherein R is ethyl, R₁ is ethyl, m is 0, R₃ is H, o is 0, and R₅ is Cl.

In a further embodiment, a sirtuin-activating compound is a compound of formula 54 and the attendant definitions wherein R is ethyl, R₁ is ethyl, m is 0, R₃ is H, o is 0, R₅ is Cl, and R₆ is H.

In a further embodiment, a sirtuin-activating compound is a compound of formula 54 and the attendant definitions wherein R is ethyl, R₁ is ethyl, m is 0, R₃ is H, o is 0, R₅ is Cl, R₆ is H, and R₇ is methyl.

In a further embodiment, a sirtuin-activating compound is a compound of formula 54 and the attendant definitions wherein R is ethyl, R₁ is ethyl, m is 0, R₃ is H, o is 0, R₅ is Cl, R₆ is H, R₇ is methyl, and L is NH.

In a further embodiment, a sirtuin-activating compound is a compound of formula 54 and the attendant definitions wherein R is ethyl, R₁ is ethyl, m is 0, R₃ is H, o is 0, R₅ is Cl, R₆ is H, R₇ is methyl, L is NH, and n is 1.

In another embodiment, a sirtuin-activating compound is a compound of formula 55: 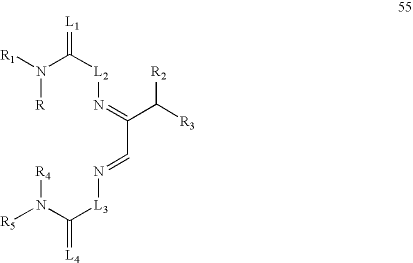
wherein, independently for each occurrence:

R, R₁, R₄, and R₅ are H or a substituted or unsubstituted alkyl, aryl, aralkyl, heterocyclyl, heterocyclylalkyl, heteroaryl, or heteroaralkyl;

R₂ and R₃ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl; and

L₁, L₂, L₃, and L₄ are O, NR, or S.

In a further embodiment, a sirtuin-activating compound is a compound of formula 55 and the attendant definitions wherein R is H.

In a further embodiment, a sirtuin-activating compound is a compound of formula 55 and the attendant definitions wherein R₁ is H.

In a further embodiment, a sirtuin-activating compound is a compound of formula 55 and the attendant definitions wherein R₂ is OEt.

In a further embodiment, a sirtuin-activating compound is a compound of formula 55 and the attendant definitions wherein R₃ is methyl.

In a further embodiment, a sirtuin-activating compound is a compound of formula 55 and the attendant definitions wherein R₄ is H.

In a further embodiment, a sirtuin-activating compound is a compound of formula 55 and the attendant definitions wherein R₅ is H.

In a further embodiment, a sirtuin-activating compound is a compound of formula 55 and the attendant definitions wherein L₁ is S.

In a further embodiment, a sirtuin-activating compound is a compound of formula 55 and the attendant definitions wherein L₂ is NH.

In a further embodiment, a sirtuin-activating compound is a compound of formula 55 and the attendant definitions wherein L₃ is NH.

In a further embodiment, a sirtuin-activating compound is a compound of formula 55 and the attendant definitions wherein L₄ is S.

In a further embodiment, a sirtuin-activating compound is a compound of formula 55 and the attendant definitions wherein R is H and R₁ is H.

In a further embodiment, a sirtuin-activating compound is a compound of formula 55 and the attendant definitions wherein R is H, R₁ is H, and R₂ is OEt.

In a further embodiment, a sirtuin-activating compound is a compound of formula 55 and the attendant definitions wherein R is H, R₁ is H, R₂ is OEt, and R₃ is methyl.

In a further embodiment, a sirtuin-activating compound is a compound of formula 55 and the attendant definitions wherein R is H, R₁ is H, R₂ is OEt, R₃ is methyl, and R₄ is H.

In a further embodiment, a sirtuin-activating compound is a compound of formula 55 and the attendant definitions wherein R is H, R₁ is H, R₂ is OEt, R₃ is methyl, R₄ is H, and R₅ is H.

In a further embodiment, a sirtuin-activating compound is a compound of formula 55 and the attendant definitions wherein R is H, R₁ is H, R₂ is OEt, R₃ is methyl, R₄ is H, R₅ is H, and L₁ is S.

In a further embodiment, a sirtuin-activating compound is a compound of formula 55 and the attendant definitions wherein R is H, R₁ is H, R₂ is OEt, R₃ is methyl, R₄ is H, R₅ is H, L₁ is S, and L₂ is NH.

In a further embodiment, a sirtuin-activating compound is a compound of formula 55 and the attendant definitions wherein R is H, R₁ is H, R₂ is OEt, R₃ is methyl, R₄ is H, R₅ is H, L₁ is S, L₂ is NH, and L₃ is NH.

In a further embodiment, a sirtuin-activating compound is a compound of formula 55 and the attendant definitions wherein R is H, R₁ is H, R₂ is OEt, R₃ is methyl, R₄ is H, R₅ is H, L₁ is S, L₂ is NH, L₃ is NH, and L₄ is S.

In another embodiment, a sirtuin-activating compound is a compound of formula 56: 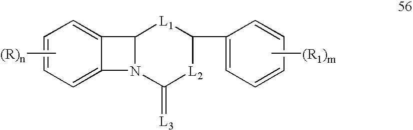
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR₂, or S;

R₂ is H or a substituted or unsubstituted alkyl, aryl, aralkyl, heterocyclyl, heterocyclylalkyl, heteroaryl, or heteroaralkyl;

n is an integer from 0 to 4 inclusive; and

m is an integer from 0 to 5 inclusive.

In a further embodiment, a sirtuin-activating compound is a compound of formula 56 and the attendant definitions wherein n is 0.

In a further embodiment, a sirtuin-activating compound is a compound of formula 56 and the attendant definitions wherein m is 0.

In a further embodiment, a sirtuin-activating compound is a compound of formula 56 and the attendant definitions wherein L₁ is NH.

In a further embodiment, a sirtuin-activating compound is a compound of formula 56 and the attendant definitions wherein L₂ is S.

In a further embodiment, a sirtuin-activating compound is a compound of formula 56 and the attendant definitions wherein L₃ is S.

In a further embodiment, a sirtuin-activating compound is a compound of formula 56 and the attendant definitions wherein m is 0 and n is 0.

In a further embodiment, a sirtuin-activating compound is a compound of formula 56 and the attendant definitions wherein m is 0, n is 0, and L₁ is NH.

In a further embodiment, a sirtuin-activating compound is a compound of formula 56 and the attendant definitions wherein m is 0, n is 0, L₁ is NH, and L₂ is S.

In a further embodiment, a sirtuin-activating compound is a compound of formula 56 and the attendant definitions wherein m is 0, n is 0, L₁ is NH, L₂ is S, and L₃ is S.

In another embodiment, a sirtuin-activating compound is a compound of formula 57: 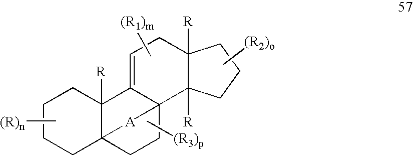
wherein, independently for each occurrence:

R, R₁, R₂, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

A is alkylene, alkenylene, or alkynylene;

n is an integer from 0 to 8 inclusive;

m is an integer from 0 to 3 inclusive;

o is an integer from 0 to 6 inclusive; and

p is an integer from 0 to 4 inclusive.

In a further embodiment, a sirtuin-activating compound is a compound of formula 57 and the attendant definitions wherein n is 2.

In a further embodiment, a sirtuin-activating compound is a compound of formula 57 and the attendant definitions wherein R is OH or methyl.

In a further embodiment, a sirtuin-activating compound is a compound of formula 57 and the attendant definitions wherein m is 1.

In a further embodiment, a sirtuin-activating compound is a compound of formula 57 and the attendant definitions wherein R₁ is methyl.

In a further embodiment, a sirtuin-activating compound is a compound of formula 57 and the attendant definitions wherein o is 1.

In a further embodiment, a sirtuin-activating compound is a compound of formula 57 and the attendant definitions wherein R₂ is C(O)CH₃.

In a further embodiment, a sirtuin-activating compound is a compound of formula 57 and the attendant definitions wherein p is 2.

In a further embodiment, a sirtuin-activating compound is a compound of formula 57 and the attendant definitions wherein R₃ is CO₂H.

In a further embodiment, a sirtuin-activating compound is a compound of formula 57 and the attendant definitions wherein A is alkenylene.

In a further embodiment, a sirtuin-activating compound is a compound of formula 57 and the attendant definitions wherein n is 2 and R is OH or methyl.

In a further embodiment, a sirtuin-activating compound is a compound of formula 57 and the attendant definitions wherein n is 2, R is OH or methyl, and m is 1.

In a further embodiment, a sirtuin-activating compound is a compound of formula 57 and the attendant definitions wherein n is 2, R is OH or methyl, m is 1, and R₁ is methyl.

In a further embodiment, a sirtuin-activating compound is a compound of formula 57 and the attendant definitions wherein n is 2, R is OH or methyl, m is 1, R₁ is methyl, and o is 1.

In a further embodiment, a sirtuin-activating compound is a compound of formula 57 and the attendant definitions wherein n is 2, R is OH or methyl, m is 1, R₁ is methyl, o is 1, and R₂ is C(O)CH₃.

In a further embodiment, a sirtuin-activating compound is a compound of formula 57 and the attendant definitions wherein n is 2, R is OH or methyl, m is 1, R₁ is methyl, o is 1, R₂ is C(O)CH₃, and p is 2.

IIn a further embodiment, a sirtuin-activating compound is a compound of formula 57 and the attendant definitions wherein n is 2, R is OH or methyl, m is 1, R₁ is methyl, o is 1, R₂ is C(O)CH₃, p is 2, and R₃ is CO₂H.

In a further embodiment, a sirtuin-activating compound is a compound of formula 57 and the attendant definitions wherein n is 2, R is OH or methyl, m is 1, R₁ is methyl, o is 1, R₂ is C(O)CH₃, p is 2, R₃ is CO₂H, and A is alkenylene.

In another embodiment, a sirtuin-activating compound is a compound of formula 58: 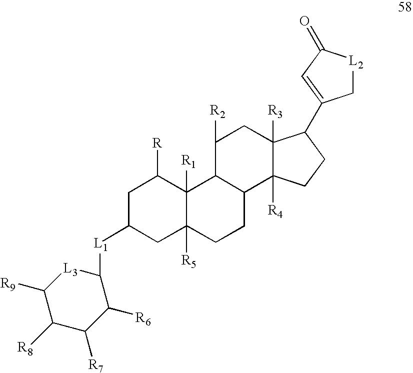
wherein, independently for each occurrence:

R, R₁, R₂, R₃, R₄, R₅, R₆, R₇, R₈, and R₉ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR₁₀, or S; and

R₁₀ is H or a substituted or unsubstituted alkyl, aryl, aralkyl, heterocyclyl, heterocyclylalkyl, heteroaryl, or heteroaralkyl.

In a further embodiment, a sirtuin-activating compound is a compound of formula 58 and the attendant definitions wherein R is OH.

In a further embodiment, a sirtuin-activating compound is a compound of formula 58 and the attendant definitions wherein R₁ is CH₂OH.

In a further embodiment, a sirtuin-activating compound is a compound of formula 58 and the attendant definitions wherein R₂ is OH.

In a further embodiment, a sirtuin-activating compound is a compound of formula 58 and the attendant definitions wherein R₃ is methyl.

In a further embodiment, a sirtuin-activating compound is a compound of formula 58 and the attendant definitions wherein R₄ is OH.

In a further embodiment, a sirtuin-activating compound is a compound of formula 58 and the attendant definitions wherein R₅ is OH.

In a further embodiment, a sirtuin-activating compound is a compound of formula 58 and the attendant definitions wherein R₆ is OH.

In a further embodiment, a sirtuin-activating compound is a compound of formula 58 and the attendant definitions wherein R₇ is OH.

In a further embodiment, a sirtuin-activating compound is a compound of formula 58 and the attendant definitions wherein R₈ is OH.

In a further embodiment, a sirtuin-activating compound is a compound of formula 58 and the attendant definitions wherein R₉ is methyl.

In a further embodiment, a sirtuin-activating compound is a compound of formula 58 and the attendant definitions wherein L₁ is O.

In a further embodiment, a sirtuin-activating compound is a compound of formula 58 and the attendant definitions wherein L₂ is O.

In a further embodiment, a sirtuin-activating compound is a compound of formula 58 and the attendant definitions wherein L₃ is O.

In a further embodiment, a sirtuin-activating compound is a compound of formula 58 and the attendant definitions wherein R is OH and R₁ is CH₂OH.

In a further embodiment, a sirtuin-activating compound is a compound of formula 58 and the attendant definitions wherein R is OH, R₁ is CH₂OH, and R₂ is OH.

In a further embodiment, a sirtuin-activating compound is a compound of formula 58 and the attendant definitions wherein R is OH, R₁ is CH₂OH, R₂ is OH, and R₃ is methyl.

In a further embodiment, a sirtuin-activating compound is a compound of formula 58 and the attendant definitions wherein R is OH, R₁ is CH₂OH, R₂ is OH, R₃ is methyl, and R₄ is OH.

In a further embodiment, a sirtuin-activating compound is a compound of formula 58 and the attendant definitions wherein R is OH, R₁ is CH₂OH, R₂ is OH, R₃ is methyl, R₄ is OH, and R₅ is OH.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, and R₆ is OH.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, and R₇ is OH.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, R₇ is OH, and R₈ is OH.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, and R₉ is methyl.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, and L₁ is O.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, L₁ is O, and L₂ is O.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, L₁ is O, L₂ is O, and L₃ is O.

In another embodiment, a sirtuin-activating compound is a compound of formula 59: 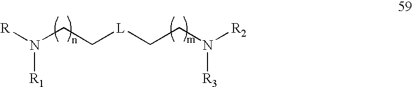
wherein, independently for each occurrence:

R, R₁, R₂, and R₃ are H or a substituted or unsubstituted alkyl, aryl, aralkyl, heterocyclyl, heterocyclylalkyl, heteroaryl, or heteroaralkyl;

L is O, NR, S, or Se; and

n and m are integers from 0 to 5 inclusive.

In a further embodiment, a sirtuin-activating compound is a compound of formula 59 and the attendant definitions wherein R is H.

In a further embodiment, a sirtuin-activating compound is a compound of formula 59 and the attendant definitions wherein R₁ is H.

In a further embodiment, a sirtuin-activating compound is a compound of formula 59 and the attendant definitions wherein R₂ is H.

In a further embodiment, a sirtuin-activating compound is a compound of formula 59 and the attendant definitions wherein R₃ is H.

In a further embodiment, a sirtuin-activating compound is a compound of formula 59 and the attendant definitions wherein L is Se.

In a further embodiment, a sirtuin-activating compound is a compound of formula 59 and the attendant definitions wherein n is 1.

In a further embodiment, a sirtuin-activating compound is a compound of formula 59 and the attendant definitions wherein m is 1.

In a further embodiment, a sirtuin-activating compound is a compound of formula 59 and the attendant definitions wherein R is H and R₁ is H.

In a further embodiment, a sirtuin-activating compound is a compound of formula 59 and the attendant definitions wherein R is H, R₁ is H, and R₂ is H.

In a further embodiment, a sirtuin-activating compound is a compound of formula 59 and the attendant definitions wherein R is H, R₁ is H, R₂ is H, and R₃ is H.

In a further embodiment, a sirtuin-activating compound is a compound of formula 59 and the attendant definitions wherein R is H, R₁ is H, R₂ is H, R₃ is H, and L is Se.

In a further embodiment, a sirtuin-activating compound is a compound of formula 59 and the attendant definitions wherein R is H, R₁ is H, R₂ is H, R₃ is H, L is Se, and n is 1.

In a further embodiment, a sirtuin-activating compound is a compound of formula 59 and the attendant definitions wherein R is H, R₁ is H, R₂ is H, R₃ is H, L is Se, n is 1, and m is 1.

In another embodiment, a sirtuin-activating compound is a compound of formula 60: 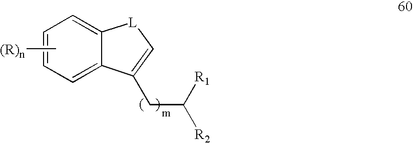
wherein, independently for each occurrence:

R is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ and R₂ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L is O, NR₃, S, or SO₂;

R₃ is H or a substituted or unsubstituted alkyl, aryl, aralkyl, heterocyclyl, heterocyclylalkyl, heteroaryl, or heteroaralkyl;

n is an integer from 0 to 4 inclusive; and

m is an integer from 1 to 5 inclusive.

In a further embodiment, a sirtuin-activating compound is a compound of formula 60 and the attendant definitions wherein n is 1.

In a further embodiment, a sirtuin-activating compound is a compound of formula 60 and the attendant definitions wherein R is Cl.

In a further embodiment, a sirtuin-activating compound is a compound of formula 60 and the attendant definitions wherein R₁ is NH₂.

In a further embodiment, a sirtuin-activating compound is a compound of formula 60 and the attendant definitions wherein R₂ is CO₂H.

In a further embodiment, a sirtuin-activating compound is a compound of formula 60 and the attendant definitions wherein L is SO₂.

In a further embodiment, a sirtuin-activating compound is a compound of formula 60 and the attendant definitions wherein m is 1.

In a further embodiment, a sirtuin-activating compound is a compound of formula 60 and the attendant definitions wherein n is 1 and R is Cl.

In a further embodiment, a sirtuin-activating compound is a compound of formula 60 and the attendant definitions wherein n is 1, R is Cl, and R₁ is NH₂.

In a further embodiment, a sirtuin-activating compound is a compound of formula 60 and the attendant definitions wherein n is 1, R is Cl, R₁ is NH₂, and R₂ is CO₂H.

In a further embodiment, a sirtuin-activating compound is a compound of formula 60 and the attendant definitions wherein n is 1, R is Cl, R₁ is NH₂, R₂ is CO₂H, and L is SO₂.

In a further embodiment, a sirtuin-activating compound is a compound of formula 60 and the attendant definitions wherein n is 1, R is Cl, R₁ is NH₂, R₂ is CO₂H, L is SO₂, and m is 1.

In another embodiment, a sirtuin-activating compound is a compound of formula 61: 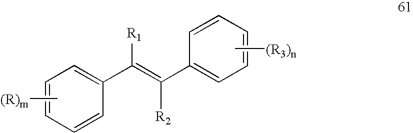
wherein, independently for each occurrence:

R, R₁, R₂, and R₃ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

n and m are integers from 0 to 5 inclusive.

In a further embodiment, a sirtuin-activating compound is a compound of formula 61 and the attendant definitions wherein n is 2.

In a further embodiment, a sirtuin-activating compound is a compound of formula 61 and the attendant definitions wherein R is 3-hydroxy and 5-hydroxy.

In a further embodiment, a sirtuin-activating compound is a compound of formula 61 and the attendant definitions wherein R₁ is H.

In a further embodiment, a sirtuin-activating compound is a compound of formula 61 and the attendant definitions wherein R₂ is H.

In a further embodiment, a sirtuin-activating compound is a compound of formula 61 and the attendant definitions wherein m is 0.

In a further embodiment, a sirtuin-activating compound is a compound of formula 61 and the attendant definitions wherein m is 1.

In a further embodiment, a sirtuin-activating compound is a compound of formula 61 and the attendant definitions wherein R₃ is 4-hydroxy.

In a further embodiment, a sirtuin-activating compound is a compound of formula 61 and the attendant definitions wherein R₃ is 4-methoxy.

In a further embodiment, a sirtuin-activating compound is a compound of formula 61 and the attendant definitions wherein n is 2 and R is 3-hydroxy and 5-hydroxy.

In a further embodiment, a sirtuin-activating compound is a compound of formula 61 and the attendant definitions wherein n is 2, R is 3-hydroxy and 5-hydroxy, and R₁ is H.

In a further embodiment, a sirtuin-activating compound is a compound of formula 61 and the attendant definitions wherein n is 2, R is 3-hydroxy and 5-hydroxy, R₁ is H, and R₂ is H.

In a further embodiment, a sirtuin-activating compound is a compound of formula 61 and the attendant definitions wherein n is 2, R is 3-hydroxy and 5-hydroxy, R₁ is H, R₂ is H, and m is 0.

In a further embodiment, a sirtuin-activating compound is a compound of formula 61 and the attendant definitions wherein n is 2, R is 3-hydroxy and 5-hydroxy, R₁ is H, R₂ is H, and m is 1.

In a further embodiment, a sirtuin-activating compound is a compound of formula 61 and the attendant definitions wherein n is 2, R is 3-hydroxy and 5-hydroxy, R₁ is H, R₂ is H, m is 1, and R₃ is 4-hydroxy.

In a further embodiment, a sirtuin-activating compound is a compound of formula 61 and the attendant definitions wherein n is 2, R is 3-hydroxy and 5-hydroxy, R₁ is H, R₂ is H, m is 1, and R₃ is 4-methoxy.

In another embodiment, a sirtuin-activating compound is a compound of formula 62: 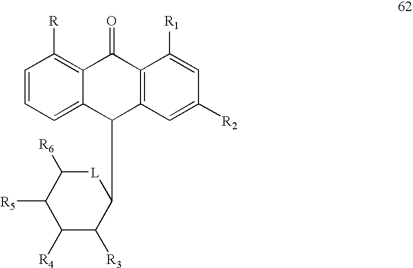
wherein, independently for each occurrence:

R, R₁, R₂, R₃, R₄, R₅, and R₆ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L is O, NR₇, or S; and

R₇ is H or a substituted or unsubstituted alkyl, aryl, aralkyl, heterocyclyl, heterocyclylalkyl, heteroaryl, or heteroaralkyl.

In a further embodiment, a sirtuin-activating compound is a compound of formula 62 and the attendant definitions wherein R is OH.

In a further embodiment, a sirtuin-activating compound is a compound of formula 62 and the attendant definitions wherein R₁ is OH.

In a further embodiment, a sirtuin-activating compound is a compound of formula 62 and the attendant definitions wherein R₂ is CH₂OH.

In a further embodiment, a sirtuin-activating compound is a compound of formula 62 and the attendant definitions wherein R₃ is OH.

In a further embodiment, a sirtuin-activating compound is a compound of formula 62 and the attendant definitions wherein R₄ is OH.

In a further embodiment, a sirtuin-activating compound is a compound of formula 62 and the attendant definitions wherein R₅ is OH.

In a further embodiment, a sirtuin-activating compound is a compound of formula 62 and the attendant definitions wherein R₆ is CH₂OH.

In a further embodiment, a sirtuin-activating compound is a compound of formula 62 and the attendant definitions wherein L is O.

In a further embodiment, a sirtuin-activating compound is a compound of formula 62 and the attendant definitions wherein R is OH and R₁ is OH.

In a further embodiment, a sirtuin-activating compound is a compound of formula 62 and the attendant definitions wherein R is OH, R₁ is OH, and R₂ is CH₂OH.

In a further embodiment, a sirtuin-activating compound is a compound of formula 62 and the attendant definitions wherein R is OH, R₁ is OH, R₂ is CH₂OH, and R₃ is OH.

In a further embodiment, a sirtuin-activating compound is a compound of formula 62 and the attendant definitions wherein R is OH, R₁ is OH, R₂ is CH₂OH, R₃ is OH, and R₄ is OH.

In a further embodiment, a sirtuin-activating compound is a compound of formula 62 and the attendant definitions wherein R is OH, R₁ is OH, R₂ is CH₂OH, R₃ is OH, R₄ is OH, and R₅ is OH.

In a further embodiment, a sirtuin-activating compound is a compound of formula 62 and the attendant definitions wherein R is OH, R₁ is OH, R₂ is CH₂OH, R₃ is OH, R₄ is OH, R₅ is OH, and R₆ is CH₂OH.

In a further embodiment, a sirtuin-activating compound is a compound of formula 62 and the attendant definitions wherein R is OH, R₁ is OH, R₂ is CH₂OH, R₃ is OH, R₄ is OH, R₅ is OH, R₆ is CH₂OH, and L is O.

In another embodiment, a sirtuin-activating compound is a compound of formula 63: 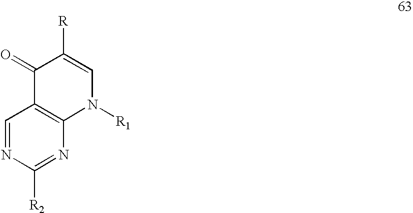
wherein, independently for each occurrence:

R, R₁, and R₂ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl.

In a further embodiment, a sirtuin-activating compound is a compound of formula 63 and the attendant definitions wherein R is CO₂H.

In a further embodiment, a sirtuin-activating compound is a compound of formula 63 and the attendant definitions wherein R₁ is ethyl.

In a further embodiment, a sirtuin-activating compound is a compound of formula 63 and the attendant definitions wherein R₂ is N-1-pyrrolidine.

In a further embodiment, a sirtuin-activating compound is a compound of formula 63 and the attendant definitions wherein R is CO₂H and R₁ is ethyl.

In a further embodiment, a sirtuin-activating compound is a compound of formula 63 and the attendant definitions wherein R is CO₂H and R₂ is N-1-pyrrolidine.

In a further embodiment, a sirtuin-activating compound is a compound of formula 63 and the attendant definitions wherein R₁ is ethyl and R₂ is N-1-pyrrolidine.

In a further embodiment, a sirtuin-activating compound is a compound of formula 63 and the attendant definitions wherein R is CO₂H, R₁ is ethyl, and R₂ is N-1-pyrrolidine.

In another embodiment, a sirtuin-activating compound is a compound of formula 64: 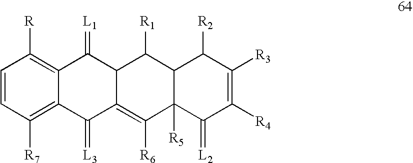
wherein, independently for each occurrence:

R, R₁, R₂, R₃, R₄, R₅, R₆, and R₇ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are CH₂, O, NR₈, or S; and

R₈ is H or a substituted or unsubstituted alkyl, aryl, aralkyl, heterocyclyl, heterocyclylalkyl, heteroaryl, or heteroaralkyl.

In a further embodiment, a sirtuin-activating compound is a compound of formula 64 and the attendant definitions wherein R is Cl.

In a further embodiment, a sirtuin-activating compound is a compound of formula 64 and the attendant definitions wherein R is H.

In a further embodiment, a sirtuin-activating compound is a compound of formula 64 and the attendant definitions wherein R₁ is OH.

In a further embodiment, a sirtuin-activating compound is a compound of formula 64 and the attendant definitions wherein R₂ is N(Me)₂.

In a further embodiment, a sirtuin-activating compound is a compound of formula 64 and the attendant definitions wherein R₃ is OH.

In a further embodiment, a sirtuin-activating compound is a compound of formula 64 and the attendant definitions wherein R₄ is C(O)NH₂.

In a further embodiment, a sirtuin-activating compound is a compound of formula 64 and the attendant definitions wherein R₅ is OH.

In a further embodiment, a sirtuin-activating compound is a compound of formula 64 and the attendant definitions wherein R₆ is OH.

In a further embodiment a sirtuin-activating compound is a compound of formula 64 and the attendant definitions wherein R₇ is OH.

In a further embodiment, a sirtuin-activating compound is a compound of formula 64 and the attendant definitions wherein L₁ is CH₂.

In a further embodiment, a sirtuin-activating compound is a compound of formula 64 and the attendant definitions wherein L₂ is O.

In a further embodiment, a sirtuin-activating compound is a compound of formula 64 and the attendant definitions wherein L₃ is O.

In a further embodiment, a sirtuin-activating compound is a compound of formula 64 and the attendant definitions wherein R is Cl and R₁ is OH.

In a further embodiment, a sirtuin-activating compound is a compound of formula 64 and the attendant definitions wherein R is Cl, R₁ is OH, and R₂ is N(Me)₂.

In a further embodiment, a sirtuin-activating compound is a compound of formula 64 and the attendant definitions wherein R is Cl, R₁ is OH, R₂ is N(Me)₂, and R₃ is OH.

In a further embodiment, a sirtuin-activating compound is a compound of formula 64 and the attendant definitions wherein R is Cl, R₁ is OH, R₂ is N(Me)₂, R₃ is OH, and R₄ is C(O)NH₂.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, and R₅ is OH.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, R₅ is OH, and R₆ is OH.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, R₅ is OH, R₆ is OH, and R₇ is OH.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, and L₁ is CH₂.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, L₁ is CH₂, and L₂ is O.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, L₁ is CH₂, L₂ is O, and L₃ is O.

In a further embodiment, a sirtuin-activating compound is a compound of formula 64 and the attendant definitions wherein R is H and R₁ is OH.

In a further embodiment, a sirtuin-activating compound is a compound of formula 64 and the attendant definitions wherein R is H, R₁ is OH, and R₂ is N(Me)₂.

In a further embodiment, a sirtuin-activating compound is a compound of formula 64 and the attendant definitions wherein R is H, R₁ is OH, R₂ is N(Me)₂, and R₃ is OH.

In a further embodiment, a sirtuin-activating compound is a compound of formula 64 and the attendant definitions wherein R is H, R₁ is OH, R₂ is N(Me)₂, R₃ is OH, and R₄ is C(O)NH₂.

In a further embodiment, a sirtuin-activating compound is a compound of formula 64 and the attendant definitions wherein R is H, R₁ is OH, R₂ is N(Me)₂, R₃ is OH, R₄ is C(O)NH₂, and R₅ is OH.

In a further embodiment, a sirtuin-activating compound is a compound of formula 64 and the attendant definitions wherein R is H, R₁ is OH, R₂ is N(Me)₂, R₃ is OH, R₄ is C(O)NH₂, R₅ is OH, and R₆ is OH.

In a further embodiment, a sirtuin-activating compound is a compound of formula 64 and the attendant definitions wherein R is H, R₁ is OH, R₂ is N(Me)₂, R₃ is OH, R₄ is C(O)NH₂, R₅ is OH, R₆ is OH, and R₇ is OH.

In a further embodiment, a sirtuin-activating compound is a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, and L₁ is CH₂.

In a further embodiment, a sirtuin-activating compound is a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, L₁ is CH₂, and L₂ is O.

In a further embodiment, a sirtuin-activating compound is a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, L₁ is CH₂, L₂ is O, and L₃ is O.

In another embodiment, a sirtuin-activating compound is a compound of formula 65: 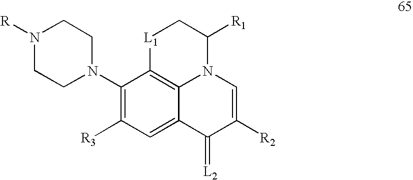
wherein, independently for each occurrence:

R is H or a substituted or unsubstituted alkyl, aryl, aralkyl, heterocyclyl, heterocyclylalkyl, heteroaryl, or heteroaralkyl;

R₁, R₂, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl; and

L₁ and L₂ are O, NR, or S.

In a further embodiment, a sirtuin-activating compound is a compound of formula 65 and the attendant definitions wherein R is methyl.

In a further embodiment, a sirtuin-activating compound is a compound of formula 65 and the attendant definitions wherein R₁ is methyl.

In a further embodiment, a sirtuin-activating compound is a compound of formula 65 and the attendant definitions wherein R₂ is CO₂H.

In a further embodiment, a sirtuin-activating compound is a compound of formula 65 and the attendant definitions wherein R₃ is F.

In a further embodiment, a sirtuin-activating compound is a compound of formula 65 and the attendant definitions wherein L₁ is O.

In a further embodiment, a sirtuin-activating compound is a compound of formula 65 and the attendant definitions wherein L₂ is O.

In a further embodiment, a sirtuin-activating compound is a compound of formula 65 and the attendant definitions wherein R is methyl and R₁ is methyl.

In a further embodiment, a sirtuin-activating compound is a compound of formula 65 and the attendant definitions wherein R is methyl, R₁ is methyl, and R₂ is CO₂H.

In a further embodiment, a sirtuin-activating compound is a compound of formula 65 and the attendant definitions wherein R is methyl, R₁ is methyl, R₂ is CO₂H, and R₃ is F.

In a further embodiment, a sirtuin-activating compound is a compound of formula 65 and the attendant definitions wherein R is methyl, R₁ is methyl, R₂ is CO₂H, R₃ is F, and L₁ is O.

In a further embodiment, a sirtuin-activating compound is a compound of formula 65 and the attendant definitions wherein R is methyl, R₁ is methyl, R₂ is CO₂H, R₃ is F, L₁ is O, and L₂ is O.

Exemplary activating compounds are those listed in the appended Tables having a ratio to control rate of more than one. A preferred compound of formula 8 is Dipyridamole; a preferred compound of formula 12 is Hinokitiol; a preferred compound of formula 13 is L-(+)-Ergothioneine; a preferred compound of formula 19 is Caffeic Acid Phenol Ester; a preferred compound of formula 20 is MCI-186 and a preferred compound of formula 21 is HBED (Supplementary Table 6). Activating compounds may also be oxidized forms of the compounds of Table 21.

Also included are pharmaceutically acceptable addition salts and complexes of the compounds of formulas 1-25, 30, 32-65, and 69-88. In cases wherein the compounds may have one or more chiral centers, unless specified, the compounds contemplated herein may be a single stereoisomer or racemic mixtures of stereoisomers.

In one embodiment, a sirtuin-activating compound is a stilbene, chalcone, or flavone compound represented by formula 7:

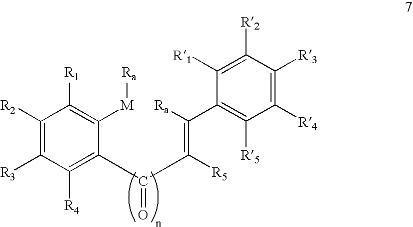

wherein, independently for each occurrence,

M is absent or O;

R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;

Rₐ represents H or the two instances of Rₐ form a bond;

R represents H, alkyl, or aryl; and

n is 0 or 1.

In a further embodiment, a sirtuin-activating compound is a compound represented by formula 7 and the attendant definitions, wherein n is 0. In a further embodiment, a sirtuin-activating compound is a compound represented by formula 7 and the attendant definitions, wherein n is 1. In a further embodiment, a sirtuin-activating compound is a compound represented by formula 7 and the attendant definitions, wherein M is absent. In a further embodiment, a sirtuin-activating compound is a compound represented by formula 7 and the attendant definitions, wherein M is O. In a further embodiment, a sirtuin-activating compound is a compound represented by formula 7 and the attendant definitions, wherein Rₐ is H. In a further embodiment, a sirtuin-activating compound is a compound represented by formula 7 and the attendant definitions, wherein M is O and the two Rₐ form a bond. In a further embodiment, a sirtuin-activating compound is a compound represented by formula 7 and the attendant definitions, wherein R₅ is H. In a further embodiment, a sirtuin-activating compound is a compound represented by formula 7 and the attendant definitions, wherein R₅ is OH. In a further embodiment, a sirtuin-activating compound is a compound represented by formula 7 and the attendant definitions, wherein R₁, R₃, and R′₃ are OH. In a further embodiment, a sirtuin-activating compound is a compound represented by formula 7 and the attendant definitions, wherein R₂, R₄, R′₂, and R′₃ are OH. In a further embodiment, a sirtuin-activating compound is a compound represented by formula 7 and the attendant definitions, wherein R₂, R′₂, and R′₃ are OH.

In a further embodiment, a sirtuin-activating compound is a compound represented by formula 7 and the attendant definitions, wherein n is O; M is absent; Rₐ is H; R₅ is H; R₁, R₃, and R′₃ are OH; and R₂, R₄, R′₁, R′₂, R′₄, and R′₅ are H. In a further embodiment, a sirtuin-activating compound is a compound represented by formula 7 and the attendant definitions, wherein n is 1; M is absent; Rₐ is H; R₅ is H; R₂, R₄, R′₂, and R′₃ are OH; and R₁, R₃, R′₁, R′₄, and R′₅ are H. In a further embodiment, a sirtuin-activating compound is a compound represented by formula 7 and the attendant definitions, wherein n is 1; M is O; the two Rₐ form a bond; R₅ is OH; R₂, R′₂, and R′₃ are OH; and R₁, R₃, R₄, R′₁, R′₄, and R′₅ are H.

In another embodiment, exemplary sirtuin-activating compounds are isonicotinamide analogs, such as, for example, the isonicotinamide analogs described in U.S. Pat. Nos. 5,985,848; 6,066,722; 6,228,847; 6,492,347; 6,803,455; and U.S. Patent Publication Nos. 2001/0019823; 2002/0061898; 2002/0132783; 2003/0149261; 2003/0229033; 2003/0096830; 2004/0053944; 2004/0110772; and 2004/0181063, the disclosures of which are hereby incorporated by reference in their entirety. In an exemplary emobidment, sirtuin-activating compounds may be an isonicotinamide analog having any of formulas 69-72 below. In one embodiment, a sirtuin-activating compound is an isonicotinamide analog compound of formula 69:

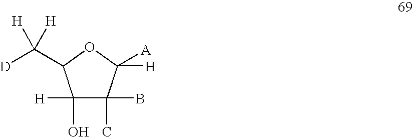

Wherein A is a nitrogen-, oxygen-, or sulfur-linked aryl, alkyl, cyclic, or heterocyclic group. The A moieties thus described, optionally have leaving group characteristics. In embodiments encompassed herein, A is further substituted with an electron contributing moiety. B and C are both hydrogen, or one of B or C is a halogen, amino, or thiol group and the other of B or C is hydrogen; and D is a primary alcohol, a hydrogen, or an oxygen, nitrogen, carbon, or sulfur linked to phosphate, a phosphoryl group, a pyrophosphoryl group, or adenosine monophosphate through a phosphodiester or carbon-, nitrogen-, or sulfur-substituted phosphodiester bridge, or to adenosine diphosphate through a phosphodiester or carbon-, nitrogen-, or sulfur-substituted pyrophosphodiester bridge.

In one example, A is a substituted N-linked aryl or heterocyclic group, an O-linked aryl or heterocyclic group having the formula —O—Y, or an S-linked aryl or heterocyclic group having the formula —O—Y; both B and C are hydrogen, or one of B or C is a halogen, amino, or thiol group and the other of B or C is hydrogen; and D is a primary alcohol or hydrogen. Nonlimiting preferred examples of A are set forth below, where each R is H or an electron-contributing moiety and Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl.

Examples of A include i-xiv below: 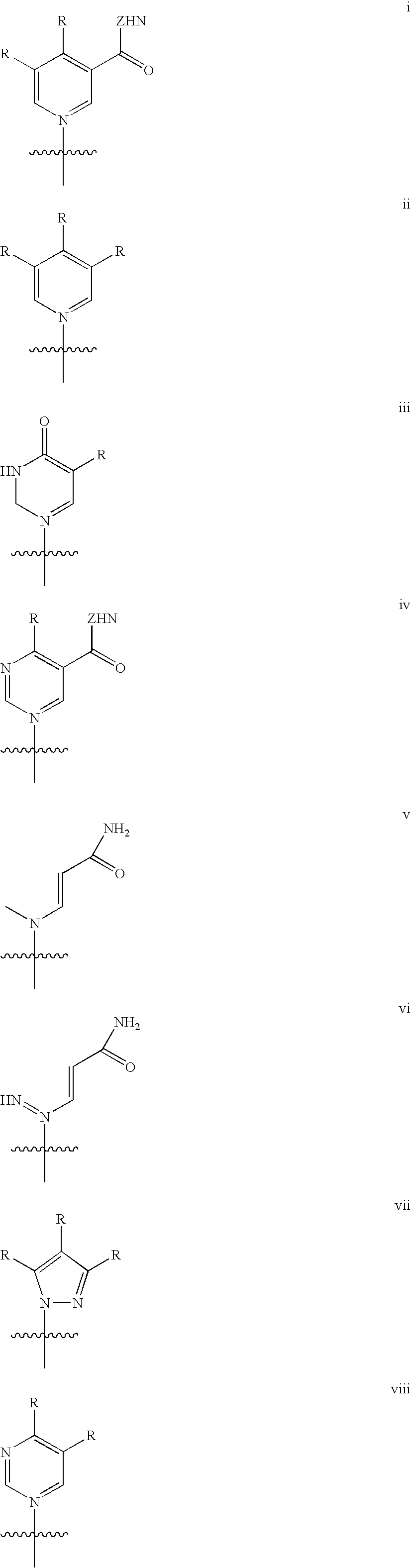 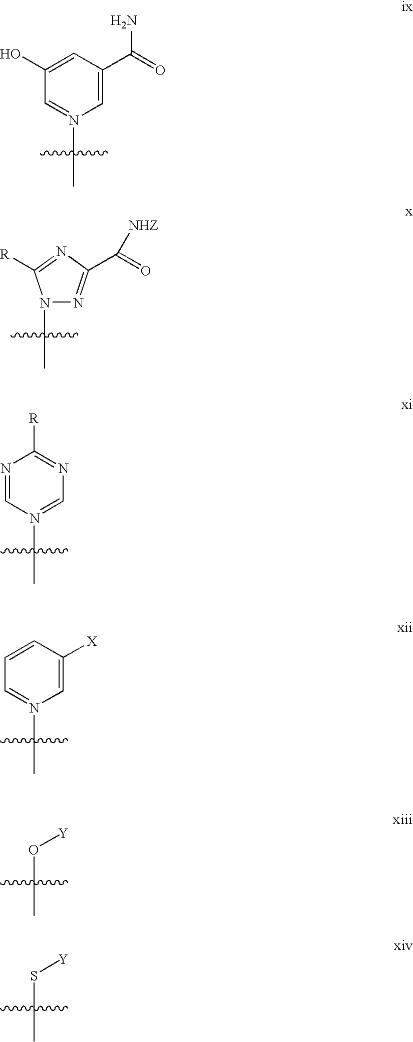
where Y=a group consistent with leaving group function.

Examples of Y include, but are not limited to, xv-xxvii below:

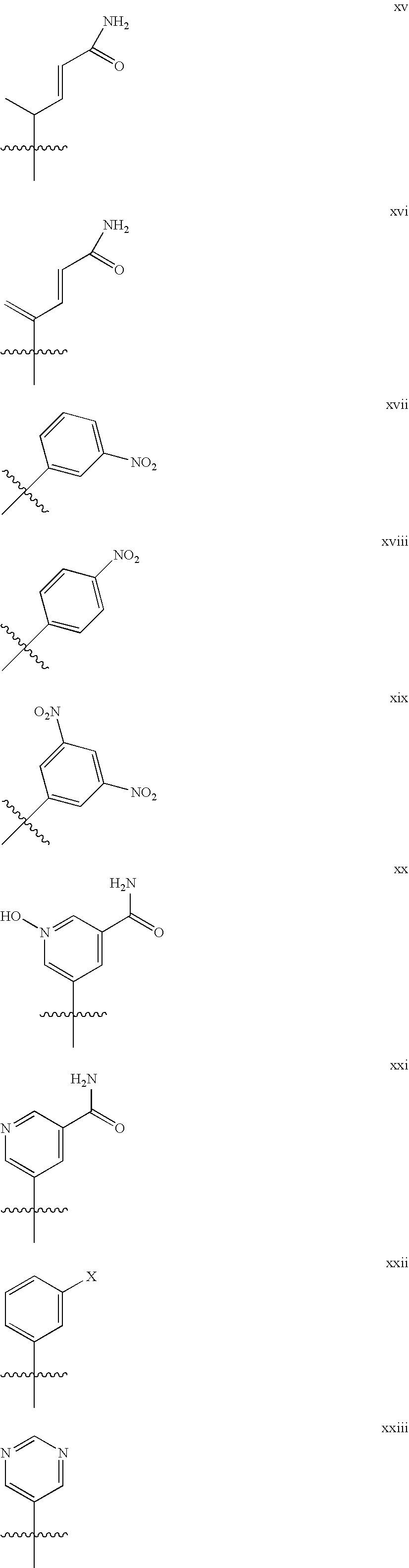

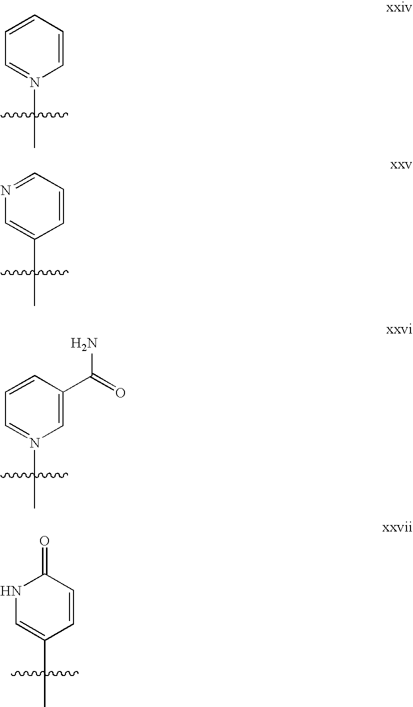

Wherein, for i-xxvii, X is halogen, thiol, or substituted thiol, amino or substituted amino, oxygen or substituted oxygen, or aryl or alkyl groups or heterocycles.

In certain embodiments, A is a substituted nicotinamide group (i above, where Z is H), a substituted pyrazolo group (vii above), or a substituted 3-carboxamid-imidazolo group (x above, where Z is H). Additionally, both B and C may be hydrogen, or one of B or C is a halogen, amino, or thiol group and the other of B or C is hydrogen; and D is a primary alcohol or hydrogen.

In other embodiments, one of B or C may be halogen, amino, or thiol group when the other of B or C is a hydrogen. Furthermore, D may be a hydrogen or an oxygen, nitrogen, carbon, or sulfur linked to phosphate, a phosphoryl group, a pyrophosphoryl group, or adenosine monophosphate through a phosphodiester or carbon-, nitrogen-, or sulfur-substituted phosphodiester bridge, or to adenosine diphosphate through a phosphodiester or carbon-, nitrogen-, or sulfur-substituted pyrophosphodiester bridge. Analogues of adenosine monophosphate or adenosine diphosphate also can replace the adenosine monophosphate or adenosine diphosphate groups.

In some embodiments, A has two or more electron contributing moieties.

In other embodiments, a sirtuin-activating compound is an isonicotinamide analog compound of formulas 70, 71, or 72 below. 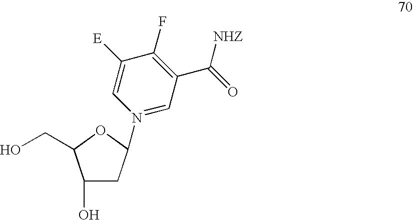
wherein Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl; E and F are independently H, CH₃, OCH.sub.3, CH₂CH₃, NH₂, OH, NHCOH, NHCOCH₃, N(CH₃)₂, C(CH₃)₂, an aryl or a C3-C10 alkyl, preferably provided that, when one of of E or F is H, the other of E or F is not H; 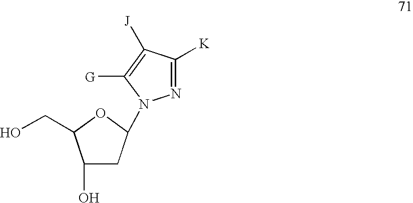
wherein G, J or K is CONHZ, Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl, and the other two of G, J and K is independently CH₃, OCH₃, CH₂CH₃, NH₂, OH, NHCOH, NHCOCH₃; 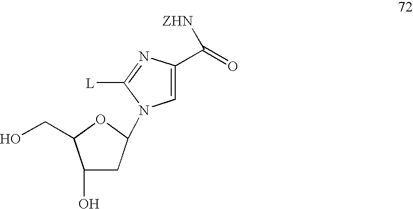
wherein Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl; and L is CH₃, OCH₃, CH₂CH₃, NH₂, OH, NHCOH, NHCOCH₃.

In an exemplary embodiment, the compound is formula 70 above, wherein E and F are independently H, CH₃, OCH₃, or OH, preferably provided that, when one of E or F is H, the other of E or F is not H.

In another exemplary embodiment, the compound is β-1′-5-methyl-nicotinamide-2′-deoxyribose, β-D-1′-5-methyl-nico-tinamide-2′-deoxyribofuranoside, β-1′-4,5-dimethyl-nicotinamide-2′-de-oxyribose or β-D-1′-4,5-dimethyl-nicotinamide-2′-deoxyribofuranoside.

In yet another embodiment, the compound is β-1′-5-methyl-nicotinamide-2′-deoxyribose.

Without being bound to any particular mechanism, it is believed that the electron-contributing moiety on A stabilizes the compounds of the invention such that they are less susceptible to hydrolysis from the rest of the compound. This improved chemical stability improves the value of the compound, since it is available for action for longer periods of time in biological systems due to resistance to hydrolytic breakdown. The skilled artisan could envision many electron-contributing moieties that would be expected to serve this stabilizing function. Non-limiting examples of suitable electron contributing moieties are methyl, ethyl, O-methyl, amino, NMe₂, hydroxyl, CMe₃, aryl and alkyl groups. Preferably, the electron-contributing moiety is a methyl, ethyl, O-methyl, amino group. In the most preferred embodiments, the electron-contributing moiety is a methyl group.

The compounds of formulas 69-72 are useful both in free form and in the form of salts. The term “pharmaceutically acceptable salts” is intended to apply to non-toxic salts derived from inorganic or organic acids and includes, for example, salts derived from the following acids: hydrochloric, sulfuric, phosphoric, acetic, lactic, fumaric, succinic, tartaric, gluconic, citric, methanesulfonic, and p-toluenesulfonic acids.

Also provided are compounds of formulas 69-72 that are the tautomers, pharmaceutically-acceptable salts, esters, and pro-drugs of the inhibitor compounds disclosed herein.

The biological availability of the compounds of formulas 69-72 can be enhanced by conversion into a pro-drug form. Such a pro-drug can have improved lipophilicity relative to the unconverted compound, and this can result in enhanced membrane permeability. One particularly useful form of pro-drug is an ester derivative. Its utility relies upon the action of one or more of the ubiquitous intracellular lipases to catalyse the hydrolysis of ester groups, to release the active compound at or near its site of action. In one form of pro-drug, one or more hydroxy groups in the compound can be O-acylated, to make an acylate derivative.

Pro-drug forms of a 5-phosphate ester derivative of compounds of formulas 69-72 can also be made. These may be particularly useful, since the anionic nature of the 5-phosphate may limit its ability to cross cellular membranes. Conveniently, such a 5-phosphate derivative can be converted to an uncharged bis(acyloxymethyl) ester derivative. The utility of such a pro-drug relies upon the action of one or more of the ubiquitous intracellular lipases to catalyse the hydrolysis of ester groups, releasing a molecule of formaldehyde and a compound of the present invention at or near its site of action. Specific examples of the utility of, and general methods for making, such acyloxymethyl ester pro-drug forms of phosphorylated carbohydrate derivatives have been described (Kang et al., 1998; Jiang et al., 1998; Li et al., 1997; Kruppa et al., 1997).

In another embodiment, exemplary sirtuin-activating compounds are O-acetyl-ADP-ribose analogs, including 2′-O-acetyl-ADP-ribose and 3′-O-acetyl-ADP-ribose, and analogs thereof. Exemplary O-acetyl-ADP-ribose analogs are described, for example, in U.S. Patent Publication Nos. 2004/0053944; 2002/0061898; and 2003/0149261, the disclosures of which are hereby incorporated by reference in their entirety. In an exemplary emobidment, sirtuin-activating compounds may be an O-acetyl-ADP-ribose analog having any of formulas 73-76 below. In one embodiment, a sirtuin-activating compound is an O-acetyl-ADP-ribose analog compound of formula 73: 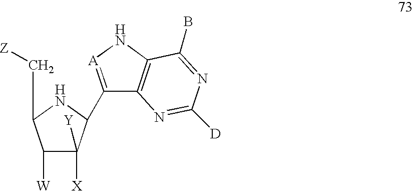
wherein:

A is selected from N, CH and CR, where R is selected from halogen, optionally substituted alkyl, aralkyl and aryl, OH, NH₂, NHR¹, NR¹R² and SR³, where R¹, R² and R³ are each optionally substituted alkyl, aralkyl or aryl groups;

B is selected from OH, NH₂, NHR⁴, H and halogen, where R⁴ is an optionally substituted alkyl, aralkyl or aryl group;

D is selected from OH, NH₂, NHR⁵, H, halogen and SCH₃, where R⁵ is an optionally substituted alkyl, aralkyl or aryl group;

X and Y are independently selected from H, OH and halogen, with the proviso that when one of X and Y is hydroxy or halogen, the other is hydrogen;

Z is OH, or, when X is hydroxy, Z is selected from hydrogen, halogen, hydroxy, SQ and OQ, where Q is an optionally substituted alkyl, aralkyl or aryl group; and

W is OH or H, with the proviso that when W is OH, then A is CR where R is as defined above;

or a tautomer thereof; or a pharmaceutically acceptable salt thereof; or an ester thereof; or a prodrug thereof.

In certain embodiments, when B is NHR₄ and/or D is NHR⁵, then R⁴ and/or R⁵ are C1-C4 alkyl.

In other embodiments, when one or more halogens are present they are chosen from chlorine and fluorine.

In another embodiment, when Z is SQ or OQ, Q is C1-C5 alkyl or phenyl.

In an exemplary embodiment, D is H, or when D is other than H, B is OH.

In another embodiment, B is OH, D is H, OH or NH₂, X is OH or H, Y is H, most preferably with Z as OH, H, or methylthio, especially OH.

In certain embodiments W is OH, Y is H, X is OH, and A is CR where R is methyl or halogen, preferably fluorine.

In other embodiments, W is H, Y is H, X is OH and A is CH.

In other embodiments, a sirtuin-activating compound is an O-acetyl-ADP-ribose analog compound of formula 74:

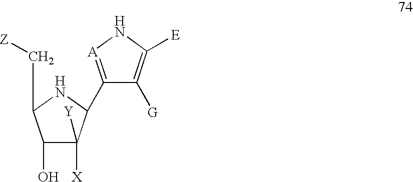

wherein A, X, Y, Z and R are defined for compounds of formula (73) where first shown above; E is chosen from CO₂H or a corresponding salt form, CO₂R, CN, CONH₂, CONHR or CONR₂; and G is chosen from NH₂, NHCOR, NHCONHR or NHCSNHR; or a tautomer thereof, or a pharmaceutically acceptable salt thereof, or an ester thereof, or a prodrug thereof.

In certain embodiments, E is CONH₂ and G is NH₂.

In other embodiments, E is CONH₂, G is NH₂, X is OH or H, is H, most preferable with Z as OH, H or methylthio, especially OH.

Exemplary sirtuin-activating compounds include the following:

(1S)-1,4-dideoxy-1-C-(4-hydroxypyrrolo[3,2-d]pyrimidin-7-yl)-1,4-imino-D-ribitol

(1S)-1-C-(2-amino-4-hydroxypyrrolo[3,2-d]pyrimidin-7-yl)-1,4-dideoxy-1,4-imino-D-ribitol

(1R)-1-C-(4-hydroxypyrrolo[3,2-d]pyrimidin-7-yl)-1,4-imino-1,2,4-trideoxy-D-erythro-pentitol

(1S)-1-C-(4-hydroxypyrrolo[3,2-d]pyrimidin-7-yl)-1,4-imino-1,4,5-trideoxy-D-ribitol

(1S)-1,4-dideoxy-1-C-(4-hydroxypyrrolo[3,2-d]pyrimidin-7-yl)-1,4-imino-5-methylthio-D-ribitol

(1S)-1,4-dideoxy-1-C-(2,4-dihydroxypyrrolo[3,2-d]pyrimidin-7-yl)-1,4-imino-D-ribitol

(1R)-1-C-(2,4-dihydroxypyrrolo[3,2-d]pyrimidin-7-yl)-1,4-imino-1,2,4-trideoxy-D-erthro-pentitol

(1S)-1-C-(2,4-dihydroxypyrrolo[3,2-d]pyrimidin-7-yl)-1,4-imino-1,4,5-trideoxy-D-ribitol

(1S)-1,4-dideoxy-1-C-(2,4-dihydroxypyrrolo[3,2-d]pyrimidin-7-yl)-1,4-imino-5-ethylthio-D-ribitol

(1R)-1-C-(2-amino4-hydroxypyrrolo[3,2-d]pyrimidin-7-yl)-1,4-imino-1,2,4-trideoxy-D-erythro-pentitol

(1S)-1-C-(2-amino4-hydroxypyrrolo[3,2-d]pyrimidin-7-yl)-1,4-imino-1,4,5-trideoxy-D-ribitol

(1S)-1-C-(2-amino-4-hydroxypyrrolo[3,2-d]pyrimidin-7-yl)-1,4-dideoxy-1,4-imino-5-methylthio-D-ribitol

(1S)-1,4-dideoxy-1-C-(7-hydroxypyrazolo[4,3-d]pyrimidin-3-yl)-1,4-imino-D-ribitol

(1R)-1-C-(7-hydroxypyrazolo[4,3-d]pyrimidin-3-yl)-1,4-imino-1,2,4-trideoxy-D-erythro-pentitol

(1S)-1-C-(7-hydroxypyrazolo[4,3-d]pyrimidin-3-yl)-1,4-imino-1,4,5-trideoxy-D-ribitol

(1S)-1,4-dideoxy-1-C-(7-hydroxypyrazolo[4,3-d]pyrimidin-3-yl)-1,4-imino-5-ethylthio-D-ribitol

(1S)-1,4-dideoxy-1-C-(5,7-dihydroxypyrazolo[4,3-d]pyrimidin-3-yl)-1,4-imino-D-ribitol

(1R)-1-C-(5,7-dihydroxypyrazolo[4,3-d]pyrimidin-3-yl)-1,4-imino-1,2,4-trideoxy-D-erythro-pentitol

(1S)-1-C-(5,7-dihydroxypyrazolo[4,3-d]pyrimidin-3-yl)-1,4-imino-1,4,5-trideoxy-D-ribitol

(1S)-1,4-dideoxy-1-C-(5,7-dihydroxypyrazolo[4,3-d]pyrimidin-3-yl)-1,4-imino-5-methylthio-D-ribitol

(1S)-1-C-(5-amino-7-hydroxypyrazolo[4,3-d]pyrimidin-3-yl)-1,4-dideoxy-1,4-imino-D-ribitol

(1R)-1-C-(S-amino-7-hydroxypyrazolo[4,3-d]pyrimidin-3-yl)-1,4-imino-1,2,4-trideoxy-D-erythro-pentitol

(1S)-1-C-(5-amino-7-hydroxypyrazolo[4,3-d]pyrimidin-3-yl)-1,4-imino-1,4,5-trideoxy-D-ribitol

(1S)-1-C-(5-amino-7-hydroxypyrazolo[4,3-d]pyrimidin-3-yl)-1,4-dideoxy-1,4-imino-5-methylthio-D-ribitol

(1S)-1-C-(3-amino-2-carboxamido-4-pyrroly)-1,4-dideoxy-1,4-imino-D-ribitol.

(1S)-1,4-dideoxy-1-C-(4-hydroxypyrrolo[3,2-d]pyrimidin-7-yl)-1,4-imino-D-ribitol 5-phosphate

(1S)-1-C-(2-amino4-hydroxypyrrolo[3,2-d]pyrimidin-7-yl)-1,4-imino-D-ribitol 5-phosphate

(1S)-1-C-(3-amino-2-carboxamido-4-pyrrolyl)-1,4-dideoxy-1,4-imino-D-ribitol

In yet other embodiments, sirtuin-activating compounds are O-acetyl-ADP-ribose analog compounds of formula 75 and 76, their tautomers and pharmaceutically acceptable salts.

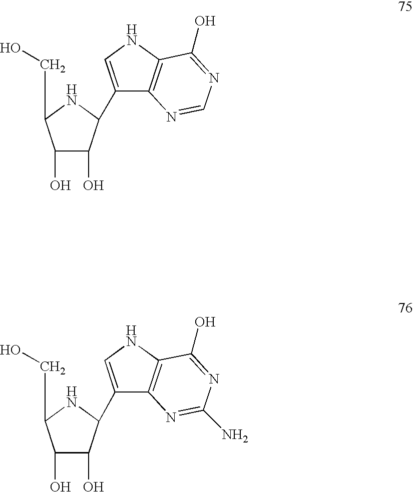

The biological availability of a compound of formula (75) or formula (76) can be enhanced by conversion into a pro-drug form. Such a pro-drug can have improved lipophilicity relative to the compound of formula (75) or formula (76), and this can result in enhanced membrane permeability. One particularly useful form of a pro-drug is an ester derivative. Its utility relies upon the action of one or more of the ubiquitous intracellular lipases to catalyse the hydrolysis of these ester group(s), to release the compound of formula (75) and formula (76) at or near its site of action.

In one form of a prodrug, one or more of the hydroxy groups in a compound of formula (75) or formula (76) can be O-acylated, to make, for example a 5-O-butyrate or a 2,3-di-O-butyrate derivative.

Prodrug forms of 5-phosphate ester derivative of a compounds of formula (75) or formula (76) can also be made and may be particularly useful, since the anionic nature of the 5-phosphate may limit its ability to cross cellular membranes. Conveniently, such a 5-phosphate derivative can be converted to an uncharged bis(acyloxymethyl) ester derivative. The utility of such a pro-drug relies upon the action of one or more of the ubiquitous intracellular lipases to catalyse the hydrolysis of these ester group(s), releasing a molecule of formaldehyde and the compound of formula (75) or formula (76) at or near its site of action.

In an exemplary embodiment, analogs of 2′-AADPR or 3′-AADPR that are designed to have increased stability from esterase action through the use of well-known substitutes for ester oxygen atoms that are subject to esterase attack. The esterase-labile oxygen atoms in 2′-AADPR and 3′-AADPR would be understood to be the ester oxygen linking the acetate group with the ribose, and the ester oxygen between the two phosphorus atoms. As is known in the art, substitution of either or both of these ester oxygen atoms with a CF₂, a NH, or a S would be expected to provide a 2′-AADPR or 3′-AADPR analog that is substantially more stable due to increased resistance to esterase action.

Thus, in some embodiments, the invention is directed to analogs 2′-O-acetyl-ADP-ribose or 3′-O-acetyl-ADP-ribose exhibiting increased stability in cells. The preferred analogs comprise a CF₂, a NH, or a S instead of the acetyl ester oxygen or the oxygen between two phosphorus atoms. The most preferred substitute is CF₂. Replacement of the acetyl ester oxygen is particularly preferred. In other preferred embodiments, both the ester oxygen and the oxygen between the two phosphorus atoms are independently substituted with a CF₂, a NH, or a S.

Also included are pharmaceutically acceptable addition salts and complexes of the sirtuin-activity compounds described herein. In cases wherein the compounds may have one or more chiral centers, unless specified, the compounds contemplated herein may be a single stereoisomer or racemic mixtures of stereoisomers.

In one embodiment, sirtuin modulators for use in the invention are represented by Formula 77 or 78:

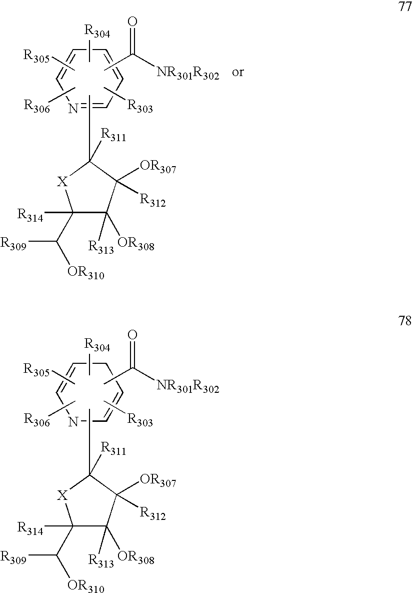

or a pharmaceutically acceptable salt thereof, where:

R₃₀₁ and R₃₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₃₀₁ and R₃₀₂ taken together form a substituted or unsubstituted non-aromatic heterocyclic group;

R₃₀₃, R₃₀₄, R₃₀₅ and R₃₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₃₀₇, R₃₀₈ and R₃₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₃₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₃₁₁, R₃₁₂, R₃₁₃ and R₃₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

A group of suitable compounds encompassed by Formulas 77 and 78 is represented by Structural Formulas 79 and 80:

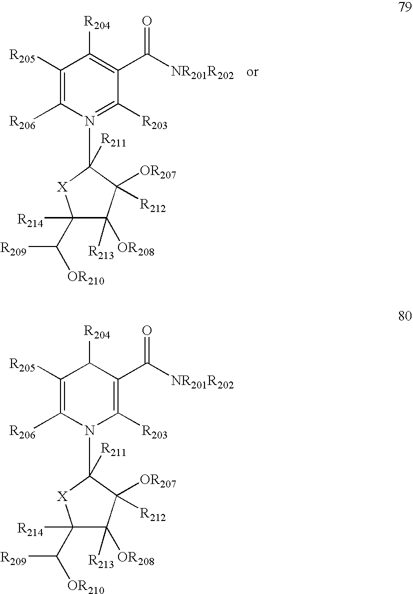

or a pharmaceutically acceptable salt thereof, where:

R₂₀₁ and R₂₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₂₀₁ and R₂₀₂ taken together form a substituted or unsubstituted non-aromatic heterocyclic group;

R₂₀₃, R₂₀₄, R₂₀₅ and R₂₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₂₀₇, R₂₀₈ and R₂₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₂₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₂₁₁, R₂₁₂, R₂₁₃ and R₂₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S, preferably O; and

n is 1 or 2.

In a particular group of compounds represented by Formula 79 or 80, at least one of R₂₀₇, R₂₀₈ and R₂₁₀ is a substituted or unsubstituted alkyl group, a substituted or unsubstituted aryl group, —C(O)R, —C(O)OR, —C(O)NHR, —C(S)R, —C(S)OR or —C(O)SR. Typically, at least one of R₂₀₇, R₂₀₈ and R₂₁₀ is —C(O)R or —C(O)OR. More typically, at least one of R₂₀₇, R₂₀₈ and R₂₁₀ is —C(O)R. In such compounds, R is preferably a substituted or unsubstituted alkyl, particularly an unsubstituted alkyl group such as methyl or ethyl.

In another particular group of compounds represented by Formula 79 or 80, R₂₀₄ is a halogen (e.g., fluorine, bromine, chlorine) or hydrogen (including a deuterium and/or tritium isotope). Suitable compounds include those where at least one of R₂₀₇, R₂₀₈ and R₂₁₀ is a substituted or unsubstituted alkyl group, a substituted or unsubstituted aryl group, —C(O)R, —C(O)OR, —C(O)NHR, —C(S)R, —C(S)OR or —C(O)SR and R₂₀₄ is a halogen or hydrogen.

Typically, for compounds represented by Formulas 79 and 80, R₂₀₃—R₂06 are —H. In addition, R₂₀₉ and R₂₁₁—R₂₁₄ are typically —H. Particular compounds represented by Formulas 79 and 80 are selected such that R₂₀₃—R₂₀₆, R₂₀₉ and R₂₁₁—R₂₁₄ are all —H. For these compounds, R₂₀₄, R₂₀₇, R₂₀₈ and R₂₁₀ have the values described above.

R₂₀₁, and R₂₀₂ are typically —H or a substituted or unsubstituted alkyl group, more typically —H. In compounds having these values of R₂₀₁ and R₂₀₂, R₂₀₃—R₂₀₆, R₂₀₉ and R₂₁₁—R₂₁₄ typically have the values described above.

In certain methods of the invention, at least one of R₂₀₁—R₂₁₄ is not —H when X is O.

In certain methods of the invention, R₂₀₆ is not —H or —NH₂ when R₂₀₁—R₂₀₅ and R₂₀₇—R₂₁₄ are each —H.

In one embodiment, a sirtuin modulator is represented by Formula 81 or 82:

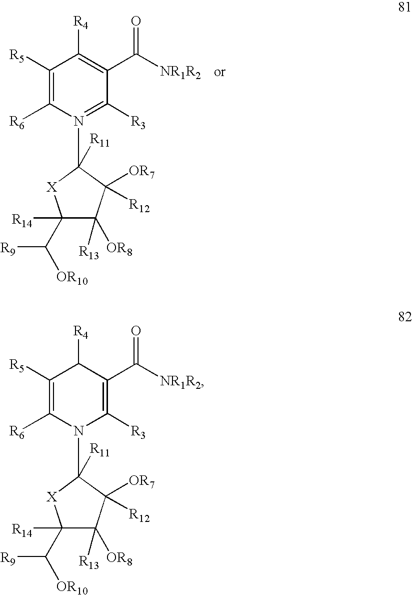

or a pharmaceutically acceptable salt thereof, wherein:

R₁ and R₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁ and R₂ taken together form a substituted or unsubstituted non-aromatic heterocyclic group, provided that when one of R₁ and R₂ is —H, the other is not an alkyl group substituted by —C(O)OCH₂CH₃;

R₃, R₄ and R₅ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₆ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRC(O)OR′, —NO₂ and —NRC(O)R′;

R₇, R₈ and R₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₉ selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₁₁, R₁₂, R₁₃ and R₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S, preferably O; and

n is 1 or 2,

provided that R₁—R₁₄ are not each —H and that R₁—R₉ and R₁₁—R₁₄ are not each —H when R₁₀ is —C(O)C₆H₅.

In certain embodiments, R₁ is —H.

In certain embodiments, R₇, R₈ and R₁₀ are independently —H, —C(O)R or —C(O)OR, typically —H or —C(O)R such as —H or —C(O)CH₃. In particular embodiments, R₁ is —H and R₇, R₈ and R₁₀ are independently —H, —C(O)R or —C(O)OR.

In certain embodiments, R₉ is —H. In particular embodiments, R₉ is —H when R₁ is —H and/or R₇, R₈ and R₁₀ are independently —H, —C(O)R or —C(O)OR.

In certain embodiments, R₂ is —H. In particular embodiments, R₂ is —H when R₉ is —H, R₁ is —H and/or R₇, R₈ and R₁₀ are independently —H, —C(O)R or —C(O)OR. Typically, R₂ is —H when R₉ is —H, R₁ is —H and R₇, R₈ and R₁₀ are independently —H, —C(O)R or —C(O)OR.

In certain embodiments, R₄ is —H or a halogen, such as deuterium or fluorine.

In one embodiment, a sirtuin modulator is represented by Formula 83 or 84:

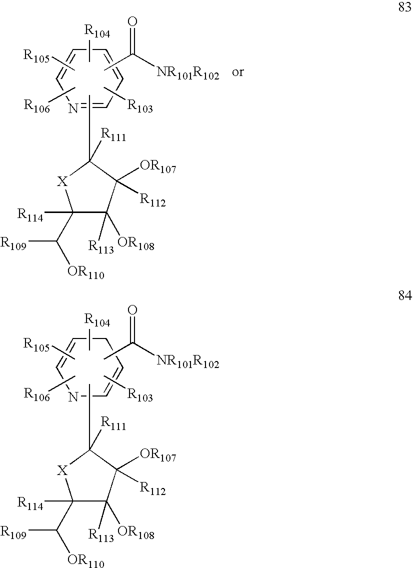

or a pharmaceutically acceptable salt thereof, wherein:

R₁₀₁ and R₁₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁₀₁ and R₁₀₂ taken together form a substituted or unsubstituted non-aromatic heterocyclic group;

R₁₀₃, R₁₀₄ R₁₀₅ and R₁₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₁₀₇ and R₁₀₈ are selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, wherein at least one of R₁₀₇ and R₁₀₈ is a substituted or unsubstituted alkyl group, a substituted or unsubstituted aryl group, —C(O)R, —C(O)OR, —C(O)NHR, —C(S)R, —C(S)OR or —C(O)SR;

R₁₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₁₁₀ is selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, provided that R₁₁₀ is not —C(O)C₆H₅;

R₁₁₁, R₁₁₂, R₁₁₃ and R₁₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

In another embodiment, a sirtuin modulator is represented by Formula 85 or 86:

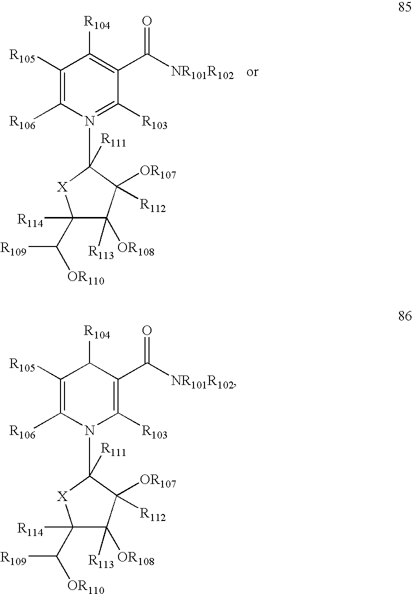

or a pharmaceutically acceptable salt thereof, where:

R₁₀₁ and R₁₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁₀₁ and R₁₀₂ taken together form a substituted or unsubstituted non-aromatic heterocyclic group;

R₁₀₃, R₁₀₄, R₁₀₅ and R₁₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₁₀₇ and R₁₀₈ are selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, wherein at least one of R₁₀₇ and R₁₀₈ is a substituted or unsubstituted alkyl group, a substituted or unsubstituted aryl group, —C(O)R, —C(O)OR, —C(O)NHR, —C(S)R, —C(S)OR or —C(O)SR;

R₁₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₁₁₀ is selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, provided that R₁₁₀ is not —C(O)C₆H₅;

R₁₁₁, R₁₁₂, R₁₁₃ and R₁₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

For compounds represented by Formulas 83-86, typically at least one of R₁₀₇ and R₁₀₈ is —C(O)R, such as —C(O)CH₃. In particular embodiments, R₁₀₇, R₁₀₈ and R₁₁₀ are independently —H or —C(O)R (e.g., —C(O)CH₃).

In certain embodiments, such as when R₁₀₇, R₁₀₈ and R₁₁₀ have the values described above, R₁₀₁ and R₁₀₂ are each —H.

In certain embodiments, R₁₀₉ is —H.

In certain embodiments, R₁₀₃—R₁₀₆ are each —H.

In certain embodiments, R₁₁₁—R₁₁₄ are each —H.

In particular embodiments, R₁₀₇, R₁₀₈ and R₁₁₀ have the values described above and R₁₀₁—R₁₀₆, R₁₀₉ and R₁₁₁—R₁₁₄ are each —H.

In certain embodiments, R₁₀₄ is —H or a halogen, typically deuterium or fluorine. The remaining values are as described above.

For sirtuin modulators represented by Formula 87 or 88: 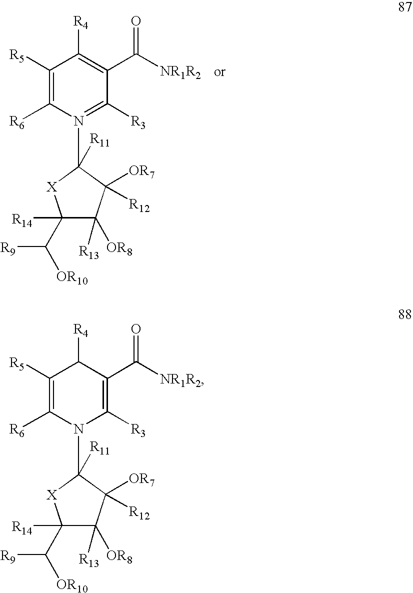
R₄ in certain embodiments is —H (e.g., deuterium, tritium) or a halogen (e.g., fluorine, bromine, chlorine).

In embodiments of the invention where R₁—R₆ can each be —H, they typically are each —H. In embodiments of the invention where one of R₁—R₆ is not —H, typically the remaining values are each —H and the non-H value is a substituted or unsubstituted alkyl group or a halogen (R₁ and R₂ are typically a substituted or unsubstituted alkyl group).

In certain embodiments, R₁₁—R₁₄ are each —H. When R₁₁—R₁₄ are each —H, R₁—R₆ typically have the values described above.

In certain embodiments, R₉ is —H. When R₉ is —H, typically R₁₁—R₁₄ are each —H and R₁—R₆ have the values described above.

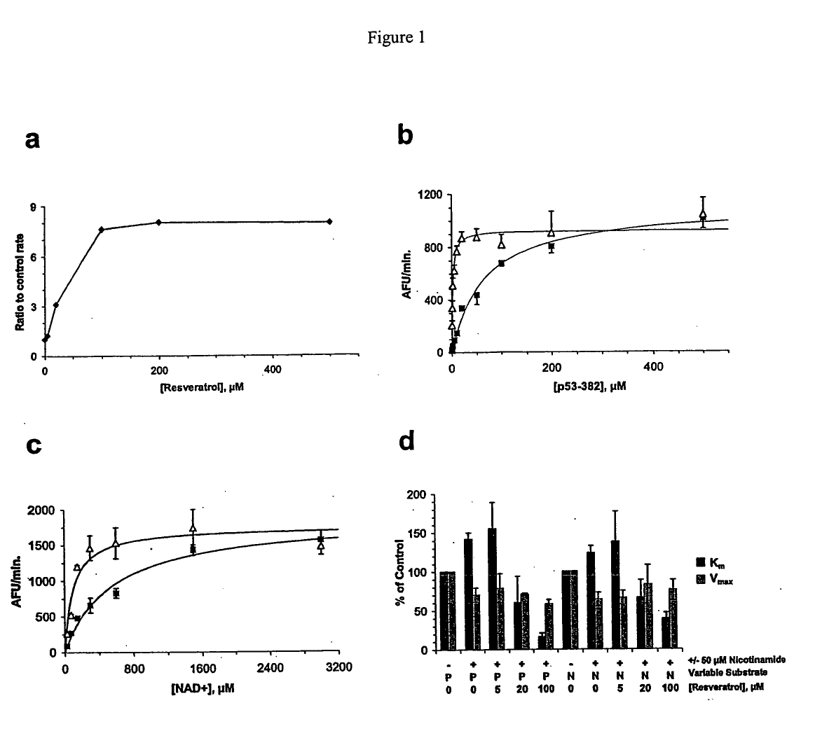

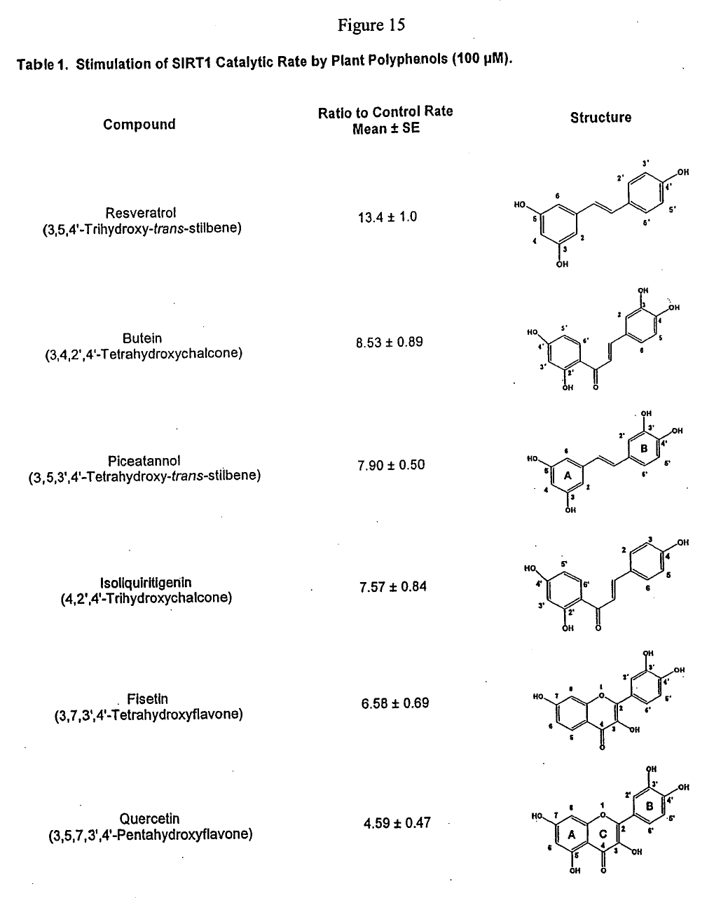

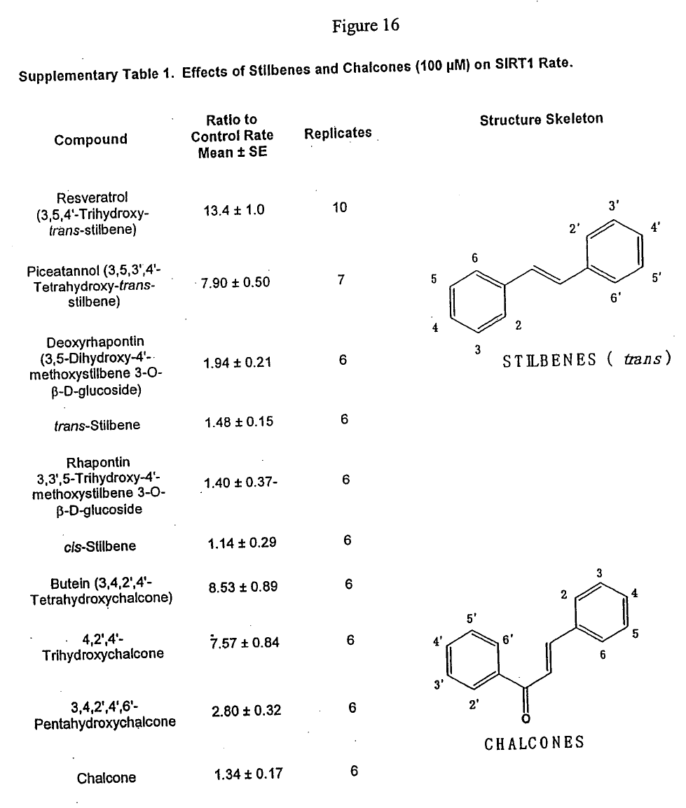

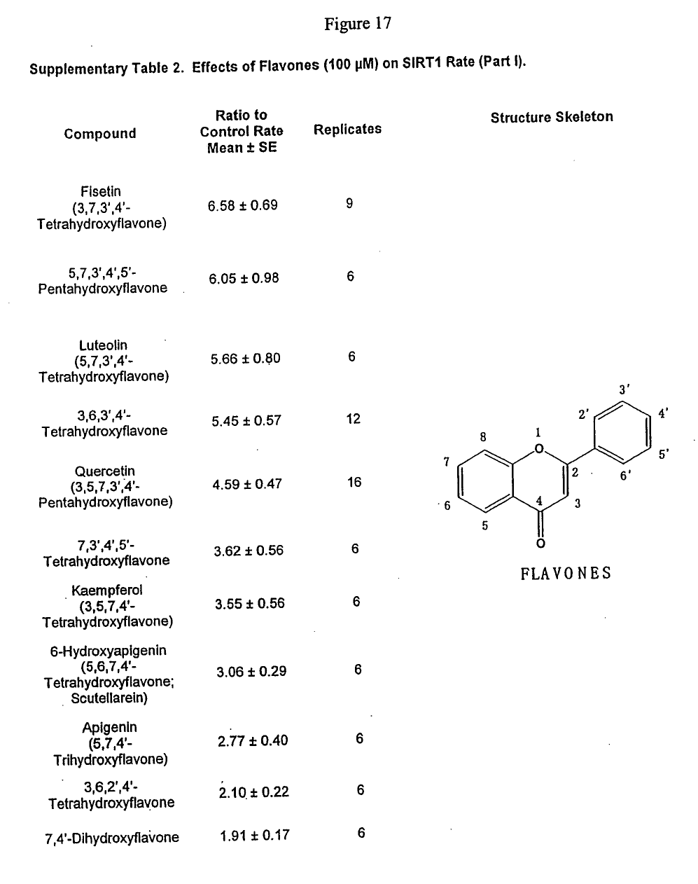

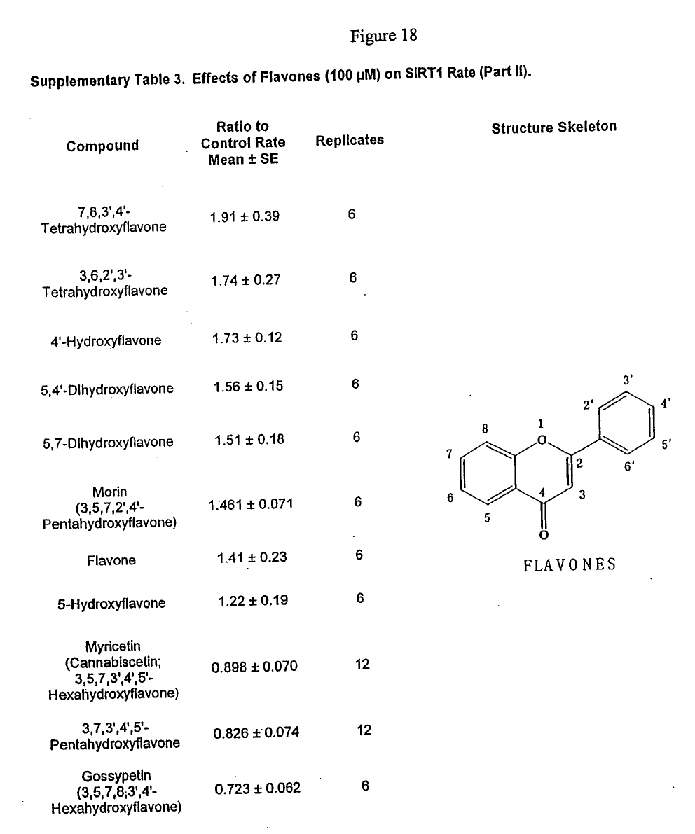

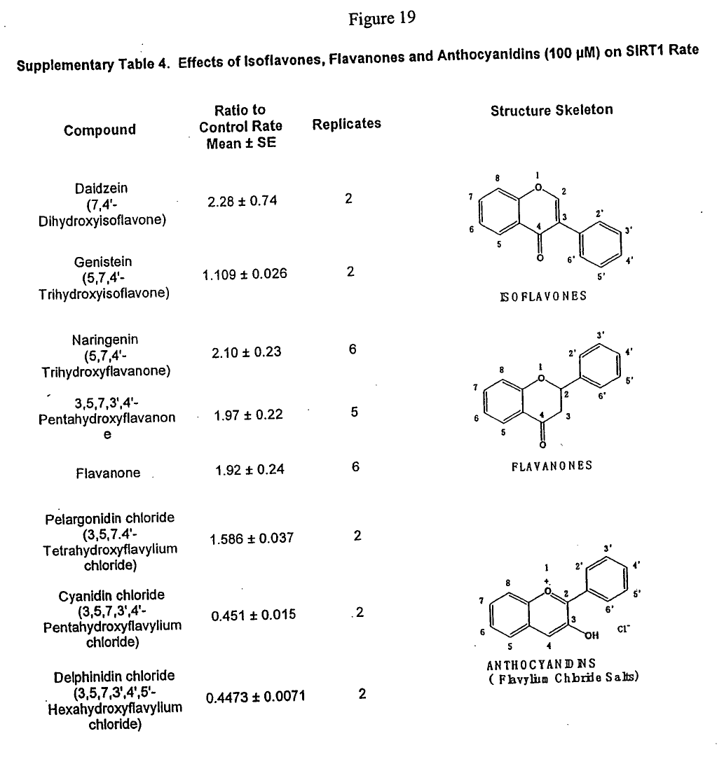

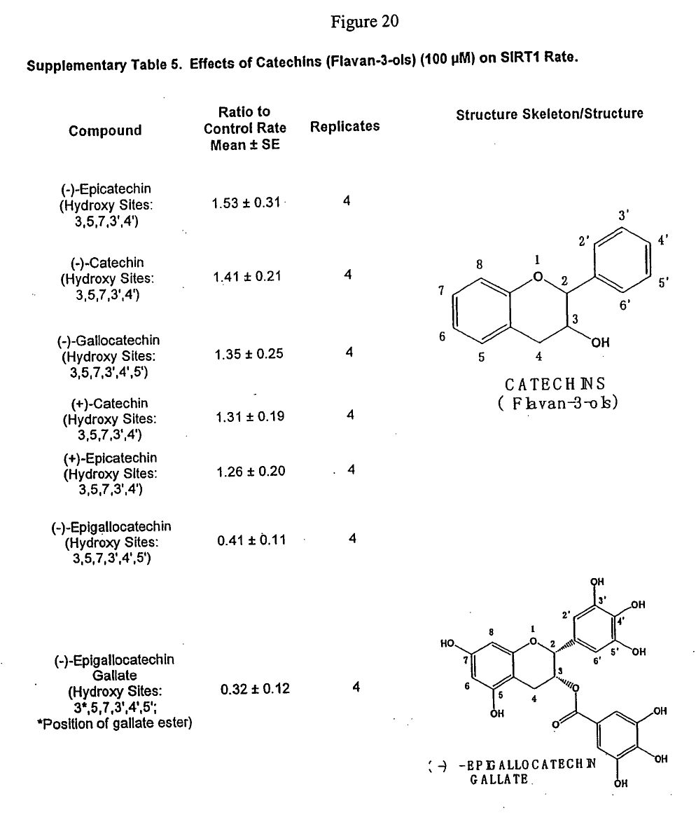

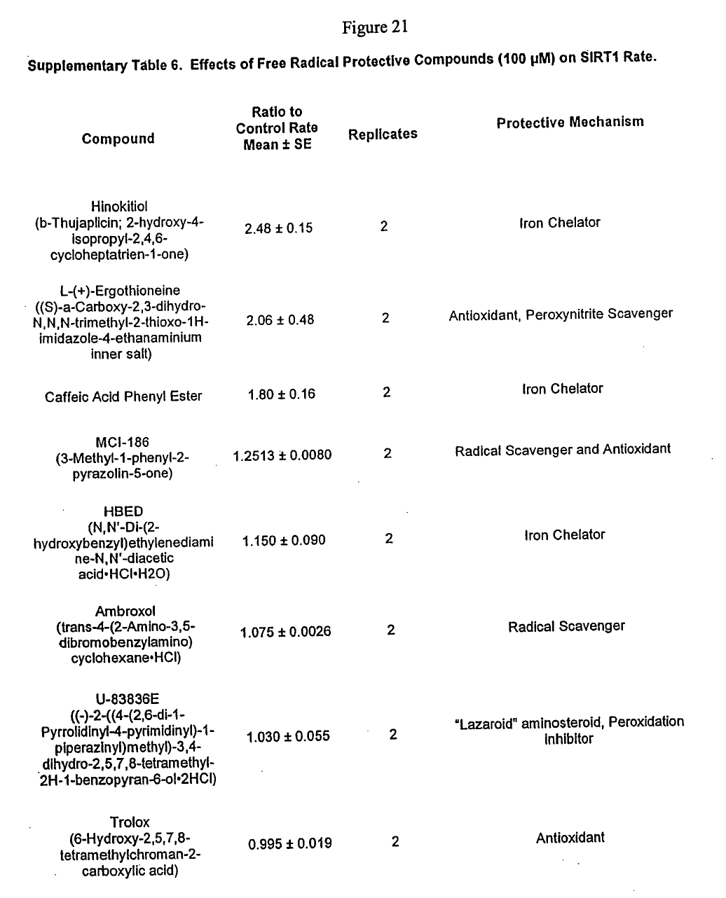

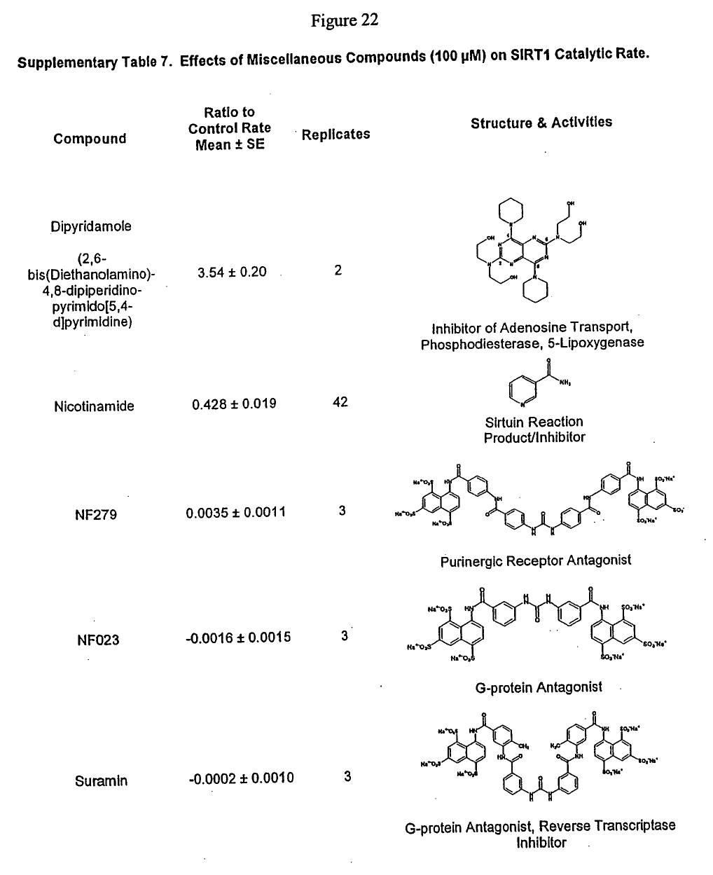

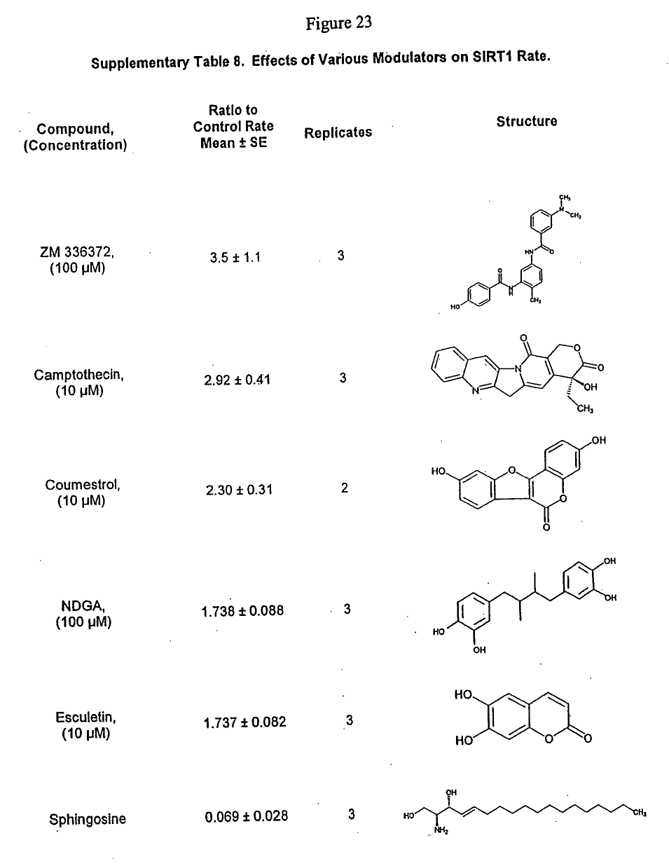

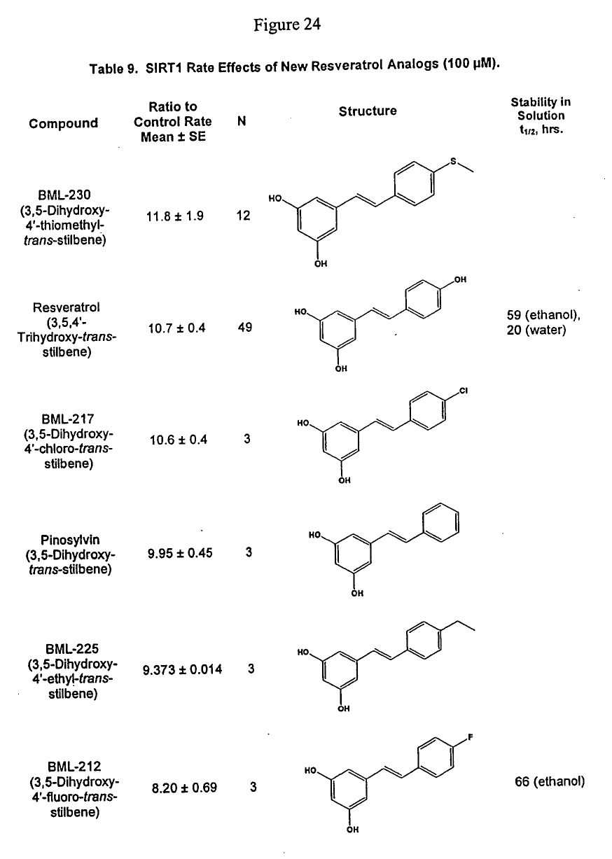

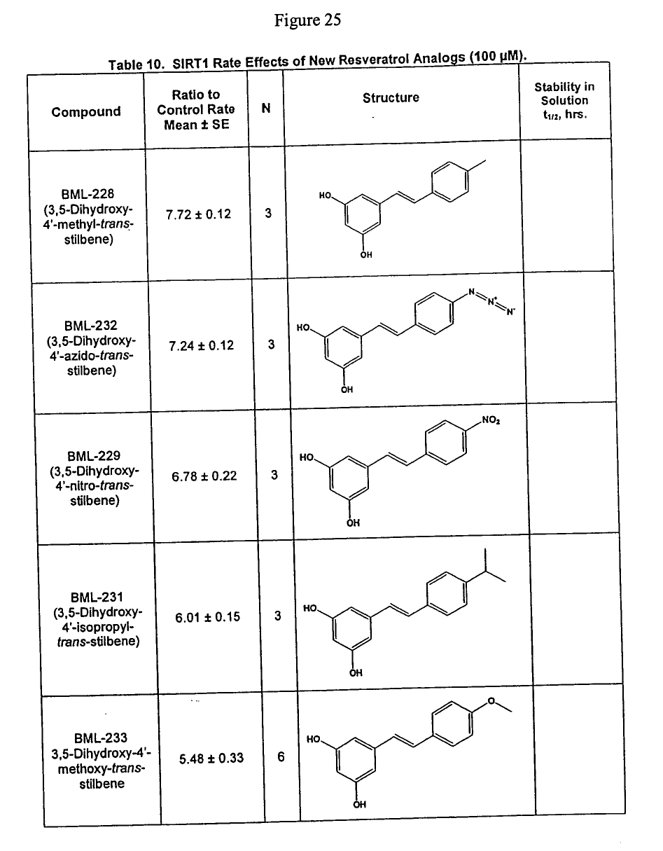

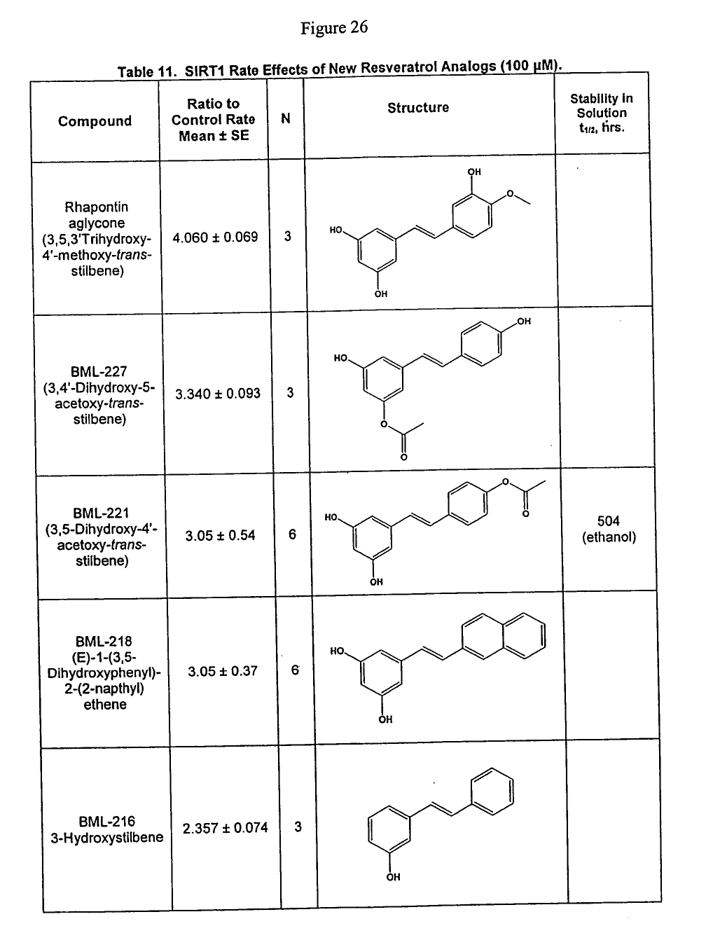

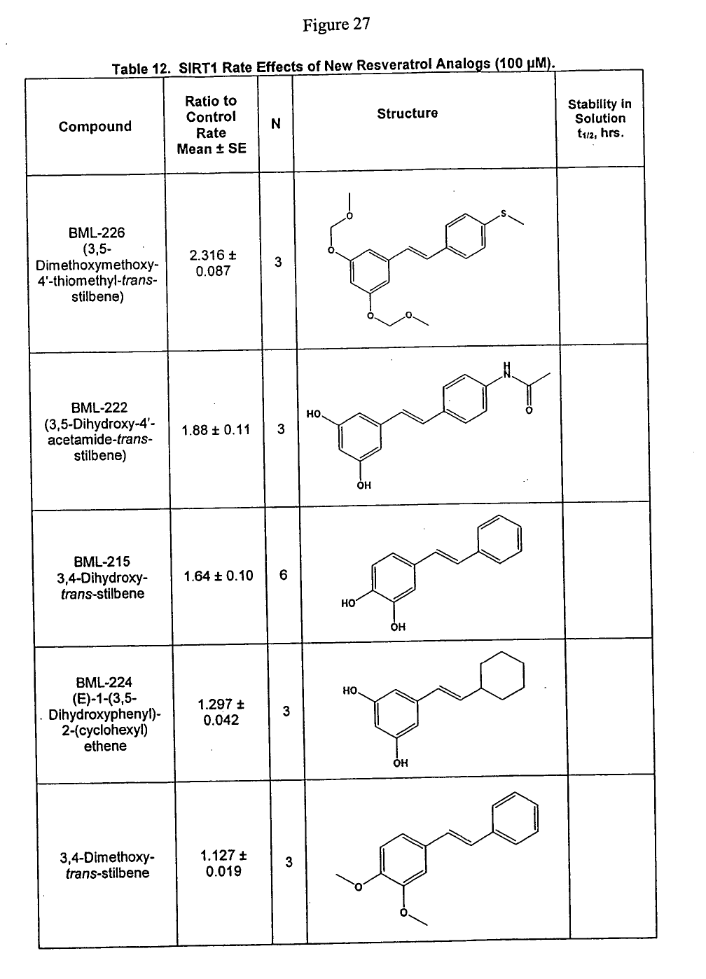

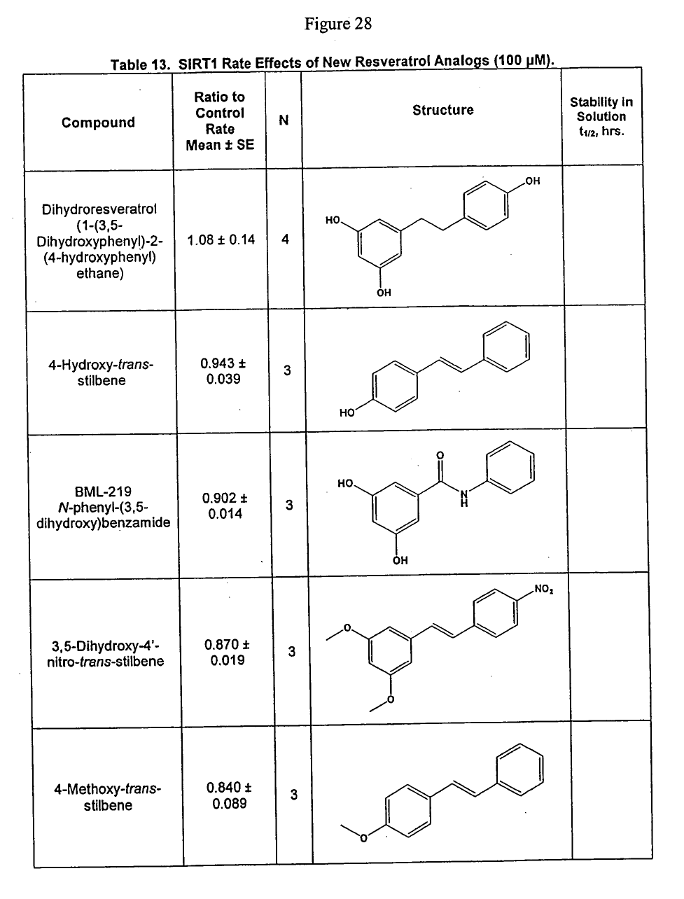

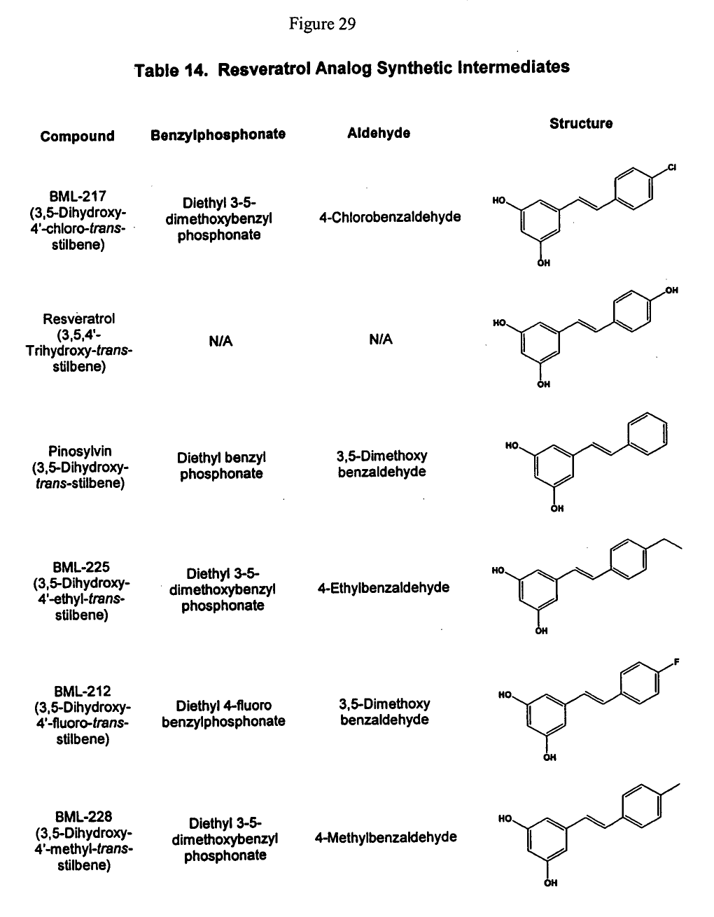

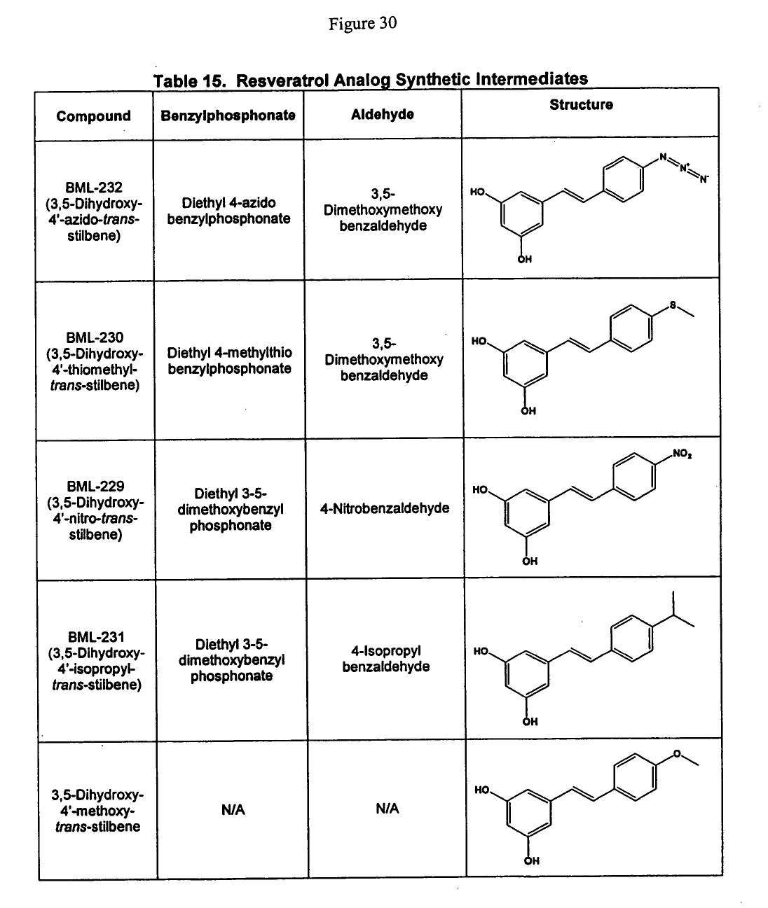

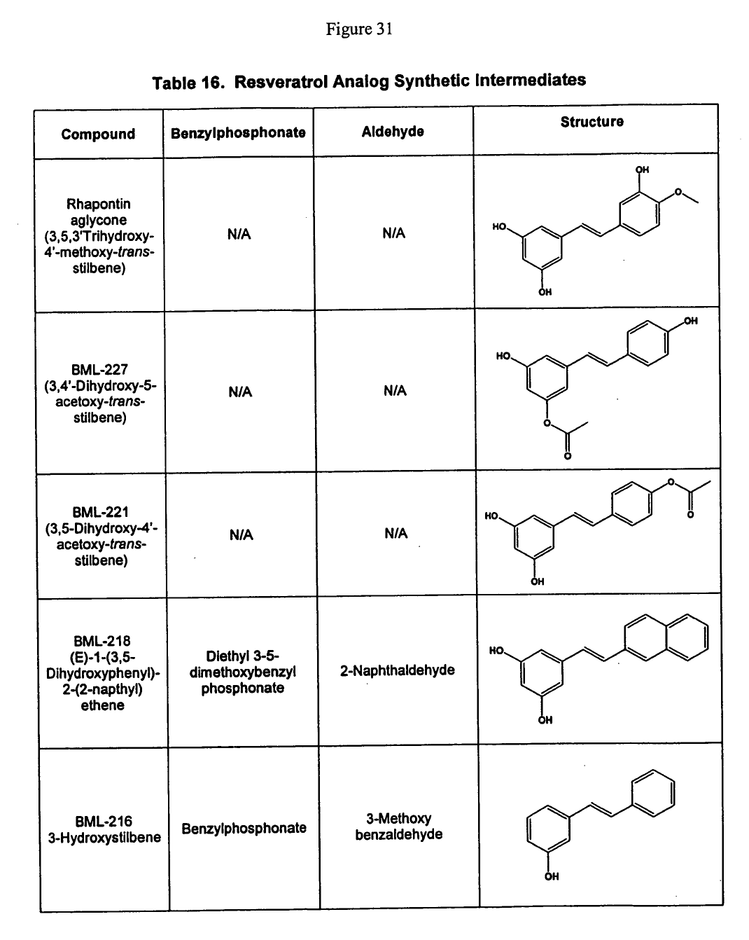

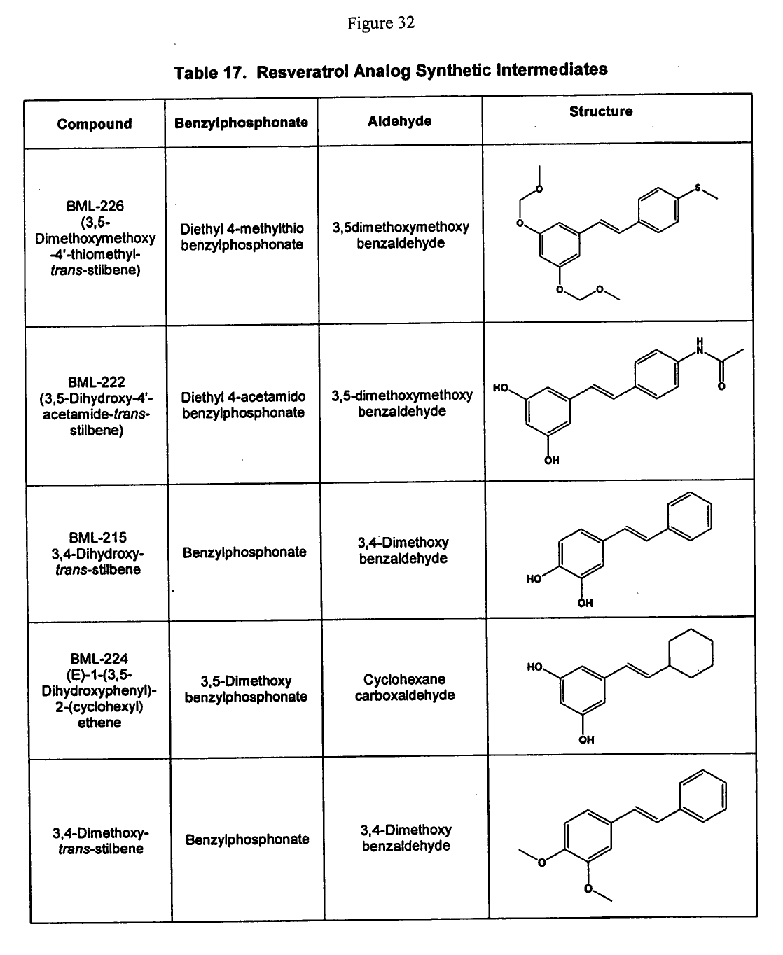

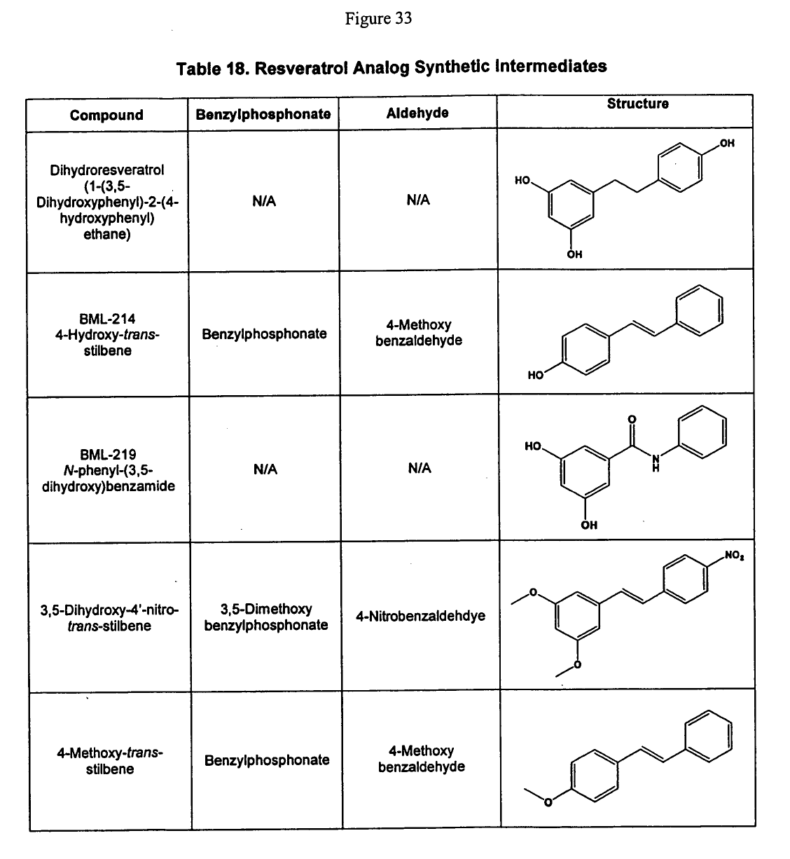

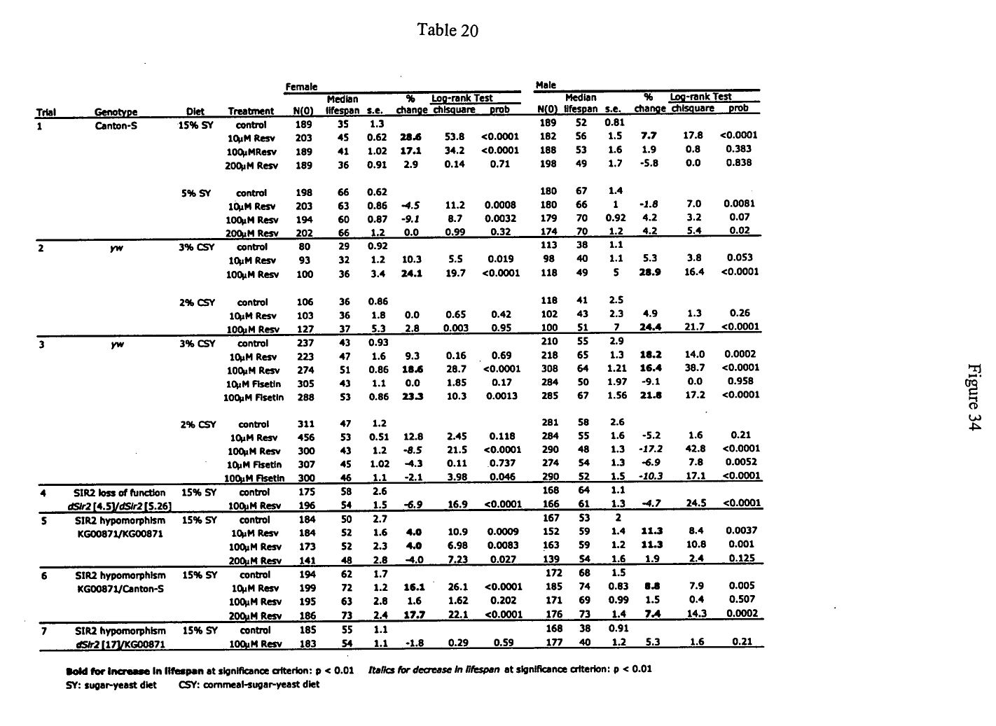

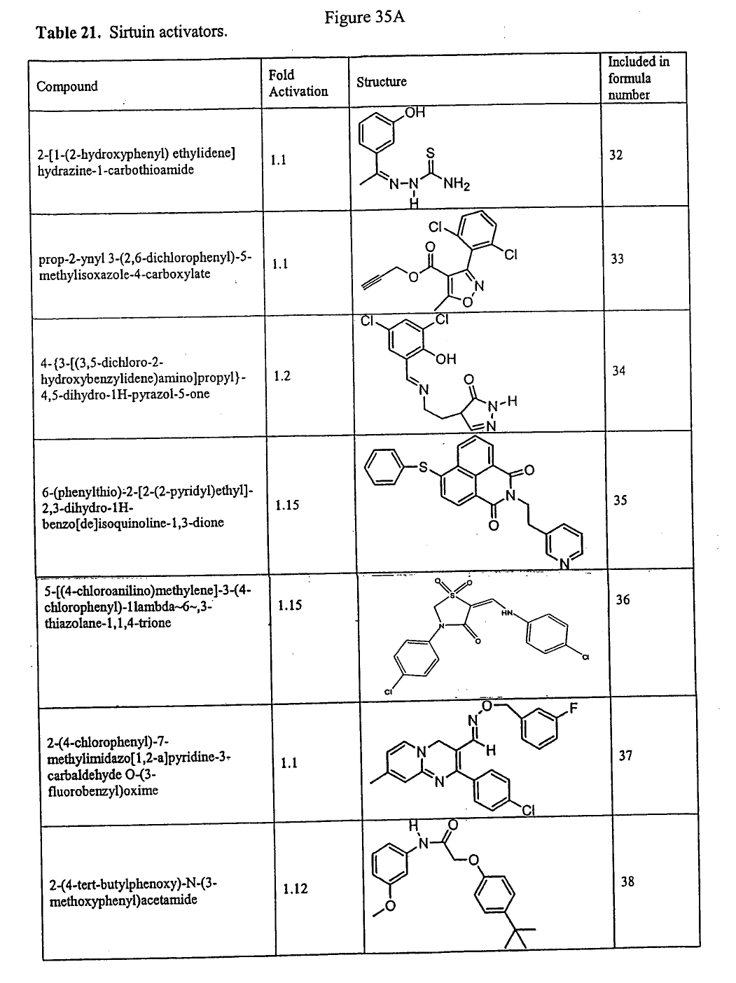

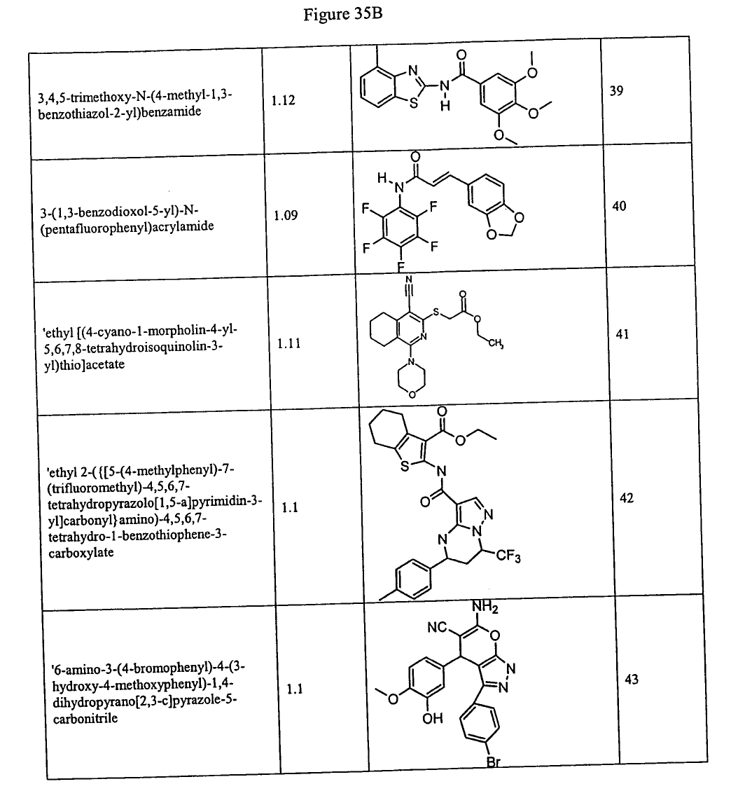

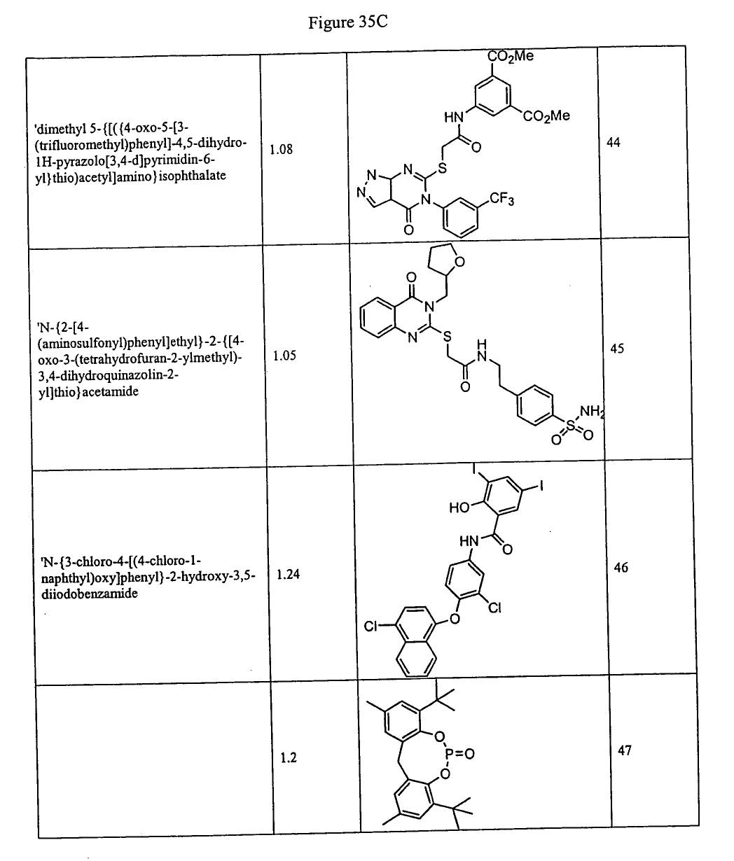

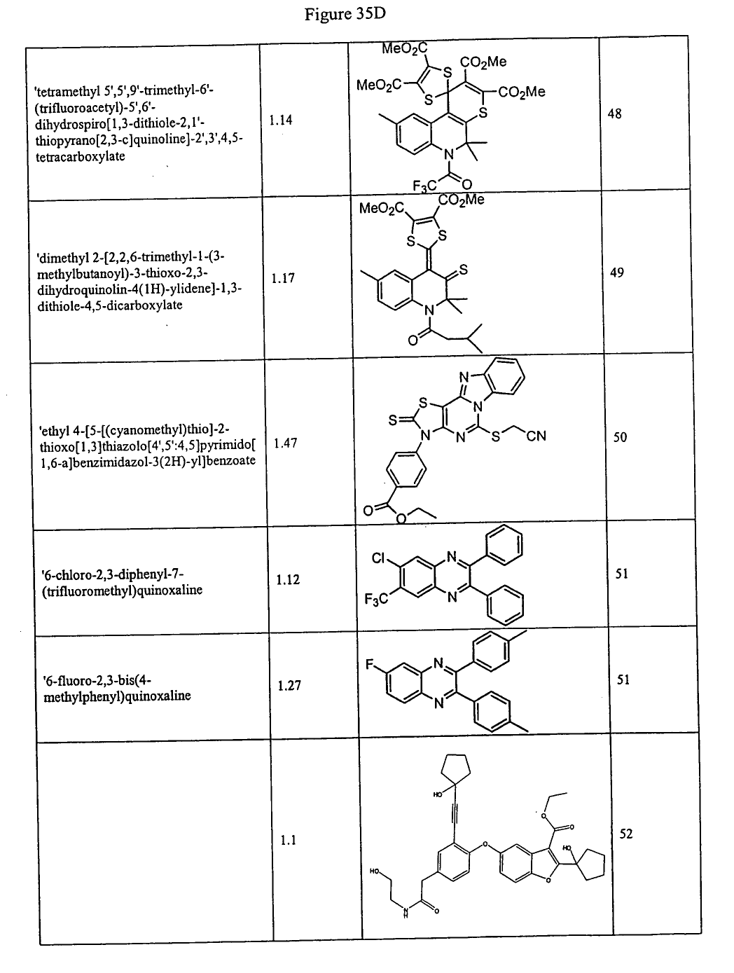

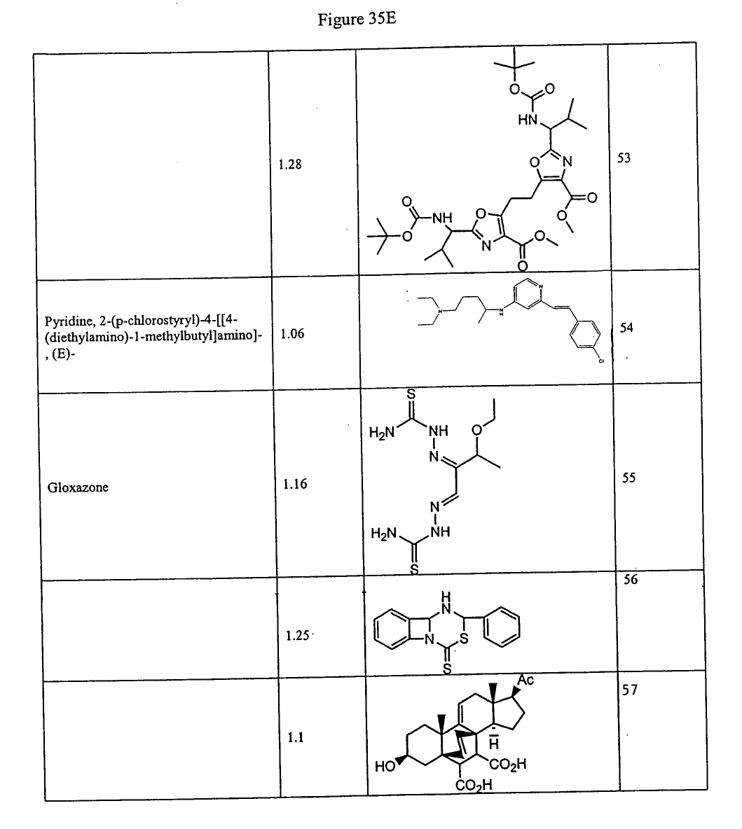

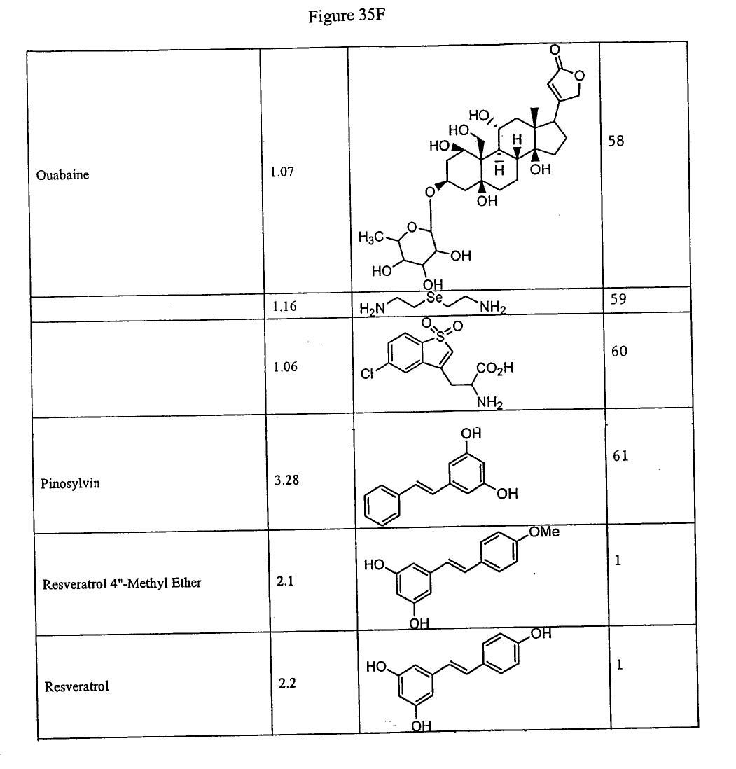

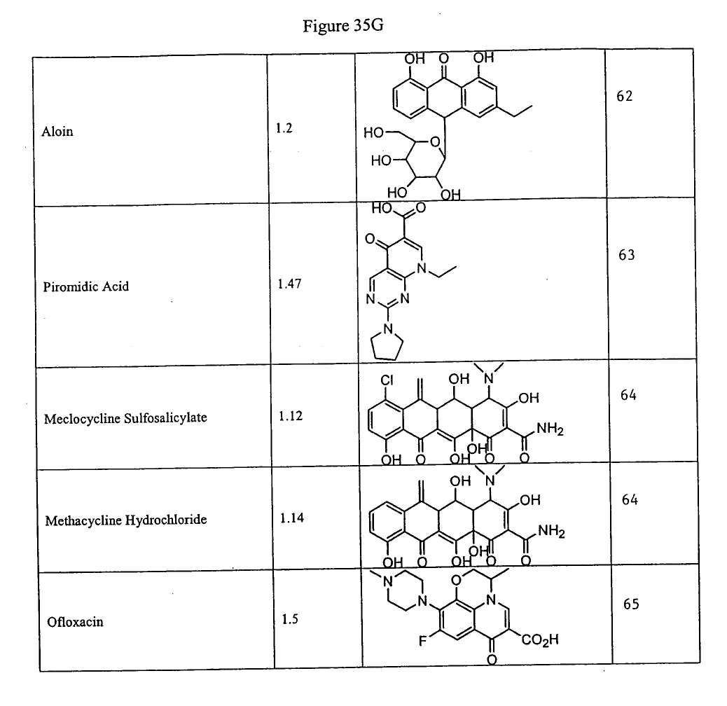

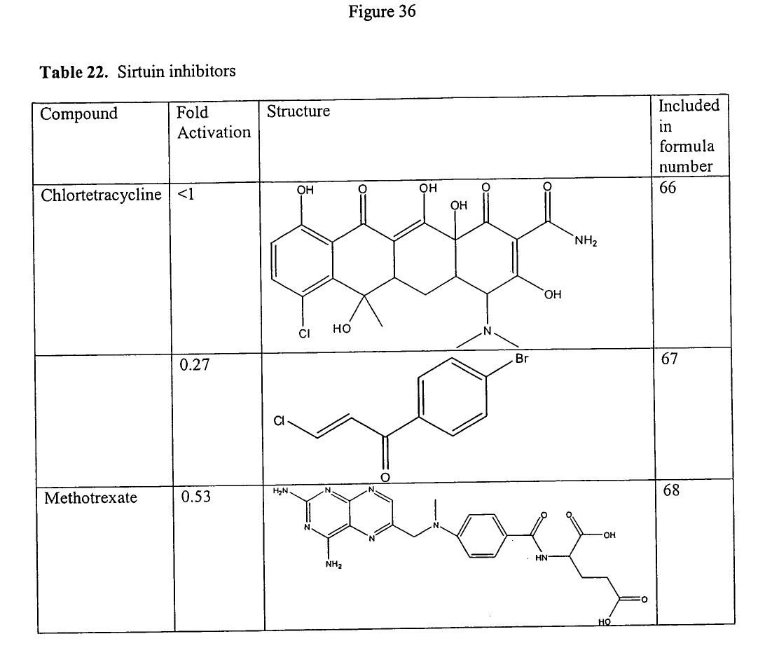

Specific examples of sirtuin modulators (e.g., sirtuin activators and sirtuin inhibitors) are shown in FIGS. 1-16.

Also included are pharmaceutically acceptable addition salts and complexes of the sirtuin modulators described herein. In cases wherein the compounds may have one or more chiral centers, unless specified, the compounds contemplated herein may be a single stereoisomer or racemic mixtures of stereoisomers.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.

Sirtuin modulating compounds also include the related secondary metabolites, such as phosphate, sulfate, acyl (e.g., acetyl, fatty acid acyl) and sugar (e.g., glucurondate, glucose) derivatives (e.g., of hydroxyl groups), particularly the sulfate, acyl and sugar derivatives. In other words, substituent groups —OH also include —OSO₃⁻M⁺, where M⁺ is a suitable cation (preferably H⁺, NH₄⁺ or an alkali metal ion such as Na⁺ or K⁺) and sugars such as 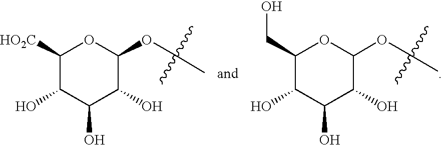
These groups are generally cleavable to —OH by hydrolysis or by metabolic (e.g., enzymatic) cleavage.

In cases in which the sirtuin-activating compounds have unsaturated carbon-carbon double bonds, both the cis (Z) and trans (E) isomers are contemplated herein. In cases wherein the compounds may exist in tautomeric forms, such as keto-enol tautomers, such as 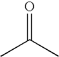
and 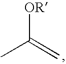
each tautomeric form is contemplated as being included within the methods presented herein, whether existing in equilibrium or locked in one form by appropriate substitution with R′. The meaning of any substituent at any one occurrence is independent of its meaning, or any other substituent's meaning, at any other occurrence.

Also included in the methods presented herein are prodrugs of the sirtuin-activating compounds described herein. Prodrugs are considered to be any covalently bonded carriers that release the active parent drug in vivo.

Analogs and derivatives of the sirtuin-activating compounds described herein can also be used for activating a member of the sirtuin protein family. For example, derivatives or analogs may make the compounds more stable or improve their ability to traverse cell membranes or being phagocytosed or pinocytosed. Exemplary derivatives include glycosylated derivatives, as described, e.g., in U.S. Pat. No. 6,361,815 for resveratrol. Other derivatives of resveratrol include cis- and trans-resveratrol and conjugates thereof with a saccharide, such as to form a glucoside (see, e.g., U.S. Pat. No. 6,414,037). Glucoside polydatin, referred to as piceid or resveratrol 3-O-beta-D-glucopyranoside, can also be used. Saccharides to which compounds may be conjugated include glucose, galactose, maltose, lactose and sucrose. Glycosylated stilbenes are further described in Regev-Shoshani et al. Biochemical J. (published on Apr. 16, 2003 as BJ20030141). Other derivatives of compounds described herein are esters, amides and prodrugs. Esters of resveratrol are described, e.g., in U.S. Pat. No. 6,572,882. Resveratrol and derivatives thereof can be prepared as described in the art, e.g., in U.S. Pat. Nos. 6,414,037; 6,361,815; 6,270,780; 6,572,882; and Brandolini et al. (2002) J. Agric. Food. Chem. 50:7407. Derivatives of hydroxyflavones are described, e.g., in U.S. Pat. No. 4,591,600. Resveratrol and other activating compounds can also be obtained commercially, e.g., from Sigma.

In certain embodiments, if a sirtuin-activating compound occurs naturally, it may be at least partially isolated from its natural environment prior to use. For example, a plant polyphenol may be isolated from a plant and partially or significantly purified prior to use in the methods described herein. An activating compound may also be prepared synthetically, in which case it would be free of other compounds with which it is naturally associated. In an illustrative embodiment, an activating composition comprises, or an activating compound is associated with, less than about 50%, 10%, 1%, 0.1%, 10⁻²% or 10⁻³% of a compound with which it is naturally associated.

In certain embodiments, a certain biological function (modulating neuronal activity or blood coagulation) is modulated by a sirtuin-activating compound with the proviso that the term sirtuin-activating compound does not include one or more specific compounds. For example, in certain embodiments, a sirtuin-activating compound may be any compound that is capable of increasing the level of expression and/or activity of a sirtuin protein with the proviso that the compound is not resveratrol, flavone, any other compound specifically cited herein, or any compound shown to be useful in treating or preventing neurodegenerative disorders and/or blood coagulation disorders prior to the priority date of this application. In an exemplary embodiment, a sirtuin-activating compound may be a compound of any one of formulas 1-25, 30, 32-65, and 69-88 with the proviso that the compound is not resveratrol, flavone, or any other compound specifically cited herein. In certain such embodiments, a sirtuin-activating compound does not include a compound of any one of formulas 69-76, any one of formulas 77-88, or anyone of formulas 69-88. In an exemplary embodiment, a sirtuin-activating compound does not include any of the compounds cited in U.S. Pat. Nos. 6,410,596 or 6,552,085, the disclosures of which are hereby incorporated by reference in their entirety.

In certain embodiments, the subject sirtuin activators, such as SIRT1 activators, do not have any substantial ability to inhibit PI3-kinase, inhibit aldoreductase and/or inhibit tyrosine protein kinases at concentrations (e.g., in vivo) effective for activating the deacetylase activity of the sirtuin, e.g., SIRT1. For instance, in preferred embodiments the sirtuin activator is chosen to have an EC₅₀ for activating sirtuin deacetylase activity that is at least 5 fold less than the EC₅₀ for inhibition of one or more of aldoreductase and/or tyrosine protein kinases, and even more preferably at least 10 fold, 100 fold or even 1000 fold less. Methods for assaying PI3-Kinase activity, aldose reductase activity, and tyrosine kinase activity are well known in the art and kits to perform such assays may be purchased commercially. See e.g., U.S. Patent Publication No. 2003/0158212 for P13-kinase assays; U.S. Patent Publication No. 2002/20143017 for aldose reductase assays; tyrosine kinase assay kits may be purchased commercially, for example, from Promega (Madison, Wis.; world wide web at promega.com), Invitrogen (Carlsbad, Calif.; world wide web at invitrogen.com) or Molecular Devices (Sunnyvale, Calif.; world wide web at moleculardevices.com).

In certain embodiments, the subject sirtuin activators do not have any substantial ability to transactivate EGFR tyrosine kinase activity at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for transactivating EGFR tyrosine kinase activity, and even more preferably at least 10 fold, 100 fold or even 1000 fold less. Methods for assaying transactivation of EGFR tyrosine kinase activity are well known in the art, see e.g., Pai et al. Nat. Med. 8: 289-93 (2002) and Vacca et al. Cancer Research 60: 5310-5317 (2000).

In certain embodiments, the subject sirtuin activators do not have any substantial ability to cause coronary dilation at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for coronary dilation, and even more preferably at least 10 fold, 100 fold or even 1000 fold less. Methods for assaying vasodilation are well known in the art, see e.g., U.S. Patent Publication No. 2004/0236153.

In certain embodiments, the subject sirtuin activators do not have any substantial spasmolytic activity at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for spasmolytic effects (such as on gastrointestinal muscle), and even more preferably at least 10 fold, 100 fold or even 1000 fold less. Methods for assaying spasmolytic activity are well known in the art, see e.g., U.S. Patent Publication No. 2004/0248987.

In certain embodiments, the subject sirtuin activators do not have any substantial ability to inhibit hepatic cytochrome P450 1B1 (CYP) at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for inhibition of P450 1B1, and even more preferably at least 10 fold, 100 fold or even 1000 fold less. Methods for assaying cytochrome P450 activity are well known in the art and kits to perform such assays may be purchased commercially. See e.g., U.S. Pat. Nos. 6,420,131 and 6,335,428 and Promega (Madison, Wis.; world wide web at promega.com).

In certain embodiments, the subject sirtuin activators do not have any substantial ability to inhibit nuclear factor-kappaB (NF-κB) at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for inhibition of NF-κB, and even more preferably at least 10 fold, 100 fold or even 1000 fold less. Methods for assaying NF-κB activity are well known in the art and kits to perform such assays may be purchased commercially (e.g., from Oxford Biomedical Research (Ann Arbor, Mich.; world wide web at oxfordbiomed.com)).

In certain embodiments, the subject sirtuin activators do not have any substantial ability to inhibit a histone deacetylase (HDACs) class I, a HDAC class II, or HDACs I and II, at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for inhibition of an HDAC I and/or HDAC II, and even more preferably at least 10 fold, 100 fold or even 1000 fold less. Methods for assaying HDAC I and/or HDAC II activity are well known in the art and kits to perform such assays may be purchased commercially. See e.g., BioVision, Inc. (Mountain View, Calif.; world wide web at biovision.com) and Thomas Scientific (Swedesboro, N.J.; world wide web at tomassci.com).

In certain embodiments, the subject SIRT1 activators do not have any substantial ability to activate SIRT1 orthologs in lower eukaryotes, particularly yeast or human pathogens, at concentrations (e.g., in vivo) effective for activating the deacetylase activity of human SIRT1. For instance, in preferred embodiments the SIRT1 activator is chosen to have an EC₅₀ for activating human SIRT1 deacetylase activity that is at least 5 fold less than the EC₅₀ for activating yeast Sir2 (such as Candida, S. cerevisiae, etc), and even more preferably at least 10 fold, 100 fold or even 1000 fold less.

In certain embodiments, the sirtuin activating compounds may have the ability to activate one or more sirtuin protein homologs, such as, for example, one or more of human SIRT1, SIRT2, SIRT3, SIRT4, SIRT5, SIRT6, or SIRT7. In other embodiments, a SIRT1 activator does not have any substantial ability to activate other sirtuin protein homologs, such as, for example, one or more of human SIRT2, SIRT3, SIRT4, SIRT5, SIRT6, or SIRT7, at concentrations (e.g., in vivo) effective for activating the deacetylase activity of human SIRT1. For instance, the SIRT1 activator may be chosen to have an EC₅₀ for activating human SIRT1 deacetylase activity that is at least 5 fold less than the EC₅₀ for activating one or more of human SIRT2, SIRT3, SIRT4, SIRT5, SIRT6, or SIRT7, and even more preferably at least 10 fold, 100 fold or even 1000 fold less.

In other embodiments, the subject sirtuin activators do not have any substantial ability to inhibit protein kinases; to phosphorylate mitogen activated protein (MAP) kinases; to inhibit the catalytic or transcriptional activity of cyclo-oxygenases, such as COX-2; to inhibit nitric oxide synthase (iNOS); or to inhibit platelet adhesion to type I collagen at concentrations (e.g., in vivo) effective for activating the deacetylase activity of the sirtuin. For instance, in preferred embodiments, the sirtuin activator is chosen to have an EC₅₀ for activating sirtuin deacetylase activity that is at least 5 fold less than the EC₅₀ for performing any of these activities, and even more preferably at least 10 fold, 100 fold or even 1000 fold less. Methods for assaying protein kinase activity, cyclo-oxygenase activity, nitric oxide synthase activity, and platelet adhesion activity are well known in the art and kits to perform such assays may be purchased commercially. See e.g., Promega (Madison, Wis.; world wide web at promega.com), Invitrogen (Carlsbad, Calif.; world wide web at invitrogen.com); Molecular Devices (Sunnyvale, Calif.; world wide web at moleculardevices.com) or Assay Designs (Ann Arbor, Mich.; world wide web at assaydesigns.com) for protein kinase assay kits; Amersham Biosciences (Piscataway, N.J.; world wide web at amershambiosciences.com) for cyclo-oxygenase assay kits; Amersham Biosciences (Piscataway, N.J.; world wide web at amershambiosciences.com) and R&D Systems (Minneapolis, Minn.; world wide web at rndsystems.com) for nitric oxide synthase assay kits; and U.S. Pat. Nos. 5,321,010; 6,849,290; and 6,774,107 for platelet adhesion assays.

In certain embodiments, the invention provides methods for treating and/or preventing neurodegenerative diseases and/or disorders, neuropathy associated with an ischemic event or disease, polyglutamine diseases, chemotherapeutic induced neuropathy, traumatic injury to a neuronal cell, and/or treating or preventing blood coagulation diseases or disorders by administering to a subject a high dose of a sirtuin activator. In certain embodiments, a high dose of a sirtuin activating compound refers to a quantity of a sirtuin activating compound having a sirtuin activating effect equal to or greater than the sirtuin activating effect of 18 mg/kg resveratrol in a human subject (or 200 mg/kg in a mouse). In certain embodiments, a high dose of a sirtuin activating compound refers to a quantity of a sirtuin activator having a sirtuin activating effect equal to or greater than the sirtuin activating effect of at least about 20, 25, 30, 35, 40, 50, 60, 75, 100, 150 mg/kg, or more, of resveratrol in a human subject. In an exemplary embodiment, at least about at least about 18, 20, 25, 30, 35, 40, 50, 60, 75, 100, 150 mg/kg, or more, of resveratrol is administered to a human subject. Human Equivalent Doses (HED) as compared to doses in a variety of animals are provided below in Table A. Methods for converting animal doses to human doses are also provided below.

A sirtuin activating effect refers to the level or extent of one or more therapeutic effects obtained upon administration of a high dose of a sirtuin activating compound. A sirtuin activating effect may be determined, for example, using the sirtuin assays described herein, including the methods provided in the Examples provided below. Therapeutic effects include, for example, (i) neuronal protection, (ii) axonal protection, (ii) improvement of clinical/neurological symptoms, (iv) prevention or slowing of progression of clinical/neurological symptoms, or (v) anti-coagulation effects in the blood. Such therapeutic effects include, for example, the therapeutic effects illustrated in the Examples.

A high dose of a sirtuin-activating compound may be administered to a subject once, or multiple times (e.g., daily) until a desired therapeutic effect is achieved. For example, a high dose may be administered daily for 1 day, 1 week, 2 weeks, 1 month, 2 months, 3 months, 6 months, 1 year, or more depending on the disease or disorder being treated. A high dose of a sirtuin activator may be administered daily in a single dosage or may be divided into multiple dosages, e.g., that are administered twice or three times per day. In an exemplary embodiment, a high dose of a sirtuin activator may be administered in a sustained release formulation.

The methods described herein may comprise administering daily, or every other day, or once a week, a high dose of a sirtuin activating compound, e.g., in the form of a pill or injection, to a subject. In embodiments where the high dose of a sirtuin activating compound is administered daily to the subject, the sirtuin activating compound may be administered once a day. In other embodiments, it is administered twice or three times a day.

In some embodiments, the high dose of a sirtuin activating compound is administered in a sustained release formulation, e.g., by embedding or encapsulating the sirtuin activator into nanoparticles for delivery over a period of at least 12 hours, to a subject. In embodiments where the sirtuin activator is administered to a subject in a sustained release formulation, a high dose of the sirtuin activator may be administered for sustained delivery over a period of for example, at least about 12, 15, 18, 24, or 36 hours, or longer. In other embodiments, it is administered for a sustained delivery over a period of one or more days. In yet other embodiments, it is administered for a sustained delivery over a period of one or more weeks.

TABLE A
Conversion of Animal Doses to Human Equivalent Doses (HED) Based
on Body Surface Area (see e.g., Guidance for Industry Reviewers:
Estimating the Safe Starting Dose in Clinical Trials for
Therapeutics in Adult Healthy Volunteers, on the world wide
web at fda.gov/ohrms/dockets/98fr/02d-0492-gdl0001-vol1.pdf).

| | To convert animal | | |
|---|---|---|---|
| | dose in mg/kg to | To convert animal dose in mg/kg | |
| | dose in mg/m², | to HED^{a} in mg/kg, either: | |
| | multiple by km | Divide animal | Multiply animal |
| Species | below: | dose by: | dose by: |
| Human | 37 | — | — |
| Human Child | 25 | — | — |
| (20 kg) | | | |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea Pig | 8 | 4.6 | 0.22 |
| Rabbit | 12 | 3.1 | 0.32 |
| Dog | 20 | 1.8 | 0.54 |
| Monkeys^{b} | 12 | 3.1 | 0.32 |
| Marmoset | 6 | 6.2 | 0.16 |
| Squirrel Monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, human equivalent dose can be calculated from the formula: HED = animal dose in mg/kg × (animal weight in kg/human weight in kg)^{0.33}.
^{b}For example, cynomolgus, rhesus, stumptail.

3. Exemplary Therapeutic Applications of Sirtuin-Activating Compounds

In certain aspects, the sirtuin-activating compounds described herein can be used to treat patients suffering from neurodegenerative diseases, and traumatic or mechanical injury to the central nervous system (CNS), spinal cord or peripheral nervous system (PNS). Neurodegenerative disease typically involves reductions in the mass and volume of the human brain, which may be due to the atrophy and/or death of brain cells, which are far more profound than those in a healthy person that are attributable to aging. Neurodegenerative diseases can evolve gradually, after a long period of normal brain function, due to progressive degeneration (e.g., nerve cell dysfunction and death) of specific brain regions. Alternatively, neurodegenerative diseases can have a quick onset, such as those associated with trauma or toxins. The actual onset of brain degeneration may precede clinical expression by many years. Examples of neurodegenerative diseases include, but are not limited to, Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, and Friedreich's ataxia. The compounds of this invention can be used to treat these disorders and others as described below.

AD is a chronic, incurable, and unstoppable CNS disorder that occurs gradually, resulting in memory loss, unusual behavior, personality changes, and a decline in thinking abilities. These losses are related to the death of specific types of brain cells and the breakdown of connections and their supporting network (e.g. glial cells) between them. AD has been described as childhood development in reverse. In most people with AD, symptoms appear after the age 60. The earliest symptoms include loss of recent memory, faulty judgment, and changes in personality. Later in the disease, those with AD may forget how to do simple tasks like washing their hands. Eventually people with AD lose all reasoning abilities and become dependent on other people for their everyday care. Finally, the disease becomes so debilitating that patients are bedridden and typically develop coexisting illnesses.

PD is a chronic, incurable, and unstoppable CNS disorder that occurs gradually and results in uncontrolled body movements, rigidity, tremor, and dyskinesia. These motor system problems are related to the death of brain cells in an area of the brain that produces dopamine, a chemical that helps control muscle activity. In most people with PD, symptoms appear after age 50. The initial symptoms of PD are a pronounced tremor affecting the extremities, notably in the hands or lips. Subsequent characteristic symptoms of PD are stiffness or slowness of movement, a shuffling walk, stooped posture, and impaired balance. There are wide ranging secondary symptoms such as memory loss, dementia, depression, emotional changes, swallowing difficulties, abnormal speech, sexual dysfunction, and bladder and bowel problems. These symptoms will begin to interfere with routine activities, such as holding a fork or reading a newspaper. Finally, people with PD become so profoundly disabled that they are bedridden.

ALS (motor neuron disease) is a chronic, incurable, and unstoppable CNS disorder that attacks the motor neurons, components of the CNS that connect the brain to the skeletal muscles. In ALS, the motor neurons deteriorate and eventually die, and though a person's brain normally remains fully functioning and alert, the command to move never reaches the muscles. Most people who get ALS are between 40 and 70 years old. The first motor neurons that weaken are those controlling to the arms or legs. Those with ALS may have trouble walking, they may drop things, fall, slur their speech, and laugh or cry uncontrollably. Eventually the muscles in the limbs begin to atrophy from disuse. This muscle weakness will become debilitating and a person will need a wheel chair or become unable to function out of bed.

The causes of these neurological diseases have remained largely unknown. They are conventionally defined as distinct diseases, yet clearly show extraordinary similarities in basic processes and commonly demonstrate overlapping symptoms far greater than would be expected by chance alone. Current disease definitions fail to properly deal with the issue of overlap and a new classification of the neurodegenerative disorders has been called for.

HD is another neurodegenerative disease resulting from genetically programmed degeneration of neurons in certain areas of the brain. This degeneration causes uncontrolled movements, loss of intellectual faculties, and emotional disturbance. HD is a familial disease, passed from parent to child through a dominant mutation in the wild-type gene. Some early symptoms of HD are mood swings, depression, irritability or trouble driving, learning new things, remembering a fact, or making a decision. As the disease progresses, concentration on intellectual tasks becomes increasingly difficult and the patient may have difficulty feeding himself or herself and swallowing.

Tay-Sachs disease and Sandhoff disease are glycolipid storage diseases caused by the lack of lysosomal β-hexosaminidase (Gravel et al., in The Metabolic Basis of Inherited Disease, eds. Scriver et al., McGraw-Hill, N.Y., pp. 2839-2879, 1995). In both disorders, GM2 ganglioside and related glycolipidssubstrates for β-hexosaminidase accumulate in the nervous system and trigger acute neurodegeneration. In the most severe forms, the onset of symptoms begins in early infancy. A precipitous neurodegenerative course then ensues, with affected infants exhibiting motor dysfunction, seizure, visual loss, and deafness. Death usually occurs by 2-5 years of age. Neuronal loss through an apoptotic mechanism has been demonstrated (Huang et al., Hum. Mol. Genet. 6: 1879-1885, 1997).

It is well-known that apoptosis plays a role in AIDS pathogenesis in the immune system. However, HIV-1 also induces neurological disease. Shi et al. (J. Clin. Invest. 98: 1979-1990, 1996) examined apoptosis induced by HIV-1 infection of the CNS in an in vitro model and in brain tissue from AIDS patients, and found that HIV-1 infection of primary brain cultures induced apoptosis in neurons and astrocytes in vitro. Apoptosis of neurons and astrocytes was also detected in brain tissue from 10/11 AIDS patients, including 5/5 patients with HIV-1 dementia and 4/5 nondemented patients.

There are four main peripheral neuropathies associated with HIV, namely sensory neuropathy, AIDP/CIPD, drug-induced neuropathy and CMV-related.

The most common type of neuropathy associated with AIDS is distal symmetrical polyneuropathy (DSPN). This syndrome is a result of nerve degeneration and is characterized by numbness and a sensation of pins and needles. DSPN causes few serious abnormalities and mostly results in numbness or tingling of the feet and slowed reflexes at the ankles. It generally occurs with more severe immunosuppression and is steadily progressive. Treatment with tricyclic antidepressants relieves symptoms but does not affect the underlying nerve damage.

A less frequent, but more severe type of neuropathy is known as acute or chronic inflammatory demyelinating polyneuropathy (AIDP/CIDP). In AIDP/CIDP there is damage to the fatty membrane covering the nerve impulses. This kind of neuropathy involves inflammation and resembles the muscle deterioration often identified with long-term use of AZT. It can be the first manifestation of HIV infection, where the patient may not complain of pain, but fails to respond to standard reflex tests. This kind of neuropathy may be associated with seroconversion, in which case it can sometimes resolve spontaneously. It can serve as a sign of HIV infection and indicate that it might be time to consider antiviral therapy. AIDP/CIDP may be auto-immune in origin.

Drug-induced, or toxic, neuropathies can be very painful. Antiviral drugs commonly cause peripheral neuropathy, as do other drugs e.g. vincristine, dilantin (an anti-seizure medication), high-dose vitamins, isoniazid, and folic acid antagonists. Peripheral neuropathy is often used in clinical trials for antivirals as a dose-limiting side effect, which means that more drugs should not be administered. Additionally, the use of such drugs can exacerbate otherwise minor neuropathies. Usually, these drug-induced neuropathies are reversible with the discontinuation of the drug.

CMV causes several neurological syndromes in AIDS, including encephalitis, myelitis, and polyradiculopathy.

Neuronal loss is also a salient feature of prion diseases, such as Creutzfeldt-Jakob disease in human, BSE in cattle (mad cow disease), Scrapie Disease in sheep and goats, and feline spongiform encephalopathy (FSE) in cats. The sirtuin activating compounds described herein may be useful for treating or preventing neuronal loss due to these prior diseases.

In an exemplary embodiment, a sirtuin activating compound may be used to treat or prevent multiple sclerosis (MS), including relapsing MS and monosymptomatic MS, and other demyelinating conditions, such as, for example, chromic inflammatory demyelinating polyneuropathy (CIDP), or symptoms associated therewith.

MS is a chronic, often disabling disease of the central nervous system. Various and converging lines of evidence point to the possibility that the disease is caused by a disturbance in the immune function, although the cause of this disturbance has not been established. This disturbance permits cells of the immune system to “attack” myelin, the fat containing insulating sheath that surrounds the nerve axons located in the central nervous system (“CNS”). When myelin is damaged, electrical pulses cannot travel quickly or normally along nerve fiber pathways in the brain and spinal cord. This results in disruption of normal electrical conductivity within the axons, fatigue and disturbances of vision, strength, coordination, balance, sensation, and bladder and bowel function.

As such, MS is now a common and well-known neurological disorder that is characterized by episodic patches of inflammation and demyelination which can occur anywhere in the CNS. However, almost always without any involvement of the peripheral nerves associated therewith. Demyelination produces a situation analogous to that resulting from cracks or tears in an insulator surrounding an electrical cord. That is, when the insulating sheath is disrupted, the circuit is “short circuited” and the electrical apparatus associated therewith will function intermittently or nor at all. Such loss of myelin surrounding nerve fibers results in short circuits in nerves traversing the brain and the spinal cord that thereby result in symptoms of MS. It is further found that such demyelination occurs in patches, as opposed to along the entire CNS. In addition, such demyelination may be intermittent. Therefore, such plaques are disseminated in both time and space.

It is believed that the pathogenesis involves a local disruption of the blood brain barrier which causes a localized immune and inflammatory response, with consequent damage to myelin and hence to neurons.

Clinically, MS exists in both sexes and can occur at any age. However, its most common presentation is in the relatively young adult, often with a single focal lesion such as a damage of the optic nerve, an area of anesthesia (loss of sensation), or paraesthesia (localize loss of feeling), or muscular weakness. In addition, vertigo, double vision, localized pain, incontinence, and pain in the arms and legs may occur upon flexing of the neck, as well as a large variety of less common symptoms.

An initial attack of MS is often transient, and it may be weeks, months, or years before a further attack occurs. Some individuals may enjoy a stable, relatively event free condition for a great number of years, while other less fortunate ones may experience a continual downhill course ending in complete paralysis. There is, most commonly, a series of remission and relapses, in which each relapse leaves a patient somewhat worse than before. Relapses may be triggered by stressful events, viral infections or toxins. Therein, elevated body temperature, i.e., a fever, will make the condition worse, or as a reduction of temperature by, for example, a cold bath, may make the condition better.

In yet another embodiment, a sirtuin activating compound may be used to treat trauma to the nerves, including, trauma due to disease, injury (including surgical intervention), or environmental trauma (e.g., neurotoxins, alcoholism, etc.).

The subject sirtuin activators may also be useful to prevent, treat, and alleviate symptoms of various PNS disorders, such as the ones described below. The PNS is composed of the nerves that lead to or branch off from the spinal cord and CNS. The peripheral nerves handle a diverse array of functions in the body, including sensory, motor, and autonomic functions. When an individual has a peripheral neuropathy, nerves of the PNS have been damaged. Nerve damage can arise from a number of causes, such as disease, physical injury, poisoning, or malnutrition. These agents may affect either afferent or efferent nerves. Depending on the cause of damage, the nerve cell axon, its protective myelin sheath, or both may be injured or destroyed.

The term “peripheral neuropathy” encompasses a wide range of disorders in which the nerves outside of the brain and spinal cord—peripheral nerves—have been damaged. Peripheral neuropathy may also be referred to as peripheral neuritis, or if many nerves are involved, the terms polyneuropathy or polyneuritis may be used. Peripheral neuropathy may be caused either by diseases of the nerve or from the side-effects of systemic illness. Peripheral neuropathies vary in their presentation and origin, and may affect the nerve or the neuromuscular junction. Peripheral neuropathy is a widespread disorder, and there are many underlying causes, including, for example, seizures, nutritional deficiencies, HIV, diabetes, leprosy, Charcot-Marie-Tooth disease, Guillain-Barré syndrome, acrylamide poisoning, certain inherited disorders, mechanical pressure from staying in one position for too long, a tumor, intraneural hemorrhage, exposing the body to extreme conditions such as radiation, cold temperatures, or toxic substances can also cause peripheral neuropathy.

Leprosy is caused by the bacterium Mycobacterium leprae, which attacks the peripheral nerves of affected people and is very rare in the United States. Diabetes is the most commonly known cause of peripheral neuropathy. It has been estimated that more than 17 million people in the United States and Europe have diabetes-related polyneuropathy. Many neuropathies are idiopathic; no known cause can be found. The most common of the inherited peripheral neuropathies in the United States is Charcot-Marie-Tooth disease, which affects approximately 125,000 persons.

Diabetic neuropathies are neuropathic disorders that are associated with diabetes mellitus. These conditions usually result from diabetic microvascular injury involving small blood vessels that supply nerves (vasa nervorum). Relatively common conditions which may be associated with diabetic neuropathy include third nerve palsy; mononeuropathy; mononeuritis multiplex; diabetic amyotrophy; a painful polyneuropathy; autonomic neuropathy; and thoracoabdominal neuropathy. Clinical manifestations of diabetic neuropathy include, for example, sensorimotor polyneuropathy such as numbness, sensory loss, dysesthesia and nighttime pain; autonomic neuropathy such as delayed gastric emptying or gastroparesis; and cranial neuropathy such as oculomotor (3rd) neuropathies or Mononeuropathies of the thoracic or lumbar spinal nerves.

Another of the better known peripheral neuropathies is Guillain-Barré syndrome, which arises from complications associated with viral illnesses, such as cytomegalovirus, Epstein-Barr virus, and human immunodeficiency virus (HIV), or bacterial infection, including Campylobacter jejuni and Lyme disease. The worldwide incidence rate is approximately 1.7 cases per 100,000 people annually. Other well-known causes of peripheral neuropathies include chronic alcoholism, infection of the varicella-zoster virus, botulism, and poliomyelitis. Peripheral neuropathy may develop as a primary symptom, or it may be due to another disease. For example, peripheral neuropathy is only one symptom of diseases such as amyloid neuropathy, certain cancers, or inherited neurologic disorders. Such diseases may affect the PNS and the CNS, as well as other body tissues.

Other PNS diseases treatable with the subject sirtuin activators include Brachial Plexus Neuropathies (diseases of the cervical and first thoracic roots, nerve trunks, cords, and peripheral nerve components of the brachial plexus). Clinical manifestations include regional pain, paresthesia; muscle weakness, and decreased sensation in the upper extremity. These disorders may be associated with trauma, including birth injuries; thoracic outlet syndrome; neoplasms, neuritis, radiotherapy; and other conditions. See Adams et al., Principles of Neurology, 6th ed, pp 1351-2); Diabetic Neuropathies (peripheral, autonomic, and cranial nerve disorders that are associated with diabetes mellitus). These conditions usually result from diabetic microvascular injury involving small blood vessels that supply nerves (vasa nervorum). Relatively common conditions which may be associated with diabetic neuropathy include third nerve palsy; mononeuropathy; mononeuritis multiplex; diabetic amyotrophy; a painful polyneuropathy; autonomic neuropathy; and thoracoabdominal neuropathy (see Adams et al., Principles of Neurology, 6th ed, p 1325); mononeuropathies (disease or trauma involving a single peripheral nerve in isolation, or out of proportion to evidence of diffuse peripheral nerve dysfunction). Mononeuritis multiplex refers to a condition characterized by multiple isolated nerve injuries. Mononeuropathies may result from a wide variety of causes, including ischemia; traumatic injury; compression; connective tissue diseases; cumulative trauma disorders; and other conditions; Neuralgia (intense or aching pain that occurs along the course or distribution of a peripheral or cranial nerve); Peripheral Nervous System Neoplasms (neoplasms which arise from peripheral nerve tissue). This includes neurofibromas; Schwannomas; granular cell tumors; and malignant peripheral nerve sheath tumors. See DeVita Jr et al., Cancer: Principles and Practice of Oncology, 5th ed, pp 1750-1); and Nerve Compression Syndromes (mechanical compression of nerves or nerve roots from internal or external causes). These may result in a conduction block to nerve impulses, due to, for example, myelin sheath dysfunction, or axonal loss. The nerve and nerve sheath injuries may be caused by ischemia; inflammation; or a direct mechanical effect; Neuritis (a general term indicating inflammation of a peripheral or cranial nerve). Clinical manifestation may include pain; paresthesias; paresis; or hyperesthesia; Polyneuropathies (diseases of multiple peripheral nerves). The various forms are categorized by the type of nerve affected (e.g., sensory, motor, or autonomic), by the distribution of nerve injury (e.g., distal vs. proximal), by nerve component primarily affected (e.g., demyelinating vs. axonal), by etiology, or by pattern of inheritance.

In another embodiment, a sirtuin activating compound may be used to treat or prevent any disease or disorder involving axonopathy. Distal axonopathy is a type of peripheral neuropathy that results from some metabolic or toxic derangement of peripheral nervous system (PNS) neurons. It is the most common response of nerves to metabolic or toxic disturbances, and as such may be caused by metabolic diseases such as diabetes, renal failure, deficiency syndromes such as malnutrition and alcoholism, or the effects of toxins or drugs. The most common cause of distal axonopathy is diabetes, and the most common distal axonopathy is diabetic neuropathy. The most distal portions of axons are usually the first to degenerate, and axonal atrophy advances slowly towards the nerve's cell body. If the noxious stimulus is removed, regeneration is possible, though prognosis decreases depending on the duration and severity of the stimulus. Those with distal axonopathies usually present with symmetrical glove-stocking sensori-motor disturbances. Deep tendon reflexes and autonomic nervous system (ANS) functions are also lost or diminished in affected areas.

In another embodiment, a sirtuin activating compound may be used to treat or prevent chemotherapeutic induced neuropathy. The sirtuin modulating compounds may be administered prior to administration of the chemotherapeutic agent, concurrently with administration of the chemotherapeutic drug, and/or after initiation of administration of the chemotherapeutic drug. If the sirtuin activating compound is administered after the initiation of administration of the chemotherapeutic drug, it is desirable that the sirtuin activating compound be administered prior to, or at the first signs, of chemotherapeutic induced neuropathy.

Chemotherapy drugs can damage any part of the nervous system. Encephalopathy and myelopathy are fortunately very rare. Damage to peripheral nerves is much more common and can be a side effect of treatment experienced by people with cancers, such as lymphoma. Most of the neuropathy affects sensory rather than motor nerves. Thus, the common symptoms are tingling, numbness or a loss of balance. The longest nerves in the body seem to be most sensitive hence the fact that most patients will report numbness or pins and needles in their hands and feet.

The chemotherapy drugs which are most commonly associated with neuropathy, are the Vinca alkaloids (anti-cancer drugs originally derived from a member of the periwinkle—the Vinca plant genus) and a platinum—containing drug called Cisplatin. The Vinca alkaloids include the drugs vinblastine, vincristine and vindesine. Many combination chemotherapy treatments for lymphoma for example CHOP and CVP contain vincristine, which is the drug known to cause this problem most frequently. Indeed, it is the risk of neuropathy that limits the dose of vincristine that can be administered.

Studies that have been performed have shown that most patients will lose some reflexes in their legs as a result of treatment with vincristine and many will experience some degree of tingling (paresthesia) in their fingers and toes. The neuropathy does not usually manifest itself right at the start of the treatment but generally comes on over a period of a few weeks. It is not essential to stop the drug at the first onset of symptoms, but if the neuropathy progresses this may be necessary. It is very important that patients should report such symptoms to their doctors, as the nerve damage is largely reversible if the drug is discontinued. Most doctors will often reduce the dose of vincristine or switch to another form of Vinca alkaloid such as vinblastine or vindesine if the symptoms are mild. Occasionally, the nerves supplying the bowel are affected causing abdominal pain and constipation.

In another embodiment, a sirtuin activating compound may be used to treat or prevent a polyglutamine disease. Huntington's Disease (HD) and Spinocerebellar ataxia type 1 (SCA1) are just two examples of a class of genetic diseases caused by dynamic mutations involving the expansion of triplet sequence repeats. In reference to this common mechanism, these disorders are called trinucleotide repeat diseases. At least 14 such diseases are known to affect human beings. Nine of them, including SCA1 and Huntington's disease, have CAG as the repeated sequence (see Table B below). Since CAG codes for an amino acid called glutamine, these nine trinucleotide repeat disorders are collectively known as polyglutamine diseases.

Although the genes involved in different polyglutamine diseases have little in common, the disorders they cause follow a strikingly similar course. Each disease is characterized by a progressive degeneration of a distinct group of nerve cells. The major symptoms of these diseases are similar, although not identical, and usually affect people in midlife. Given the similarities in symptoms, the polyglutamine diseases are hypothesized to progress via common cellular mechanisms. In recent years, scientists have made great strides in unraveling what the mechanisms are.

Above a certain threshold, the greater the number of glutamine repeats in a protein, the earlier the onset of disease and the more severe the symptoms. This suggests that abnormally long glutamine tracts render their host protein toxic to nerve cells.

To test this hypothesis, scientists have generated genetically engineered mice expressing proteins with long polyglutamine tracts. Regardless of whether the mice express full-length proteins or only those portions of the proteins containing the polyglutamine tracts, they develop symptoms of polyglutamine diseases. This suggests that a long polyglutamine tract by itself is damaging to cells and does not have to be part of a functional protein to cause its damage.

For example, it is thought that the symptoms of SCAL are not directly caused by the loss of normal ataxin-1 function but rather by the interaction between ataxin-1 and another protein called LANP. LANP is needed for nerve cells to communicate with one another and thus for their survival. When the mutant ataxin-1 protein accumulates inside nerve cells, it “traps” the LANP protein, interfering with its normal function. After a while, the absence of LANP function appears to cause nerve cells to malfunction.

TABLE B
Summary of Polyglutamine Diseases.

| | | | | | Normal | Disease |
|---|---|---|---|---|---|---|
| | Gene | Chromosomal | Pattern of | | repeat | repeat |
| Disease | name | location | inheritance | Protein | length | length |
| Spinobulbar | AR | Xq13-21 | X-linked | androgen | 9-36 | 38-62 |
| muscular atrophy | | | recessive | receptor | | |
| (Kennedy | | | | (AR) | | |
| disease) | | | | | | |
| Huntington's | HD | 4p16.3 | autosomal | huntingtin | 6-35 | 36-121 |
| disease | | | dominant | | | |
| Dentatorubral- | DRPLA | 12p13.31 | autosomal | atrophin-1 | 6-35 | 49-88 |
| pallidoluysian | | | dominant | | | |
| atrophy (Haw | | | | | | |
| River syndrome) | | | | | | |
| Spinocerebellar | SCA1 | 6p23 | autosomal | ataxin-1 | 6-44 | 39-82 |
| ataxia type 1 | | | dominant | | | |
| Spinocerebellar | SCA2 | 12q24.1 | autosomal | ataxin-2 | 15-31 | 36-63 |
| ataxia type 2 | | | dominant | | | |
| Spinocerebellar | SCA3 | 14q32.1 | autosomal | ataxin-3 | 12-40 | 55-84 |
| ataxia type 3 | | | dominant | | | |
| (Machado- | | | | | | |
| Joseph disease) | | | | | | |
| Spinocerebellar | SCA6 | 19p13 | autosomal | α1_{A}- | 4-18 | 21-33 |
| ataxia type 6 | | | dominant | voltage-dependent | | |
| | | | | calcium channel | | |
| | | | | subunit | | |
| Spinocerebellar | SCA7 | 3p12-13 | autosomal | ataxin-7 | 4-35 | 37-306 |
| ataxia type 7 | | | dominant | | | |
| Spinocerebellar | SCA17 | 6q27 | autosomal | TATA binding | 25-42 | 45-63 |
| ataxia type 17 | | | dominant | protein | | |

Many transcription factors have also been found in neuronal inclusions in different diseases. It is possible that these transcription factors interact with the polyglutamine-containing proteins and then become trapped in the neuronal inclusions. This in turn might keep the transcription factors from turning genes on and off as needed by the cell. Another observation is hypoacetylation of histones in affected cells. This has led to the hypothesis that Class I/II Histone Deacetylase (HDAC I/II) inhibitors, which are known to increase histone acetylation, may be a novel therapy for polyglutamine diseases (U.S. patent application Ser. No. 10/476,627; “Method of treating neurodegenerative, psychiatric, and other disorders with deacetylase inhibitors”).

In yet another embodiment, the invention provides a method for treating or preventing neuropathy related to ischemic injuries or diseases, such as, for example, coronary heart disease (including congestive heart failure and myocardial infarctions), stroke, emphysema, hemorrhagic shock, peripheral vascular disease (upper and lower extremities) and transplant related injuries.

In certain embodiments, the invention provides a method to treat a central nervous system cell to prevent damage in response to a decrease in blood flow to the cell. Typically the severity of damage that may be prevented will depend in large part on the degree of reduction in blood flow to the cell and the duration of the reduction. By way of example, the normal amount of perfusion to brain gray matter in humans is about 60 to 70 mL/100 g of brain tissue/min. Death of central nervous system cells typically occurs when the flow of blood falls below approximately 8-10 mL/100 g of brain tissue/min, while at slightly higher levels (i.e. 20-35 mL/100 g of brain tissue/min) the tissue remains alive but not able to function. In one embodiment, apoptotic or necrotic cell death may be prevented. In still a further embodiment, ischemic-mediated damage, such as cytoxic edema or central nervous system tissue anoxemia, may be prevented. In each embodiment, the central nervous system cell may be a spinal cell or a brain cell.

Another aspect encompasses administrating a sirtuin activating compound to a subject to treat a central nervous system ischemic condition. A number of central nervous system ischemic conditions may be treated by the sirtuin activating compounds described herein. In one embodiment, the ischemic condition is a stroke that results in any type of ischemic central nervous system damage, such as apoptotic or necrotic cell death, cytoxic edema or central nervous system tissue anoxia. The stroke may impact any area of the brain or be caused by any etiology commonly known to result in the occurrence of a stroke. In one alternative of this embodiment, the stroke is a brain stem stroke. Generally speaking, brain stem strokes strike the brain stem, which control involuntary life-support functions such as breathing, blood pressure, and heartbeat. In another alternative of this embodiment, the stroke is a cerebellar stroke. Typically, cerebellar strokes impact the cerebellum area of the brain, which controls balance and coordination. In still another embodiment, the stroke is an embolic stroke. In general terms, embolic strokes may impact any region of the brain and typically result from the blockage of an artery by a vaso-occlusion. In yet another alternative, the stroke may be a hemorrhagic stroke. Like ischemic strokes, hemorrhagic stroke may impact any region of the brain, and typically result from a ruptured blood vessel characterized by a hemorrhage (bleeding) within or surrounding the brain. In a further embodiment, the stroke is a thrombotic stroke. Typically, thrombotic strokes result from the blockage of a blood vessel by accumulated deposits.

In another embodiment, the ischemic condition may result from a disorder that occurs in a part of the subject's body outside of the central nervous system, but yet still causes a reduction in blood flow to the central nervous system. These disorders may include, but are not limited to a peripheral vascular disorder, a venous thrombosis, a pulmonary embolus, arrhythmia (e.g. atrial fibrillation), a myocardial infarction, a transient ischemic attack, unstable angina, or sickle cell anemia. Moreover, the central nervous system ischemic condition may occur as result of the subject undergoing a surgical procedure. By way of example, the subject may be undergoing heart surgery, lung surgery, spinal surgery, brain surgery, vascular surgery, abdominal surgery, or organ transplantation surgery. The organ transplantation surgery may include heart, lung, pancreas, kidney or liver transplantation surgery. Moreover, the central nervous system ischemic condition may occur as a result of a trauma or injury to a part of the subject's body outside the central nervous system. By way of example, the trauma or injury may cause a degree of bleeding that significantly reduces the total volume of blood in the subject's body. Because of this reduced total volume, the amount of blood flow to the central nervous system is concomitantly reduced. By way of further example, the trauma or injury may also result in the formation of a vaso-occlusion that restricts blood flow to the central nervous system.

Of course it is contemplated that the sirtuin activating compounds may be employed to treat the central nervous system ischemic condition irrespective of the cause of the condition. In one embodiment, the ischemic condition results from a vaso-occlusion. The vaso-occlusion may be any type of occlusion, but is typically a cerebral thrombosis or an embolism. In a further embodiment, the ischemic condition may result from a hemorrhage. The hemorrhage may be any type of hemorrhage, but is generally a cerebral hemorrhage or a subararachnoid hemorrhage. In still another embodiment, the ischemic condition may result from the narrowing of a vessel. Generally speaking, the vessel may narrow as a result of a vasoconstriction such as occurs during vasospasms, or due to arteriosclerosis. In yet another embodiment, the ischemic condition results from an injury to the brain or spinal cord.

In yet another aspect, a sirtuin activating compound may be administered to reduce infarct size of the ischemic core following a central nervous system ischemic condition. Moreover, a sirtuin activating compound may also be beneficially administered to reduce the size of the ischemic penumbra or transitional zone following a central nervous system ischemic condition.

In other aspects, the sirtuin-activating compounds described herein can be used to treat or prevent blood coagulation disorders (or hemostatic disorders). As used interchangeably herein, the terms “hemostasis”, “blood coagulation,” and “blood clotting” refer to the control of bleeding, including the physiological properties of vasoconstriction and coagulation. Blood coagulation assists in maintaining the integrity of mammalian circulation after injury, inflammation, disease, congenital defect, dysfunction or other disruption. After initiation of clotting, blood coagulation proceeds through the sequential activation of certain plasma proenzymes to their enzyme forms (see, for example, Coleman, R. W. et al. (eds.) Hemostasis and Thrombosis, Second Edition, (1987)). These plasma glycoproteins, including Factor XII, Factor XI, Factor IX, Factor X, Factor VII, and prothrombin, are zymogens of serine proteases. Most of these blood clotting enzymes are effective on a physiological scale only when assembled in complexes on membrane surfaces with protein cofactors such as Factor VIII and Factor V. Other blood factors modulate and localize clot formation, or dissolve blood clots. Activated protein C is a specific enzyme that inactivates procoagulant components. Calcium ions are involved in many of the component reactions. Blood coagulation follows either the intrinsic pathway, where all of the protein components are present in blood, or the extrinsic pathway, where the cell-membrane protein tissue factor plays a critical role. Clot formation occurs when fibrinogen is cleaved by thrombin to form fibrin. Blood clots are composed of activated platelets and fibrin.

Further, the formation of blood clots does not only limit bleeding in case of an injury (hemostasis), but may lead to serious organ damage and death in the context of atherosclerotic diseases by occlusion of an important artery or vein. Thrombosis is thus blood clot formation at the wrong time and place. It involves a cascade of complicated and regulated biochemical reactions between circulating blood proteins (coagulation factors), blood cells (in particular platelets), and elements of an injured vessel wall.

Accordingly, the present invention provides anticoagulation and antithrombotic treatments aimed at inhibiting the formation of blood clots in order to prevent or treat blood coagulation disorders, such as myocardial infarction, stroke, loss of a limb by peripheral artery disease, or pulmonary embolism.

As used interchangeably herein, “modulating or modulation of hemostasis” and “regulating or regulation of hemostasis” includes the induction (e.g., stimulation or increase) of hemostasis, as well as the inhibition (e.g., reduction or decrease) of hemostasis.

In one aspect of the invention, the invention provides a method for reducing or inhibiting hemostasis in a subject by administering a sirtuin-activating compound. The compositions and methods disclosed herein are useful for the treatment or prevention of thrombotic disorders. As used herein, the term “thrombotic disorder” includes any disorder or condition characterized by excessive or unwanted coagulation or hemostatic activity, or a hypercoagulable state. Thrombotic disorders include diseases or disorders involving platelet adhesion and thrombus formation, and may manifest as an increased propensity to form thromboses, e.g., an increased number of thromboses, thrombosis at an early age, a familial tendency towards thrombosis, and thrombosis at unusual sites. Examples of thrombotic disorders include, but are not limited to, thromboembolism, deep vein thrombosis, pulmonary embolism, stroke, arrhythmia (e.g. atrial fibrillation), myocardial infarction, miscarriage, thrombophilia associated with anti-thrombin III deficiency, protein C deficiency, protein S deficiency, resistance to activated protein C, dysfibrinogenemia, fibrinolytic disorders, homocystinuria, pregnancy, inflammatory disorders, myeloproliferative disorders, arteriosclerosis, angina, e.g., unstable angina, disseminated intravascular coagulation, thrombotic thrombocytopenic purpura, cancer metastasis, sickle cell disease, glomerular nephritis, and drug induced thrombocytopenia (including, for example, heparin induced thrombocytopenia). In addition, the subject sirtuin-activating compounds are administered to prevent thrombotic events or to prevent re-occlusion during or after therapeutic clot lysis or procedures such as angioplasty or surgery.

In certain aspects, the sirtuin-activating compounds described herein may be taken alone or in combination with other compounds. The other compounds may be other sirtuin activators. For example, it has been shown by the inventors that Longevinex™ which is a red wine extract and contains, in addition to resveratrol, other sirtuin activators such as quercetin. Longevinex™ can be obtained on the worldwide web at longevinex.com.

In one embodiment, a combination drug regimen may include drugs or compounds for the treatment or prevention of neurodegenerative disorders or secondary conditions associated with these conditions. Thus, a combination drug regimen may include one or more sirtuin activators and one or more anti-neurodegeneration agents. For example, one or more sirtuin-activating compounds can be combined with an effective amount of one or more of: L-DOPA; a dopamine agonist; an adenosine A_{2A} receptor antagonist; a COMT inhibitor; a MAO inhibitor; an N-NOS inhibitor; a sodium channel antagonist; a selective N-methyl D-aspartate (NMDA) receptor antagonist; an AMPA/kainate receptor antagonist; a calcium channel antagonist; a GABA-A receptor agonist; an acetyl-choline esterase inhibitor; a matrix metalloprotease inhibitor; a PARP inhibitor; an inhibitor of p38 MAP kinase or c-jun-N-terminal kinases; TPA; NDA antagonists; beta-interferons; growth factors; glutamate inhibitors; and/or as part of a cell therapy.

Exemplary N-NOS inhibitors include 4-(6-amino-pyridin-2-yl)-3-methoxyphenol 6-[4-(2-dimethylamino-ethoxy)-2-methoxy-phenyl]-pyridin-2-yl-amine, 6-[4-(2-dimethylamino-ethoxy)-2,3-dimet-hyl-phenyl]-pyridin-2-yl-amine, 6-[4-(2-pyrrolidinyl-ethoxy)-2,3-dimethyl-p-henyl]-pyridin-2-yl-amine, 6-[4-(4-(n-methyl)piperidinyloxy)-2,3-dimethyl-p-henyl]-pyridin-2-yl-amine, 6-[4-(2-dimethylamino-ethoxy)-3-methoxy-phenyl]-pyridin-2-yl-amine, 6-[4-(2-pyrrolidinyl-ethoxy)-3-methoxy-phenyl]-pyridin-2-yl-amine, 6-{4-[2-(6,7-dimethoxy-3,4-dihydro-1h-isoquinolin-2-yl)-ethoxy]-3-methoxy-phenyl}-pyridin-2-yl-amine, 6-{3-methoxy-4-[2-(4-phenethyl-piper-azin-1-yl)-ethoxy]-phenyl}-pyridin-2-yl-amine, 6-{3-methoxy-4-[2-(4-methyl-piperazin-1-yl)-ethoxy]-phenyl}-pyridin-2-yl-amine, 6-{4-[2-(4-dimethylamin-o-piperidin-1-yl)-ethoxy]-3-methoxy-phenyl}-pyridin-2-yl-amine, 6-[4-(2-dimethylamino-ethoxy)-3-ethoxy-phenyl]-pyridin-2-yl-amine, 6-[4-(2-pyrrolidinyl-ethoxy)-3-ethoxy-phenyl]-pyridin-2-yl-amine, 6-[4-(2-dimethylamino-ethoxy)-2-isopropyl-phenyl]-pyridin-2-yl-amine, 4-(6-amino-pyridin-yl)-3-cyclopropyl-phenol 6-[2-cyclopropyl-4-(2-dimethy-lamino-ethoxy)-phenyl]-pyridin-2-yl-amine, 6-[2-cyclopropyl-4-(2-pyrrolidin-1-yl-ethoxy)-phenyl]-pyridin-2-yl-amine, 3-[3-(6-amino-pyridin-2yl)4-cycl-opropyl-phenoxy]-pyrrolidine-1-carboxylic acid tert-butyl ester 6-[2-cyclopropyl-4-(1-methyl-pyrrolidin-3-yl-oxy)-phenyl]-pyridin-2-yl-amine, 4-(6-amino-pyridin-2-yl)-3-cyclobutyl-phenol 6-[2-cyclobutyl-4-(2-dime-thylamino-ethoxy)-phenyl]-pyridin-2-yl-amine, 6-[2-cyclobutyl-4-(2-pyrrolid-in-1-yl-ethoxy)-phenyl]-pyridin-2-yl-amine, 6-[2-cyclobutyl-4-(1-methyl-pyrrolidin-3-yl-oxy)-phenyl]-pyridin-2-yl-amine, 4-(6-amino-pyridin-2-yl)-3-cy-clopentyl-phenol 6-[2-cyclopentyl-4-(2-dimethylamino-ethoxy)-phenyl]-pyrid-in-2-yl-amine, 6-[2-cyclopentyl-4-(2-pyrrolidin-1yl-ethoxy)-phenyl]-pyridin-2-yl-amine, 3-[4-(6-amino-pyridin-2yl)-3-methoxy-phenoxy]-pyrrolidine-1-ca-rboxylic acid tert butyl ester 6-[4-(1-methyl-pyrrolidin-3-yl-oxy)-2-metho-xy-phenyl]-pyridin-2-yl-amine, 4-[4-(6-amino-pyridin-2yl)-3-methoxy-phenoxy-]-piperidine-1-carboxylic acid tert butyl ester 6-[2-methoxy-4-(1-methyl-p-iperidin-4-yl-oxy)-phenyl]-pyridin-2-yl-amine, 6-[4-(allyloxy)-2-methoxy-ph-enyl]-pyridin-2-yl-amine, 4-(6-amino-pyridin-2-yl)-3-methoxy-6-allyl-phenol 12 and 4-(6-amino-pyridin-2-yl)-3-methoxy-2-allyl-phenol 13 4-(6-amino-pyridin-2-yl)-3-methoxy-6-propyl-phenol 6-[4-(2-dimethylamino-ethoxy)-2-methoxy-5-propyl-phenyl]-pyridin-yl-amine, 6-[2-isopropyl-4-(pyrrolidin-3-yl-oxy)-phenyl]-pyridin-2-yl-amine, 6-[2-isopropyl-4-(piperidin-3-yl-oxy)-phenyl]-pyridin-2-yl-amine, 6-[2-isopropyl-4-(1-methyl-azetidin-3-yl-oxy)-phenyl]-pyridin-2-yl-amine, 6-[2-isopropyl-4-(1-methyl-piperidin-4-yl-oxy)-phenyl]-pyridin-2-yl-amine, 6-[2-isopropyl-4-(1-methyl-pyrrolidin-3-yl-oxy)-phenyl]-pyridin-2-yl-amin-e 6-[2-isopropyl-4-(1-methyl-pyrrolidin-3-yl-oxy)-phenyl]-pyridin-2-yl-amine, 6-[2-isopropyl4-(2-methyl-2-aza-bicyclo[2.2.1 ]hept-5-yl-oxy)-phenyl]-p-yridin-2-yl-amine, 6-[4-(2-dimethylamino-ethoxy)-2-methoxy-phenyl]-pyridin-2-yl-amine, 6-{4-[2-(benzyl-methyl-amino)-ethoxy]-2-methoxy-phenyl}-pyridin-2-yl-amine, 6-[2-methoxy-4-(2-pyrrolidin-1-yl-ethoxy)-phenyl]-pyridin-2-yl-amine, 2-(6-amino-pyridin-2-yl)-5-(2-dimethylamino-ethoxy)-phenol 2-[4-(6-amino-pyridin-2-yl)-3-methoxy-phenoxy]-acetamide 6-[4-(2-amino-ethoxy)-2-methoxy-phenyl]-pyridin-2-yl-amine, 6-{4-[2-(3,4-dihydro-1h-isoquinolin-2-yl)-ethoxy]-2-methoxy-phenyl}-pyrid-in-2-yl-amine, 2-[4-(6-amino-pyridin-2-yl)-3-methoxy-phenoxy]-ethanol 6-{2-methoxy-4-[2-(2,2,6,6-tetramethyl-piperidin-1-yl)-ethoxy]-phenyl}-py-ridin-2-yl-amine, 6-{4-[2-(2,5-dimethyl-pyrrolidin-1-yl)-ethoxy]-2-methoxy-phenyl}-pyridin-2-yl-amine, 6-{4-[2-(2,5-dimethyl-pyrrolidin-1-yl)-ethoxy]-2-methoxy-phenyl}-pyridin-2-yl-amine, 2-[4-(6-amino-pyridin-2-yl)-3-methoxy-phenoxy]-1-(2,2,6,6-tetramethyl-piperidin-1-yl)-ethanone 6-[2-methoxy-4-(1-methyl-pyrrolidin-2-yl-methoxy)-phenyl]-pyridin-2-yl-amine, 6-[4-(2-dimethylamino-ethoxy)-2-propoxy-phenyl]-pyridin-2-yl-amine, 6-{4-[2-(benzyl-methyl-amino)-ethoxy]-2-propoxy-phenyl}-pyridin-2-yl-amin-e 6-[4-(2-ethoxy-ethoxy)-2-methoxy-phenyl]-pyridin-2-yl-amine, 6-[4-(2-dimethylamino-ethoxy)-2-isopropoxy-phenyl]-pyridin-2-yl-amine, 6-[4-(2-ethoxy-ethoxy)-2-isopropoxy-phenyl]-pyridin-2-yl-amine, 6-[2-methoxy-4-(3-methyl-butoxy)-phenyl]-pyridin-2-yl-amine, 6-[4-(2-dimethylamino-ethoxy)-2-ethoxy-phenyl]-pyridin-2-yl-amine, 6-{4-[2-(benzyl-methyl-amino)-ethoxy]-2-ethoxy-phenyl}-pyridin-2-yl-amine, 6-[2-ethoxy-4-(3-methyl-butoxy)-phenyl]-pyridin-2-yl-amine, 1-(6-amino-3-aza-bicyclo[3.1.0]hex-3-yl)-2-[4-(6-amino-pyridin-2-yl)-3-et-hoxy-phenoxy]-ethanone 6-[2-ethoxy-4-(2-pyrrolidin-1-yl-ethoxy)-phenyl]-py-ridin-2-yl-amine, 3-{2-[4-(6-amino-pyridin-2-yl)-3-ethoxy-phenoxy]-ethyl}-3-aza-bicyclo[3.1.0]hex-6-yl-amine, 1-(6-amino-3-aza-bicyclo[3.1.0]hex-3-yl)-2-[4-(6-amino-pyridin-2-yl)-3-methoxy-phenoxy]-ethanone 3-{2-[4-(6-amino-pyridin-2-yl)-3-methoxy-phenoxy]-ethyl}-3-aza-bicyclo[3.1.0]hex-6-yl-amine, 6-[2-isopropoxy-4-(2-pyrrolidin-1-yl-ethoxy)-phenyl]-py-ridin-2-yl-amine, 6-{4-[2-(benzyl-methyl-amino)-ethoxy]-2-isopropoxy-phenyl-}-pyridin-2-yl-amine, 6-[4-(2-dimethylamino-ethoxy)-2-methoxy-5-propyl-phen-yl]-pyridin-2-yl-amine, 6-[5-allyl4-(2-dimethylamino-ethoxy)-2-methoxy-phe-nyl]-pyridin-2-yl-amine, 6-[5-allyl-2-methoxy4-(2-pyrrolidin-1-yl-ethoxy)-phenyl]-pyridin-2-yl-amine, 6-[3-allyl-4-(2-dimethylamino-ethoxy)-2-methoxy-phenyl]-pyridin-2-yl-amine, 6-[2-methoxy-4-(pyrrolidin-3-yl-oxy)-phenyl]-p-yridin-2-yl-amine, 6-[2-methoxy4-(1-methyl-pyrrolidin-3-yl-oxy)-phenyl]-py-ridin-2-yl-amine, 6-[2-ethoxy-4-(pyrrolidin-3-yl-oxy)-phenyl]-pyridin-2-yl-amine, 6-[2-isopropoxy-4-(pyrrolidin-3-yl-oxy)-phenyl]-pyridin-2-yl-amine, 6-[2-methoxy-4-(piperidin-4-yl-oxy)-phenyl]-pyridin-2-yl-amine, 6-[2-methoxy-4-(2,2,6,6-tetramethyl-piperidin-4-yl-oxy)-phenyl]-pyridin-2-yl-amine, 6-[2-isopropoxy-4-(pyrrolidin-3-yl-oxy)-phenyl]-pyridin-2-yl-amine, 3-[4-(6-amino-pyridin-2-yl)-3-methoxy-phenoxy]-azetidine-1-carboxylic acid tert-butyl ester 6-[4-(azetidin-3-yl-oxy)-2-methoxy-phenyl]-pyridin-2-yl-amine, 6-[2-methoxy-4-(1-methyl-azetidin-3-yl-oxy)-phenyl]-pyridin-2-y-1-amine, 6-[2-isopropoxy-4-(pyrrolidin-3-yl-oxy)-phenyl]-pyridin-2-yl-amine, 6-[2-isopropoxy-4-(pyrrolidin-3-yl-oxy)-phenyl]-pyridin-2-yl-amine, 6-[2-methoxy-4-(pyrrolidin-3-yl-oxy)-phenyl]-pyridin-2-yl-amine, 6-[2-methoxy-4-(1-methyl-pyrrolidin-3-yl-oxy)-phenyl]-pyridin-2-yl-amine, 6-[2-methoxy-4-(1-methyl-pyrrolidin-3-yl-oxy)-phenyl]-pyridin-2-yl-amine, 6-[2-methoxy-4-(2-methyl-2-aza-bicyclo[2.2.1]hept-5-yl-oxy)-phenyl]-pyrid-in-2-yl-amine, 6-[2-methoxy-4-(1-methyl-piperidin-4-yl-oxy)-phenyl]-pyridin-2-yl-amine, 6-[4-(1-ethyl-piperidin-4-yl-oxy)-2-methoxy-phenyl]-pyridin-2-yl-amine, 6-[5-allyl-2-methoxy-4-(1-methyl-pyrrolidin-3-yl-oxy)-phenyl]-pyr-idin-2-yl-amine, 6-[4-(2-dimethylamino-ethoxy)-2,6-dimethyl-phenyl]-pyridin-2-yl-amine, 6-[2,6-dimethyl-4-(3-piperidin-1-yl-propoxy)-phenyl]-pyridin-2-yl-amine, 6-[2,6-dimethyl-4-(2-pyrrolidin-1-yl-ethoxy)-phenyl]-pyridin-2-y-l-amine, 6-{2,6-dimethyl-4-[3-(4-methyl-piperazin-1-yl)-propoxy]-phenyl}-py-ridin-2-yl-amine, 6-[2,6-dimethyl-4-(2-morpholin-4-yl-ethoxy)-phenyl]-pyrid-in-2-yl-amine, 6-{4-[2-(benzyl-methyl-amino)-ethoxy]-2,6-dimethyl-phenyl}-p-yridin-2-yl-amine, 2-[4-(6-amino-pyridin-2-yl)-3,5-dimethyl-phenoxy]-acetam-ide 6-[4-(2-amino-ethoxy)-2,6-dimethyl-phenyl]-pyridin-2-yl-amine, 6-[2-isopropyl-4-(2-pyrrolidin-1-yl-ethoxy)-phenyl]-pyridin-2-yl-amine, 2-(2,5-dimethyl-pyrrolidin-1-yl)-6-[2-isopropyl-4-(2-pyrrolidin-1-yl-etho-xy)-phenyl]-pyridine 6-{4-[2-(3,5-dimethyl-piperidin-1-yl)-ethoxy]-2-isopr-opyl-phenyl}-pyridin-2-yl-amine, 6-[4-(2-dimethylamino-ethoxy)-2-isopropyl-phenyl]-pyridin-2-yl-amine, 6-[2-tert-butyl4-(2-dimethylamino-ethoxy)-phen-yl]-pyridin-2-yl-amine, 6-[2-tert-butyl4-(2-pyrrolidin-1-yl-ethoxy)-phenyl-]-pyridin-2-yl-amine, 6-[4-(2-pyrrolidinyl-ethoxy)-2,5-dimethyl-phenyl]-pyr-idin-2-yl-amine, 6-[4-(2-dimethylamino-ethoxy)-2,5-dimethyl-phenyl]-pyridin-2-yl-amine, 6-[4-(2-(4-phenethylpiperazin-1-yl)-ethoxy)-2,5-dimethyl-pheny-1]-pyridin-2-yl-amine, 6-[2-cyclopropyl4-(2-dimethylamino-1-methyl-ethoxy)-phenyl]-pyridin-2-yl-amine, 6-[cyclobutyl-4-(2-dimethylamino-1-methyl-etho-xy)-phenyl]-pyridin-2-yl-amine, 6-[4-(allyloxy)-2-cyclobutyl-phenyl]-pyridi-n-2ylamine, 2-allyl-4-(6-amino-pyridin-2-yl)-3-cyclobutyl-phenol and 2-allyl4-(6-amino-pyridin-2-yl)-5-cyclobutyl-phenol 4-(6-amino-pyridin-2yl)-5-cyclobutyl-2-propyl-phenol 4-(6-amino-pyridin-2yl)-3-cyclobutyl-2-propyl-phenol 6-[2-cyclobutyl-4-(2-dimethylamino-1-methyl-ethoxy)-5-propyl-phenyl]-pyri-din-2-yl-amine, 6-[2-cyclobutyl-4-(2-dimethylamino-1-methyl-ethoxy)-3-propy-l-phenyl]-pyridin-2-yl-amine, 6-[2-cyclobutyl-4-(2-dimethylamino-ethoxy)-5-propyl-phenyl]-pyridin-2-yl-amine, 6-[2-cyclobutyl-4-(2-dimethylamino-ethox-y)-3-propyl-phenyl]-pyridin-2-yl-amine, 6-[2-cyclobutyl-4-(1-methyl-pyrroli-din-3-yl-oxy)-5-propyl-phenyl]-pyridin-2-yl-amine, 6-[cyclobutyl-4-(1-methy-1-pyrrolidin-3-yl-oxy)-3-propyl-phenyl]-pyridin-2-yl-amine, 2-(4-benzyloxy-5-hydroxy-2-methoxy-phenyl)-6-(2,5-dimethyl-pyrrol-1-yl)-p-yridine 6-[4-(2-dimethylamino-ethoxy)-5-ethoxy-2-methoxy-phenyl]-pyridin-2-yl-amine, 6-[5-ethyl-2-methoxy-4-(1-methyl-piperidin4-yl-oxy)-phenyl]-pyr-idin-2-yl-amine, 6-[5-ethyl-2-methoxy-4-(piperidin-4-yl-oxy)-phenyl]-pyridi-n-2-yl-amine, 6-[2,5-dimethoxy-4-(1-methyl-pyrrolidin-3-yl-oxy)-phenyl]-pyr-idin-2-yl-amine, 6-[4-(2-dimethylamino-ethoxy)-5-ethyl-2-methoxy-phenyl]-py-ridin-2-yl-amine.

Exemplary NMDA receptor antagonist include (+)-(1S,2S)-1-(4-hydroxy-phenyl)-2-(4-hydroxy-4-phenylpiperidino)-1-pro-panol, (1S,2S)-1-(4-hydroxy-3-methoxyphenyl)-2-(4-hydroxy-4-phenylpiperi-dino)-1-propanol, (3R,4S)-3-(4-(4-fluorophenyl)-4-hydroxypiperidin-1-yl-)-chroman-4,7-diol, (1R*,2R*)-1-(4-hydroxy-3-methylphenyl)-2-(4-(4-fluoro-phenyl)-4-hydroxypiperidin-1-yl)-propan-1-ol-mesylate or a pharmaceutically acceptable acid addition salt thereof.

Exemplary dopamine agonist include ropininole; L-dopa decarboxylase inhibitorss such as carbidopa or benserazide, bromocriptine, dihydroergocryptine, etisulergine, AF-14, alaptide, pergolide, piribedil; dopamine D1 receptor agonists such as A-68939, A-77636, dihydrexine, and SKF-38393; dopamine D2 receptor agonists such as carbergoline, lisuride, N-0434, naxagolide, PD-1 18440, pramipexole, quinpirole and ropinirole; dopamine/β-adrenegeric receptor agonists such as DPDMS and dopexamine; dopamine/5-HT uptake inhibitor/5-HT-1A agonists such as roxindole; dopamine/opiate receptor agonists such as NIH-10494; α2-adrenergic antagonist/dopamine agonists such as terguride; α2-adrenergic antagonist/dopamine D2 agonists such as ergolines and talipexole; dopamine uptake inhibitors such as GBR-12909, GBR-13069, GYKI-52895, and NS-2141; monoamine oxidase-B inhibitors such as selegiline, N-(2-butyl)-N-methylpropargylamine, N-methyl-N-(2-pentyl)propargylamine, AGN-1133, ergot derivatives, lazabemide, LU-53439, MD-280040 and mofegiline; and COMT inhibitors such as CGP-28014.

Exemplary acetyl cholinesterase inhibitors include donepizil, 1-(2-methyl-1H-benzimida-zol-5-yl)-3-[1-(phenylmethyl)-4-piperidinyl]-1-propanone; 1-(2-phenyl-1H-benzimidazol-5-yl)-3-[1-(phenylmethyl)-4-piperidinyl]-1-pr-opanone; 1-(1-ethyl-2-methyl-1H-benzimidazol-5-yl)-3-[1-(phenylmethyl)-4-p-iperidinyl]-1-propanone; 1-(2-methyl-6-benzothiazolyl)-3-[1-(phenylmethyl)-4-piperidinyl]-1-propanone; 1-(2-methyl-6-benzothiazolyl)-3-[1-[(2-methyl-4-thiazolyl)methyl]-4-piperidinyl]-1-propanone; 1-(5-methyl-benzo[b]thie-n-2-yl)-3-[1-(phenylmethyl)-4-piperidinyl]-1-propanone; 1-(6-methyl-benzo[b]thien-2-yl)-3-[1-(phenylmethyl)-4-piperidinyl]-1-prop-anone; 1-(3,5-dimethyl-benzo[b]thien-2-yl)-3-[1-(phenylmethyl)-4-piperidin-yl]-1-propanone; 1-(benzo[b]thien-2-yl)-3-[1-(phenylmethyl)-4-piperidinyl]-1-propanone; 1-(benzofuran-2-yl)-3-[1-(phenylmethyl)-4-piperidinyl]-1-pro-panone; 1-(1-phenylsulfonyl-6-methyl-indol-2-yl)-3-[1-(phenylmethyl)-4-pip-eridinyl]-1-propanone; 1-(6-methyl-indol-2-yl)-3-[1-(phenylmethyl)-4-piper-idinyl]-1-propanone; 1-(1-phenylsulfonyl-5-amino-indol-2-yl)-3-[1-(phenylm-ethyl)-4-piperidinyl]-1-propanone; 1-(5-amino-indol-2-yl)-3-[1-(phenylmet-hyl)-4-piperidinyl]-1-propanone; and 1-(5-acetylamino-indol-2-yl)-3-[1-(ph-enylmethyl)-4-piperidinyl]-1-propanone. 1-(6-quinolyl)-3-[1-(phenylmethyl)-4-piperidinyl]-1-propanone; 1-(5-indolyl)-3-[1-(phenylmethyl)-4-piperidiny-l]-1-propanone; 1-(5-benzthienyl)-3-[1-(phenylmethyl)-4-piperidinyl]-1-pro-panone; 1-(6-quinazolyl)-3-[1-(phenylmethyl)-4-piperidinyl]-1-propanone; 1-(6-benzoxazolyl)-3-[1-(phenylmethyl)-4-piperidinyl]-1-propanone; 1-(5-benzofuranyl)-3-[1-(phenylmethyl)-4-piperidinyl]-1-propanone; 1-(5-methyl-benzimidazol-2-yl)-3-[1-(phenylmethyl)-4-piperidinyl]-1-propa-none; 1-(6-methyl-benzimidazol-2-yl)-3-[1-(phenylmethyl)-4-piperidinyl]-1-propanone; 1-(5-chloro-benzo[b]thien-2-yl)-3-[1-(phenylmethyl)-4-piperidin-yl]-1-propanone; 1-(5-azaindol-2-yl)-3-[1-(phenylmethyl)-4-piperidinyl]-1-p-ropanone; 1-(6-azabenzo[b]thien-2-yl)-3-[1-(phenylmethyl)-4-piperidinyl]-1-propanone; 1-(1H-2-oxo-pyrrolo[2′,3′,5,6]benzo[b]thieno-2-yl)-3-[1-(phenylmethyl)-4-1-propanone; 1-(6-methyl-benzothiazol-2-yl)-3-[1-(phenylmethyl)-4-piperidinyl]-1-propanone; 1-(6-methoxy-indol-2-yl)-3-[1-(phenylmethyl)-4-piperidinyl]-1-propanone; 1-(6-methoxy-benzo[b]thien-2-yl)-3-[1-(phenylmethyl)-4-piperidinyl]-1-pro-panone; 1-(6-acetylamino-benzo[b]thien-2-yl)-3-[1-(phenylmethyl)-4-piperid-inyl]-1-propanone; 1-(5-acetylamino-benzo[b]thien-2-yl)-3-[1-(phenylmethyl-)-4-piperidinyl]-1-propanone; 6-hydroxy-3-[2-[1-(phenylmethyl)-4-piperidin-yl]ethyl]-1,2-benzisoxazole; 5-methyl-3-[2-[1-(phenylmethyl)-4-piperidinyl-]ethyl]-1,2-benzisoxazole; 6-methoxy-3[2-[1(phenylmethyl)-4-piperidinyl]et-hyl]-1,2-benzisoxazole; 6-acetamide-3-[2-[1-(phenylmethyl)-4-piperidinyl]-ethyl]-1,2-benzisoxazole; 6-amino-3-[2-[1-(phenymethyl)-4-piperidinyl]ethy-1]-1,2-benzisoxazole; 6-(4-morpholinyl)-3-[2-[1-(phenylmethyl)-4-piperidin-yl]ethyl]-1,2-benzisoxazole; 5,7-dihydro-3-[2-[ 1-(phenylmethyl)-4-piperidi-nyl]ethyl]-6H-pyrrolo[4,5-f]-1,2-benzisoxazol-6-one; 3-[2-[1-(phenylmethyl)-4-piperidinyl]ethyl]-1,2-benzisothiazole; 3-[2-[1-(phenylmethyl)-4-piperidinyl]ethenyl]-1,2-benzisoxazole; 6-phenylamino-3-[2-[1-(phenylmethyl)4-piperidinyl]ethyl]-1,2,-benzisoxaz-ole; 6-(2-thiazoly)-3-[2-[1-(phenylmethyl)-4-piperidinyl]ethyl]-1,2-benzis-oxazole; 6-(2-oxazolyl)-3-[2-[1-(phenylmethyl)4-piperidinyl]ethyl]-1,2-be-nzisoxazole; 6-pyrrolidinyl-3-[2-[1-(phenylmethyl)-4-piperidinyl]ethyl]-1,-2-benzisoxazole; 5,7-dihydro-5,5-dimethyl-3-[2-[1-(phenylmethyl)-4-piperid-inyl]ethyl]-6H-pyrrolo[4,5-f]-1,2-benzisoxazole-6-one; 6,8-dihydro-3-[2-[1-(phenylmethyl)-4-piperidinyl]ethyl]-7H-pyrrolo[5,4-g]-1,2-benzisoxazole-7-one; 3-[2-[1-(phenylmethyl)-4-piperidinyl]ethyl]-5,6,-8-trihydro-7H-isoxazolo[4,5-g]-quinolin-7-one; 1-benzyl-4-((5,6-dimethoxy-1-indanon)-2-yl)methylpiperidine, 1-benzyl-4-((5,6-dimethoxy-1-indanon)-2-ylidenyl)methylpiperidine, 1-benzyl-4-((5-methoxy-1-indanon)-2-yl)methylp-iperidine, 1-benzyl-4-((5,6-diethoxy-1-indanon)-2-yl)methylpiperidine, 1-benzyl-4-((5,6-methnylenedioxy-1-indanon)-2-yl)methylpiperidine, 1-(m-nitrobenzyl)-4-((5,6-dimethoxy-1-indanon)-2-yl)methylpiperidine, 1-cyclohexymethyl-4-((5,6-dimethoxy-1-indanon)-2-yl)methylpiperidine, 1-(m-florobenzyl)4-((5,6-dimethoxy-1-indanon)-2-yl)methylpiperidine, 1-benzyl-4-((5,6-dimethoxy-1-indanon)-2-yl)propylpiperidine, and 1-benzyl-4-((5-isopropoxy-6-methoxy-1-indanon)-2-yl)methylpiperidine. Exemplary calcium channel antagonists include diltiazem, omega-conotoxin GVIA, methoxyverapamil, amlodipine, felodipine, lacidipine, and mibefradil.

Exemplary GABA-A receptor modulators include clomethiazole; IDDB; gaboxadol (4,5,6,7-tetrahydroisoxazolo[5,4-c]pyridin-3-ol); ganaxolone (3.alpha.-hydroxy-3.beta.-methyl-5.alpha.-pregnan-20-one); fengabine (2-[(butylimino)-(2-chlorophenyl)methyl]4-chlorophenol); 2-(4-methoxyphenyl)-2,5,6,7,8,9-hexahydro-pyrazolo[4,3-c]cinnolin-3-one; 7-cyclobutyl-6-(2-methyl-2H-1,2,4-triazol-3-ylmethoxy)-3-phenyl-1,2,4-triazolo[4,3-b]pyridazine; (3-fluoro4-methylphenyl)-N-({-1-[(2-methylphenyl)methyl]-benzimidazol-2-yl}methyl)-N-pentylcarboxamide; and 3-(aminomethyl)-5-methylhexanoic acid.

Exemplary potassium channel openers include diazoxide, flupirtine, pinacidil, levcromakalim, rilmakalim, chromakalim, PCO-400 and SKP-450 (2-[2″(1″,3″-dioxolone)-2-methyl]-4-(2′-oxo-1′-pyrrolidinyl)-6-nitro-2H-1-benzopyra-n).

Exemplary AMPA/kainate receptor anatagonists include 6-cyano-7-nitroquinoxalin-2,3-di-one (CNQX); 6-nitro-7-sulphamoylbenzo[f]quinoxaline-2,3-dione (NBQX); 6,7-dinitroquinoxaline-2,3-dione (DNQX); 1-(4-aminophenyl)4-methyl-7,8-m-ethylenedioxy-5H-2,3-benzodiazepine hydrochloride; and 2,3-dihydroxy-6-nitro-7-sulfamoylbenzo-[f]quinoxaline.

Exemplary sodium channel antagonists include ajmaline, procainamide, flecainide and riluzole.

Exemplary matrix-metalloprotease inhibitors include 4-[4-(4-fluorophenoxy)benzenesulfon-ylamino]tetrahydropyran-4-carboxylic acid hydroxyamide; 5-Methyl-5-(4-(4′-fluorophenoxy)-phenoxy)-pyrimidine-2,4,6-trione; 5-n-Butyl-5-(4-(4′-fluorophenoxy)-phenoxy)-pyrimidine-2,4,6-trione and prinomistat.

Poly(ADP ribose) polymerase (PARP) is an abundant nuclear enzyme which is activated by DNA strand single breaks to synthesize poly (ADP ribose) from NAD. Under normal conditions, PARP is involved in base excision repair caused by oxidative stress via the activation and recruitment of DNA repair enzymes in the nucleus. Thus, PARP plays a role in cell necrosis and DNA repair. PARP also participates in regulating cytokine expression that mediates inflammation. Under conditions where DNA damage is excessive (such as by acute excessive exposure to a pathological insult), PARP is over-activated, resulting in cell-based energetic failure characterized by NAD depletion and leading to ATP consumption, cellular necrosis, tissue injury, and organ damage/failure. PARP is thought to contribute to neurodegeneration by depleting nicotinamide adenine dinucleotide (NAD+) which then reduces adenosine triphosphate (ATP; Cosi and Marien, Ann. N.Y. Acad. Sci., 890:227, 1999) contributing to cell death which can be prevented by PARP inhibitors. Exemplory PARP inhibitors can be found in Southan and Szabo, Current Medicinal Chemistry, 10:321, 2003.

Exemplary inhibitors of p38 MAP kinase and c-jun-N-terminal kinases include pyridyl imidazoles, such as PD 169316, isomeric PD 169316, SB 203580, SB 202190, SB 220026, and RWJ 67657. Others are described in U.S. Pat. No. 6,288,089, and incorporated by reference herein.

In an exemplary embodiment, a combination therapy for treating or preventing MS comprises a therapeutically effective amount of one or more sirtuin activating compounds and one or more of Avonex® (interferon beta-1a), Tysabri® (natalizumab), or Fumaderm® (BG-12/Oral Fumarate).

In another embodiment, a combination therapy for treating or preventing diabetic neuropathy or conditions associated therewith comprises a therapeutically effective amount of one or more sirtuin activating compounds and one or more of tricyclic antidepressants (TCAs) (including, for example, imipramine, amytriptyline, desipramine and nortriptyline), serotonin reuptake inhibitors (SSRIs) (including, for example, fluoxetine, paroxetine, sertralene, and citalopram) and antiepileptic drugs (AEDs) (including, for example, gabapentin, carbamazepine, and topimirate).

In another embodiment, the invention provides a method for treating or preventing a polyglutamine disease using a combination comprising at least one sirtuin activating compound and at least one HDAC I/II inhibitor. Examples of HDAC I/II inhibitors include hydroxamic acids, cyclic peptides, benzamides, short-chain fatty acids, and depudecin.

Examples of hydroxamic acids and hydroxamic acid derivatives, but are not limited to, trichostatin A (TSA), suberoylanilide hydroxamic acid (SAHA), oxamflatin, suberic bishydroxamic acid (SBHA), m-carboxy-cinnamic acid bishydroxamic acid (CBHA), valproic acid and pyroxamide. TSA was isolated as an antifungi antibiotic (Tsuji et al (1976) J. Antibiot (Tokyo) 29:1-6) and found to be a potent inhibitor of mammalian HDAC (Yoshida et al. (1990) J. Biol. Chem. 265:17174-17179). The finding that TSA-resistant cell lines have an altered HDAC evidences that this enzyme is an important target for TSA. Other hydroxamic acid-based HDAC inhibitors, SAHA, SBHA, and CBHA are synthetic compounds that are able to inhibit HDAC at micromolar concentration or lower in vitro or in vivo. Glick et al. (1999) Cancer Res. 59:4392-4399. These hydroxamic acid-based HDAC inhibitors all possess an essential structural feature: a polar hydroxamic terminal linked through a hydrophobic methylene spacer (e.g. 6 carbon at length) to another polar site which is attached to a terminal hydrophobic moiety (e.g., benzene ring). Compounds developed having such essential features also fall within the scope of the hydroxamic acids that may be used as HDAC inhibitors.

Cyclic peptides used as HDAC inhibitors are mainly cyclic tetrapeptides. Examples of cyclic peptides include, but are not limited to, trapoxin A, apicidin and depsipeptide. Trapoxin A is a cyclic tetrapeptide that contains a 2-amino-8-oxo-9,10-epoxy-decanoyl (AOE) moiety. Kijima et al. (1993) J. Biol. Chem. 268:22429-22435. Apicidin is a fungal metabolite that exhibits potent, broad-spectrum antiprotozoal activitity and inhibits HDAC activity at nanomolar concentrations. Darkin-Rattray et al. (1996) Proc. Natl. Acad. Sci. USA. 93;13143-13147. Depsipeptide is isolated from Chromobacterium violaceum, and has been shown to inhibit HDAC activity at micromolar concentrations.

Examples of benzamides include but are not limited to MS-27-275. Saito et al. (1990) Proc. Natl. Acad. Sci. USA. 96:4592-4597. Examples of short-chain fatty acids include but are not limited to butyrates (e.g., butyric acid, arginine butyrate and phenylbutyrate (PB)). Newmark et al. (1994) Cancer Lett. 78:1-5; and Carducci et al. (1997) Anticancer Res. 17:3972-3973. In addition, depudecin which has been shown to inhibit HDAC at micromolar concentrations (Kwon et al. (1998) Proc. Natl. Acad. Sci. USA. 95:3356-3361) also falls within the scope of histone deacetylase inhibitor of the present invention.

In another embodiment, a combination drug regimen may include drugs or compounds for the treatment or prevention of blood coagulation disorders or secondary conditions associated with these conditions. Thus, a combination drug regimen may include a sirtuin activator and an anti-coagulation or anti-thrombotic agent. For example, one or more sirtuin-activating compounds can be combined with an effective amount of one or more of: aspirin, heparin, and oral Warfarin that inhibits Vit K-dependent factors, low molecular weight heparins that inhibit factors X and II, thrombin inhibitors, inhibitors of platelet GP IIbIIIa receptors, inhibitors of tissue factor (TF), inhibitors of human von Willebrand factor, inhibitors of one or more factors involved in hemostasis (in particular in the coagulation cascade). In addition, sirtuin-activating compounds can be combined with thrombolytic agents, such as t-PA, streptokinase, reptilase, TNK-t-PA, and staphylokinase.

In certain embodiments, methods for reducing, preventing or treating neurodegeneration disorders or blood coagulation disorders may also comprise increasing the protein level of a sirtuin, such as SIRT1 in a human cell or a homologue of any of the sirtuins in other organisms. Increasing protein levels can be achieved by introducing into a cell one or more copies of a nucleic acid that encodes a sirtuin. For example, the level of SIRT1 can be increased in a mammalian cell by introducing into the mammalian cell a nucleic acid encoding SIRT1, e.g., having the amino acid sequence set forth in SEQ ID NO: 2. The nucleic acid may be under the control of a promoter that regulates the expression of the SIRT1 nucleic acid. Alternatively, the nucleic acid may be introduced into the cell at a location in the genome that is downstream of a promoter. Methods for increasing the level of a protein using these methods are well known in the art.

A nucleic acid that is introduced into a cell to increase the protein level of a sirtuin may encode a protein that is at least about 80%, 85%, 90%, 95%, 98%, or 99% identical to the sequence of a sirtuin, e.g., SEQ ID NO: 2. For example, the nucleic acid encoding the protein may be at least about 80%, 85%, 90%, 95%, 98%, or 99% identical to SEQ ID NO: 1. The nucleic acid may also be a nucleic acid that hybridizes, preferably under stringent hybridization conditions, to a nucleic acid encoding a wild-type sirtuin, e.g., SEQ ID NO: 1. Stringent hybridization conditions may include hybridization and a wash in 0.2×SSC at 65° C. When using a nucleic acid that encodes a protein that is different from a wild-type sirtuin protein, such as a protein that is a fragment of a wild-type sirtuin, the protein is preferably biologically active, e.g., is capable of deacetylation. It is only necessary to express in a cell a portion of the sirtuin that is biologically active. For example, a protein that differs from wild-type SIRT1 having SEQ ID NO: 2, preferably contains the core structure thereof. The core structure sometimes refers to amino acids 62-293 of SEQ ID NO: 2, which are encoded by nucleotides 237 to 932 of SEQ ID NO: 1, which encompasses the NAD binding as well as the substrate binding domains. The core domain of SIRT1 may also refer to about amino acids 261 to 447 of SEQ ID NO: 2, which are encoded by nucleotides 834 to 1394 of SEQ ID NO: 1; to about amino acids 242 to 493 of SEQ ID NO: 2, which are encoded by nucleotides 777 to 1532 of SEQ ID NO: 1; or to about amino acids 254 to 495 of SEQ ID NO: 2, which are encoded by nucleotides 813 to 1538 of SEQ ID NO: 1. Whether a protein retains a biological function, e.g., deacetylation capabilities, can be determined according to methods known in the art.

Methods for increasing sirtuin protein levels also include methods for stimulating the transcription of genes encoding sirtuins, methods for stabilizing the corresponding mRNAs, methods, and other methods known in the art.

4. Mitochondrial-Associated Diseases and Disorders

In certain embodiments, the invention provides methods for treating neurodegenerative diseases or disorders that would benefit from increased mitochondrial activity. The methods involve administering to a subject in need thereof a therapeutically effective amount of a sirtuin activating compound. Increased mitochondrial activity refers to increasing activity of the mitochondria while maintaining the overall numbers of mitochondria (e.g., mitochondrial mass), increasing the numbers of mitochondria thereby increasing mitochondrial activity (e.g., by stimulating mitochondrial biogenesis), or combinations thereof. In an exemplary embodiment, the methods involve administering a high dose of a sirtuin activating compound. In certain embodiments, diseases and disorders that would benefit from increased mitochondrial activity include diseases or disorders associated with mitochondrial dysfunction.

In certain embodiments, methods for treating neurodegenerative diseases or disorders that would benefit from increased mitochondrial activity may comprise identifying a subject suffering from a mitochondrial dysfunction. Methods for diagnosing a mitochondrial dysfunction may involve molecular genetic, pathologic and/or biochemical analysis are summarized in Cohen and Gold, Cleveland Clinic Journal of Medicine, 68: 625-642 (2001). One method for diagnosing a mitochondrial dysfunction is the Thor-Byrne-ier scale (see e.g., Cohen and Gold, supra; Collin S. et al., Eur Neurol. 36: 260-267 (1996)). A wide variety of methods for determining mitochondrial mass and/or activity are described in U.S. Patent Application Publication No. 2002/0049176.

Mitochondria are critical for the survival and proper function of almost all types of eukaryotic cells. Mitochondria in virtually any cell type can have congenital or acquired defects that affect their function. Thus, the clinically significant signs and symptoms of mitochondrial defects affecting respiratory chain function are heterogeneous and variable depending on the distribution of defective mitochondria among cells and the severity of their deficits, and upon physiological demands upon the affected cells. Nondividing tissues with high energy requirements, e.g. nervous tissue, skeletal muscle and cardiac muscle are particularly susceptible to mitochondrial respiratory chain dysfunction, but any organ system can be affected.

Neurodegenerative diseases and disorders associated with mitochondrial dysfunction include diseases and disorders in which deficits in mitochondrial respiratory chain activity contribute to the development of pathophysiology of such diseases or disorders in a mammal. This includes 1) congenital genetic deficiencies in activity of one or more components of the mitochondrial respiratory chain; and 2) acquired deficiencies in the activity of one or more components of the mitochondrial respiratory chain, wherein such deficiencies are caused by a) oxidative damage during aging; b) elevated intracellular calcium; c) exposure of affected cells to nitric oxide; d) hypoxia or ischemia; e) microtubule-associated deficits in axonal transport of mitochondria, or f) expression of mitochondrial uncoupling proteins.

Neurodegenerative diseases or disorders that would benefit from increased mitochondrial activity generally include for example, diseases in which free radical mediated oxidative injury leads to tissue degeneration, diseases in which cells inappropriately undergo apoptosis, and diseases in which cells fail to undergo apoptosis. Exemplary diseases or disorders that would benefit from increased mitochondrial activity include, for example, AD (Alzheimer's Disease), multiple sclerosis (MS), ADPD (Alzheimer's Disease and Parkinsons's Disease), HD (Huntington's Disease), PD (Parkinson's Disease), Friedreich's ataxia and other ataxias, amyotrophic lateral sclerosis (ALS) and other motor neuron diseases.

In certain embodiments, the invention provides methods for treating a disease or disorder that would benefit from increased mitochondrial activity that involves administering to a subject in need thereof one or more sirtuin activating compounds in combination with another therapeutic agent such as, for example, an agent useful for treating mitochondrial dysfunction (such as antioxidants, vitamins, or respiratory chain cofactors), an agent useful for reducing a symptom associated with a neurodegenerative disease or disorder involving mitochondrial dysfunction (such as, an anti-seizure agent or an agent useful for alleviating neuropathic pain), or an anti-neurodegeneration agent (as described further above). In an exemplary embodiment, the invention provides methods for treating a neurodegenerative disease or disorder that would benefit from increased mitochondrial activity that involves administering to a subject in need thereof one or more sirtuin activating compounds in combination with one or more of the following: coenzyme Q₁₀, L-carnitine, thiamine, riboflavin, niacinamide, folate, vitamin E, selenium, lipoic acid, or prednisone. Compositions comprising such combinations are also provided herein.

In exemplary embodiments, the invention provides methods for treating neurodegenerative diseases or disorders that would benefit from increased mitochondrial acitivty by administering to a subject a therapeutically effective amount of a sirtuin activating compound. Exemplary neurodegenerative diseases or disorders include, for example, neuromuscular disorders (e.g., Friedreich's Ataxia, muscular dystrophy, multiple sclerosis, etc.), disorders of neuronal instability (e.g., seizure disorders, migrane, etc.), developmental delay, degenerative disorders (e.g., Alzheimer's Disease, Parkinson's Disease, amyotrophic lateral sclerosis, etc.), ischemia, age-related neurodegeneration and cognitive decline.

A gene defect underlying Friedreich's Ataxia (FA), the most common hereditary ataxia, was recently identified and is designated “frataxin”. In FA, after a period of normal development, deficits in coordination develop which progress to paralysis and death, typically between the ages of 30 and 40. The tissues affected most severely are the spinal cord, peripheral nerves, myocardium, and pancreas. Patients typically lose motor control and are confined to wheel chairs, and are commonly afflicted with heart failure and diabetes. The genetic basis for FA involves GAA trinucleotide repeats in an intron region of the gene encoding frataxin. The presence of these repeats results in reduced transcription and expression of the gene. Frataxin is involved in regulation of mitochondrial iron content. When cellular frataxin content is subnormal, excess iron accumulates in mitochondria, promoting oxidative damage and consequent mitochondrial degeneration and dysfunction. When intermediate numbers of GAA repeats are present in the frataxin gene intron, the severe clinical phenotype of ataxia may not develop. However, these intermediate-length trinucleotide extensions are found in 25 to 30% of patients with non-insulin dependent diabetes mellitus, compared to about 5% of the nondiabetic population. In certain embodiments, sirtuin activating compounds may be used for treating patients with disorders related to deficiencies or defects in frataxin, including Friedreich's Ataxia, myocardial dysfunction, diabetes mellitus and complications of diabetes like peripheral neuropathy.

Muscular dystrophy refers to a family of diseases involving deterioration of neuromuscular structure and function, often resulting in atrophy of skeletal muscle and myocardial dysfunction. In the case of Duchenne muscular dystrophy, mutations or deficits in a specific protein, dystrophin, are implicated in its etiology. Mice with their dystrophin genes inactivated display some characteristics of muscular dystrophy, and have an approximately 50% deficit in mitochondrial respiratory chain activity. A final common pathway for neuromuscular degeneration in most cases is calcium-mediated impairment of mitochondrial function. In certain embodiments, sirtuin activating compounds may be used for reducing the rate of decline in muscular functional capacities and for improving muscular functional status in patients with muscular dystrophy.

Multiple sclerosis (MS) is a neuromuscular disease characterized by focal inflammatory and autoimmune degeneration of cerebral white matter. Periodic exacerbations or attacks are significantly correlated with upper respiratory tract and other infections, both bacterial and viral, indicating that mitochondrial dysfunction plays a role in MS. Depression of neuronal mitochondrial respiratory chain activity caused by Nitric Oxide (produced by astrocytes and other cells involved in inflammation) is implicated as a molecular mechanism contributing to MS. In certain embodiments, sirtuin activating compounds may be used for treatment of patients with multiple sclerosis, both prophylactically and during episodes of disease exacerbation.

Epilepsy is often present in patients with mitochondrial cytopathies, involving a range of seizure severity and frequency, e.g. absence, tonic, atonic, myoclonic, and status epilepticus, occurring in isolated episodes or many times daily. In certain embodiments, sirtuin activating compounds may be used for treating patients with seizures secondary to mitochondrial dysfunction, including reducing frequency and severity of seizure activity.

Metabolic studies on patients with recurrent migraine headaches indicate that deficits in mitochondrial activity are commonly associated with this disorder, manifesting as impaired-oxidative phosphorylation and excess lactate production. Such deficits are not necessarily due to genetic defects in mitochondrial DNA. Migraineurs are hypersensitive to nitric oxide, an endogenous inhibitor of Cytochrome c Oxidase. In addition, patients with mitochondrial cytopathies, e.g. MELAS, often have recurrent migraines. In certain embodiments, sirtuin activating compounds may be used for treating patients with recurrent migraine headaches, including headaches refractory to ergot compounds or serotonin receptor antagonists.

Delays in neurological or neuropsychological development are often found in children with mitochondrial diseases. Development and remodeling of neural connections requires intensive biosynthetic activity, particularly involving synthesis of neuronal membranes and myelin, both of which require pyrimidine nucleotides as cofactors. Uridine nucleotides are involved inactivation and transfer of sugars to glycolipids and glycoproteins. Cytidine nucleotides are derived from uridine nucleotides, and are crucial for synthesis of major membrane phospholipid constituents like phosphatidylcholine, which receives its choline moiety from cytidine diphosphocholine. In the case of mitochondrial dysfunction (due to either mitochondrial DNA defects or any of the acquired or conditional deficits like exicitoxic or nitric oxide-mediated mitochondrial dysfunction) or other conditions resulting in impaired pyrimidine synthesis, cell proliferation and axonal extension is impaired at crucial stages in development of neuronal interconnections and circuits, resulting in delayed or arrested development of neuropsychological functions like language, motor, social, executive function, and cognitive skills. In autism for example, magnetic resonance spectroscopy measurements of cerebral phosphate compounds indicates that there is global undersynthesis of membranes and membrane precursors indicated by reduced levels of uridine diphospho-sugars, and cytidine nucleotide derivatives involved in membrane synthesis. Disorders characterized by developmental delay include Rett's Syndrome, pervasive developmental delay (or PDD-NOS “pervasive developmental delay not otherwise specified” to distinguish it from specific subcategories like autism), autism, Asperger's Syndrome, and Attention Deficit/Hyperactivity Disorder (ADHD), which is becoming recognized as a delay or lag in development of neural circuitry underlying executive functions. In certain embodiments, sirtuin activating compounds may be useful for treating treating patients with neurodevelopmental delays (e.g., involving motor, language, executive function, and cognitive skills), or other delays or arrests of neurological and neuropsychological development in the nervous system and somatic development in non-neural tissues like muscle and endocrine glands.

The two most significant severe neurodegenerative diseases associated with aging, Alzheimer's Disease (AD) and Parkinson's Disease (PD), both involve mitochondrial dysfunction in their pathogenesis. Complex I deficiencies in particular are frequently found not only in the nigrostriatal neurons that degenerate in Parkinson's disease, but also in peripheral tissues and cells like muscle and platelets of Parkinson's Disease patients. In Alzheimer's Disease, mitochondrial respiratory chain activity is often depressed, especially Complex IV (Cytochrome c Oxidase). Moreover, mitochondrial respiratory function altogether is depressed as a consequence of aging, further amplifying the deleterious sequelae of additional molecular lesions affecting respiratory chain function. Other factors in addition to primary mitochondrial dysfunction underlie neurodegeneration in AD, PD, and related disorders. Excitotoxic stimulation and nitric oxide are implicated in both diseases, factors which both exacerbate mitochondrial respiratory chain deficits and whose deleterious actions are exaggerated on a background of respiratory chain dysfunction. Huntington's Disease also involves mitochondrial dysfunction in affected brain regions, with cooperative interactions of excitotoxic stimulation and mitochondrial dysfunction contributing to neuronal degeneration. In certain embodiments, sirtuin activating compounds may be useful for treating and attenuating progression of age-related neurodegenerative disease including AD and PD.

One of the major genetic defects in patients with Amyotrophic Lateral Sclerosis (ALS or Lou Gehrig's Disease) is mutation or deficiency in Copper-Zinc Superoxide Dismutase (SOD 1), an antioxidant enzyme. Mitochondria both produce and are primary targets for reactive oxygen species. Inefficient transfer of electrons to oxygen in mitochondria is the most significant physiological source of free radicals in mammalian systems. Deficiencies in antioxidants or antioxidant enzymes can result in or exacerbate mitochondrial degeneration. Mice transgenic for mutated SOD1 develop symptoms and pathology similar to those in human ALS. The development of the disease in these animals has been shown to involve oxidative destruction of mitochondria followed by functional decline of motor neurons and onset of clinical symptoms. Skeletal muscle from ALS patients has low mitochondrial Complex I activity. In certain embodiments, sirtuin activating compounds may be useful for treating ALS, for reversing or slowing the progression of clinical symptoms.

Oxygen deficiency results in both direct inhibition of mitochondrial respiratory chain activity by depriving cells of a terminal electron acceptor for Cytochrome c reoxidation at Complex IV, and indirectly, especially in the nervous system, via secondary post-anoxic excitotoxicity and nitric oxide formation. In conditions like cerebral anoxia, angina or sickle cell anemia crises, tissues are relatively hypoxic. In such cases, compounds that increase mitochondrial activity provide protection of affected tissues from deleterious effects of hypoxia, attenuate secondary delayed cell death, and accelerate recovery from hypoxic tissue stress and injury. In certain embodiments, sirtuin activating compounds may be useful for preventing delayed cell death (apoptosis in regions like the hippocampus or cortex occurring about 2 to 5 days after an episode of cerebral ischemia) after ischemic or hypoxic insult to the brain.

During normal aging, there is a progressive decline in mitochondrial respiratory chain function. Beginning about age 40, there is an exponential rise in accumulation of mitochondrial DNA defects in humans, and a concurrent decline in nuclear-regulated elements of mitochondrial respiratory activity. Many mitochondrial DNA lesions have a selection advantage during mitochondrial turnover, especially in postmitotic cells. The proposed mechanism is that mitochondria with a defective respiratory chain produce less oxidative damage to themselves than do mitochondria with intact functional respiratory chains (mitochondrial respiration is the primary source of free radicals in the body). Therefore, normally-functioning mitochondria accumulate oxidative damage to membrane lipids more rapidly than do defective mitochondria, and are therefore “tagged” for degradation by lysosomes. Since mitochondria within cells have a half life of about 10 days, a selection advantage can result in rapid replacement of functional mitochondria with those with diminished respiratory activity, especially in slowly dividing cells. The net result is that once a mutation in a gene for a mitochondrial protein that reduces oxidative damage to mitochondria occurs, such defective mitochondria will rapidly populate the cell, diminishing or eliminating its respiratory capabilities. The accumulation of such cells results in aging or degenerative disease at the organismal level. This is consistent with the progressive mosaic appearance of cells with defective electron transport activity in muscle, with cells almost devoid of Cytochrome c Oxidase (COX) activity interspersed randomly amidst cells with normal activity, and a higher incidence of COX-negative cells in biopsies from older subjects. The organism, during aging, or in a variety of mitochondrial diseases, is thus faced with a situation in which irreplaceable postmitotic cells (e.g. neurons, skeletal and cardiac muscle) must be preserved and their function maintained to a significant degree, in the face of an inexorable progressive decline in mitochondrial respiratory chain function. Neurons with dysfunctional mitochondria become progressively more sensitive to insults like excitotoxic injury. Mitochondrial failure contributes to most degenerative diseases (especially neurodegeneration) that accompany aging. Congenital mitochondrial diseases often involve early-onset neurodegeneration similar in fundamental mechanism to disorders that occur during aging of people born with normal mitochondria. In certain embodiments, sirtuin activating compounds may be useful for treating or attenuating cognitive decline and other degenerative consequences of aging.

In certain embodiments, sirtuin modulating compounds may be useful for treatment mitochondrial myopathies. Mitochondrial myopathies range from mild, slowly progressive weakness of the extraocular muscles to severe, fatal infantile myopathies and multisystem encephalomyopathies. Some syndromes have been defined, with some overlap between them. Established syndromes affecting muscle include progressive external ophthalmoplegia, the Kearns-Sayre syndrome (with ophthalmoplegia, pigmentary retinopathy, cardiac conduction defects, cerebellar ataxia, and sensorineural deafness), the MELAS syndrome (mitochondrial encephalomyopathy, lactic acidosis, and stroke-like episodes), the MERFF syndrome (myoclonic epilepsy and ragged red fibers), limb-girdle distribution weakness, and infantile myopathy (benign or severe and fatal). Muscle biopsy specimens stained with modified Gomori's trichrome stain show ragged red fibers due to excessive accumulation of mitochondria. Biochemical defects in substrate transport and utilization, the Krebs cycle, oxidative phosphorylation, or the respiratory chain are detectable. Numerous mitochondrial DNA point mutations and deletions have been described, transmitted in a maternal, nonmendelian inheritance pattern. Mutations in nuclear-encoded mitochondrial enzymes occur.

In certain embodiments, sirtuin activating compounds may be useful for treating patients suffering from toxic damage to mitochondria, such as, toxic damage due to nitric oxide exposure, drug induced toxic damage, or hypoxia.

Excessive stimulation of neurons with excitatory amino acids is a common mechanism of cell death or injury in the central nervous system. Activation of glutamate receptors, especially of the subtype designated NMDA receptors, results in mitochondrial dysfunction, in part through elevation of intracellular calcium during excitotoxic stimulation. Conversely, deficits in mitochondrial respiration and oxidative phosphorylation sensitizes cells to excitotoxic stimuli, resulting in cell death or injury during exposure to levels of excitotoxic neurotransmitters or toxins that would be innocuous to normal cells.

Nitric oxide (about 1 micromolar) inhibits cytochrome oxidase (Complex IV) and thereby inhibits mitochondrial respiration; moreover, prolonged exposure to nitric oxide (NO) irreversibly reduces Complex I activity. Physiological or pathophysiological concentrations of NO thereby inhibit pyrimidine biosynthesis. Nitric oxide is implicated in a variety of neurodegenerative disorders including inflammatory and autoimmune diseases of the central nervous system, and is involved in mediation of excitotoxic and post-hypoxic damage to neurons.

In yet other embodiments, provided are methods (e.g., assays such as screening assays or high throughput screens) for identifying agents, such as sirtuin modulating compounds, that are useful for modulating mitochondrial mass and/or mitochondrial function in cells of an animal or human subject. In certain embodiments, candidate agents are screened for their ability to increase mitochondrial mass and/or improve mitochondrial function. In an exemplary embodiment, the methods described herein may be used to identify an agent that increases mitochondrial mass and/or improves mitochondrial function in cells, such as, for example, a sirtuin-activating compound.

5. PPAR Agonists

In aother aspect, the invention provides methods for treating patients suffering from neurodegenerative diseases or disorders by administering to a patient in need thereof a PPAR delta agonist. In another aspect, the invention provides methods for treating patients suffereing from neurodegenerative diseases or disorders by administering to a patient in need thereof at least one PPAR-alpha, PPAR-gamma, or PPAR-delta agonist in combination with at least one sirtuin-activating compound. Neurodegenerative diseases or disorders that may be treated using PPAR-delta or PPAR-gamma, -alpha, or -delta in combination with a sirtuin-activating compound include, for example, neurodegenerative diseases, mitochondrial-associated neurodegenerative diseases, traumatic or mechanical injury to the central nervous system (CNS), spinal cord or peripheral nervous system (PNS), drug-induced or toxic neuropathies, axonopathy, peripheral neuropathy, trauma to the nerves (e.g., from disease, injury, or the environment), chemotherapeutic induced neuropathy, polyglutamine diseases, and/or neuropathy related to ischemic injuries or diseases. Examples of neurodegenerative diseases include, but are not limited to, Alzheimer's disease (AD), Parkinson's disease (PD), Huntington's disease (HD), multiple sclerosis (MS), amyotrophic lateral sclerosis (ALS; Lou Gehrig's disease), diffuse Lewy body disease, chorea-acanthocytosis, primary lateral sclerosis, and Friedreich's ataxia. These and other neurodegenerative diseases and disorders that may be treated using PPAR agonists or a combination of a PPAR agonist and a sirtuin-activating compound are described more fully above.

In one embodiment, a PPAR-delta agonist or a combination of at least one PPAR agonist and at least one sirtuin-activating compound may be used to treat a neurodegenerative disorder that is a PPAR-mediated disease or condition, e.g., a disease or condition in which the biological activity and/or expression level of a PPAR affects the development and/or course of the disease or condition, and/or in which an increase in the biological activity and/or expression level of a PPAR improves or ameliorates the development, course, and/or symptoms of the disease or condition. In some cases the disease or condition may be mediated by one or more of the PPAR isoforms, e.g., PPAR gamma, PPAR alpha, and PPAR delta. In exemplary embodiments, treatment of a neurodegenerative disease or disorder with a PPAR delta agonist or a combination of at least one PPAR agonist and at least one sirtuin-activating compound results in a reduction in the severity and/or duration of the neurodegenerative disease or disorder, reduces the likelihood or delays the onset of the neurodegenerative disease or disorder, and/or causes an improvement in one or more symptoms of the neurodegenerative disease or disorder.

In certain embodiments, the amount of a PPAR-delta agonist that is administered to a patient suffering from a neurodegenerative disease or disorder results in a serum level that is less than about 50 μM, 10 μM, 1 μM, 100 nM, 10 nM, 1 nM, 0.1 nM or lower.

In certain embodiments, administration of at least one PPAR agonist in combination with at least one sirtuin-activating compound permits a desired therapeutic effect while utilizing a lower dose of a PPAR agonist than would be necessary in the absence of the sirtuin-activating compound. For example, when administering the PPAR agonist in combination with a sirtuin-activating compound it may be possible to reduce the dose of the PPAR agonist needed to obtain a therapeutic effect by at least about 1%, 5%, 10%, 25%, 30%, 50%, 60%, 70%, 75%, 80%, 85%, 90%, or more, as compared to the dose needed to obtain the same, or a similar, level of a therapeutic effect in the absence of the sirtuin-activating compound. Such combinations may permit the avoidance of one or more undesirable side effects associated with the administration of a higher dose of a PPAR agonist (see e.g., Michalik et al., Nature Reviews Cancer 4, 61-70 (2004); Gupta et al., Nature Medicine 10, 245-247 (2004); Stephen et al., Cancer Research 64, 3162-3170, 2004) The peroxisome proliferator activated receptors (PPARs) are considered members of the nuclear receptor (nuclear hormone receptor) super family. Currently, three kinds of PPAR subtypes called PPAR alpha, PPAR delta (also called NUC-1, PPAR beta or FAAR) and PPAR gamma have been identified, and their genes (cDNA) have been cloned (Lemberger et al., Annu. Rev. Cell. Dev. Biol., 12, 335-363 (1996)). PPARs are nuclear receptors that regulate the expression of genes involved in lipid and glucose metabolism. PPAR-alpha, -gamma, and -delta are distinguishable from each other based on tissue distribution and cell activation. All PPARs are, to different extents, activated by fatty acids and derivatives thereof. Recently, it has been shown that PPAR-gamma serves as a widespread regulator of fat burning, suggesting that it might be a potential target in the treatment of obesity and type 2 diabetes.

The PPARs are ligand-dependent transcription factors that regulate target gene expression by binding to specific peroxisome proliferator response elements (PPREs) in enhancer sites of regulated genes. PPARs possess a modular structure composed of functional domains that include a DNA binding domain (DBD) and a ligand binding domain (LBD). The DBD specifically binds PPREs in the regulatory region of PPAR-responsive genes. The DBD, located in the C-terminal half of the receptor contains the ligand-dependent activation domain, AF-2. Each receptor binds to its PPRE as a heterodimer with a retinoid X receptor (RXR). Upon binding an agonist, the conformation of a PPAR is altered and stabilized such that a binding cleft, made up in part of the AF-2 domain, is created and recruitment of transcriptional coactivators occurs. Coactivators augment the ability of nuclear receptors to initiate the transcription process. The result of the agonist-induced PPAR-coactivator interaction at the PPRE is an increase in gene transcription. Downregulation of gene expression by PPARs appears to occur through indirect mechanisms. (Bergen & Wagner, 2002, Diabetes Tech. & Ther., 4:163-174).

The first cloning of a PPAR (PPAR alpha) occurred in the course of the search for the molecular target of rodent hepatic peroxisome proliferating agents. Since then, numerous fatty acids and their derivatives including a variety of eicosanoids and prostaglandins have been shown to serve as ligands of the PPARs. Thus, these receptors may play a central role in the sensing of nutrient levels and in the modulation of their metabolism. In addition, PPARs are the primary targets of selected-classes of synthetic compounds that have been used in the successful treatment of diabetes and dyslipidemia. As such, an understanding of the molecular and physiological characteristics of these receptors has become extremely important to the development and utilization of drugs used to treat metabolic disorders. In addition, due to the great interest within the research community, a wide range of additional roles for the PPARs have been discovered; PPAR alpha and PPAR gamma may play a role in a wide range of events involving the vasculature, including atherosclerotic plaque formation and stability, thrombosis, vascular tone, angio-genesis, and cancer.

Among the synthetic ligands identified for PPARs are Thiazolidinediones (TZDs). These compounds were originally developed on the basis of their insulin-sensitizing effects in animal pharmacology studies. Subsequently, it was found that TZDs induced adipocyte differentiation and increased expression of adipocyte genes, including the adipocyte fatty acid-binding protein aP2. Independently, it was discovered that PPAR gamma interacted with a regulatory element of the aP2 gene that controlled its adipocyte-specific expression. On the basis of these seminal observations, experiments were performed that determined that TZDs were PPAR gamma ligands and agonists and demonstrated a definite correlation between their in vitro PPAR gamma activities and their in vivo insulin-sensitizing actions (Bergen & Wagner, 2002, Diabetes Tech. & Ther., 4:163-174).

Several TZDs, including troglitazone, rosiglitazone, and pioglitazone, have insulin-sensitizing and anti-diabetic activity in humans with type 2 diabetes and impaired glucose tolerance. Farglitazar is a very potent non-TZD PPAR gamma-selective agonist that was recently shown to have antidiabetic as well as lipid-altering efficacy in humans. In addition to these potent PPAR gamma ligands, a subset of the non-steroidal antiinflammatory drugs (NSAIDs), including indomethacin, fenoprofen, and ibuprofen, have displayed weak PPAR gamma and PPAR alpha activities (Bergen & Wagner, 2002, Diabetes Tech. & Ther., 4:163-174).

The fibrates, amphipathic carboxylic acids that have been proven useful in the treatment of hypertriglyceridemia, are PPAR alpha ligands. The prototypical member of this compound class, clofibrate, was developed prior to the identification of PPARs, using in vivo assays in rodents to assess lipid-lowering efficacy (Bergen & Wagner, 2002, Diabetes Tech. & Ther., 4:163-174).

It was reported that, fibrate agents having a ligand effect on PPAR alpha, among the three kinds of PPARs, clinically show a strong lowering effect on serum triacylglycerol levels (Forman et al., Proc. Natl. Acad. Sci. USA, 94, 4312-4317 (1997)).

PPAR gamma is highly expressed in adipose tissues and has been implicated in regulating differentiation of adipocytes (Tontonoz et al., Genes and Development, 8, 1224-1234 (1994); and Tontonoz et al., Cell, 79, 1147-1156 (1994)). Various kinds of thiazolidinedione derivatives show a hypoglycemic effect in an animal model of non-insulin-dependent diabetes mellitus (NIDDM) and are expected as new therapeutic agents for NIDDM having an insulin resistance breaking effect. A recent study demonstrated that the thiazolidinedione derivatives are ligands of PPAR gamma and specifically activate PPAR gamma (Lehman et al., J. Biol. Chem., 270, 12953-12956 (1995)).

Several physiological functions have been proposed for PPAR delta including a blood HDL increasing effect and a chlosteral lowering effect (see e.g., WO 97/28149, WO 99/04815, and Willson et al., J. Med. Chem., 43 (4), 527-550 (2000)). The role of PPAR delta in inflammation and obesity has also been examined (see e.g., Henson, Proc Natl Acad Sci USA. 100(11): 6295-6296 (2003), Evans et al, Nature Medicine 10, 355, 2004, and Shin et al., Diabetes 53:847-851, 2004). Two exemplary PPAR delta agonists are GW0742 and GW501516 (Sznaidman et al., Bioorganic & Medicinal Chemistry Letters, 13: 1517-1521 (2003); Wei et al, J. Org. Chem. 68: 9116 (2003)). A lipid response study of GW501516 using Affymetrix arrays has been described (Tanaka et al., Proc Natl Acad Sci USA 100(26): 15924-15929, 2003).

6. Antiinflammatory Agents

In aother aspect, the invention provides methods for treating patients suffering from neurodegenerative diseases or disorders that involve inflammation by administering to a patient in need thereof at least one anti-inflammatory agent in combination with at least one sirtuin-activating compound. Neurodegenerative diseases or disorders that involve inflammation include, for example, Alzheimer's disease (AD) and multiple sclerosis (MS). These and other neurodegenerative diseases and disorders that may be treated using a combination of an anti-inflammatory agent and a sirtuin-activating compound are described more fully above.

In certain embodiments, administration of at least one anti-inflammatory agent in combination with at least one sirtuin-activating compound permits a desired therapeutic effect while utilizing a lower dose of an anti-inflammatory agent than would be necessary in the absence of the sirtuin-activating compound. For example, when administering the anti-inflammatory agent in combination with a sirtuin-activating compound it may be possible to reduce the dose of the anti-inflammatory agent needed to obtain a therapeutic effect by at least about 1%, 5%, 10%, 25%, 30%, 50%, 60%, 70%, 75%, 80%, 85%, 90%, or more, as compared to the dose needed to obtain the same, or a similar, level of a therapeutic effect in the absence of the sirtuin-activating compound. Such combinations may permit the avoidance of one or more undesirable side effects associated with the administration of a higher dose of an anti-inflammatory agent.

Inflammation is a life-saving mechanism that enables the body to launch a defensive attack against bacteria, parasites and viruses. The process ends once the foreign agent is eliminated and healing begins. Occasionally, however, if this process fails, inflammation becomes chronic, leading to permanent damage. Accumulated evidence suggests that chronic inflammation might be involved in neurodegenerative diseases of old age including AD and MS (Marchetti, B., and M. P. Abbracchio. 2005. Trends Pharmacol Sci 26:517-25; McGeer, E. G., et al. 2005. Neurobiol Aging 26 (Supp 1): 94-97). Inflammatory hallmarks associated with AD include activation of the complement cascade, up-regulation of a whole host of acute phase proteins, cytokines and chemokines, their receptors, as well as a reactive astrogliosis and microgliosis in the vicinity of diffuse Aβ and amyloid deposits (McGeer, E. G., A. Klegeris, and P. L. McGeer. 2005. Inflammation, the complement system and the diseases of aging. Neurobiol Aging). Moreover, inheritance of polymorphisms of various inflammatory mediators which enhance their expression have been reported to increase the risk of AD (Du et al. 2000. Neurology 55:480-3; Nicoll et al. 2000. Ann Neurol 47:365-8; Papassotiropoulos et al 1999. Ann Neurol 45:666-8; Rainero et al 2004. Neurobiol Aging 25:1293-8). Further evidence for the role of inflammatory reaction in AD is provided by epidemiological studies, indicating that the chronic use of nonsteroidal anti-inflammatory drugs (NSAIDs) greatly reduces the risk of AD (McGeer, P. L., M. Schulzer, and E. G. McGeer. 1996. Neurology 47:425-32; 75). Even though the role of inflammation in AD is controversial it is highly likely that neuroinflammation, at the very least, exacerbates AD pathogenesis.

Several lines of evidence implicate the inflammatory mediator NF-κB in the pathogenesis of AD. In AD brains, NF-κB immunoreactivity is increased in neurons and astrocytes surrounding amyloid plaques (Kaltschmidt et al. 1997. Proc Natl Acad Sci U S A 94:2642-7.), and in cultured neurons and glia, Aβ stimulation lead to NF-κB activation (Akama et al. 1998. Proc Natl Acad Sci U S A 95:5795-800; Bales et al. 2000. Neurobiol Aging 21:427-32; discussion 451-3.; Kaltschmidt et al. 1997. Proc Natl Acad Sci U S A 94:2642-7.; Mattson, M. P., and S. Camandola. 2001. J Clin Invest 107:247-54.). A recent study by Chen et al. (Chen et al. 2005. SIRT1 protects against microglia-dependent beta amyloid toxicity through inhibiting NF-kappa B signaling. J Biol Chem. 280 (48) 40364) established a causal link between NF-κB signaling in microglia and neurotoxicity of Aβ in mixed cortical cultures and found that NF-κB signaling in microglia is critically involved in neuronal death induced by Aβ peptides, which are presumed to be the cause of AD.

NF-κB is a central pro-inflammatory transcription factor that regulates the expression of a diverse set of inflammatory markers including TNFα, IL-1β, and IL-6. Transcriptionally active NF-κB is a heterodimeric complex that contains a DNA-binding domain and a transactivation domain. The most abundant form of NF-κB exists as a heterodimer composed of p50 and RelA/p65 subunits. In unstimulated cells, NF-κB is localized in the cytoplasm bound by its inhibitory proteins, members of the IκB family. Upon cellular stimulation, IκB is phosphorylated and thus targeted for ubiquitination and degradation by the proteosome. Liberated NF-κB translocates to the nucleus where it interacts with promoter gene targets to enhance transcription (Verma, I. M. 2004. Ann Rheum Dis 63 Suppl 2:ii57-ii61).

In certain embodiments, sirtuin activating compounds may be administered in combination with one or more anti-inflammatory agents, including, for example, steroidal anti-inflammatory agents, non-steroidal anti-inflammatory agents, and/or nonsteroidal immunomodulating agents.

Exemplary steroidal anti-inflammatory agents that may be used in accordance with the methods described herein, include, for example: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortarnate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, and triamcinolone hexacetonide.

Exemplary non-steroidal anti-inflammatory agents that may be used in accordance with the methods described herein, include, for example: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lomoxicam, piroxicam, tenoxicam), .epsilon.-acetamidocaproic acid, s-adenosylmethionine, 3-amino4-hydroxybutyric acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, and zileuton.

Exemplary non-steroidal immunomodulating agents that may be used in accordance with the methods described herein, include, for example: (i) a COX-1 inhibitor, a COX-2 inhibitor or a non-selective nonsteroidal immunomodulating agent, which simultaneously inhibits both COX-1 and COX-2; (ii) an indole, an indende acetic acid, a heteraryl acetic acid, an arylpropionic acid, an anthranilic acids, a fenamate, an enolic acid, a pyrazolidinediones and an alkanones; and salts and derivatives thereof; (iii) salicylic acid, aspirin, sodium salicylate, choline magnesium trislicylate, salsalate, diflunisal, salicylsalicylic acid, sulfasalazine, olsalazine, esters of salicylic acid with a carboxylic acid, esters of salicylic acid with a dicarboxylic acid, esters of salicylic acid with a fatty acid, esters of salicylic acid with a hydroxyl fatty acid, esters of salicylic acid with an essential fatty acid, esters of salicylic acid with a polycarboxylic acid, para-aminophenol, indole, indomethacin, sulindac, etodolac, tolmetin, diclofenac, ketorolac, ibuprofen, naproxen, flubiprofen, ketoprofen, fenoprofen, oxaprozin, mefenamic acid, meclofenamic acid, oxicams, piroxicam, tenoxicam, pyrazolidinediones, phenylbutazone, oxyphenthratrazone, nabumetone, diaryl-substituted furanones, Rofecoxib), diaryl-substituted pyrazoles, Celecoxib, indole acetic acids, Etodolac, sulfonanilides, Nimesulide and salts, derivatives and analogs thereof; (iv) an imidazole or triazole compounds which possess anti-inflammatory properties; (v) ketoconazole; (vi) agents which inhibit pro-inflammatory cytokines from T cells and/or pro-inflammatory mediators from mast cells; (vii) agents which inhibit TNF-alpha, TNF-beta, interleukin-1, interleukin-4, interleukin-6, interleukin-10, interleukin-12 or IFN-gamma; (viii) xanthine, pentoxifylline, propentofylline, torbafylline, amiloride, chloroquine, thalidomide and salts, derivatives and analogs thereof; (ix) immunosuppressant agents, immunoregulating agents and immunomodulators such as Copaxone (glatiramer acetate), Avonex® (interferon beta-1a), Tysabri® (natalizumab), or Fumaderm® (BG-12/Oral Fumarate); (x) immunomodulating cyclic peptides, cyclosporine, tacrolimus, tresperimus, pimecrolimus, sirolimus, verolimus, laflunimus, laquinimod and imiquimod; (xi) a calcineurin inhibitor; (xii) a nitric oxide synthase inhibitor; (xiii) a leucocyte chemotaxis inhibitor; (xiv) a dicarboxylic acid, having between about 6 and about 14 carbon atoms its carbon atom skeleton, and salts and derivatives thereof; (xv) adipic acid, pimelic acid, suberic acid, azelaic acid, sebacic acid, 1,11-undecanedioic acid, 1,12-dodecanedioic acid, 1,13-tridecanedioic acid and 1,14-tetradecanedioic acid; (xvi) azelaic acid; (xvii) A dicarboxylic acid is covalently linked with at least one moiety, selected from the group consisting of alpha-hydroxy acid, beta-hydroxy acid, hydroxybenzoic acid, alkylhydroxybenzoate, dihydroxy benzene, cresol, alcohol derivatives of Vitamin A (retinoic acid), retinal, steroid hormones, corticosteroids, vitamin E and vitamin D, and derivatives and analogs thereof.

7. Exemplary Sirtuin-Inhibiting Compounds

In another embodiment, the present invention relates to sirtuin-inhibitory compounds. Exemplary sirtuin inhibitory compounds include compounds that inhibit the activity of a class III histone deacetylase, such as, for example, nicotinamide (NAM), suranim; NF023 (a G-protein antagonist); NF279 (a purinergic receptor antagonist); Trolox (6-hydroxy-2,5,7,8,tetramethylchroman-2-carboxylic acid); (−)-epigallocatechin (hydroxy on sites 3,5,7,3′,4′,5′); (−)-epigallocatechin gallate (Hydroxy sites 5,7,3′,4′,5′ and gallate ester on 3); cyanidin choloride (3,5,7,3′,4′-pentahydroxyflavylium chloride); delphinidin chloride (3,5,7,3′,4′,5′-hexahydroxyflavylium chloride); myricetin (cannabiscetin; 3,5,7,3′,4′,5′-hexahydroxyflavone); 3,7,3′,4′,5′-pentahydroxyflavone; and gossypetin (3,5,7,8,3′,4′-hexahydroxyflavone), all of which are further described in Howitz et al. (2003) Nature 425:191. Other inhibitors, such as sirtinol and splitomicin, are described in Grozinger et al. (2001) J. Biol. Chem. 276:38837, Dedalov et al. (2001) PNAS 98:15113 and Hirao et al. (2003) J. Biol. Chem 278:52773. Analogs and derivatives of these compounds can also be used.

Yet other sirtuin inhibitory compounds may have any one of the following formulas:

A sirtuin inhibitory compound may have a formula selected from the group of formulas 26-29, 31, and 66-68:

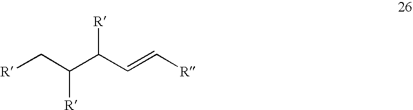

wherein, independently for each occurrence,

R′ represents H. halogen, NO₂, SR, OR, NR₂, alkyl, aryl, aralkyl, or carboxy;

R represents H, alkyl, aryl, aralkyl, or heteroaralkyl; and

R″ represents alkyl, alkenyl, or alkynyl;

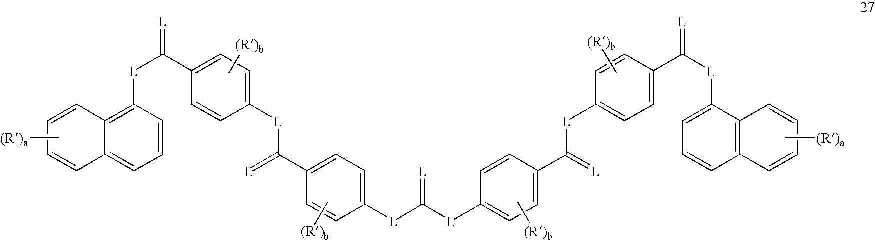

wherein, independently for each occurrence,

L represents O, NR, or S;

R represents H, alkyl, aryl, aralkyl, or heteroaralkyl;

R′ represents H, halogen, NO₂, SR, SO₃, OR, NR₂, alkyl, aryl, aralkyl, or carboxy;

a represents an integer from 1 to 7 inclusive; and

b represents an integer from 1 to 4 inclusive; 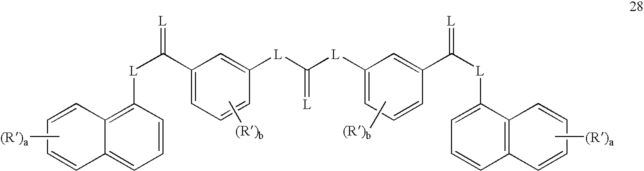
wherein, independently for each occurrence,

L represents O, NR, or S;

R represents H, alkyl, aryl, aralkyl, or heteroaralkyl;

R′ represents H, halogen, NO₂, SR, SO₃, OR, NR₂, alkyl, aryl, or carboxy;

a represents an integer from 1 to 7 inclusive; and

b represents an integer from 1 to 4 inclusive;

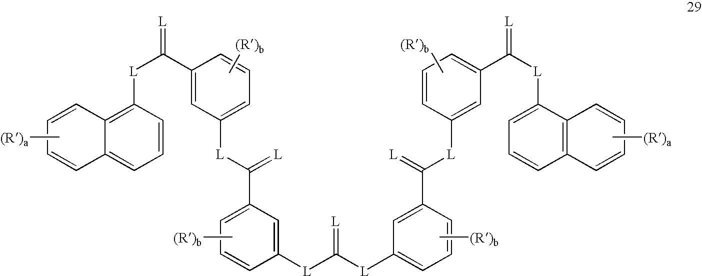

wherein, independently for each occurrence,

L represents O, NR, or S;

R represents H, alkyl, aryl, aralkyl, or heteroaralkyl;

R′ represents H, halogen, NO₂, SR, SO₃, OR, NR₂, alkyl, aryl, aralkyl, or carboxy;

a represents an integer from 1 to 7 inclusive; and

b represents an integer from 1 to 4 inclusive;

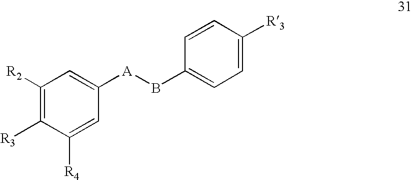

wherein, independently for each occurrence,

R₂, R₃, and R₄ are H, OH, or O-alkyl;

R′₃ is H or NO₂; and

A-B is an ethenylene or amido group.

In a further embodiment, the inhibiting compound is represented by formula 31 and the attendant definitions, wherein R₃ is OH, A-B is ethenylene, and R′₃ is H.

In a further embodiment, the inhibiting compound is represented by formula 31 and the attendant definitions, wherein R₂ and R₄ are OH, A-B is an amido group, and R′₃ is H.

In a further embodiment, the inhibiting compound is represented by formula 31 and the attendant definitions, wherein R₂ and R₄ are OMe, A-B is ethenylene, and R′₃ is NO₂.

In a further embodiment, the inhibiting compound is represented by formula 31 and the attendant definitions, wherein R₃ is OMe, A-B is ethenylene, and R′₃ is H.

In another embodiment, a sirtuin inhibitor is a compound of formula 66: 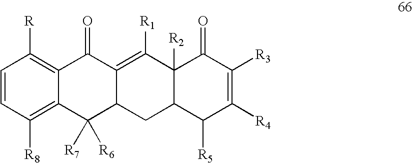
wherein, independently for each occurrence:

R, R₁, R₂, R₃, R₄, R₅, R₆, R₇, and R₈ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl.

In a further embodiment, a sirtuin inhibitor is a compound of formula 66 and the attendant definitions wherein R is OH.

In a further embodiment, a sirtuin inhibitor is a compound of formula 66 and the attendant definitions wherein R₁ is OH.

In a further embodiment, a sirtuin inhibitor is a compound of formula 66 and the attendant definitions wherein R₂ is OH.

In a further embodiment, a sirtuin inhibitor is a compound of formula 66 and the attendant definitions wherein R₃ is C(O)NH₂.

In a further embodiment, a sirtuin inhibitor is a compound of formula 66 and the attendant definitions wherein R₄ is OH.

In a further embodiment, a sirtuin inhibitor is a compound of formula 66 and the attendant definitions wherein R₅ is NMe₂.

In a further embodiment, a sirtuin inhibitor is a compound of formula 66 and the attendant definitions wherein R₆ is methyl.

In a further embodiment, a sirtuin inhibitor is a compound of formula 66 and the attendant definitions wherein R₇ is OH.

In a further embodiment, a sirtuin inhibitor is a compound of formula 66 and the attendant definitions wherein R₈ is Cl.

In a further embodiment, a sirtuin inhibitor is a compound of formula 66 and the attendant definitions wherein R is OH and R₁ is OH.

In a further embodiment, a sirtuin inhibitor is a compound of formula 66 and the attendant definitions wherein R is OH, R₁ is OH, and R₂ is OH.

In a further embodiment, a sirtuin inhibitor is a compound of formula 66 and the attendant definitions wherein R is OH, R₁ is OH, R₂ is OH, and R₃ is C(O)NH₂.

In a further embodiment, a sirtuin inhibitor is a compound of formula 66 and the attendant definitions wherein R is OH, R₁ is OH, R₂ is OH, R₃ is C(O)NH₂, and R₄ is OH.

In a further embodiment, a sirtuin inhibitor is a compound of formula 66 and the attendant definitions wherein R is OH, R₁ is OH, R₂ is OH, R₃ is C(O)NH₂, R₄ is OH, and R₅ is NMe₂.

In a further embodiment, a sirtuin inhibitor is a compound of formula 66 and the attendant definitions wherein R is OH, R₁ is OH, R₂ is OH, R₃ is C(O)NH₂, R₄ is OH, R₅ is NMe₂, and R₆ is methyl.

In a further embodiment, a sirtuin inhibitor is a compound of formula 66 and the attendant definitions wherein R is OH, R₁ is OH, R₂ is OH, R₃ is C(O)NH₂, R₄ is OH, R₅ is NMe₂, R₆ is methyl, and R₇ is OH.

In a further embodiment, a sirtuin inhibitor is a compound of formula 66 and the attendant definitions wherein R is OH, R₁ is OH, R₂ is OH, R₃ is C(O)NH₂, R₄ is OH, R₅ is NMe₂, R₆ is methyl, R₇ is OH, and R₈ is Cl.

In another embodiment, a sirtuin inhibitor is a compound of formula 67: 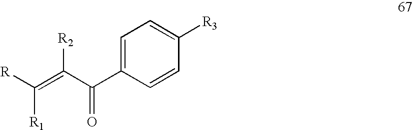
wherein, independently for each occurrence:

R, R₁ , R₂, and R₃ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl.

In a further embodiment, a sirtuin inhibitor is a compound of formula 67 and the attendant definitions wherein R is Cl.

In a further embodiment, a sirtuin inhibitor is a compound of formula 67 and the attendant definitions wherein R₁ is H.

In a further embodiment, a sirtuin inhibitor is a compound of formula 67 and the attendant definitions wherein R₂ is H.

In a further embodiment, a sirtuin inhibitor is a compound of formula 67 and the attendant definitions wherein R₃ is Br.

In a further embodiment, a sirtuin inhibitor is a compound of formula 67 and the attendant definitions wherein R is Cl and R₁ is H.

In a further embodiment, a sirtuin inhibitor is a compound of formula 67 and the attendant definitions wherein R is Cl, R₁ is H, and R₂ is H.

In a further embodiment, a sirtuin inhibitor is a compound of formula 67 and the attendant definitions wherein R is Cl, R₁ is H, R₂ is H, and R₃ is Br.

In another embodiment, a sirtuin inhibitor is a compound of formula 68: 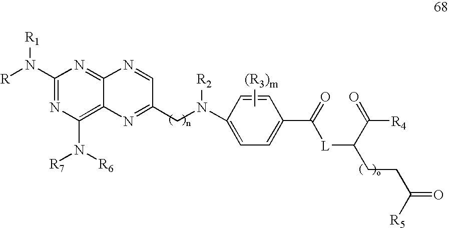
wherein, independently for each occurrence:

R, R₁ , R₂, R_{6,} and R₇ are H or a substituted or unsubstituted alkyl, aryl, aralkyl, heterocyclyl, heterocyclylalkyl, heteroaryl, or heteroaralkyl;

R₃, R_{4,} and R₅ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L is O, NR, or S;

m is an integer from 0 to 4 inclusive; and

n and o are integers from 0 to 6 inclusive.

In a further embodiment, a sirtuin inhibitor is a compound of formula 68 and the attendant definitions wherein R is H.

In a further embodiment, a sirtuin inhibitor is a compound of formula 68 and the attendant definitions wherein R₁ is H.

In a further embodiment, a sirtuin inhibitor is a compound of formula 68 and the attendant definitions wherein R₂ is methyl.

In a further embodiment, a sirtuin inhibitor is a compound of formula 68 and the attendant definitions wherein m is 0.

In a further embodiment, a sirtuin inhibitor is a compound of formula 68 and the attendant definitions wherein R₄ is OH.

In a further embodiment, a sirtuin inhibitor is a compound of formula 68 and the attendant definitions wherein R₅ is OH.

In a further embodiment, a sirtuin inhibitor is a compound of formula 68 and the attendant definitions wherein R₆ is H.

In a further embodiment, a sirtuin inhibitor is a compound of formula 68 and the attendant definitions wherein R₇ is H.

In a further embodiment, a sirtuin inhibitor is a compound of formula 68 and the attendant definitions wherein L is NH.

In a further embodiment, a sirtuin inhibitor is a compound of formula 68 and the attendant definitions wherein n is 1.

In a further embodiment, a sirtuin inhibitor is a compound of formula 68 and the attendant definitions wherein o is 1.

In a further embodiment, a sirtuin inhibitor is a compound of formula 68 and the attendant definitions wherein R is H and R₁ is H.

In a further embodiment, a sirtuin inhibitor is a compound of formula 68 and the attendant definitions wherein R is H, R₁ is H, and R₂ is methyl.

In a further embodiment, a sirtuin inhibitor is a compound of formula 68 and the attendant definitions wherein R is H, R₁ is H, R₂ is methyl, and m is 0.

In a further embodiment, a sirtuin inhibitor is a compound of formula 68 and the attendant definitions wherein R is H, R₁ is H, R₂ is methyl, m is 0, and R₄ is OH.

In a further embodiment, a sirtuin inhibitor is a compound of formula 68 and the attendant definitions wherein R is H, R₁ is H, R₂ is methyl, m is 0, R₄ is OH, and R₅ is OH.

In a further embodiment, a sirtuin inhibitor is a compound of formula 68 arid the attendant definitions wherein R is H, R₁ is H, R₂ is methyl, m is 0, R₄ is OH, R₅ is OH, and R₆ is H.

In a further embodiment, a sirtuin inhibitor is a compound of formula 68 and the attendant definitions wherein R is H, R₁ is H, R₂ is methyl, m is 0, R₄ is OH, R₅ is OH, R₆ is H, and R₇ is H.

In a further embodiment, a sirtuin inhibitor is a compound of formula 68 and the attendant definitions wherein R is H, R₁ is H, R₂ is methyl, m is 0, R₄ is OH, R₅ is OH, R₆ is H, R₇ is H, and L is NH.

In a further embodiment, a sirtuin inhibitor is a compound of formula 68 and the attendant definitions wherein R is H, R₁ is H, R₂ is methyl, m is 0, R₄ is OH, R₅ is OH, R₆ is H, R₇ is H, L is NH, and n is 1.

In a further embodiment, a sirtuin inhibitor is a compound of formula 68 and the attendant definitions wherein R is H, R₁ is H, R₂ is methyl, m is 0, R₄ is OH, R₅ is OH, R₆ is H, R₇ is H, L is NH, n is 1, and o is 1.

Inhibitory compounds may also be oxidized forms of the compounds of Table 22. An oxidized form of chlortetracyclin may be an activator.

Also included are pharmaceutically acceptable addition salts and complexes of the compounds of formulas 26-29, 31 and 66-68. In cases wherein the compounds may have one or more chiral centers, unless specified, the compounds contemplated herein may be a single stereoisomer or racemic mixtures of stereoisomers.

Exemplary inhibitory compounds are those set forth in the appended Tables for which the “ratio to control rate” is lower than one.

In cases in which the compounds have unsaturated carbon-carbon double bonds, both the cis (Z) and trans (E) isomers are contemplated herein. In cases wherein the compounds may exist in tautomeric forms, such as keto-enol tautomers, such as 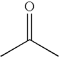
and 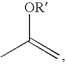
each tautomeric form is contemplated as being included within the methods presented herein, whether existing in equilibrium or locked in one form by appropriate substitution with R′. The meaning of any substituent at any one occurrence is independent of its meaning, or any other substituent's meaning, at any other occurrence.

Also included in the methods presented herein are prodrugs of the compounds of formulas 26-29, 31 and 66-68. Prodrugs are considered to be any covalently bonded carriers that release the active parent drug in vivo.

Whether in vitro or in vivo, a sirtuin inhibitory compound may be administered either alone or in combination with other therapeutic agents. In one embodiment, more than one sirtuin inhibitory compound may be administered, for example, at least 2, 3, 5, 10, or more different sirtuin inhibitory compounds may be administered. In another embodiment, a sirtuin inhibitory compound may be administered as part of a combination therapy with another therapeutic agent. Such combination therapies may be administered simultaneously (e.g., more than one therapeutic agent administered at the same time) or sequentially (e.g., different therapeutic agents administered at different times during a treatment regimen).

8. Exemplary Therapeutic Applications of Sirtuin-Inhibitory Compounds

In certain embodiments, the sirtuin-inhibiting compounds described herein can be used to induce hemostasis (blood clotting) in a subject presenting insufficient hemostatic function, such as a subject having, or at risk of developing a disorder associated with hypocoagulation. As used herein, the term “hypocoagulation” refers to a decreased ability or inability to form blood clots. Such disorders include hemorrhagic disorders, e.g., hemophilia (e.g., hemophilia A or B) and disorders resulting from a deficiency in clotting factors or platelet ligands (e.g., a deficiency in von Willebrand's factor resulting in von Willebrand disease). The induction of a procoagulant state would prevent or stop spontaneous bleeding and would also be beneficial preceding surgical intervention in a patient, or to promote wound healing.

The sirtuin-inhibiting compounds of the present invention are also useful for the treatment of a vasculature-associated disease. As used herein, a “vasculature-associated disease” is a disease having a pathology that is dependent on a vascular blood supply. Thus, it is contemplated that achieving coagulation in the vasculature of the disease site, e.g., in the intratumoral vasculature of a solid tumor, would prove beneficial. Such vasculature-associated diseases include benign and malignant tumors or growths, such as BPH, diabetic retinopathy, vascular restenosis, arteriovenous malformations (AVM), meningioma, hemangioma, neovascular glaucoma and psoriasis. Also included within this group are synovitis, dermatitis, endometriosis, angiofibroma, rheumatoid arthritis, atherosclerotic plaques, corneal graft neovascularization, hemophilic joints, hypertrophic scars, osler-weber syndrome, pyogenic granuloma retrolental fibroplasia, scleroderma, trachoma, and vascular adhesions.

In an exemplary embodiment, the methods and compositions described herein may include a combination therapy comprising other pro-coagulation compounds/treatments. For example, “replacement therapy” has been administered to patients having hemophilia A and B by administration of supplemental factor VIII or IX, respectively. Other hemophilia treatment methods have involved therapy with recombinant factor VIIa. There is recognition that certain membrane settings may assist procoagulant complexes. For example, certain activated aggregated platelets are thought to provide procoagulant phospholipid-equivalent surfaces upon which the complex-dependent reactions of the blood coagulation cascade are localized. See K. Mann, Thrombosis and Haemostasis, 82(2): 165-174, (1999).

In another exemplary embodiment, the subject sirtuin-inhibiting compounds may be combined with an inducer of P-selectin activity to induce thrombosis of tumor blood vessels in order to potentiate tumor necrosis. Such therapies utilize strategies for targeting coagulation factors to the tumor vasculature, for example, as described in U.S. Pat. No. 5,877,289. Markers of tumor vasculature or stroma may be specifically induced and then targeted using a binding ligand, such as an antibody. Exemplary inducible antigens include E-selectin, P-selectin, MHC Class II antigens, VCAM-1, ICAM-1, endoglin, ligands reactive with LAM-1, vascular addressins and other adhesion molecules.

Methods for inducing hemostasis (blood clotting) may also comprise decreasing the protein level of a sirtuin in the cell. Decreasing a protein level can be achieved according to methods known in the art. For example, an siRNA, an antisense or ribozyme targeted to the sirtuin can be expressed in the cell. A dominant negative sirtuin mutant, e.g., a mutant that is not capable of deacetylating, may be used. For example, mutant H363Y of SIRT1, described, e.g., in Luo et al. (2001) Cell 107:137 can be used. Alternatively, agents that inhibit transcription can be used.

9. Exemplary Drug Screening Assays

In certain aspects, the present invention provides screening methods for identifying compounds (agents) for treating or preventing neurodegenerative disorders or blood coagulation disorders. Candidate compounds identified by the subject screening methods can be administered to a subject, such as a subject in need thereof. A subject in need of such a treatment may be a subject who suffers from neurodegenerative disorders or blood coagulation disorders, or who has, or is, likely to have these disorders, as predicted, e.g., from family history. Exemplary agents are those described herein.

In certain embodiments, a compound described herein, e.g., a sirtuin activator or inhibitor, does not have significant or detectable anti-oxidant activities, as determined by any of the standard assays known in the art. For example, a compound does not significantly scavenge free-radicals, such as O₂ radicals. A compound may have less than about 2, 3, 5, 10, 30 or 100 fold anti-oxidant activity relative to another compound, e.g., resveratrol.

A compound may also have a binding affinity for a sirtuin of about 10⁻⁹ M, 10⁻¹⁰ M, 10⁻¹¹ M, 10⁻¹² M or less. A compound may reduce the Kₘ of a sirtuin for its substrate or NAD⁺ by a factor of at least about 2, 3, 4, 5, 10, 20, 30, 50 or 100. A compound may increase the Vₘₐₓ of a sirtuin by a factor of at least about 2, 3, 4, 5, 10, 20, 30, 50 or 100. Exemplary compounds that may increase the Vmax of a sirtuin include, for example, analogs of isonicotinamide, such as, for example, compounds of formulas 69-72, and/or analogs of O-acetyl-ADP-ribose, such as, for example, compounds of formulas 73-76. A compound may have an EC₅₀ for activating the deacetylase activity of a sirtuin of less than about 1 nM, less than about 10 nM, less than about 100 nM, less than about 1 μM, less than about 10 μM, less than about 100 μM, or from about 1-10 nM, from about 10-100 nM, from about 0.1-1 μM, from about 1-10 μM or from about 10-100 μM. A compound may activate the deacetylase activity of a sirtuin by a factor of at least about 5, 10, 20, 30, 50, or 100, as measured in an acellular assay or in a cell based assay as described in the Examples. A compound may cause at least a 10%, 30%, 50%, 80%, 2 fold, 5 fold, 10 fold, 50 fold or 100 fold greater induction of the deacetylase activity of SIRT1 relative to the same concentration of resveratrol or other compound described herein. A compound may also have an EC₅₀ for activating SIRT5 that is at least about 10 fold, 20 fold, 30 fold, 50 fold greater than that for activating SIRT1.

In an exemplary embodiment, the methods and compositions described herein may include a combination therapy comprising (i) at least one sirtuin-activating compound that reduce the Kₘ of a sirtuin for its substrate or NAD⁺ by a factor of at least about 2, 3, 4, 5, 10, 20, 30, 50 or 100, and (ii) at least one sirtuin-activating compound that increases the Vₘₐₓ of a sirtuin by a factor of at least about 2, 3, 4, 5, 10, 20, 30, 50 or 100. In one embodiment, a combination therapy may comprise at least two of the following: (i) at least one sirtuin-activating compound of formula 1-25, 30, and 32-65, (ii) at least one sirtuin-activating compound of formula 69-76, and (iii) at least one sirtuin-activating compound of formula 77-88.

A compound may traverse the cytoplasmic membrane of a cell. For example, a compound may have a cell-permeability of at least about 20%, 50%, 75%, 80%, 90% or 95%.

Compounds described herein may also have one or more of the following characteristics: the compound may be essentially non-toxic to a cell or subject; the compound may be an organic molecule or a small molecule of 2000 amu or less, 1000 amu or less; a compound may have a half-life under normal atmospheric conditions of at least about 30 days, 60 days, 120 days, 6 months or 1 year; the compound may have a half-life in solution of at least about 30 days, 60 days, 120 days, 6 months or 1 year; a compound may be more stable in solution than resveratrol by at least a factor of about 50%, 2 fold, 5 fold, 10 fold, 30 fold, 50 fold or 100 fold; a compound may promote deacetylation of the DNA repair factor Ku70; a compound may promote deacetylation of RelA/p65; a compound may increase general turnover rates and enhance the sensitivity of cells to TNF-induced apoptosis.

The effect of a compound on the activity of a sirtuin, such as SIRT1, may be determined as described, e.g., in Howitz et al., supra, or U.S. Patent Application Publication Nos. 2005/0136537 and 2005/0099173, or as follows. For instance, sirtuin proteins may be contacted with a compound in vitro, e.g., in a solution or in a cell. In one embodiment, a sirtuin protein is contacted with a compound in a solution and an activity of the sirtuin, e.g., its ability to deacetylate a protein, such as a histone, p53, or portions thereof, is determined. Generally, a sirtuin is activated or inhibited by a compound when at least one of its biological activities, e.g., deacetylation activity, is higher or lower, respectively, in the presence of the compound than in its absence. Activation or inhibition may be by a factor of at least about 10%, 30%, 50%, 100% (i.e., a factor of two), 3, 10, 30, or 100.

Whether a sirtuin is activated or inhibited can be determined, e.g., by contacting the sirtuin or a cell or cell extract containing the sirtuin with a deacetylation target, such as a histone, p53 protein, or portions thereof, and determining the level of acetylation of the deacetylation target. A higher level of acetylation of the target incubated with the sirtuin that is being tested relative to the level of acetylation of a control sirtuin indicates that the sirtuin that is being tested is activated. Conversely, a lower level of acetylation of the target incubated with the sirtuin that is being tested relative to the level of acetylation of a control sirtuin indicates that the sirtuin that is being tested is inhibited. The control sirtuin may be a recombinantly produced sirtuin that has not been contacted with a sirtuin-activating or -inhibiting compound.

Assays for determining the likelihood that a subject has or will develop neurodegenerative disorders or blood coagulation disorders are well known in the art. For example, such assays may comprise determining the level of activity or expression (e.g., mRNA, pre-mRNA or protein) of a sirtuin such as SIRT1 in a subject. A low level of sirtuin activity or expression in a subject is likely to indicate that the subject has or is likely to develop neurodegenerative disorders or blood coagulation disorders or secondary conditions thereof. Alternatively, a higher level of sirtuin activity or expression in a subject is likely to indicate that the subject has or is likely to be protected from developing neurodegenerative disorders or blood coagulation disorders. Other assays include determining the activity or level of expression of a sirtuin.

In certain embodiments, a method may comprise contacting a sirtuin with a test agent and determining the effect of the test agent on the activity of the sirtuin, e.g., SIRT1, as described, e.g., in Howitz et al., supra. The first step of the method may also comprise contacting a cell comprising a sirtuin with a test agent and determining the effect of the test agent on the activity of or expression level of the sirtuin. Expression levels of a sirtuin may be determined by measuring the mRNA, pre-mRNA or protein level of the sirtuin. Other steps of the method may comprise testing the agent in an animal model for neurodegenerative disorders or blood coagulation disorders. Such animal models are well known in the art. Screening methods may further comprise a step to determine the toxicity or adverse effects of the agents.

10. Pharmaceutical Formulations and Modes of Administration

Pharmaceutical compositions for use in accordance with the present methods may be formulated in conventional manner using one or more physiologically acceptable carriers or excipients. Thus, sirtuin-activating or -inhibiting compounds and their physiologically acceptable salts and solvates may be formulated for administration by, for example, injection (e.g. SubQ, IM, IP), inhalation or insufflation (either through the mouth or the nose) or oral, buccal, parenteral, sublingual or rectal administration. In one embodiment, the compound is administered locally, at the site where the target cells, e.g., neuronal cells or blood cells.

Compounds can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets, lozanges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

Polyphenols such as resveratrol can oxidize and lose sirtuin-stimulatory activity, especially in a liquid or semi-solid form. To prevent oxidation and preserve the sirtuin-stimulatory activity of polyphenol-containing compounds, the compounds may be stored in a nitrogen atmosphere or sealed in a type of capsule and/or foil package that excludes oxygen (e.g., Capsugel™).

For administration by inhalation, the compounds may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Controlled release formula also includes patches.

In certain embodiments, pharmaceutical compositions can be administered with medical devices known in the art. For example, a pharmaceutical composition described herein can be administered with a needle-less hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules also are known.

In certain embodiments, the compounds described herein can be formulated for delivery to the central nervous system (CNS) (reviewed in Begley, Pharmacology & Therapeutics 104: 2945 (2004)). Conventional approaches for drug delivery to the CNS include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide).

In certain embodiments, the compounds described herein can be formulated to ensure proper distribution in vivo. For example, the blood-brain barrier (BBB) excludes many highly hydrophilic compounds. To ensure that a therapeutic can cross the BBB (if desired), it can be formulated, for example, in a liposome. For methods of manufacturing liposomes, see, e.g., U.S. Pat. Nos. 4,522,811; 5,374,548; and 5,399,331. The liposomes may include one or more moieties which are selectively transported into specific cells or organs, thus enhance targeted drug delivery (see, e.g., V. V. Ranade (1989) J. Clin. Pharmacol. 29:685).

When the subject compounds are used to treat neurological disorders, the compositions may be administered by routes and methods resulting in exposure of the afflicted neuronal tissue and cells to the sirtuin modulators. This consideration is especially important in treating the central nervous system because of the blood-brain barrier (BBB), which limits delivery of therapeutic compounds into the brain. In demyelinating diseases, the compromised state of the blood-brain barrier may allow delivery of active agents by systemic administration (e.g., subcutaneous, intravenous, or oral). Where a more directed delivery is beneficial or required, methods for delivering the subject compounds into the CNS may be used. The method of administration may involve direct infusion into the cerebrospinal fluid via intrathecal or intraventricular route or implantation into the CNS area. Direct intracerebral infusion into particular neuronal populations is also contemplated. For example, see Gill et al., Nat. Med., 9:589-595, 2003.

Other methods for treating affected neuronal cells located in the brain utilize an implantable device such as an indwelling catheter through which the sirtuin modulator, in an appropriate formulation, can be infused directly onto the neuronal cells. Catheters used in intracranial penetration are typically fabricated so that their introduction to the brain is as minimally traumatic as possible. In addition to being minimally traumatic during insertion, certain inserted catheters must also be able to remain implanted without causing injury through unintended movement. In some uses, a catheter may be implanted and remain in the patient's brain for weeks or longer. Changes in the positioning of the catheter often occur during placement or during such extended periods. Therefore, the catheter must be capable of precise placement and as biocompatible as possible. In response to these requirements, state of the art intracranial catheters are typically thin, flexible pieces with smooth surfaces to minimize the amount of brain tissue contacted and to minimize damage to contacted brain tissue. For delivery within the CNS intrathecal delivery can be used with for example an Ommaya reservoir. U.S. Pat. No. 5,455,044 provides for use of a dispersion system for CNS delivery or see U.S. Pat. No. 5,558,852 for a discussion of CNS delivery.

In another embodiment, the sirtuin modulators are coupled to a drug transporter or carriers, as described above, which permit transport across the blood-brain barrier (see also, Bickel, U. Adv. Drug Deliv. Rev. 46: 247-79 (2001)). Drug transporters and carriers useful for this purpose include lipids, cationized albumin, insulin receptor antibody, transferrin receptor antibody, OX26 MAb (Partridge et al., Pharm. Res. 15: 576-82 (1998); Deguchi et al., Bioconjug. Chem. 10: 32-37 (1999)), liposomes, microparticles, or nanoparticles. These carriers undergo absorptive uptake or internalization by receptor mediated endocytosis, resulting in passage across the blood brain barrier. Immunoliposomes (antibody-directed liposomes) have also been prepared which purportedly can deliver the anti-neoplastic agent, daunomycin, to a rat brain (Huwyler et al., Proc. Nat'l Acad. Sci. USA 93: 14164-14169 (1996)). Biomolecular lipophilic complexes have also been described, which purportedly can deliver active agents to mammalian brains (U.S. Pat. No. 5,716,614). Conjugating avidin to the carriers or directly to the sirtuin modulator allows absorptive-mediated endocytosis of the conjugate, thus providing a useful method for drug delivery. These formulations allow systemic administration of the sirtuin modulators while targeting damaging immune reactions in the nervous system. Alternatively, the conjugates and carriers containing the sirtuin modulators may be delivered directly to the CNS.

Delivery of the sirtuin modulators to the CNS may also rely on disruptions to the blood brain barrier, such as intracranial infusion with hypertonic mannitol solutions. Alternatively, it may be preferable to administer the sirtuin modulators in combination with agents that increase transport across the blood brain barrier. These compounds have the effect of increasing permeability across the blood brain barrier and may or may not be conjugated to the subject sirtuin modulators. These agents include, but are not limited to, bradykinin and agonist derivatives (U.S. Pat. No. 5,112,596) and receptor mediated permeabilizers (U.S. Pat. Nos. 5,268,164 and 5,506,206). The solution is introduced intravenously (e.g., via the carotid artery) or by other acceptable routes. Concomitant with or subsequent to disruption, the pharmaceutically acceptable carriers, for example nanoparticles or liposomes, are introduced into the host to deliver the sirtuin modulators to the brain.

Administration of a pharmaceutically effective amount to the brain may also be achieved through the olfactory neural pathway, as provided in U.S. Pat. Nos. 6,342,478 and 5,624,898 and PCT Publication Nos. WO 033813A1, WO 09901229A1 and WO 044350A1. Delivery of the sirtuin modualtors via the olfactory system in a pharmaceutically acceptable carrier bypasses the blood brain barrier to permit delivery of the agents directly to the brain. Since there is no significant dilution of the sirtuin modulators by physiological fluids, concentrated delivery of sirtuin modulators are possible. Administration is done by intranasal application of the sirtuin modulators in a suitable carrier in the form of drops, spray, or powder. Compounds that are hydrophilic, charged and/or larger than 300 Dalton may be not delivered in therapeutic effective amounts by the olfactory methods described above. These compounds, but also all other compounds may be delivered more rapidly and more effectively by means of a physical enhancement technique such as electrotransport and/or phonophoresis (sonophoresis). The use of an enhancement technique such as electrotransport has the additional advantage that it can provide a dose- and rate-controlled delivery of the biologically active agent and the dose can be pre-programmed according to individual needs.

An alternate BBB circumventing pathway to the brain is provided by the optic nerve. The optic nerve, which is about 4 cm long, is directed backwards and medially through the posterior part of the orbital cavity. It then runs through the optic canal into cranial cavity and joins the optic chiasma. The optic nerve is enclosed in three sheaths, which are continuous with the membranes of the brain, and are prolonged as far as the back of the eyeball. Therefore, there is a direct connection between the optic nerve and the brain structures. Itaya and van Hoessen described transneuronal retrograde labeling of neurons in the stratum griseum superficiale of the superior colliculus following intra-ocular injection of wheat germ agglutinin-horseradish peroxidase. A study of the distribution of wheat germ agglutinin-horseradish peroxidase in the visual system following intra-ocular injections in the chick, rat and monkey confirmed early findings of transneural transport of this conjugate in vivo. It is therefore envisioned that a sirtuin modulator can be delivered direct to the CNS by a non-invasive delivery method and apparatus that utilizes the ocular pathway to circumvent the BBB.

One possibility to achieve sustained release kinetics is embedding or encapsulating the active compound into nanoparticles. Nanoparticles can be administrated as powder, as a powder mixture with added excipients or as suspensions. Colloidal suspensions of nanoparticles can easily be administrated through a cannula with small diameter.

Nanoparticles are particles with a diameter from about 5 nm to up to about 1000 nm. The term “nanoparticles” as it is used hereinafter refers to particles formed by a polymeric matrix in which the active compound is dispersed, also known as “nanospheres”, and also refers to nanoparticles which are composed of a core containing the active compound which is surrounded by a polymeric membrane, also known as “nanocapsules”. In certain embodiments, nanoparticles are preferred having a diameter from about 50 nm to about 500 nm, in particular from about 100 nm to about 200 nm.

Nanoparticles can be prepared by in situ polymerization of dispersed monomers or by using preformed polymers. Since polymers prepared in situ are often not biodegradable and/or contain toxicological serious byproducts, nanoparticles from preformed polymers are preferred. Nanoparticles from preformed polymers can be prepared by different techniques, e.g., by emulsion evaporation, solvent displacement, salting-out, mechanical grinding, microprecipitation, and by emulsification diffusion.

With the methods described above, nanoparticles can be formed with various types of polymers. For use in the method of the present invention, nanoparticles made from biocompatible polymers are preferred. The term “biocompatible” refers to material that after introduction into a biological environment has no serious effects to the biological environment. From biocompatible polymers those polymers are especially preferred which are also biodegradable. The term “biodegradable” refers to material that after introduction into a biological environment is enzymatically or chemically degraded into smaller molecules, which can be eliminated subsequently. Examples are polyesters from hydroxycarboxylic acids such as poly(lactic acid) (PLA), poly(glycolic acid) (PGA), polycaprolactone (PCL), copolymers of lactic acid and glycolic acid (PLGA), copolymers of lactic acid and caprolactone, polyepsilon caprolactone, polyhyroxy butyric acid and poly(ortho)esters, polyurethanes, polyanhydrides, polyacetals, polydihydropyrans, polycyanoacrylates, natural polymers such as alginate and other polysaccharides including dextran and cellulose, collagen and albumin.

Suitable surface modifiers can preferably be selected from known organic and inorganic pharmaceutical excipients. Such excipients include various polymers, low molecular weight oligomers, natural products and surfactants. Preferred surface modifiers include nonionic and ionic surfactants. Representative examples of surface modifiers include gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, e.g., macrogol ethers such as cetomacrogol 1000, polyoxyethylene castor oil derivatives, polyoxyethylene sorbitan fatty acid esters, e.g., the commercially available Tweens™, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxy propylcellulose, hydroxypropylmethylcellulose phthalate, noncrystalline cellulose, magnesium aluminum silicate, triethanolamine, polyvinyl alcohol, and polyvinylpyrrolidone (PVP). Most of these surface modifiers are known pharmaceutical excipients and are described in detail in the Handbook of Pharmaceutical Excipients, published jointly by the American Pharmaceutical Association and The Pharmaceutical Society of Great Britain, the Pharmaceutical Press, 1986.

Further description on preparing nanoparticles can be found, for example, in US Pat. No. 6,264,922, the contents of which are incorporated herein by reference.

Liposomes are a further drug delivery system which is easily injectable. Accordingly, in the method of invention the active compounds can also be administered in the form of a liposome delivery system. Liposomes are well-known by a person skilled in the art. Liposomes can be formed from a variety of phospholipids, such as cholesterol, stearylamine of phosphatidylcholines. Liposomes being usable for the method of invention encompass all types of liposomes including, but not limited to, small unilamellar vesicles, large unilamellar vesicles and multilamellar vesicles.

Liposomes are used for a variety of therapeutic purposes, and in particular, for carrying therapeutic agents to target cells. Advantageously, liposome-drug formulations offer the potential of improved drug-delivery properties, which include, for example, controlled drug release. An extended circulation time is often needed for liposomes to reach a target region, cell or site. In particular, this is necessary where the target region, cell or site is not located near the site of administration. For example, when liposomes are administered systemically, it is desirable to coat the liposomes with a hydrophilic agent, for example, a coating of hydrophilic polymer chains such as polyethylene glycol (PEG) to extend the blood circulation lifetime of the liposomes. Such surface-modified liposomes are commonly referred to as “long circulating” or “sterically stabilized” liposomes.

One surface modification to a liposome is the attachment of PEG chains, typically having a molecular weight from about 1000 daltons (Da) to about 5000 Da, and to about 5 mole percent (%) of the lipids making up the liposomes (see, for example, Stealth Liposomes, CRC Press, Lasic, D. and Martin, F., eds., Boca Raton, Fla., (1995)), and the cited references therein. The pharmacokinetics exhibited by such liposomes are characterized by a dose-independent reduction in uptake of liposomes by the liver and spleen via the mononuclear phagocyte system (MPS), and significantly prolonged blood circulation time, as compared to non-surface-modified liposomes, which tend to be rapidly removed from the blood and accumulated in the liver and spleen.

In certain embodiments, the complex is shielded to increase the circulatory half-life of the complex or shielded to increase the resistance of nucleic acid to degradation, for example degradation by nucleases.

As used herein, the term “shielding”, and its cognates such as “shielded”, refers to the ability of “shielding moieties” to reduce the non-specific interaction of the complexes described herein with serum complement or with other species present in serum in vitro or in vivo. Shielding moieties may decrease the complex interaction with or binding to these species through one or more mechanisms, including, for example, non-specific steric or non-specific electronic interactions. Examples of such interactions include non-specific electrostatic interactions, charge interactions, Van der Waals interactions, steric-hindrance and the like. For a moiety to act as a shielding moiety, the mechanism or mechanisms by which it may reduce interaction with, association with or binding to the serum complement or other species does not have to be identified. One can determine whether a moiety can act as a shielding moiety by determining whether or to what extent a complex binds serum species.

It should be noted that “shielding moieties” can be multifunctional. For example, a shielding moiety may also function as, for example, a targeting factor. A shielding moiety may also be referred to as multifunctional with respect to the mechanism(s) by which it shields the complex. While not wishing to be limited by proposed mechanism or theory, examples of such a multifunctional shielding moiety are pH sensitive endosomal membrane-disruptive synthetic polymers, such as PPAA or PEAA. Certain poly(alkylacrylic acids) have been shown to disrupt endosomal membranes while leaving the-outer cell surface membrane intact (Stayton et al. (2000) J. Controll. Release 65:203-220; Murthy et al. (1999) J. Controll. Release 61:137-143; WO 99/34831), thereby increasing cellular bioavailability and functioning as a targeting factor. However, PPAA reduces binding of serum complement to complexes in which it is incorporated, thus functioning as a shielding moiety.

Another way to produce a formulation, particularly a solution, of a sirtuin modulator such as resveratrol or a derivative thereof, is through the use of cyclodextrin. By cyclodextrin is meant α-, β-, or γ-cyclodextrin. Cyclodextrins are described in detail in Pitha et al., U.S. Pat. No. 4,727,064, which is incorporated herein by reference. Cyclodextrins are cyclic oligomers of glucose; these compounds form inclusion complexes with any drug whose molecule can fit into the lipophile-seeking cavities of the cyclodextrin molecule.

The cyclodextrin of the compositions according to the invention may be α-, β-, or γ-cyclodextrin. α-cyclodextrin contains six glucopyranose units; β-cyclodextrin contains seven glucopyranose units; and γ-cyclodextrin contains eight glucopyranose units. The molecule is believed to form a truncated cone having a core opening of 4.7-5.3 angstroms, 6.0-6.5 angstroms, and 7.5-8.3 angstroms in α-, β-, or γ-cyclodextrin respectively. The composition according to the invention may comprise a mixture of two or more of the α-, β-, or γ-cyclodextrins. Typically, however, the composition according to the invention will comprise only one of the α-, β-, or γ-cyclodextrins.

Most preferred cyclodextrins in the compositions according to the invention are amorphous cyclodextrin compounds. By amorphous cyclodextrin is meant non-crystalline mixtures of cyclodextrins wherein the mixture is prepared from α-, β-, or γ-cyclodextrin. In general, the amorphous cyclodextrin is prepared by non-selective alkylation of the desired cyclodextrin species. Suitable alkylation agents for this purpose include but are not limited to propylene oxide, glycidol, iodoacetarnide, chloroacetate, and 2-diethylaminoethlychloride. Reactions are carried out to yield mixtures containing a plurality of components thereby preventing crystallization of the cyclodextrin. Various alkylated cyclodextrins can be made and of course will vary, depending upon the starting species of cyclodextrin and the alkylating agent used. Among the amorphous cyclodextrins suitable for compositions according to the invention are hydroxypropyl, hydroxyethyl, glucosyl, maltosyl and maltotriosyl derivatives of β-cyclodextrin, carboxyamidomethyl-β-cyclodextrin, carboxymethyl-β-cyclodextrin, hydroxypropyl-β-cyclodextrin and diethylamino-β-cyclodextrin.

One example of resveratrol dissolved in the presence of a cyclodextrin is provided in Marier et al., J. Pharmacol. Exp. Therap. 302:369-373 (2002), the contents of which are incorporated herein by reference, where a 6 mg/mL solution of resveratrol was prepared using 0.9% saline containing 20% hydroxylpropyl-β-cyclodextrin.

As mentioned above, the compositions of matter of the invention comprise an aqueous preparation of preferably substituted amorphous cyclodextrin and one or more sirtuin modulators. The relative amounts of sirtuin modulators and cyclodextrin will vary depending upon the relative amount of each of the sirtuin modulators and the effect of the cyclodextrin on the compound. In general, the ratio of the weight of compound of the sirtuin modulators to the weight of cyclodextrin compound will be in a range between 1:1 and 1:100. A weight to weight ratio in a range of 1:5 to 1:50 and more preferably in a range of 1:10 to 1:20 of the compound selected from sirtuin modulators to cyclodextrin are believed to be the most effective for increased circulating availability of the sirtuin modulator.

Importantly, if the aqueous solution comprising the sirtuin modulators and a cyclodextrin is to be administered parenterally, especially via the intravenous route, a cyclodextrin will be substantially free of pyrogenic contaminants. Various forms of cyclodextrin, such as forms of amorphous cyclodextrin, may be purchased from a number of vendors including Sigma-Aldrich, Inc. (St. Louis, Mo., USA). A method for the production of hydroxypropyl-β-cyclodextrin is disclosed in Pitha et al., U.S. Pat. No. 4,727,064 which is incorporated herein by reference.

Additional description of the use of cyclodextrin for solubilizing compounds can be found in US 2005/0026849, the contents of which are incorporated herein by reference.

Rapidly disintegrating or dissolving dosage forms are useful for the rapid absorption, particularly buccal and sublingual absorption, of pharmaceutically active agents. Fast melt dosage forms are beneficial to patients, such as aged and pediatric patients, who have difficulty in swallowing typical solid dosage forms, such as caplets and tablets. Additionally, fast melt dosage forms circumvent drawbacks associated with, for example, chewable dosage forms, wherein the length of time an active agent remains in a patient's mouth plays an important role in determining the amount of taste masking and the extent to which a patient may object to throat grittiness of the active agent.

To overcome such problems manufacturers have developed a number of fast melt solid dose oral formulations. These are available from manufacturers including Cima Labs, Fuisz Technologies Ltd., Prographarm, R. P. Scherer, Yamanouchi-Shaklee, and McNeil-PPC, Inc. All of these manufacturers market different types of rapidly dissolving solid oral dosage forms. See e.g., patents and publications by Cima Labs such as U.S. Pat. Nos. 5,607,697, 5,503,846, 5,223,264, 5,401,513, 5,219,574, and 5,178,878, WO 98/46215, WO 98/14179; patents to Fuisz Technologies, now part of BioVail, such as U.S. Pat. Nos. 5,871,781, 5,869,098, 5,866,163, 5,851,553, 5,622,719, 5,567,439, and 5,587,172; U.S. Pat. No. 5,464,632 to Prographarm; patents to R. P. Scherer such as U.S. Pat. Nos. 4,642,903, 5,188,825, 5,631,023 and 5,827,541; patents to Yamanouchi-Shaklee such as U.S. Pat. Nos. 5,576,014 and 5,446,464; patents to Janssen such as U.S. Pat. Nos. 5,807,576, 5,635,210, 5,595,761, 5,587,180 and 5,776,491; U.S. Pat. Nos. 5,639,475 and 5,709,886 to Eurand America, Inc.; U.S. Pat. Nos. 5,807,578 and 5,807,577 to L.A.B. Pharmaceutical Research; patents to Schering Corporation such as U.S. Pat. Nos. 5,112,616 and 5,073,374; U.S. Pat. No. 4,616,047 to Laboratoire L. LaFon; U.S. Pat. No. 5,501,861 to Takeda Chemicals Inc., Ltd.; and U.S. Pat. No. 6,316,029 to Elan.

In one example of fast melt tablet preparation, granules for fast melt tablets made by either the spray drying or pre-compacting processes are mixed with excipients and compressed into tablets using conventional tablet making machinery. The granules can be combined with a variety of carriers including low density, high moldability saccharides, low moldability saccharides, polyol combinations, and then directly compressed into a tablet that exhibits an improved dissolution and disintegration profile.

The tablets according to the present invention typically have a hardness of about 2 to about 6 Strong-Cobb units (scu). Tablets within this hardness range disintegrate or dissolve rapidly when chewed. Additionally, the tablets rapidly disentegrate in water. On average, a typical 1.1 to 1.5 gram tablet disintegrates in 1-3 minutes without stirring. This rapid disintegration facilitates delivery of the active material.

The granules used to make the tablets can be, for example, mixtures of low density alkali earth metal salts or carbohydrates. For example, a mixture of alkali earth metal salts includes a combination of calcium carbonate and magnesium hydroxide. Similarly, a fast melt tablet can be prepared according to the methods of the present invention that incorporates the use of A) spray dried extra light calcium carbonate/maltodextrin, B) magnesium hydroxide and C) a eutectic polyol combination including Sorbitol Instant, xylitol and mannitol. These materials have been combined to produce a low density tablet that dissolves very readily and promotes the fast disintegration of the active ingredient. Additionally, the pre-compacted and spray dried granules can be combined in the same tablet.

For fast melt tablet preparation, a sirtuin modulator useful in the present invention can be in a form such as solid, particulate, granular, crystalline, oily or solution. The sirtuin modulator for use in the present invention may be a spray dried product or an adsorbate that has been pre-compacted to a harder granular form that reduces the medicament taste. A pharmaceutical active ingredient for use in the present invention may be spray dried with a carrier that prevents the active ingredient from being easily extracted from the tablet when chewed.

In addition to being directly added to the tablets of the present invention, the medicament drug itself can be processed by the pre-compaction process to achieve an increased density prior to being incorporated into the formulation.

The pre-compaction process used in the present invention can be used to deliver poorly soluble pharmaceutical materials so as to improve the release of such pharmaceutical materials over traditional dosage forms. This could allow for the use of lower dosage levels to deliver equivalent bioavailable levels of drug and thereby lower toxicity levels of both currently marketed drug and new chemical entities. Poorly soluble pharmaceutical materials can be used in the form of nanoparticles, which are nanometer-sized particles.

In addition to the active ingredient and the granules prepared from low density alkali earth metal salts and/or water soluble carbohydrates, the fast melt tablets can be formulated using conventional carriers or excipients and well established pharmaceutical techniques. Conventional carriers or excipients include, but are not limited to, diluents, binders, adhesives (i.e., cellulose derivatives and acrylic derivatives), lubricants (i.e., magnesium or calcium stearate, vegetable oils, polyethylene glycols, talc, sodium lauryl sulphate, polyoxy ethylene monostearate), disintegrants, colorants, flavorings, preservatives, sweeteners and miscellaneous materials such as buffers and adsorbents.

Additional description of the preparation of fast melt tablets can be found, for example, in U.S. Pat. No. 5,939,091, the contents of which are incorporated herein by reference.

Pharmaceutical compositions (including cosmetic preparations) may comprise from about 0.00001 to 100% such as from 0.001 to 10% or from 0.1% to 5% by weight of one or more compounds described herein.

In one embodiment, a compound described herein, is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Formulations may be colorless, odorless ointments, lotions, creams, microemulsions and gels.

Compounds may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington's, cited in the preceding section, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Exemplary water-soluble ointment bases are prepared from polyethylene glycols (PEGs) of varying molecular weight; again, reference may be had to Remington's, supra, for further information.

Compounds may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. An exemplary lotion formulation for use in conjunction with the present method contains propylene glycol mixed with a hydrophilic petrolatum such as that which may be obtained under the trademark Aquaphor^{RTM} from Beiersdorf, Inc. (Norwalk, Conn.).

Compounds may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

Compounds may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

Compounds may be incorporated into gel formulations, which generally are semisolid systems consisting of either suspensions made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients and preservatives. An optimum topical formulation comprises approximately: 2 wt. % to 60 wt. %, preferably 2 wt. % to 50 wt. %, solubilizer and/or skin permeation enhancer; 2 wt. % to 50 wt. %, preferably 2 wt. % to 20 wt. %, emulsifiers; 2 wt. % to 20 wt. % emollient; and 0.01 to 0.2 wt. % preservative, with the active agent and carrier (e.g., water) making of the remainder of the formulation.

A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide (C₁₀ MSO) and tetradecylmethyl sulfboxide; pyrrolidones such as 2-pyrrolidone, N-methyl-2-pyrrolidone and N-(-hydroxyethyl)pyrrolidone; urea; N,N-diethyl-m-toluamide; C₂-C₆ alkanediols; miscellaneous solvents such as dimethyl formamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram; available under the trademark Azone^{RTM} from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol^{RTM}) and diethylene glycol monoethyl ether oleate (available commercially as Softcutol^{RTM}); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasoi^{RTM}); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition as herein defined.

In an alternative embodiment, a pharmaceutical formulation is provided for oral or parenteral administration, in which case the formulation may comprise an activating compound-containing microemulsion as described above, and may contain alternative pharmaceutically acceptable carriers, vehicles, additives, etc. particularly suited to oral or parenteral drug administration. Alternatively, an activating compound-containing microemulsion may be administered orally or parenterally substantially as described above, without modification.

Administration of a sirtuin activator or inhibitor may be followed by measuring a factor in the subject, such as measuring the activity of the sirtuin. In an illustrative embodiment, a cell is obtained from a subject following administration of an activating or inhibiting compound to the subject, such as by obtaining a biopsy, and the activity of the sirtuin or sirtuin expression level is determined in the biopsy. Alternatively, biomarkers, such as plasma biomarkers may be followed. The cell may be any cell of the subject, but in cases in which an activating compound is administered locally, the cell is preferably a cell that is located in the vicinity of the site of administration. For example, the cell may be a neuronal cell or a blood cell.

Introduction and expression of a nucleic acid encoding a sirtuin or molecules (e.g., an siRNA) that will reduced the protein level of a sirtuin in a cell may be accomplished using an expression vector. Exemplary expression vectors include adenoviral vectors or adenoviral-associated viruses (AAV). These vectors, as well as others and methods for infecting target cells are well known in the art. Alternatively, nucleic acids may also be introduced into cells using liposomes or similar technologies.

11. Exemplary Kits

Also provided herein are kits, e.g., kits for therapeutic purposes, including kits for treating or preventing neurodegenerative disorders or blood coagulation disorders or secondary conditions thereof. A kit may comprise one or more agent that modulates sirtuin protein activity or level, e.g., sirtuin activating or inhibitory compounds, such as those described herein, and optionally devices for contacting cells with the agents. Devices include syringes, stents and other devices for introducing a compound into a subject or applying it to the skin of a subject.

Further, a kit may also contain components for measuring a factor, e.g., described above, such as the activity of sirtuin proteins, e.g., in tissue samples.

Other kits include kits for diagnosing the likelihood of having or developing neurodegenerative disorders or blood coagulation disorders or secondary conditions thereof. A kit may comprise an agent for measuring the activity and or expression level of a sirtuin.

Kits for screening assays are also provided. Exemplary kits comprise one or more agents for conducting a screening assay, such as a sirtuin or a biologically active portion thereof, or a cell or cell extract comprising such. Any of the kits may also comprise instructions for use.

The present description is further illustrated by the following examples, which should not be construed as limiting in any way. The contents of all cited references (including literature references, issued patents, published patent applications and GenBank Accession numbers as cited throughout this application) are hereby expressly incorporated by reference.

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Exemplification

The invention now being generally described, it will be more readily understood by reference to the following examples which are included merely for purposes of illustration of certain aspects and embodiments of the present invention, and are not intended to limit the invention in any way. Examples 1-16 provide methods for assaying sirtuin activity and may be used for testing a variety of compounds for sirtuin modulating activity.

### EXAMPLE1

### Small Molecule Activators of SIRT1

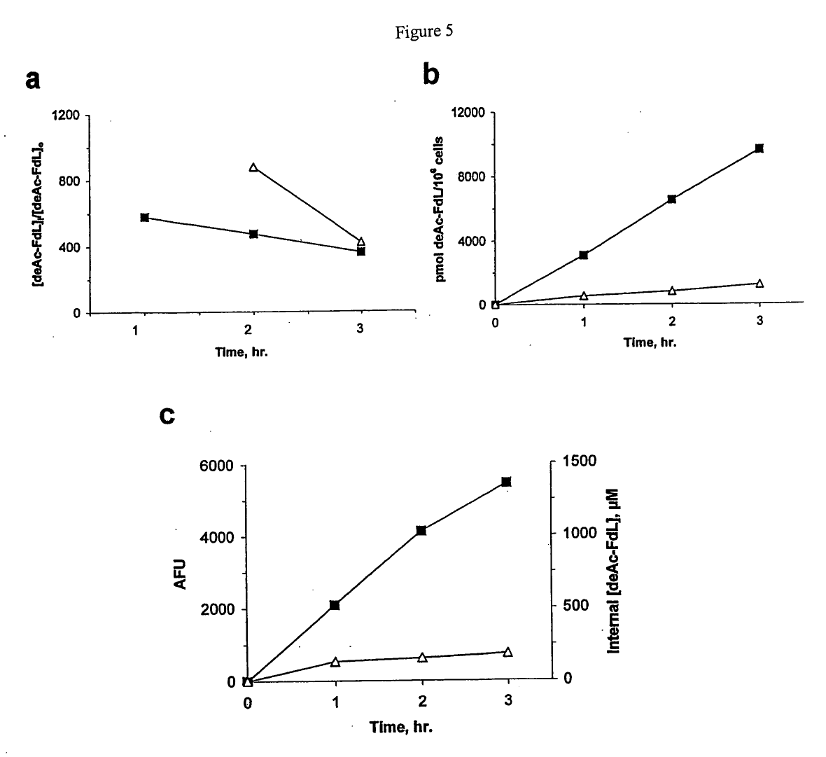

To identify compounds that modulate SIRT1 activity, a number of small molecule libraries were screened using a fluorescent deacetylation assay in 96-well plates²⁶. The substrate used in the assay was a fluorogenic peptide based on the sequence encompassing the p53-K382 acetylation site, a known target of SIRT1 in vivo^{20,21,27}. This substrate was preferred over a variety of other fluorogenic peptide substrates that were based on other known HDAC targets (FIG. 5). The small molecule libraries included analogues of nicotinamide, ε-acetyl lysine, NAD⁺, nucleotides, nucleotide analogues and purinergic ligands. From the initial screen, several sirtuin inhibitors were found (Supplementary Table 7). However, the most striking outcome was the identification of two compounds, quercetin and piceatannol, that stimulated SIRT1 activity five and eight-fold, respectively (Table 1). Both quercetin and piceatannol have been previously identified as protein kinase inhibitors^{28,29}.

Comparison of the structures of the two activating compounds suggested a possible structure-activity relationship. Piceatannol comprises two phenyl groups trans to one another across a linking ethylene moiety. The trans-stilbene ring structures of piceatannol are superimposable on the flavonoid A and B rings of quercetin, with the ether oxygen and carbon-2 of the C ring aligning with the ethylene carbons in piceatannol (see structures, Table 1). Further, the 5, 7, 3′ and 4′ hydroxyl group positions in quercetin can be aligned, respectively, with the 3, 5, 3′ and 4′ hydroxyls of piceatannol.

Given the demonstrated longevity-enhancing effects of sirtuin activity in S. cerevisiae⁷ and C. elegans¹⁹, it was naturally of interest to further explore the structure-activity relationship among compounds that stimulate SIRT1. Both quercetin and piceatannol are polyphenols, members of a large and diverse group of plant secondary metabolites that includes flavones, stilbenes, flavanones, isoflavones, catechins (flavan-3-ols), chalcones, tannins and anthocyanidins^{30,31}. Polyphenols noteworthy with respect to potential longevity-enhancing effects include resveratrol, a stilbene found in red wine and epigallocatechin gallate (EGCG) from green tea. Both have been suggested on the basis of epidemiological and mechanistic investigations to exert cancer chemopreventive and cardioprotective effects³⁰⁻³². Therefore, a secondary screen encompassing resveratrol was performed, EGCG and additional representatives from a number of the polyphenol classes listed above. The screen emphasized flavones due to the great number of hydroxyl position variants available in this group³¹. The results of this screen are summarized in Supplementary Tables 1-6. In the tables, a “ratio to control rate” above 1 indicates that a compound with such a rate is an activator of the sirtuin tested and a number under 1 indicates that a compound is an inhibitor.

Additional potent SIRT1 activators were found among the stilbenes, chalcones and flavones (Table 1, Supplementary Tables 1 and 2). The six most active flavones had 3′ and 4′ hydroxyls (Supplementary Table 2), although it should be noted that the most active compound overall, resveratrol (3,5,4′-trihydroxystilbene), was more active than piceatannol, which differs only by its additional 3′-hydroxyl (Table 1). The importance of the 4′-hydroxyl to activity is underscored by the fact that each of the 12 most stimulatory flavones share this feature (Supplementary Tables 1 and 2).

Many, but not all of the most active compounds include hydroxyls in the two meta positions (e.g. 5,7-dihydroxylated flavones) of the ring (A ring), trans to that with the 4′ or 3′,4′ pattern (B ring, see Table 1, Supplementary Tables 1 and 2). A potentially coplanar orientation of the trans phenyl rings may be important for activity since catechins and flavanones, which lack the 2,3 double-bond, have weak activity despite having equivalent hydroxylation patterns to various stimulatory flavones (compare Supplementary Tables 2 and 3 with 4 and 5). The absence of activity in the isoflavone genistein, although hydroxylated in an equivalent way to the stimulatory compounds apigenin and resveratrol (see Supplementary Tables 1, 2 and 4), is consistent with the idea that the trans positioning and spacing of the hydroxylated rings contributes strongly to activity.

The biological effects of polyphenols are frequently attributed to antioxidant, metal ion chelating and/or free-radical scavenging activity^{30,32}. It may be possible that the apparent polyphenol stimulation of SIRT1 might simply represent the repair of oxidative and/or metal-ion induced damage incurred during preparation of the recombinant protein. Two features of the results argue against this being the case. First, a variety of free-radical protective compounds, including antioxidants, chelators and radical scavengers, failed to stimulate SIRT1 (see Supplementary Table 6.). Second, among various polyphenols of equivalent antioxidant capacity diverse SIRT1 stimulating activity was observed (e.g. compare resveratrol, quercetin and the epicatechins in Supplementary Tables 1, 2 and 5 and see ³³).

### EXAMPLE 2

### Resveratrol's Effects on SIRT1 Kinetics

Detailed enzyme kinetic investigations were performed using the most potent activator, resveratrol. Dose-response experiments performed under the conditions of the polyphenol screening assays (25 μM NAD⁺, 25 μM p53-382 acetylated peptide), showed that the activating effect doubled the rate at ˜11 μM and was essentially saturated at 100 μM resveratrol (FIG. 1a). Initial enzyme rates, in the presence or absence of 100 μM resveratrol, were determined either as a function of acetyl-peptide concentration with high NAD⁺ (3 mM NAD⁺, FIG. 1b) or as a function of NAD⁺ concentration with high acetyl-peptide (1 mM p53-382 acetylated peptide, FIG. 1c). Although resveratrol had no significant effect on the two Vₘₐₓ determinations (FIGS. 1b, 1c), it had pronounced effects on the two apparent Kₘs. Its effect on the acetylated peptide Kₘ was particularly striking, amounting to a 35-fold decrease (FIG. 1b). Resveratrol also lowered the Kₘ for NAD⁺ over 5-fold (FIG. 1c). Since resveratrol acts only on Kₘ, it could be classified as an allosteric effector of ‘K system’ type³⁴. This can imply that only the substrate binding affinity of the enzyme has been altered, rather than a rate-limiting catalytic step.

Previous kinetic analysis of SIRT1 and Sir2²⁶ and genetic analysis of Sir2's role in yeast lifespan extension^{6,35} have implicated nicotinamide (a product of the sirtuin reaction) as a physiologically important inhibitor of sirtuin activity. Therefore the effects of resveratrol on nicotinamide inhibition were tested. In experiments similar to those of FIGS. 1b and 1c, kinetic constants in the presence of 50 μM nicotinamide were determined either by varying the concentration of NAD⁺ or that of the p53-382 acetylated peptide (FIG. 1d). Nicotinamide, in contrast to resveratrol, affects the SIRT1 Vₘₐₓ (note 30% and 36% Vₘₐₓ decreases in absence of resveratrol, FIG. 1d and see ref.²⁶). In the presence of 50 μM nicotinamide, resveratrol appears to have complex, concentration-dependent effects on the kinetics of SIRT1 (FIG. 1d). Apparent Kₘ for NAD⁺ and acetylated substrate appear to actually be raised by 5 μM resveratrol when nicotinamide is present. At 20 and 100 μM, in the presence of 50 μM nicotinamide, resveratrol lowers the Kₘ for both NAD⁺ and acetylated peptide, without reversing the nicotinamide-induced Vₘₐₓ decrease. It has been proposed that sirtuins may bind nicotinamide at a second site, known as “the C pocket”, distinct from the “B” site that interacts with the nicotinamide moiety of NAD⁺²⁶. In light of this potential complexity, further kinetic studies, supplemented by structural/crystallographic information, will likely be necessary to fully elucidate the interplay between the effects of nicotinamide and polyphenols.

### EXAMPLE 3

### Activating Compounds Extend Yeast Lifespan

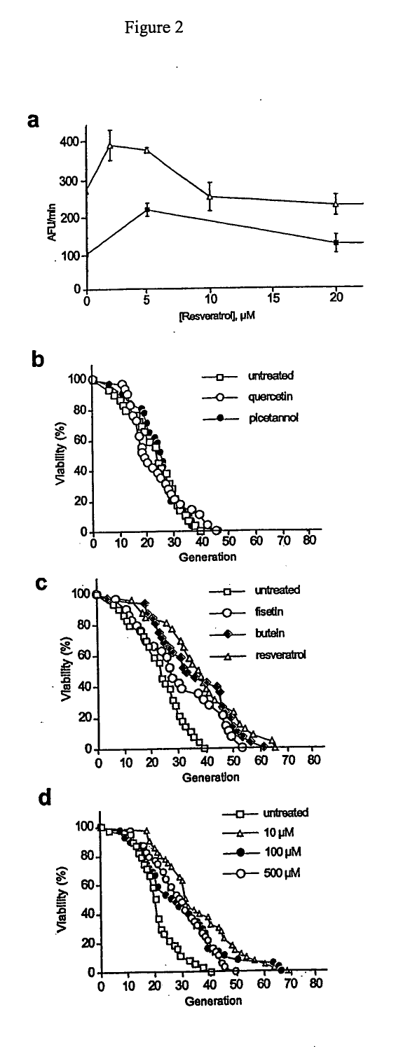

To investigate whether these compounds could stimulate sirtuins in vivo, S. cerevisiae, an organism in which the upstream regulators and downstream targets of Sir2 are relatively well understood, was used. A resveratrol dose-response study of Sir2 deacetylation rates (FIG. 2a) indeed reveals that resveratrol stimulates Sir2 in vitro, with the optimum concentration of activator being 2-5 μM. Levels of activation were somewhat lower than those for SIRT1, and unlike SIRT1, inhibition was seen at concentrations greater than ˜100 μM.

Resveratrol and four other potent sirtuin activators, representatives of the stilbene, flavone, and chalcone families, were tested for their effect on yeast lifespan. Due to the potential impediment by the yeast cell wall or plasma membrane and suspected slow oxidation of the compound in the medium, a concentration (10 μM) was used which is slightly higher than the optimal resveratrol concentration in vitro. As shown in FIG. 2b, quercetin and piceatannol had no significant effect on lifespan. In contrast, butein, fisetin and resveratrol increased average lifespan by 31, 55 and 70%, respectively, and all three significantly increased maximum lifespan (FIG. 2c). Concentrations of resveratrol higher than 10 μM provided no added lifespan benefit and there was no lasting effect of the compound on the lifespan of pre-treated young cells (FIG. 2d and data not shown).

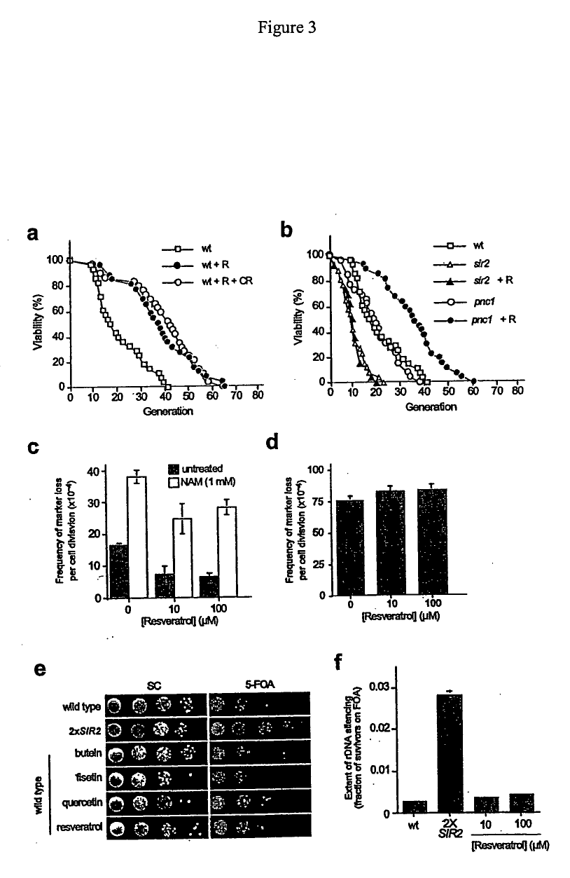

For subsequent yeast genetic experiments resveratrol was used because it was the most potent SIRT1 activator and provided the greatest lifespan extension. Glucose restriction, a form of CR in yeast, resulted in no significant extension of the long-lived resveratrol-treated cells (FIG. 3a), indicating that resveratrol likely acts via the same pathway as CR. Consistent with this, resveratrol had no effect on the lifespan of a sir2 null mutant (FIG. 3b). Given that resveratrol is reported to have fungicidal properties at high concentrations³⁶, and that mild stress can extend yeast lifespan by activating PNC1⁶, it was plausible that resveratrol was extending lifespan by inducing PNC1, rather than acting on Sir2 directly. However, resveratrol extended the lifespan of a pnc1 null mutant nearly as well as it did wild type cells (FIG. 3b). Together these data show that resveratrol acts downstream of PNC1 and requires SIR₂ for its effect. Thus, the simplest explanation is that resveratrol increases lifespan by directly stimulating Sir2 activity.

A major cause of yeast aging is thought to stem from the inherent instability of the repetitive rDNA locus^{2,5,37-39}. Homologous recombination between rDNA repeats can generate an extrachromosomal circular form of rDNA (ERC) that is replicated until it reaches toxic levels in old cells. Sir2 is thought to extend lifespan by suppressing recombination at the replication fork barrier of rDNA⁴⁰. Consistent with the lifespan extension, it was observed for resveratrol that this compound reduced the frequency of rDNA recombination by ˜60% (FIG. 3c), in a SIR₂-dependent manner (FIG. 3d). In the presence of the Sir2 inhibitor nicotinamide, recombination was also decreased by resveratrol (FIG. 3c), in agreement with the kinetic data (see FIG. 1d). Interestingly, it was found that resveratrol and other sirtuin activators had only minor effects on rDNA silencing (FIGS. 3e and f). Work is underway to elucidate how these various compounds can differentially affect rDNA stability and silencing.

Another measure of lifespan in S. cerevisiae is the length of time cells can survive in a metabolically active but nutrient deprived state. Aging under these conditions (i.e. chronological aging) is primarily due to oxidative damage⁴¹. Resveratrol (10 μM or 100 μM) failed to extend chronological lifespan (not shown), indicating that the sirtuin-stimulatory effect of resveratrol may be more relevant in vivo than its antioxidant activity^{30,31}.

### EXAMPLE 4

### Effects of Activators in Human Cells

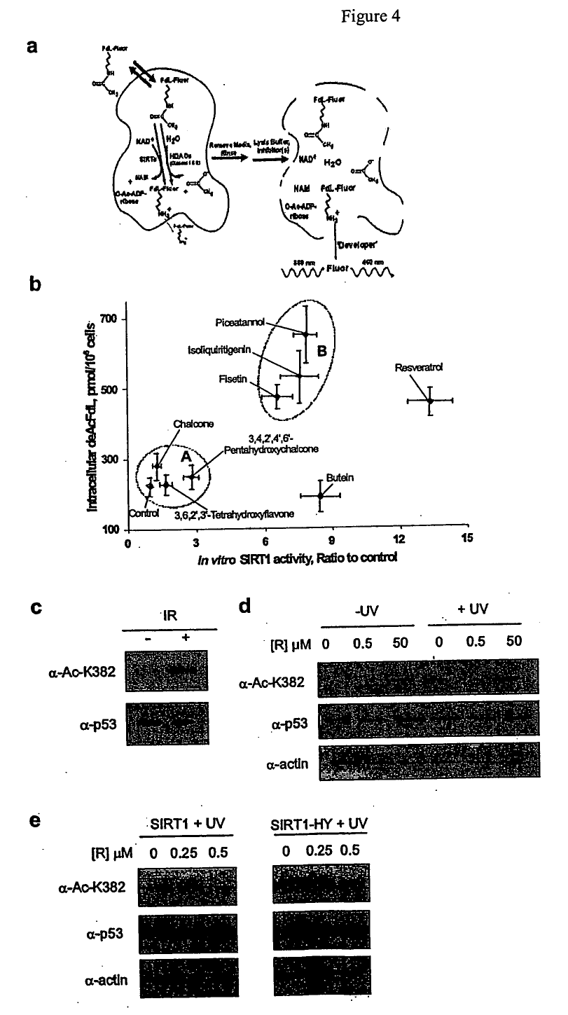

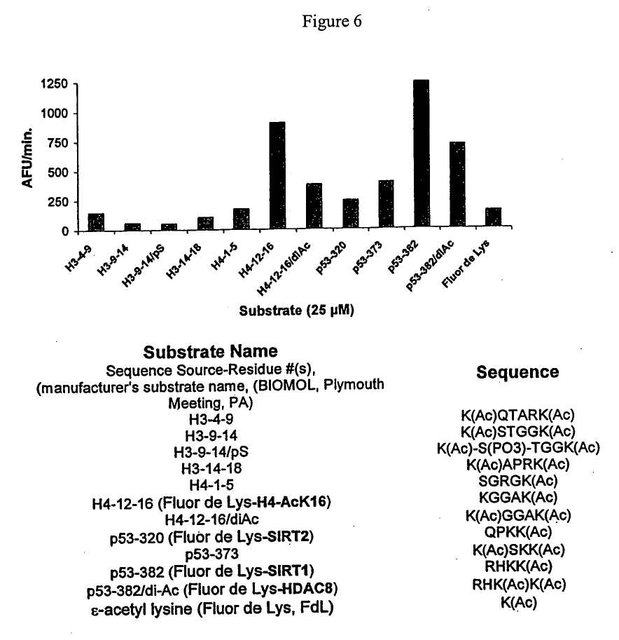

To test whether these compounds could stimulate human SIRT1 in vivo, a cellular deacetylase assay was employed. A schematic of the assay procedure is depicted in FIG. 4a. Cells are incubated with media containing the fluorogenic ε-acetyl-lysine substrate, ‘Fluor de Lys’ (FdL). This substrate, neutral when acetylated, becomes positively charged upon deacetylation and accumulates within cells (see FIG. 6a). Lysis of the cells and addition of the non-cell-permeable ‘Developer’ reagent releases a fluorophor specifically from those substrate molecules that have been deacetylated (FIG. 4a and see Methods). With HeLa cells growing adherently, 5-10% of the signal produced in this assay is insensitive to 1 μM trichostatin A (TSA), a potent inhibitor of class I and II HDACs but not sirtuins (class III)⁴² (FIGS. 6b and 6c).

A selection of SIRT1-stimulatory and non-stimulatory polyphenols were tested for their effects on this TSA-insensitive signal (FIG. 4b). Cellular deacetylation signals in the presence of each compound (y-axis, FIG. 4b) were plotted against their fold-stimulations of SIRT1 in vitro (x-axis, FIG. 4b, data from Supplementary Tables 1-3). For most of the compounds, the in vitro activity roughly corresponded to the cellular signal. Compounds with little or no in vitro activity clustered around the negative control (Group A, FIG. 4b). Another grouping, of strong in vitro activators is clearly distanced from the low activity cluster in both dimensions (Group B, FIG. 4b). A notable outlier was butein, a potent activator of SIRT1 in vitro which had no effect on the cellular signal. With allowances for possible variation among these compounds in properties unrelated to direct sirtuin stimulation, such as cell-permeability and rates of metabolism, these data are consistent with the idea that certain polyphenols can activate native sirtuins in vivo.

One known target of SIRT1 in vivo is lysine 382 of p53. Deacetylation of this residue by SIRT1 decreases the activity and half-life of p53^{20,21,27}. To follow the acetylation status of K382 a rabbit polyclonal antibody was generated that recognizes the acetylated form of K382 (Ac-K382) on Western blots of whole cell lysates. As a control it was shown that the signal was specifically detected in extracts from cells exposed to ionizing radiation (FIG. 4c), but not in extracts from cells lacking p53 or where arginine had been substituted for lysine 382 (data not shown). U2OS osteosarcoma cells were pre-treated for 4 hours with resveratrol (0.5 and 50 μM) and exposed to UV radiation. It was consistently observed a marked decrease in the level of Ac-K382 in the presence of 0.5 μM resveratrol, compared to untreated cells (FIG. 4d). At higher concentrations of resveratrol (>50 μM) the effect was reversed (FIG. 4d and data not shown), consistent with previous reports of increased p53 activity at such concentrations⁴³. The ability of low concentrations of resveratrol to promote deacetylation of p53 was diminished in cells expressing a dominant-negative SIRT1 allele (H363Y) (FIG. 4e), demonstrating that SIRT1 is necessary for this effect. This biphasic dose-response of resveratrol could explain the dichotomy in the literature regarding the effects of resveratrol on cell survival^{30,43,44}.

Thus, the first known class of small molecule sirtuin activators has been discovered, all of which are plant polyphenols. These compounds can dramatically stimulate sirtuin activity in vitro and promote effects consistent with increased sirtuin activity in vivo. In human cells, resveratrol promotes SIRT1-mediated p53 deacetylation of K382. In yeast, the effect of resveratrol on lifespan is as great as any longevity-promoting genetic manipulation⁶ and has been linked convincingly to the direct activation of Sir2. The correlation between lifespan and rDNA recombination, but not silencing, adds to the body of evidence that yeast aging is due to DNA instability2,5,37-39 not gene dysregulation⁴⁵.

How can the activation of the yeast and human sirtuins by so many plant metabolites be explained? Sirtuins have been found in diverse eukaryotes, including fungi, protozoans, metazoans and plants^{46,47}, and likely evolved early in life's history¹. Plants are known to produce a variety of polyphenols, including resveratrol, in response to stresses such as dehydration, nutrient deprivation, UV radiation and pathogens^{48,49}. Therefore it is plausible that these compounds may be synthesized to regulate a sirtuin-mediated plant stress response. This would be consistent with the recently discovered relationship between environmental stress and Sir2 activity in yeast⁶. Perhaps these compounds have stimulatory activity on sirtuins from fungi and animals because they mimic an endogenous activator, as is the case for the opiates/endorphins, cannabinols/endocannabinoids and various polyphenols with estrogen-like activity30,31. Alternatively, animal and fungal sirtuins may have retained or developed an ability to respond to these plant metabolites because they are a useful indicator of a deteriorating environment and/or food supply.

### EXAMPLE 5

### Materials and Methods for Examples 1-4

Compound Libraries and Deacetylation Assays

His₆-tagged recombinant SIRT1 and GST-tagged recombinant Sir2 were prepared as previously described²⁶. From 0.1 to 1 μg of SIRT1 and 1.5 μg of Sir2 were used per deacetylation assay (in 50 μl total reaction) as previously described²⁶. SIRT1 assays and certain of those for Sir2 employed the p53-382 acetylated substrate (° Fluor de Lys-SIRT1′, BIOMOL) rather than FdL.

Themed compound libraries (BIOMOL) were used for primary and secondary screening. Most polyphenol compounds were dissolved at 10 mM in dimethylsulfoxide (DMSO) on the day of the assay. For water soluble compounds and negative controls, 1% v/v DMSO was added to the assay. In vitro fluorescence assay results were read in white ½-volume 96-well microplates (Coming Costar 3693) with a CytoFluor™. II fluorescence plate reader (PerSeptive Biosystems, Ex. 360 nm, Em. 460 nm, gain=85). HeLa cells were grown and the cellular deacetylation assays were performed and read, as above, but in full-volume 96-well microplates (Coming Costar 3595). Unless otherwise indicated all initial rate measurements were means of three or more replicates, obtained with single incubation times, at which point 5% or less of the substrate initially present had been deacetylated. Calculation of net fluorescence increases included subtraction of a blank value, which in the case of Sir2 was obtained by omitting the enzyme from the reaction and in the case of SIRT1 by adding an inhibitor (200 μM suramin or 1 mM nicotinamide) to the reaction prior to the acetylated substrate. A number of the polyphenols partially quenched the fluorescence produced in the assay and correction factors were obtained by determining the fluorescence increase due to a 3 μM spike of an FdL deacetylated standard (BIOMOL, catalog number KI-142). All error bars represent the standard error of the mean.

Media and Strains

All yeast strains were grown at 30° C. in complete yeast extract/bactopeptone, 2.0% (w/v) glucose (YPD) medium except where stated otherwise. Calorie restriction was induced in 0.5% glucose. Synthetic complete (SC) medium consisted of 1.67% yeast nitrogen base, 2% glucose, 40 mg/litre each of auxotrophic markers. SIR₂ was integrated in extra copy and disrupted as described⁵. Other strains are described elsewhere²⁶. For cellular deacetylation assays, HeLa S3 cells were used. U20S osteosarcoma and human embryonic kidney (HEK 293) cells were cultured adherently in Dulbecco's Modified Eagle's Medium (DMEM) containing 10% fetal calf serum (FCS) with 1.0% glutamine and 1.0% penecillin/streptomycin. HEK 293 overexpressing dominant negative SIRT1 H363Y was a gift of R. Frye (U. Pittsburgh).

Lifespan Determinations

Lifespan measurements were performed using PSY316AT MATα as previously described³⁵. All compounds for lifespan analyses were dissolved in 95% ethanol and plates were dried and used within 24 hours. Prior to lifespan analysis, cells were pre-incubated on their respective media for at least 15 hours. Following transfer to a new plate, cells were equilibrated on the medium for a minimum of 4 hours prior to micro-manipulating them. At least 30 cells were examined per experiment and each experiment was performed at least twice. Statistical significance of lifespan differences was determined using the Wilcoxon rank sum test. Differences are stated to be significant when the confidence is higher than 95%.

Silencing and Recombination Assays

Ribosomal DNA silencing assays using the URA3 reporters were performed as previously described²⁶. Ribosomal DNA recombination frequencies were determined by plating W303AR cells³⁷ on YPD medium with low adenine/histidine and counting the fraction of half-red sectored colonies using Bio-Rad Quantity One software as previously described³⁵. At least 6000 cells were analyzed per experiment and all experiments were performed in triplicate. All strains were pre-grown for 15 hours with the relevant compound prior to plating.

Proteins and Western Analyses

Recombinant Sir2-GST was expressed and purified from E. coli as previously described except that lysates were prepared using sonication²⁶. Recombinant SIRT1 from E. coli was prepared as previously described²⁶. Polyclonal antiserum against p53-AcK382 was generated using an acetylated peptide antigen as previously described²⁰, with the following modifications. Anti-Ac-K382 antibody was affinity purified using non-acetylated p53-K382 peptides and stored in PBS at −70° C. and recognized an acetylated but not a non-acetylated p53 peptide. Western hybridizations using anti-acetylated K382 or anti-actin (Chemicon) antibody were performed at 1:1000 dilution of antibody. Hybridizations with polyclonal p53 antibody (Santa Cruz Biotech.) used 1:500 dilution of antibody. Whole cell extracts were prepared by lysing cells in buffer containing 150 mM NaCl, 1 mM MgCl₂, 10% glycerol, 1% NP40, 1 mM DTT and anti-protease cocktail (Roche).

### REFERENCES FOR EXAMPLES 1-4 AND BACKGROUND

1. Kenyon, C. Cell 105, 165-168 (2001).
2. Sinclair, D. A. Mech Ageing Dev 123, 857-67 (2002).
3. Hekimi, S. & Guarente Science 299, 13514 (2003).
4. Guarente, L. & Kenyon, C. Nature 408, 255-62. (2000).
5. Lin et al. Science 289, 2126-8. (2000).
6. Anderson et al. Nature 423, 181-5 (2003).
7. Kaeberlein et al. Genes Dev 13, 2570-80. (1999).
8. Landry et al. Proc Natl Acad Sci U S A 97, 5807-11. (2000).
9. Imai et al. Nature 403, 795-800 (2000).
10. Smith et al. Proc Natl Acad Sci U S A 97, 6658-63. (2000).
11. Tanner et al. Proc Natl Acad Sci U S A 97, 14178-82. (2000).
12. Tanny et al. Cell 99, 735-45. (1999).
13. Tanny et al. Proc Natl Acad Sci U S A 98, 415-20. (2001).
14. Laurenson et al. Microbiol Rev 56, 543-60. (1992).
15. Smith et al. Genes Dev 11, 241-54. (1997).
16. Bryk, M. et al. Genes Dev 11, 255-69. (1997).
17. Gottlieb et al. Cell 56, 771-6. (1989).
18. Aguilaniu et al. Science (2003).
19. Tissenbaum et al. Nature 410, 227-30. (2001).
20. Vaziri et al. Cell 107, 149-59. (2001).
21. Luo et al. Cell 107, 137-48. (2001).
22. Vergnes et al. Gene 296, 139-50 (2002).
23. Holzenberger et al. Nature 421, 182-7 (2003).
24. Shimokawa et al. Faseb J 17, 1108-9 (2003).
25. Tatar et al. Science 299, 1346-51(2003).
26. Bitterman et al. J Biol Chem 277, 45099-107. (2002).
27. Langley et al. EMBO J 21, 2383-2396 (2002).
28. Glossmann et al. Naunyn Schmiedebergs Arch Pharmacol 317, 100-2 (1981).
29. Oliver et al. J Biol Chem 269, 29697-703 (1994).
30. Ferguson et al. Mutat Res 475, 89-111 (2001).
31. Middleton et al. Pharmacol Rev 52, 673-751 (2000).
32. Jang et al. Science 275, 218-20 (1997).
33. Stojanovic et al. Arch Biochem Biophys 391, 79-89 (2001).
34. Monod et al. J. Mol. Biol. 12, 88-118 (1965).
35. Anderson et al. J Biol Chem 277, 18881-90. (2002).
36. Pont et al. J Phytopathol 130, 1-8 (1990).
37. Sinclair et al. Cell 91, 103342. (1997).
38. Defossez et al. Mol Cell 3, 447-55 (1999).
39. Park et al. Mol Cell Biol 19, 3848-56 (1999).
40. Benguria et al. Nucleic Acids Res 31, 893-8 (2003).
41. Longo et al. Science 299, 1342-6 (2003).
42. Denu et al. Trends Biochem Sci 28, 41-8 (2003).
43. Dong et al. Mutat Res 523-524, 145-50 (2003).
44. Nicolini et al. Neurosci Lett 302, 414 (2001).
45. Jazwinski, S. M. et al. Ann N Y Acad Sci 908, 21-30 (2000).
46. Pandey et al. Nucleic Acids Res 30, 5036-55 (2002).
47. Frye, R. A. Biochem Biophys Res Commun 273, 793-8. (2000).
48. Soleas et al. Clin Biochem 30, 91-113 (1997).
49. Coronado et al. Plant Physiol 108, 533-542 (1995).
50. Masoro, E. J. Exp Gerontol 35, 299-305. (2000).

### EXAMPLE 6

### Localization of the Activation Domain of Sirtuins to their N-Terminus

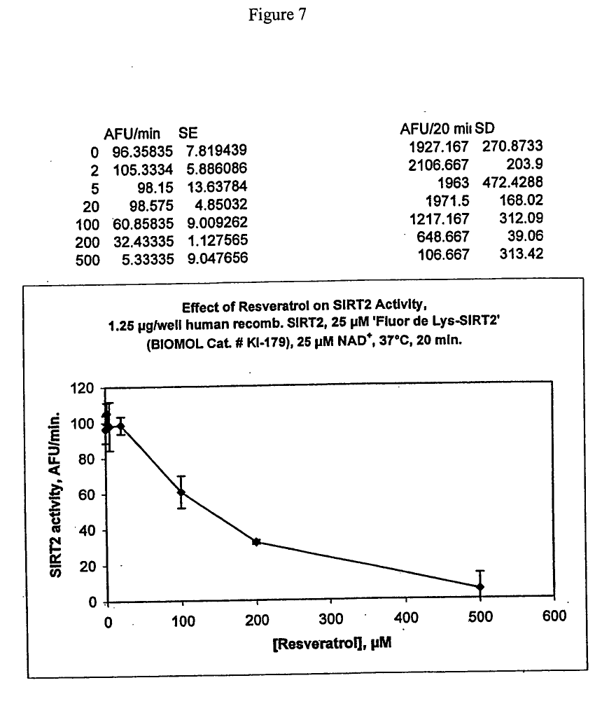

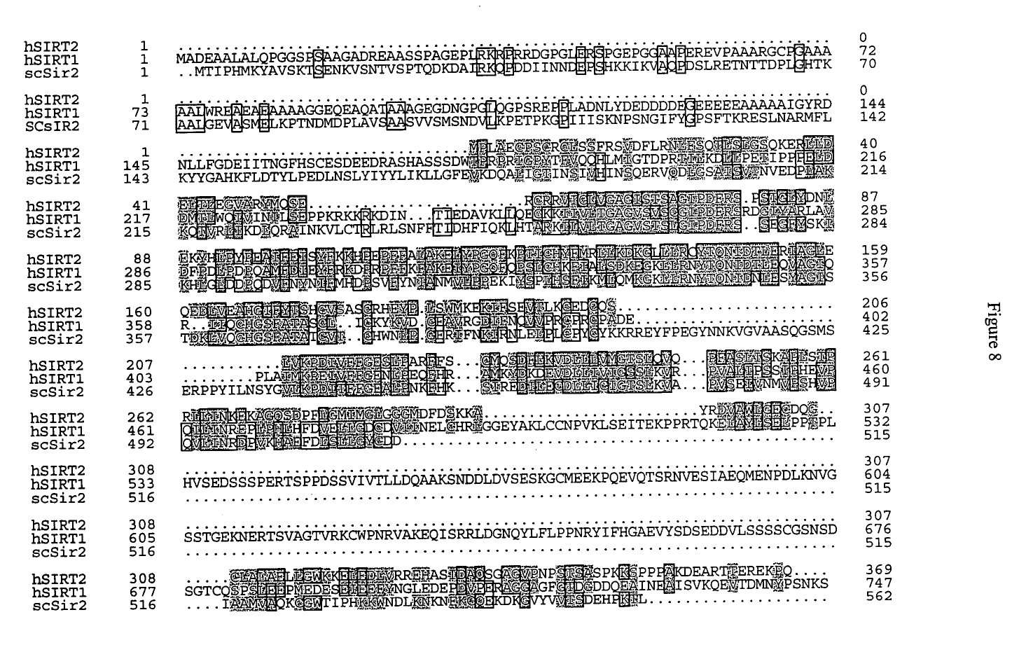

Yeast Sir2 and human SIRT1 are very homologous and differ from human SIRT2 by the addition of an N-terminal domain that is absent in SIRT2. The effect of resveratrol was assayed on human recombinant SIRT2 as follows. Human recombinant SIRT2 was incubated at a concentration of 1.25 μg/well with 25 μM of Fluor de Lys-SIRT2 (BIOMOL cat. # KI-179) and 25 μM NAD+ for 20 minutes at 37° C., as described above. The results, which are shown in FIG. 7, indicate that, in contrast to SIRT1, increasing concentrations of resveratrol decrease SIRT2 activity. Thus, based on the difference in structure of SIRT1 and SIRT2, i.e., the absence of an N-terminal domain (see FIG. 8), it is believed that the N-terminal domain of SIRT1 and Sir2 is necessary for activation by the compounds described herein. In particular, it is likely that the activator compounds described herein interact with the N-terminal portion of sirtuins. The N-terminal portion of SIRT1 that is necessary for the action of the compounds is from about amino acid 1 to about amino acid 176, and that of Sir2 is from about amino acid 1 to about amino acid 175.

### EXAMPLE 7

### Resveratrol Extends the Lifespan of C. Elegans

50 C. elegans worms (strain N2) were grown in the presence or absence of 100 μM resveratrol for 17 days. On day 17, only 5 worms in the control group without resveratrol were alive, whereas 17 worms were alive in the group that was treated with resveratrol. Thus, the presence of resveratrol in the growth media of C. elegans extends their lifespan.

### EXAMPLE 8

### Identification of Additional Activators of Sirtuins

Using the screening assay described in Example 1, five more sirtuin activators have been identified. These are set forth in supplementary Table 8.

### EXAMPLE 9

### Identification of Inhibitors of Sirtuins

Using the screening assay described in Example 1, more inhibitors were identified. These are set forth in the appended supplementary Table 8, and correspond to the compounds having a ratio to control rate of less than 1.

### EXAMPLE 10

### Identification of Further Activators and Inhibitors of Sirtuins

Additional activators and inhibitors of sirtuins were identified, and are listed in Tables 9-13. In these Tables, “SE” stands for Standard error of the mean and N is the number of replicates used to calculate mean ratio to the control rate and standard error.

All SIRT1 rate measurements used in the calculation of “Ratio to Control Rate” were obtained with 25 μM NAD⁺ and 25 μM p53-382 acetylated peptide substrate were performed as described above and in K. T. Howitz et al. Nature (2003) 425: 191. All ratio data were calculated from experiments in which the total deacetylation in the control reaction was 0.25-1.25 μM peptide or 1-5% of the initial concentration of acetylated peptide.

Stability determinations (t_{1/2}) derived from SIRT1 rate measurements performed in a similar way to those described above, except that 5 μM p53-382 acetylated peptide substrate was used rather than 25 μM. The fold-stimulation (ratio to control) obtained with a compound diluted from an aged stock solution was compared to an identical dilution from a stock solution freshly prepared from the solid compound. “t_{1/2}” is defined as the time required for the SIRT1 fold-stimulation of the compound from the aged solution to decay to one-half of that obtained from a freshly prepared solution. Ethanol stocks of resveratrol, BML-212 and BML-221 were prepared at 2.5 mM and the compounds were assayed at a final concentration of 50 μM. The water stock of resveratrol was 100 μM and the assay performed at 10 μM. Stocks were aged by storage at room temperature, in glass vials, under a nitrogen atmosphere.

The effect of some of these compounds on lifespan was determined in yeast and C. elegans, as described above. The results are set forth below in Table 19:

| | % change in yeast | % change in C. elegans | |
|---|---|---|---|
| | replicative lifespan | lifespan relative to | |
| | relative to untreated | untreated organisms | |
| Compound | organisms (10 μM)^{a} | (100/500 μM)^{b} | |
| untreated | 100% | 100% | |
| Resveratrol | 170-180% | 110% | |
| 3,5,4′-Trihydroxy- | | | |
| trans-stilbene | | | |
| Pinosylvin | 114% | ND | |
| 3,5-Dihydroxy- | | | |
| trans-stilbene | | | |
| BML-212 | 98% | ND | |
| 3,5-Dihydroxy-4′- | | | |
| fluoro-trans- | | | |
| stilbene | | | |
| BML-217 | 90% | ND | |
| 3,5-Dihydroxy- | | | |
| 4′-chloro-trans- | | | |
| stilbene | | | |
| BML-221 | 165% | >100% | (ongoing) |
| 3,4′-Dihydroxy- | | | |
| 5-acetoxy-trans- | | | |
| stilbene | | | |
| BML-233 | ND | 70% | (10) |
| 3,5-Dihydroxy- | | 50% | (500) |
| 4′-methoxy- | | | |
| trans-stilbene | | | |

^{a}Replicative lifespans performed using 2% (w/v) glucose standard yeast compete medium (YPD) under standard conditions.
^{b}Lifespan assays performed on N2 worms using E. coli as food under standard conditions.
ND. Not determined.

The results indicate that resveratrol significantly extends lifespan in yeast and in C. elegans. Since BML-233 was shown to be a strong activator of sirtuins (see above), the results obtained in C. elegans may indicate that the compound is toxic to the cells.

Without wanting to be limited to particular structures, it appears that the following structure/activity relationships exist. SIRT1 activation results from several of these new analogs confirmed the importance of planarity, or at least the potential for planarity, between and within the two rings of the active compounds. Reduction of the double bond of the ethylene function, between, the two rings essentially abolishes activity (compare Resveratrol, Table A and Dihydroresveratrol, Table E). Replacement of a phenyl moiety with a cyclohexyl group is nearly as detrimental to SIRT1 stimulating activity (compare Pinosylvin, Table 9 and BML-224, Table 12). Amide bonds are thought to have a partially double bond character. However, replacement of the ethylene function with a carboxamide abolished activity (compare Pinosylvin, Table 9, with BML-219, Table 13). It is possible that this effect could be due in part to the position that carbonyl oxygen must assume in the conformation that places the two rings trans to one another. If so, a compound in which the positions of the amide nitrogen and carbonyl are reversed might be expected to have greater activity.

In twelve of the analogs resveratrol's 4′-hydroxy was replaced with various functionalities (see Tables 9 and 10, BML-221 in Table 11, BML-222 in Table 12). Although none of the replacements tried led to substantial increases in SIRT1 stimulating activity, this parameter was, in general, remarkably tolerant of substitutions at this position. Small groups (H— in Pinosylvin, Cl— in BML-217,-CH₃ in BML-228) did the least to decrease activity. There is some evidence of a preference in the enzyme's stilbene binding/activation site for unbranched (ethyl in BML-225, azido in BML-232, —SCH₃ in BML-230) and hydrophobic functions (compare isopropyl in BML-231 to acetoxy in BML-221, acetamide in BML-222). Solution stability relative to resveratrol was strongly increased by one of the two 4′-substitutions (acetoxy, BML-221) tested for this so far.

Resveratrol is currently one of the most potent known activator of SIRT1. The collection of analogs described above, particularly the group entailing substitutions at the 4′ position, may be instrumental in informing the design of new SIRT1 ligands with improved pharmacological properties. One parameter that may be of interest in this regard is stability. One 4′-substituted analog, BML-221, displays a vast improvement in solution stability relative to resveratrol and although diminished in in vitro SIRT1 activating ability, retains much of resveratrol's biological activity (see lifespan data). The 4′-hydroxyl of resveratrol is thought to be of primary importance to resveratrol's free-radical scavenging reactivity (S. Stojanovic et al. Arch. Biochem. Biophys. 2001 391 79). Most of the 4′-substituted analogs have yet to be tested for solution stability, but if resveratrol's instability in solution is due to redox reactivity, many of the other analogs would be expected to also exhibit improved stability.

The results obtained with 4′-substituted analogs may indicate promising routes to explore while seeking to increase SIRT1 binding affinity. For example, the efficacy of the 4′-ethyl compound (BML-225) might indicate the presence of a narrow, hydrophobic binding pocket at the SIRT1 site corresponding to the 4′ end of resveratrol. Several new series of 4′-substituted analogs are planned, the simplest comprising straight-chain aliphatic groups of various lengths.

### EXAMPLE 11

### Methods of Synthesis of the Compounds in Tables 9-13

Most of the resveratrol analogs were synthesized by the same general procedure, from a pair of intermediates, a benzylphosphonate and an aldehyde. The synthesis or sources of these intermediates are described in section II below. Section III below describes the procedures for synthesizing the final compounds from any of the benzylphosphonate/aldehyde pairs. The coupling reaction (Section III A below) is followed by one of two deprotection reactions depending on whether the intermediates contained methoxymethyl (Section III B below) or methoxy (Section III C below) protecting groups. Section IV below corresponds to Tables 14-18, which list the particular benzylphosphonate and aldehyde used in the synthesis of particular final compounds. Seven of the compounds—Resveratrol, 3,5-Dihydroxy-4′-methoxy-trans-stilbene, Rhapontin aglycone, BML-227, BML-22 1, Dihydroresveratrol, BML-219—were not synthesized by the general procedure and “N/A” appears next to their entries in the table. Resveratrol was from BIOMOL and the syntheses of the remaining compounds are described in Section V below.

II. Synthetic Intermediates

A. Benzylphosphonates (Synthesized)

Synthesis of Diethyl 4-Acetamidobenzylphosphonate: To diethyl 4-aminobenzylphosphonate in 1:1 methylene chloride/pyridine was added catalytic DMAP and acetic anhydride (1.1 eq.). After 3 hours, the reaction was evaporated to dryness and purified via flash chromatography (silica gel).

Synthesis of Diethyl 4-Methylthiobenzylphosphonate: 4-Methylthiobenzyl chloride was heated with triethylphosphite (as solvent) at 120° C. overnight. Excess triethyl phosphite was distilled off under high vacuum and heat. Flash chromatography (silica gel) yielded the desired product.

Synthesis of Diethyl 3,5-Dimethoxybenzylphosphonate: From 3-5-Dimethoxybenzyl bromide. See synthesis of Diethyl 4-Methylthiobenzylphosphonate.

Synthesis of Diethyl 4-Fluorobenzylphosphonate: From 4-Fluorobenzylphosphonate. See synthesis of Diethyl 4-Methylthiobenzylphosphonate.

Synthesis of Diethyl 4-azidobenzylphosphonate: To diethyl 4-aminobenzylphosphonate in acetonitrile (2.5 mL) at 0° C. was added 6M HCl (1 mL). Sodium nitrite (1.12 eq.) in water (1 mL) was added drop wise and the resulting solution stirred at 0° C. for 30 mins. Sodium azide (8 eq.) in water (1 mL) added drop wise (bubbling) and the solution stirred at 0° C. for 30 mins., then at room temperature for 1 hour. The reaction was diluted with ethyl acetate and washed with water and brine and dried over sodium sulfate. Flash chromatography (silica gel) yielded the desired product.

B. Aldehydes (Synthesized)

Synthesis of 3,5-Dimethoxymethoxybenzaldehyde: To 3,5-dihydroxybenzaldehyde in DMF at 0° C. was added sodium hydride (2.2 eq.). The reaction was stirred for 30 min. at 0° C. Chloromethylmethyl ether (2.2 eq.) was added neat, drop wise and the reaction allowed to warm to room temperature over 1.5 hrs. The reaction mixture was diluted with diethyl ether and washed with water (2×) and brine (1×) and dried over sodium sulfate. Flash chromatography (silica gel) yielded the desired product.

C. Purchased Intermediates: Unless Listed Above, all Synthetic Intermediates were Purchase from Sigma-Aldrich.

III. General Procedure for the Synthesis of Resveratrol Analogues

A. Benzylphosphonate/Aldehyde Coupling Procedure

To the appropriate benzylphosphonate (1.2 eq.) in dimethylformamide (DMF) at room temperature was added sodium methoxide (1.2 eq.). This solution was allowed to stir at room temperature for approximately 45 minutes. The appropriate aldehyde (1 eq.) was then added (neat or in a solution of dimethylformamide). The resulting solution was then allowed to stir overnight at room temperature. Thin layer chromatography (TLC) was used to determine completeness of the reaction. If the reaction was not complete, the solution was heated at 45-50° C. until complete. The reaction mixture was poured into water and extracted with ethyl acetate (2×). The combined organic layers were washed with brine and dried over sodium sulfate. Flash chromatography (silica gel) yielded the desired products.

B. General Procedure for the Deprotection of Methoxymethylresveratrol Analogues

To the appropriate methoxymethylstilbene derivative in methanol was added two drops of concentrated HCl. The resulting solution was heated overnight at 50° C. The solution was evaporated to dryness upon completion of the reaction. Flash chromatography (silica gel) yielded the desired product.

C. General Procedure for the Deprotection of Methoxyresveratrol Analogues

To the appropriate methoxystilbene derivative in methylene chloride was added tetrabutylammonium iodide (1.95 eq. per methoxy group). The reaction was cooled to 0° C. and boron trichloride (1 M in methylene chloride; 2 eq. per methoxy group) was added dropwise. Following the addition of boron trichloride, the cooling bath was removed and the reaction allowed to stir at room temperature until complete (as indicated by TLC). Saturated sodium bicarbonate solution was added and the reaction vigorously stirred for 1 hour. The reaction was poured into cold 1M HCl and extracted with ethyl acetate (3×). The combined organic layers were washed with water (1×) and brine (1×) and dried over sodium sulfate. Flash chromatography (silica gel) yielded the desired products.

V. Special Syntheses

Synthesis of BML-219 (N-(3,5-Dihydroxyphenyl)benzamide): To benzoyl chloride (1 eq.) in dry methylene chloride at room temperature was added triethylamine (1.5 eq.) and a catalytic amount of DMAP followed by 3,5-dimethoxyaniline (1 eq.). The reaction was allowed to stir overnight at room temperature. Upon completion, the reaction was diluted with ethyl acetate and washed with 1M HCl, water and brine and dried over sodium sulfate. Flash chromatography (silica gel) yielded the methoxystilbene derivative. To the methoxystilbene in dry methylene chloride at 0° C. was added tetrabutylammonium iodide (3.95 eq.) followed by boron trichloride (4 eq.; 1M in methylene chloride). Upon completion of the reaction (TLC), saturated sodium bicarbonate was added and the mixture was vigorously stirred for 1 hour. The reaction was diluted with ethyl acetate and washed with 1M HCl and brine and dried over sodium sulfate. Flash chromatography (silica gel) yielded the desired product.

Synthesis of BML-220 (3,3′,5-trihydroxy-4′-methoxystilbene): To Rhapontin in methanol was added catalytic p-toluenesulfonic acid. The reaction was refluxed overnight. Upon completion of the reaction (TLC), the reaction mixture was evaporated to dryness and taken up in ethyl acetate. The organics were washed with water and brine and dried over sodium sulfate. Flash chromatography (silica gel) yielded the desired product.

Synthesis of BML-233 (3,5-Dihydroxy4′-methoxystilbene): To deoxyrhapontin in methanol was added catalytic p-toluenesulfonic acid. The reaction was refluxed overnight. Upon completion of the reaction (TLC), the reaction mixture was evaporated to dryness and taken up in ethyl acetate. The organics were washed with water and brine and dried over sodium sulfate. Flash chromatography (silica gel) yielded the desired product.

Synthesis of BML-221 and 227 (4′ and 3 monoacetylresveratrols): To resveratrol in tetrahydrofuran at room temperature was added pyridine (1 eq.) followed by acetic anhydride (1 eq.). After stirring for 48 hrs., another 0.25 eq. acetic anhydride added followed by 24 hrs. of stirring. The reaction was diluted with methylene chloride (reaction was not complete) and washed with cold 0.5M HCl, water and brine. Organics were dried over sodium sulfate. Flash chromatography yielded a mixture of 4′- and 3-acetyl resveratrols. Preparative HPLC yielded both monoacetyl resveratrols.

Synthesis of Dihydroresveratrol: To resveratrol in argon-purged ethyl acetate in a Parr shaker was added 10% palladium on carbon (10 wt%). The mixture was shaken under an atmosphere of hydrogen (30 psi) for 5 hours. Filtration through a pad of celite yielded the desired material.

### EXAMPLE 12

### Dose-Response Analysis of SIRT1 Deacetylation by Resveratrol and BML-230

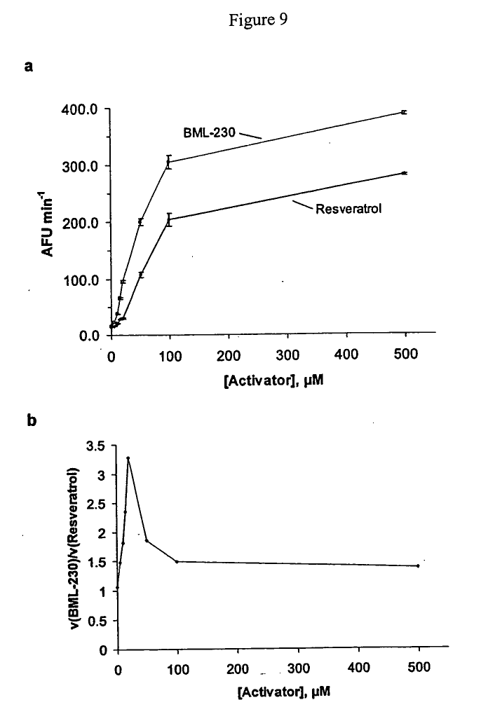

SIRT1 initial rates as a function of activator concentration were determined at 25 μM each of NAD⁺ and p53-382 acetylated peptide, with 20 minutes incubations. Plots of the dose responses of SIRT1 to BML-230 and resveratrol show that the BML-230-stimulated activity exceeds that stimulated by resveratrol at all concentrations tested (FIG. 9a). This could be due to a greater binding affinity of SIRT1 for BML-230, greater activity of the SIRT1/BML-230 complex or some combination of the two. A plot of the ratio of the rates of BML-230-stimulated enzyme to that of resveratrol-stimulated enzyme suggests that increased binding affinity does contribute to the improvement in activity of BML-230 (FIG. 9b). A simple two state model of the binding and activation process assumes that the observed rate (v) is the sum of the fractional contributions of the unliganded and liganded enzymes, where v₀ is the unstimulated rate, v₁ is the rate of the enzyme with bound ligand-1 (L1) and K_{L1} is the dissociation constant of the enzyme/ligand-1 complex:
v=v₀(1−[L1]/(K_{L1}+[L1]))+v₁(−[L1]/(K_{L1}+[L1])
A similar equation can be prepared for ligand-2 and the ratio (R) of the two rates calculated, an equation which will include, given the conditions of FIG. 9, the substitution [L]=[L1]=[L2]. It can be shown that if the two ligand dissociation constants were equal (K_{L1}=K_{L2}=K_{L}), this ratio would be:
R=(v₀K_{L}+v₁[L])/(v₀K_{L}+v₂[L])
If K_{L1}≠K_{L2}, this ratio would instead be:
R=(v₁[L]²+(v₀K_{L1}+v₁K_{L2})[L]+v₀K_{L1}K_{L2})/(v₂[L]² +(v₀K_{L2}+v₂K_{L1})[L]+v₀K_{L1}K_{L2})
In the first case the plot of R vs. [L] would be a simple hyperbola that monotonically approaches v₁/v₂ as [L] increases. In the second case, as in FIG. 9b, the plot would pass through a maximum before approaching v₁/v₂ at higher [L] values. The data of FIG. 9b would imply that v₁/v₂ (rate for pure SIRT1/BML-230 divided by that for pure SIRT1/resveratrol) is no more than ˜1.4 (R at 500 μM) and that the SIRT1/BML-230 complex indeed has a lower dissociation constant than SIRT1/resveratrol (K_{L1}<K_{L2}).

One of the difficulties in the use of resveratrol as a pharmacologic agent is the relatively low serum concentrations of the aglycone form that can be achieved and maintained when it is administered orally (<<1 μM; see for example D. M Goldberg et al. Clin. Biochem. 2003 36 79). Increasing the SIRT1 binding affinity of synthetic derivatives will improve this aspect of the drug. As sest forth above, various replacements of the resveratrol 4′-hydroxyl, e.g. the H— of pinosylvin or Cl— of BML-217, did not significantly diminish the SIRT1 activating effect. The results obtained with BML-230 indicate that it will be possible to actually increase SIRT1/activator binding affinity by modifications at that site. The 4′-thiomethyl of BML-230 therefore represents a new starting point in seeking further improvements in SIRT1 binding affinity by the synthesis of related derivatives (e.g. 4′-thioethyl etc.).

### EXAMPLE 13

### Survival Rates

Human 293 were grown to exponential phase under standard conditions and subjected to a dose of compound (50 micromolar) for 96 hours. The number of live cells each time point was counted using a Coulter counter.

TABLE 24
Survival statistics of 293 cells:

| | Resver- | Thio-Methyl | Ethyl | Methyl | Isopropyl |
|---|---|---|---|---|---|
| Time (h) | atrol | BML-230 | BML-225 | BML-228 | BML-231 |
| 0 | 100% | 100% | 100% | 100% | 100% |
| 48 | 5% | 55% | 5% | 46% | 0% |
| 96 | 0% | 57% | 8% | 32% | 0% |

The results indicate that thiomethyl (BML-230) was the least toxic on 293 cells.

### EXAMPLE 14

### Sirtuin Activators Mimic Calorie Restriction and Delay Aging in Metazoans

Caloric restriction (CR) extends lifespan in numerous species. In the budding yeast S. cerevisiae, this effect requires Sir2¹, a member of the sirtuin family of NAD⁺-dependent deacetylases^{2,3}. Sirtuin activating compounds (STACs) can promote the survival of human cells and extend the replicative lifespan of yeast⁴. Here it is shown that resveratrol and other STACs activate sirtuins from Caenorhabditis elegans and Drosophila melanogaster and extend the lifespan of these animals up to 29% without reducing fecundity. Lifespan extension is dependent on functional Sir2 and is not observed when nutrients are restricted. Together these data indicate that STACs slow metazoan ageing by mechanisms related to CR.

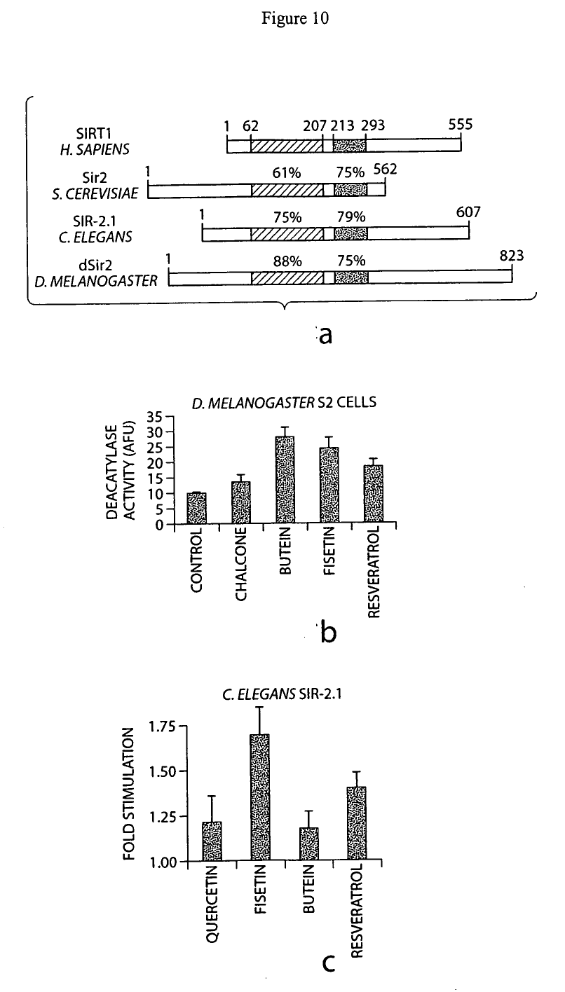

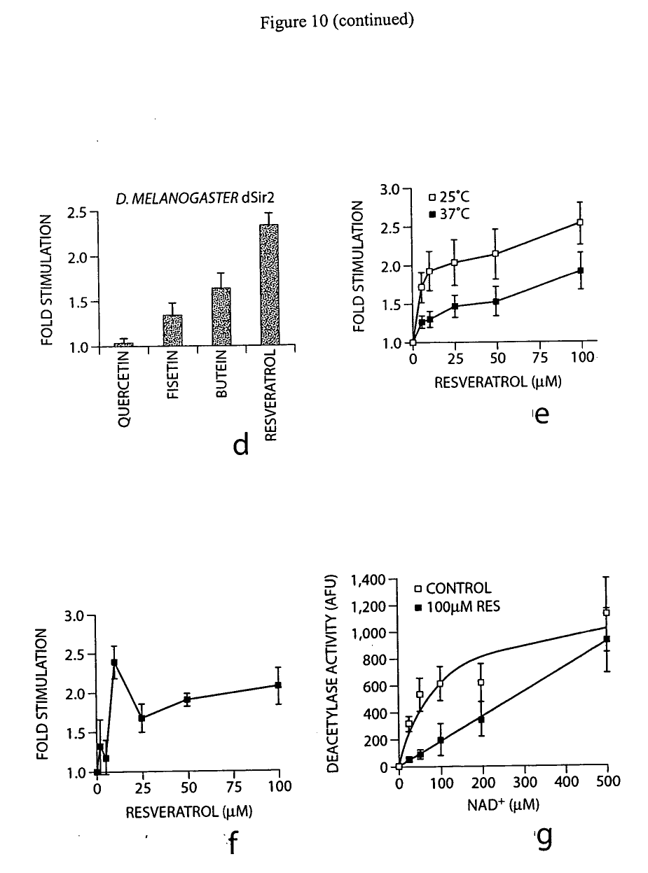

Sir2-like proteins (sirtuins) are a family of NAD⁺-dependent deacetylases conserved from E. coli to humans⁵⁻⁹ (FIG. 10a) that play important roles in gene silencing, DNA repair, rDNA recombination and ageing in model organisms^{2,10-12}. When diet is restricted (calorie restriction, CR), lifespan is extended in diverse species, suggesting there is a conserved mechanism for nutrient regulation of ageing¹³⁻¹⁷. In budding yeast, extra copies this gene extend lifespan by 30% apparently by mimicking CR^{1,18}. Recently a group of compounds (STACs) has been described that stimulate the catalytic activity of yeast and human sirtuins, and extend the replicative lifespan of yeast cells up to 60%⁴.

To establish whether STACs could activate sirtuins from multicellular animals, a cell-based deacetylation assay was developed for D. melanogaster S2 cells. Several classes of polyphenolic STACs, including chalcones, flavones and stilbenes, increased the rate of deacetylation in an NAD⁺-dependent manner (FIG. 10b). To determine whether this activity was due to direct stimulation of a Sir2 homolog, recombinant SIR-2.1 of C. elegans and dSir2 of D. melanogaster were purified and the effect of various STACs on enzymatic activity in vitro (FIGS. 10c, d) was deteremined. In a dose-dependent manner, resveratrol stimulated deacetylation up to 2.5-fold for SIR-2.1 (FIG. 10e) and 2.4-fold for dSir2 (FIG. 10f). As previously observed with the yeast and human Sir2 enzymes, resveratrol lowered the Kₘ of SIR-2.1 for the co-substrate NAD⁺ (FIG. 10g).

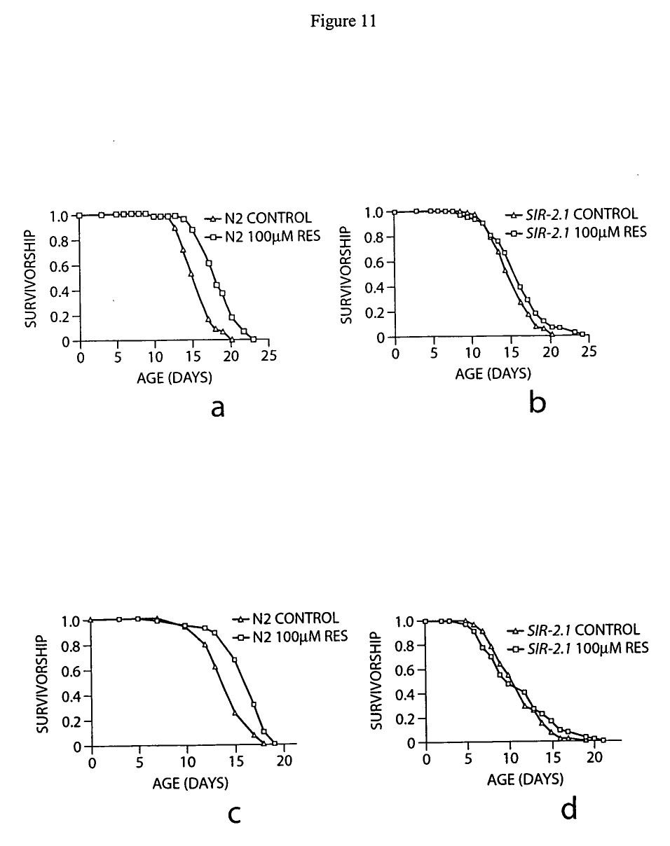

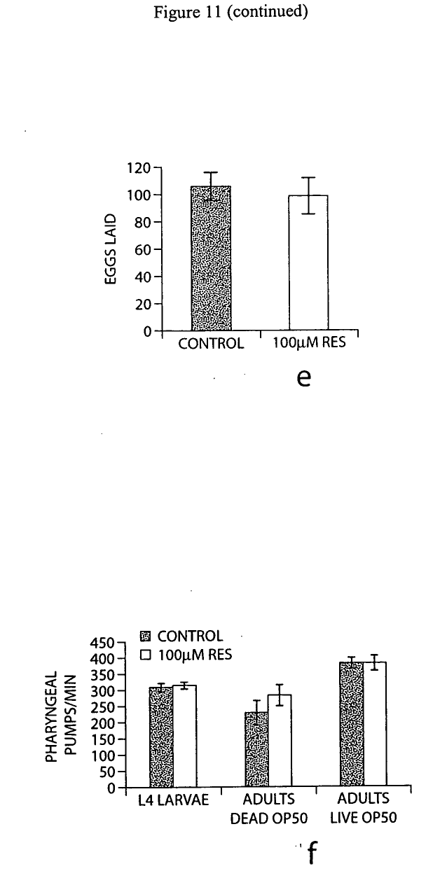

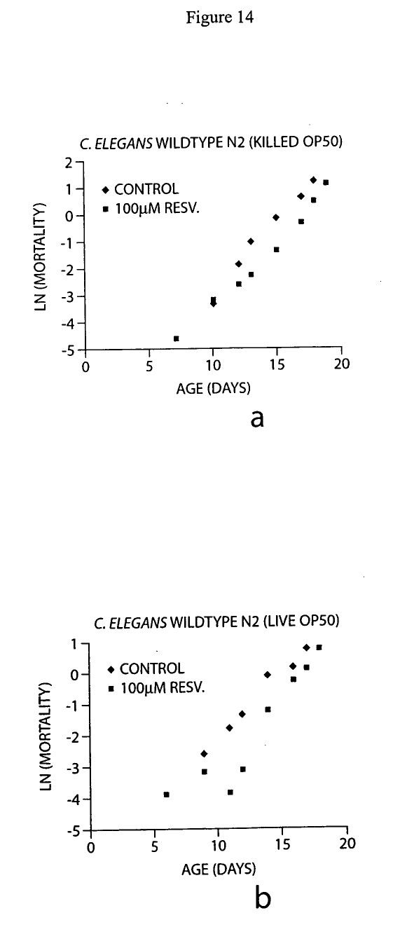

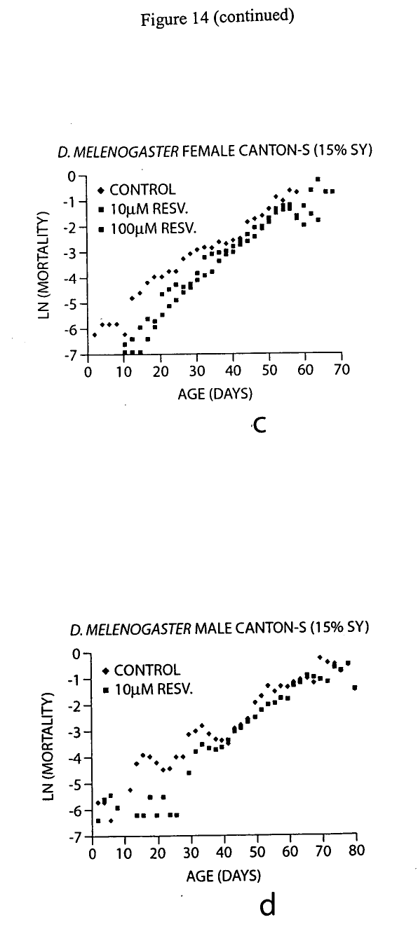

Because resveratrol can significantly extend replicative lifespan in yeast⁴, it was investigated whether STACs could also extend lifespan in the metazoans C. elegans and D. melanogaster. Wild-type worms were transferred to plates containing 0 or 100 μM of resveratrol shortly after reaching adulthood. Lifespan was reproducibly extended up to 15%, using either heat-killed or live E. coli as food supply (FIGS. 11a, c respectively) and mortality was decreased across all adult ages (FIG. 14). To test whether the lifespan extension depends on functional SIR-2.1, a sir-2.1 null mutant was constructed. The lifespan of this strain was not appreciably shorter than the wildtype N2 control and adults treated with resveratrol did not exhibit a significant lifespan extension relative to untreated worms (FIGS. 11b, d). There was no decrease in fecundity associated with resveratrol treatment (FIG. 11e). To rule out the possibility that resveratrol was causing the animals to eat less, thereby inducing a CR effect indirectly, feeding rates of both L4 larval and adult worms was measured with or without resveratrol and found no differences (FIG. 11f).

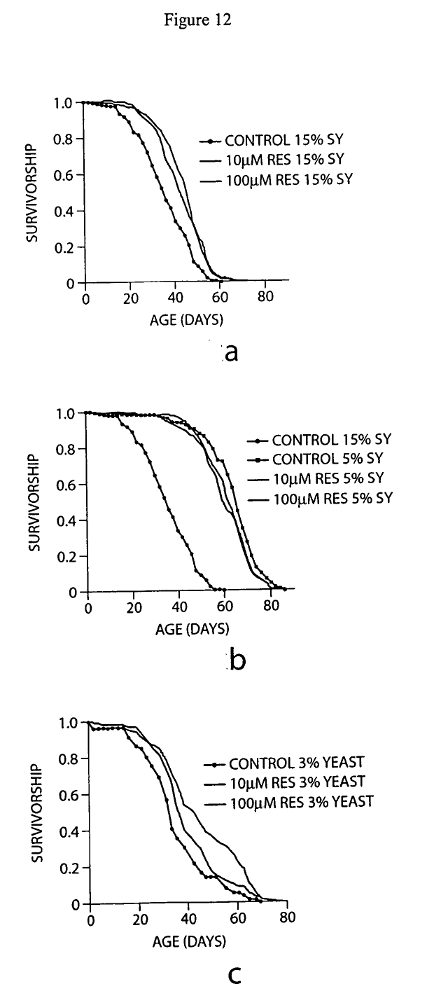

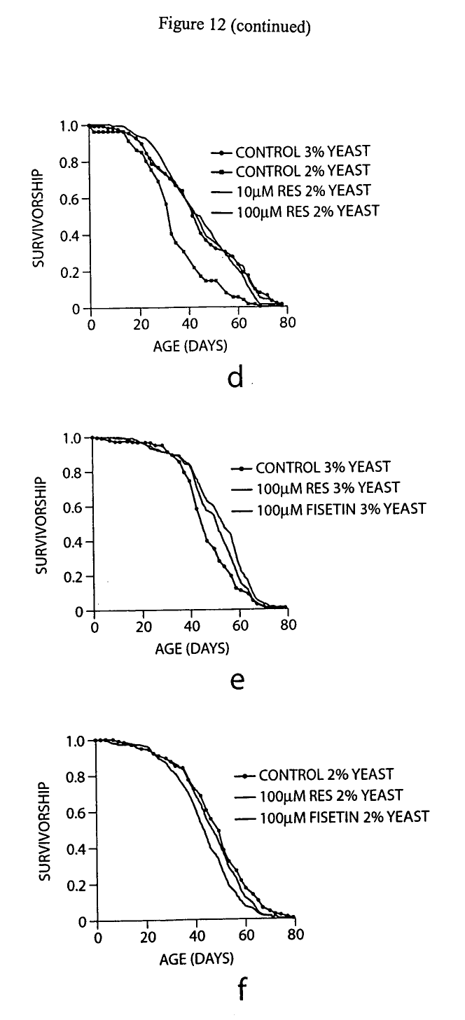

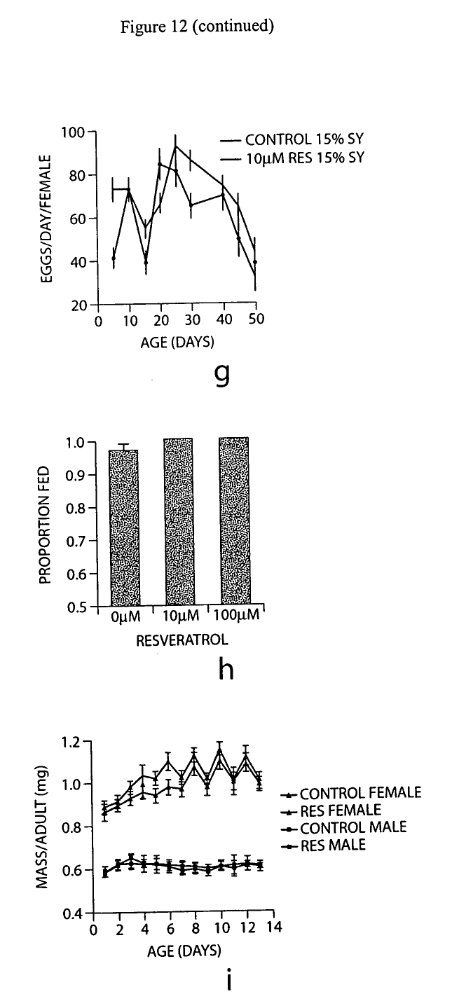

Whether STACs could extend lifespan in D. melanogaster was also tested using the standard laboratory wild type strain Canton-S and normal fly culturing conditions (vials), and a yw marked wild type strain and demographic culturing conditions (cages) (Table 20). Across independent tests in males and females, lifespan was extended up to 23% with fisetin and up to 29% with resveratrol (FIGS. 12a, c, e). Increased longevity was associated with reduced mortality prior to day 40 (FIG. 14). A restricted diet increased lifespan by 40% in females and by 14% in males (averaged across trials), and under these conditions neither resveratrol nor fisetin further increased longevity (FIGS. 12b, d, f), suggesting that resveratrol extends lifespan through a mechanism related to CR.

Surprisingly, while diet manipulations that extend D. melanogaster longevity typically reduce fecundity^{19,20}, longevity-extending doses of resveratrol modestly increased egg production(10 μM resveratrol: 69.8 eggs/5 days, s.e.=2.2; control: 59.9 eggs/5 days, s.e.=2.2; t=3.17, P=0.0017), particularly in the earliest days of adult life (FIG. 12g). The increase in egg production suggests that the lifespan extending effect of resveratrol in D. melanogaster was not due to CR induced by food aversion or lack of appetite. Consistent with this, no decrease in food uptake was seen with resveratrol-fed flies (FIG. 12h). Furthermore, resveratrol-fed flies maintained normal weight (FIG. 12i), except during days 3 through when resveratrol fed females were laying significantly more eggs than control fed females.

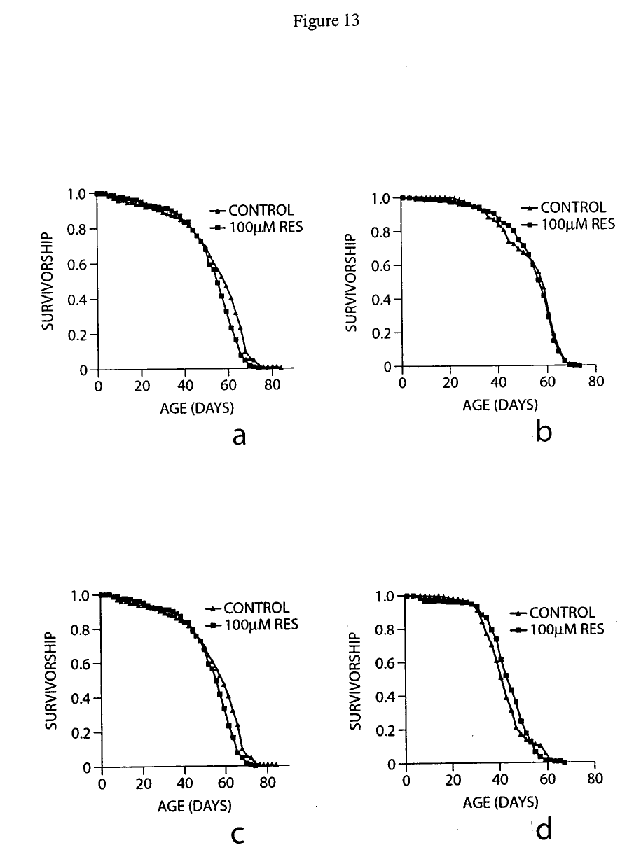

To determine whether resveratrol extends fly lifespan in a Sir2-dependent manner, a dSir2 allelic series was analyzed with increasing amounts of dSir2. Adult offspring from crosses between independently derived alleles of dSir2 were tested. Resveratrol failed to extend lifespan in flies completely lacking functional dSir2 (dSir2^{4.5}/dSir2^{5.26}) (FIGS. 13a, b) or in flies in which dSir2 is severely decreased (dSir2¹⁷/dSir2^{KG00871}) (FIGS. 13c, d). Resveratrol increased longevity a small but statistically significant amount in flies homozygous for a hypomorphic dSir2 allele (dSir2^{KG0087}/dSir2^{KG0087}) (Table 20, Trial 6) and increased lifespan up to 17% in flies with one copy of the hypomorphic allele and one copy of a wild-type dSir2 (Canton-S/dSir2^{KG0087}) (Table 20, Trial 7). These data demonstrate that the ability of resveratrol to extend fly lifespan requires functional Sir2.

It has previously been reported that STACs extend the lifespan of replicating yeast cells by mimicking CR⁴. In yeast, chronological and reproductive aging are inseparable in the measure of replicative lifespan. Here it is shown show that STACs can extend lifespan in C. elegans and D. melanogaster, both of which are comprised of primarily non-dividing (post-mitotic) cells as adults, and whose somatic and reproductive aging are independent measures of senescence. In both species, resveratrol increases lifespan in a Sir2-dependent manner and, at least for the fly, this action appears to function through a pathway common to CR.

The observation that resveratrol can increase longevity without an apparent cost of reproduction is counter to prevalent concepts of senescence evolution. However, STACs may still entail trade-offs under some environmental conditions^{21,22} or in the context of selection acting upon the network of traits that determine fitness^{23,24}. Plants synthesize STACs such as resveratrol in response to stress and nutrient limitation²⁵, possibly to activate their own sirtuin pathways⁴. These molecules may activate animal sirtuins because they serve as plant defense mechanisms against consumers or because they are ancestrally orthologous to endogenous activators within metazoans. Alternatively, animals may use plant stress molecules as a cue to prepare for a decline in their environment or food supply⁴. Understanding the adaptive significance, endogenous function, and evolutionary origin of sirtuin activators will lead to further insights into the underlying mechanisms of longevity regulation and aid in the development of interventions that provide the health benefits of CR.

### EXAMPLE 15

### Materials and Methods for Example 14

Sirtuin Purification

His₆-tagged recombinant SIR-2.1 and dSir2 were purified from E. coli BL21(DE3) plysS cells harboring either pET28a-sir-2.1 or pRSETc-dSir2 plasmids. Cells were grown in LB medium containing kanamycin (50 μg/mL) for pET28a-sir-2.1 or ampicillin (100 μg/ml) and chloramphenicol (25 μg/ml) for pRSETc-dSir2 at 30° C. (dSir2) or 37° C. (SIR-2.1) to an OD₆₀₀ of 0.6-0.8. After addition of IPTG (1 mM), flasks were shifted to 16° C. for 20 h. Cell pellets were resuspended in cold PBS buffer containing 300 mM NaCl, 0.5 mM DTT, 0.5 mM PMSF and EDTA-free protease inhibitor tablets and lysed by sonication. Ni²⁺-NTA beads were added to the clarified extract and after 1-3 hours they were loaded on a column, washed with buffer (50 mM Tris. Cl pH 7.4, 200 mM NaCl, 30 mM imidazole) then eluted with the same buffer containing 600 mM imidazole.

Deacetylation Assays

From 0.1 to 1 μg of SIR-2.1 and 1 μg of dSir2 were used per deacetylation assay as previously described with modifications (SIR-2.1: 200 μM NAD⁺, 10 μM Fluor de Lys, FdL; dSir2: 25 μM NAD⁺, 10 μM FdL)²⁶. STACs were dissolved at 10 mM in dimethylsulfoxide (DMSO) the day of the assay. In vitro fluorescence assay results were read in 96-well microplates (Coming Costar 3693) with a Wallac Victor Multilabel counter (Perkin Elmer, excitation at 360 nm, emission at 450 nm). Drosophila S2 cells were grown in Schneider media with fetal calf serum at 23-28° C., seeded at 9×10⁴ cells/well, grown overnight and then exposed to 1 μM TSA, 500 μM polyphenols, and 200 μM FdL for 2 hr. Deacetylation of FdL with lysate from whole cells was determined as described⁴. Unless otherwise indicated all initial rate measurements were means of three or more replicates obtained with single incubation times, at which point 5% or less of the substrate initially present was deacetylated.

C. Elegans Media, Strains, Lifespan, and Feeding Assays

Bristol N2 (Caenorhabditis Genetics Center) was used as the wild-type strain. The sir-2.1 mutant strain was generated by backcrossing VC199 (sir-2.1(ok434)) to N2 four times. Cultures were grown on standard NGM media and maintained on E. coli strain OP50. For the lifespan assays, synchronized animals were transferred to treatment plates as young adults (2 d after hatching, day 0 of assay), and were transferred to fresh treatment plates every 2 days for the first 6 to 8 days of the assay. Treatment plates were standard NGM media with the reproductive suppressant FUdR (Sigma; 100 mg/L) containing resveratrol or solvent (DMSO, which does not affect lifespan) added either directly into the agar before pouring (for live OP50 trials) or diluted into PBS and added to the surface of a dry plate to the indicated final concentration (for dead OP50 trials). For some lifespan trials, heat-killed OP50 were used as a food source. OP50 cultures were heated to 65° C. for 30 minutes, then pelleted and resuspended in 1/10 volume in S Basal supplemented with 10 mM MgSO₄. In all assays, worms were monitored daily for mortality by gently probing with a platinum pick. Assays were performed at 24° C. To assay worm feeding rates, worms at the indicated stages were placed on treatment plates (no FUdR) for 4-5 hours, then videoed for 1 minute using a Pixelink PL-662 camera. The frame rate was slowed and the pumping rate of the pharynx was counted. To assay fecundity, gravid hermaphrodites (5 per plate, raised from synchronized L1s on normal or treatment plates) were allowed to lay eggs on their respective media for 5 hours, and the total number of eggs was counted.

D. Melanogaster Media, Strains, Feeding Assay and Lifespan Assays

Survival assays were conducted independently with adult D. melanogaster in two laboratories. In the first laboratory, all trials used an yw marked wild-type strain. Larvae were reared on standard cornmeal-sugar-yeast (CSY) agar diet (cornmeal 5%, sucrose 10.5%, SAF yeast 2%, and agar 0.7%). Newly eclosed adults were placed in 1 L demography cages with approximately 75 males and 75 females. Three to four replicate 1 L demography cages were used for each treatment group in each trial. Every two days, dead flies were removed and scored, and food vials were replenished. Food vials contained cornmeal-sugar-yeast diet with SAF yeast as either 2% or 3% by weight. Test compounds in 100 μl of EtOH (or blank EtOH in controls) were mixed into melted aliquots of the adult food media to make a final concentration of 0, 10 or 100 μM. Fresh stock solutions and adult media were prepared weekly. In the second laboratory, lifespan trials were conducted with the wild type strain Canton-S, dSir2^{4.5} and dSir2^{5.26} (S. Smolik, University of Oregon), dSir2¹⁷ (S. Astrom, Stockholm University, Sweden), and dSir2^{KG00871} (Drosophila Stock Center, Bloomington, Ind.). Larvae for all tests were reared on standard cornmeal-sugar-yeast diet. Newly eclosed adults were incubated in plastic shell vials containing 5 ml of 15% sugar-yeast diet (15% SY) or 5% sugar-yeast (5% SY) diet (15% SY: 15% yeast, 15% sucrose, 2% agar; 5% SY: 5% yeast, 5% sucrose, 2% agar as per Ref. ²⁰). In all trials, ˜20 males with ˜20 females were placed into each of 10 vials/treatment group. Every two days, flies were passed into new vials and dead flies were counted. Resveratrol in EtOH (or EtOH alone in controls) was added to the media during its preparation after it had cooled to 65° C. and mixed vigorously. Final compound concentrations were 0, 10, 100 or 200 μM. Fresh stock solution and adult media was prepared weekly.

Feeding rate was measured in yw females with the crop-filling assay²⁷. Females were held overnight with water and placed on 2% CSY diet containing food colour (FDA Blue 1) and 0, 10 or 100 μM resveratrol with EtOH. The presence of dye-marked food in the crop was scored in sets of 20 females across five 5-minute intervals. For body mass measurements, 10 vials with 20 males and 20 females each of wild type CS-5 flies were kept on 15% SY diet with EtOH or with resveratrol in EtOH (10 μM). Males and females were weighed daily.

### REFERENCES FOR EXAMPLES 14 AND 15

1. Lin, S. J., Defossez, P. A. & Guarente, L. Requirement of NAD and SIR₂ for life-span extension by calorie restriction in Saccharomyces cerevisiae. Science 289, 2126-8. (2000).
2. Gasser, S. C. M. The molecular biology of the SIR proteins. Gene 279, 1-16 (2001).
3. Hekimi, S. & Guarente, L. Genetics and the specificity of the aging process. Science 299, 13514 (2003).
4. Howitz, K. T. et al. Small molecule activators of sirtuins extend Saccharomyces cerevisiae lifespan. Nature 425, 191-6 (2003).
5. Landry, J. et al. The silencing protein SIR₂ and its homologs are NAD-dependent protein deacetylases. Proc Natl Acad Sci U S A 97, 5807-11. (2000).
6. Imai, S., Armstrong, C. M., Kaeberlein, M. & Guarente, L. Transcriptional silencing and longevity protein Sir2 is an NAD- dependent histone deacetylase. Nature 403, 795-800 (2000).
7. Smith, J. S. et al. A phylogenetically conserved NAD+-dependent protein deacetylase activity in the Sir2 protein family. Proc Natl Acad Sci U S A 97, 6658-63. (2000).
8. Tanner, K. G., Landry, J., Stemglanz, R. & Denu, J. M. Silent information regulator 2 family of NAD- dependent histone/protein deacetylases generates a unique product, 1-O-acetyl-ADP-ribose. Proc Natl Acad Sci U S A 97, 14178-82. (2000).
9. Tanny, J. C., Dowd, G. J., Huang, J., Hilz, H. & Moazed, D. An enzymatic activity in the yeast Sir2 protein that is essential for gene silencing. Cell 99, 735-45. (1999).
10. Guarente, L. Sir2 links chromatin silencing, metabolism, and aging. Genes Dev 14, 1021-6. (2000).
11. Tissenbaum, H. A. & Guarente, L. Increased dosage of a sir-2 gene extends lifespan in Caenorhabditis elegans. Nature 410, 227-30. (2001).
12. Rogina, B., Helfand, S. L. & Frankel, S. Longevity regulation by Drosophila Rpd3 deacetylase and caloric restriction. Science 298, 1745. (2002).
13. Jiang, J. C., Jaruga, E., Repnevskaya, M. V. & Jazwinski, S. M. An intervention resembling caloric restriction prolongs life span and retards aging in yeast. Faseb J 14, 2135-7. (2000).
14. Kenyon, C. A conserved regulatory mechanism for aging. Cell 105, 165-168 (2001).
15. Masoro, E. J. Caloric restriction and aging: an update. Exp Gerontol 35, 299-305. (2000).
16. Koubova, J. & Guarente, L. How does calorie restriction work? Genes Dev 17, 313-21 (2003).
17. Sinclair, D. A. Paradigms and pitfalls of yeast longevity research. Mech Ageing Dev 123, 857-67 (2002).
18. Kaeberlein, M., McVey, M. & Guarente, L. The SIR_{2/3/4} complex and SIR₂ alone promote longevity in Saccharomyces cerevisiae by two different mechanisms. Genes Dev 13, 2570-80. (1999).
19. Chippindale, A. K., Leroi, Armand M., Kim, Sung B., and Rose, Michael R.

Phenotypic plasticity and selection in Drosophila life-history evolution. Journal of Evolutionary Biology 6, 171-193 (1993).
20. Chapman, T. & Partridge, L. Female fitness in Drosophila melanogaster: an interaction between the effect of nutrition and of encounter rate with males. Proc R Soc Lond B Biol Sci 263, 755-9 (1996).
21. Walker, D. W., McColl, G., Jenkins, N. L., Harris, J. & Lithgow, G. J. Evolution of lifespan in C. elegans. Nature 405, 296-7 (2000).
22. Marden, J. H., Rogina, B., Montooth, K. L. & Helfand, S. L. Conditional tradeoffs between aging and organismal performance of Indy long-lived mutant flies. Proc Natl Acad Sci U S A 100, 3369-73 (2003).
23. Schmid-Hempel, P. On the evolutionary ecology of host-parasite interactions: addressing the question with regard to bumblebees and their parasites. Naturwissenschaften 88, 147-58 (2001).
24. Ebert, D. & Bull, J. J. Challenging the trade-off model for the evolution of virulence: is virulence management feasible? Trends Microbiol 11, 15-20 (2003).
25. Soleas, G. J., Diamandis, E. P. & Goldberg, D. M. Resveratrol: a molecule whose time has come? And gone? Clin Biochem 30, 91-113 (1997).
26. Bitterman, K. J., Anderson, R. M., Cohen, H. Y., Latorre-Esteves, M. & Sinclair, D. A. Inhibition of silencing and accelerated aging by nicotinamide, a putative negative regulator of yeast sir2 and human SIRT1. J Biol Chem 277, 45099-107. (2002).
27. Edgecomb, R. S., Harth, C. E. & Schneiderman, A. M. Regulation of feeding behavior in adult Drosophila melanogaster varies with feeding regime and nutritional state. J Exp Biol 197, 215-35 (1994).

### EXAMPLE 16

### Identification of Additional Activators and Inhibitors or Sirtuins

The following high-throughput screening protocol was used to identify additional small molecule sirtuin activators and inhibitors from an ICCB library.

The following wells were designated for control reactions: a) with enzyme; DMSO blank, b) with enzyme; with resveratrol (50 μM) positive control. The reaction mixture contains (final): 0.5 units/reaction SIRT1 deacetylase (BIOMOL); 200 μM NAD⁺; 5 μM Fluor de Lys-SIRT1 substrate (BIOMOL); buffer (25 mM Tris/Cl, pH 8.0, 137 mM NaCl, 2.7 mM KCl, 1 mM MgCl₂, and 1 mg/ml BSA). In addition, a reaction mixture containing no enzyme was made so that each well receiving compound has a corresponding “no enzyme control” well. Reactions were performed in black 384 well plates (NUNC) in a final volume of 25 μl/well.

The reactions were started by combining enzyme and substrate in a reaction mixture immediately prior to aliquoting in plates (or substrate only for “no enzyme control” plates). Mixture were aliquoted to plates using Biotek μfill (Biotek Instruments). Control mixtures were manually added to designated wells. A library compound was added at a desired concentration by pin transfer to both “with enzyme” and “no enzyme” plates. Compounds were added in at least triplicate (with enzyme reaction in duplicate and no enzyme controls) at a final concentration of roughly 50 μM. The plates were incubated at 37° C for 30-60 minutes. Then 25 μl of 1× Developer II (BIOMOL) plus 2 mM nicotinamide were added to all wells to stop the reactions. The reactions were left for at least 30 minutes at 37° C. for the signal to develop. The plates were read in a microplate-reading fluorometer capable of excitation at a wavelength in the range of 350-380 nm and detection of emitted light in the range of 440-460 nm. A read time of 0.1 sec per well was used.

The following positive controls were used: resveratrol, resveratrol 4″-methyl ether (3,5-dihydroxy-4′-methoxy-trans-stilbene, also referred to herein as BML-233, and set forth in Table 10), and pinosylvin, which activated SIRT1 2.2 fold, 2.1 fold and 3.28 fold, respectively. The activators are listed in Table 21 and the inhibitors are listed in Table 22.

### EXAMPLE 17

### Treatment of Amyotrophic Lateral Sclerosis (ALS) (Murine Model) Using Sirtuin Modulators

ALS is a rapidly progressive motor neuron disease that invariably leads to death. In the United States alone, as many as 20,000 people are affected, and an estimated additional 5,000 people are diagnosed with the disease each year. ALS most commonly affects people between 40 and 60 years of age. In the vast majority of patients, ALS is sporadic and occurs apparently at random with no clearly associated risk factors. A particularly devastating effect of ALS is that a person's mind, personality, intelligence or memory is not affected, but their ability to react, communicate, and to control voluntary and involuntary muscles is lost.

CNS Penetration and Distribution of Radiolabeled Compound

For a compound to exhibit efficacy in an animal model of ALS, it must achieve therapeutic concentrations within the CNS and reach the sites within the CNS that are relevant to the degeneration observed. In the mouse models of ALS, the primary site of neuronal loss is the lumbar spinal cord that innervates the hind limbs and tail. To confirm that the compound of interest reaches the CNS, brain and spinal cord penetration and distribution are studied. The compound of interest is radiolabeled and administered to mice. Distribution of the compound within the CNS is determined by autoradiography and extraction.

Briefly, male Swiss Webster mice weighing 20-25 g at the time of the experiment are maintained under a light-dark cycle of 12 h-12 h at a room temperature of 21±2° C., with 50±15% humidity. The mice have free access to commercial mouse food and tap water.

The ¹⁴C-labeled sirtuin modulator is administered as intraperitoneal (i.p.) injections to mice every 12 h for 2 days. The amount of ¹⁴C-labeled compound injected is determined based on its specific activity and in vitro activity.

Following administration, animals are sacrificed at 30 minutes, 3 hours and 6 hours. The brains and spinal cords are rapidly removed and frozen in 2-methylbutane at −20° C., then kept below −70° C. until sectioning or solid phase extraction.

Frozen brains are mounted on cryostat chucks and cut into 20 μm thick coronal sections at −20° C. in a Microm HM 500 O microtome cryostat. Sections are thaw-mounted near the edge of slides and dried overnight under a gentle stream of air. The slides are exposed to ¹⁴C-sensitive film (Hyperfilm MP, Amersham Biosciences) at 5° C. for 3 days. Images are analyzed using an HP Scanjet 8200C scanner and analyzed using an image analysis software package (Image, NIH software). ¹⁴C standards (¹⁴C-microscales) (30-860 nCi/g) are used for quantifying the autoradiograms. Density readings for standards of known radioactivity are taken for comparison of optical density to isotope levels on each sheet of film. Standard curves for converting optical density to nCi/g values are best-fit by linear transformation. Background readings of optical density are used in determining the relative amount of drug bound to each section. Different regions of the brain selected are examined for labeling with ¹⁴C-labeled compound. Regions are identified using an atlas of the brain (Paxinos G., Franklin K. B. J., The mouse brain in stereotaxic coordinates Academic Press, New-York, 2003). The amount of ¹⁴C-labeled compound bound to each area is expressed as the mean for each slide (3 sections per slide). Data taken from areas found in both the left and right hemispheres are pooled from each section to determine the overall mean for that region of brain.

To determine compound exposure to the spinal cord, the spinal cord is homogenized and centrifuged to remove any solids from the sample. An aliquot of the sample is combined with 1% phosphoric acid with water in a 96-well plate and mixed. The sample is added to a Phenomenex StrataX extraction plate that has been equilibrated with methanol and water. Following washing, the sample is eluted with 100% acetonitrile into a clean 96-well plate. The samples are evaporated under a stream of N₂ and the residue reconstituted in solvent. The quantity of compound is assessed by mass spectrometry (LC-MS/MS).

Data are analyzed for statistical significance by ANOVA and Dunnett's t-test using the software Statview (BrainPower, Calabasas, Calif., U.S.A.). Statistical significance is taken as p<0.05.

Compound Efficacy in an Animal Model of Progressive Motor Neuron Disease (pmn/pmn)

The pmn mouse model is a widely used genetic animal model for studying degeneration of motor neurons. The mice carry a spontaneous autosomal recessive mutation that leads to progressive motor neuronopathy (Schmalbruch, H., et al. J Neuropathol Exp Neurol, 1991. 50(3): p. 192-204). pmn homozygous mice develop weakness in the hind limbs during the third week of life and die at approximately 6 weeks of age. At this latter age, the animals show a severe muscle wasting particularly in those muscles of the thoracic and pelvic regions. Heterozygous pmn mice are phenotypically normal. Histological studies have revealed that the sciatic and phrenic nerves of pmn animals are severely affected (Schmalbruch, H., et al., supra; Sagot, Y., et al. Eur J Neurosci, 1995. 7(6): p. 1313-22; Sagot, Y., et al. J Neurosci, 1995. 15(11): p. 7727-33; and Sagot, Y., et al. J Neurosci, 1996. 16(7): p. 2335-41) and that 30% of the facial nucleus motor neurons degenerate (Sendtner, M., et al. Nature, 1992. 358(6386): p. 502-4). The pmn mouse model of motor neuron disease is used to examine the potential neuroprotective properties of sirtuin modulators. The effects of sirtuin modulators on disease onset, motor function, motor neuron loss, and survival of the pmn/pmn mouse are determined.

Heterozygous pmn mice are obtained from the laboratory of Dr. Ann Kato from the Centre Medical Universitaire (Geneva, Switzerland). A large colony of pmn mice is generated; pmn/pmn homozygotes are infertile and are obtained from double heterozygous crosses at the Mendelian ratio of 25%. Starting at 12 days of age, the mice are examined for grasp activity of the hind limb paws. The first clinical signs of weakness usually appear between days 14 and 16. Animals are divided into groups at two weeks of age. Controls and treated pmn mice have access to commercial food and tap water ad libitum throughout the study. When it is determined by examiners that the mice are unable to reach dry food and/or water, a water-based nutrient gel will be placed on the bottom of the cage, and a longer spout will be attached to the water bottle.

The mice are divided into four test groups: Group A: negative-control animals (heterozygote and wild type mice) treated with vehicle; group B: positive-control animals (pmn/pmn homozygotes) treated with vehicle; group C: pmn/pmn homozygotes treated with sirtuin modulator (dose 1); and group D: pmn/pmn homozygotes treated with sirtuin modulator (dose 2).

Briefly, Group A serves as negative-control animals that do not exhibit motor neuron loss (heterozygote and wild type mice). Group A is treated with vehicle daily throughout the study. Group B is the positive-control animals and is dosed with vehicle daily throughout the study. Groups C and D are treated with the sirtuin modulating compound at 2 different doses. The dose is determined based on compound activity in vitro and CNS penetration determined using radiolabeled compound as described above. For these studies, test compounds or vehicle is administered i.p. twice a day with 10 to 12 hours between injections. The treatment is administered from two weeks of age throughout the study. Animals from each group are used for histological evaluation. These mice are sacrificed at a late disease stage (35 days) to assess the extent of motor neuron loss and the extent of gliosis.

The parameters followed for this study are body weight, behavior, motor neuron loss, gliosis, and life span. Throughout the study, body weight is determined daily by weighing the animals at the same time each morning prior to the administration of the sirtuin modulator or vehicle. The body weight evolution is expressed as the cumulative sum of the variation in the percentage of the initial body weight.

For the behavioural assessment, the mice are tested for their ability to execute the following behavioural tests: back leg grasping, bar crossing, inclinded plande test and grip test.

Back leg grasping. This test measures the ability of pmn mice to hold onto the side of their cage with their hind limbs. The mice, held head-down by the tail, will be allowed to grasp the cage and remain suspended. As early as day 15, pmn homozygous animals can be diagnosed by their inability to grasp onto the side of the cage. The mice are tested every 2 days.

Bar crossing. In this test, the time to cross a 25 cm long cylindrical bar is measured. If the mice fall from the bar, the test is considered unsuccessful and is repeated three times. The mice are tested every 2 days.

Inclined plane test. The mice are tested 1 time per week for their ability to stay on an inclined plane within a maximum of 5 seconds. The slope that each animal remains on the plane is recorded.

Grip test. The mice are tested 1 time per week for their ability to hold a horizontal bar two times, within a maximum of 30 seconds. The time each animal remains on the bar is recorded.

For histological and stereological analysis, mice are perfused with phosphate buffered saline followed by paraformaldehyde. The spinal cords are dissected and the lumbar segments identified. Tissues are postfixed and blocks will be cryoprotected. To quantify motor neurons numbers, high-precision stereological analysis are performed. Serial coronal sections are cut through the lumbar (L1 to L4) spinal cord. The sections are mounted onto slides and stained for Nissl substance using cresyl violet. A separate set of sections are collected as free-floating sections and processed for immunohistochemistry, which is aimed at determining the extent of gliosis or astrocyte and microglial involvement. The sections are immunostained with CD40 (microglial marker) and GFAP (astrocyte marker) antibodies using double label immunofluorescence.

Life span is determined for each test group. In order to reduce animal suffering, new guidelines have been established to determine endpoint (survival); animals are euthanized when they are unable to do any of the following: right themselves within 15 seconds when placed on their sides, groom their faces (as determined by infection in one or both eyes), or move around the cage, even by use of front limbs, to reach food placed at the bottom of the cage. Negative control animals are euthanized at the end of the study by CO₂ inhalation.

For statistical evaluation of the data, the life span results are submitted to a Kaplan-Meier test. Two different tests of measuring statistical significance are used; the Log-Rank test and the Wilcoxon test. Data related to quantitative behavioral assessments are analyzed with Kruskal-Wallis followed by non parametric Mann-Whitney U-test. Significance is considered as p<0.05.

Compound Efficacy in an Animal Model of ALS Disease (SOD1^{G93A})

The SOD1^{G93A} mice are obtained from the Jackson Laboratories (Gurney, M. E., et al. Science, 1994. 264(5166): p. 1772-5). The mice express high levels of human SOD1 containing a substitution of glycine to alanine at position 93. This mutation is found mutated in 20% of familial ALS patients and thus represents a useful and relevant model for studying the efficacy of sirtuin modulators. The effects of the sirtuin modulator across standard experimental parameters are examined: disease onset, motor function, motor neuron loss, gliosis, and survival of the SOD^{G93A} mouse.

The specific mouse strain, designated G1H, is maintained as a heterozygous hybrid line which is a cross between C57B6/J and SJL mice. Transgenic males are crossed with nontransgenic B6SJLF1 females. Animals are genotyped at weaning, approximately 21-30 days of age by PCR amplification from DNA extracted from tail biopsies while the animals are temporarily anesthetized by inhalation of isoflurane. For the DNA extraction, a QIAamp Tissue Kit from Qiagen is used. PCR amplification is performed using a primer pair specific for exon 4 of the human SOD1 gene. At 30 days of age, the mice are randomized into three different treatment arms. All animals have access to commercial food and tap water ad libitum throughout the study. When it is determined by examiners that the mice are unable to reach dry food and/or water, a water-based nutrient gel will be placed on the bottom of the cage and a longer spout will be attached to the water bottle.

The following three test groups are studied: Group A: SOD1^{G93A} mice treated with vehicle serve as the positive control group; Group B: SOD1^{G93A} mice treated with the sirtuin modulator (dose 1); and Group C: SOD1^{G93A} mice treated with the sirtuin modulator (dose 2).

Briefly, Group A serves as positive-control animals that exhibit motor neuron loss. Group A is treated with vehicle daily throughout the study. Groups B and C are treated with the sirtuin modualting compound at 2 different doses. The dose is determined based on compound activity in vitro and CNS penetration. For these studies, test compounds or vehicle are administered i.p. twice daily with 10 to 12 hours between injections. The treatment is initiated on day 30 and continues throughout the study. Animals from each group will be used for histological evaluation. These mice are sacrificed at a late stage in the disease (120 days) to assess the extent of motor neurons loss and the extent of gliosis.

The parameters followed for this study are body weight, disease onset, gait, life span, motor neuron loss, and gliosis. Throughout the study body weight is determined daily by weighing the animals at the same time each morning prior to the administration of the test compound or vehicle. The body weight evolution is expressed as the cumulative sum of the variation in the percentage of the initial body weight.

The mice are examined twice weekly to determine disease onset. Onset is defined as the day of the first appearance of limb tremor when the animals are held suspended briefly by their tails. This usually begins unilaterally, followed by bilateral tremulousness and weakness in the affected limb(s). Following initial diagnosis, animals are examined daily for early stages of hind-limb paralysis.

Gait analysis is performed to assess motor functioning of the test groups. Briefly, footprint patterns are studied using mouse fore- and hindpaws dipped in blue and red non-toxic, water based paint, respectively. The mice are placed in a clear Perspex runway that has a black goal box fixed to one of the distal ends. White paper is used to line the runway floor. Mice are permitted to walk to the goal box from the opposite end of the runway thus allowing their footprints to leave patterns on the paper. Five separate parameters are measured; stride length, hind- and forepaw base width, overlap between fore and hindpaws, and latency to travel the runway.

Life span determination, histological analysis, stereological analysis and statistical evaluation are carried out as described above.

### EXAMPLE 18

### Treatment of Multiple Sclerosis (MS) (Murine Modulator) using Sirtuin Modulators

Multiple Sclerosis (MS) is the most common cause of non-traumatic neurological disability affecting young adults. An estimated 2.5 million people have MS worldwide and approximately 400,000 in the U.S (source: NINDS). MS is an inflammatory disease of the central nervous system (CNS) in which demyelination and axonal injury result in a permanent neurological disability. The disease can present in different forms, such as primary progressive (accumulation of disability without remission) or relapsing remitting (acute attacks followed by periods of recovery). About 40% of patients enter a secondary progressive stage (attacks with incomplete recovery that lead to progressive disability between exacerbations). There is no cure for MS. Recently approved drugs focus on the inflammatory autoimmune components of the disease, and they appear to control relapses and may be effective in slowing progression from relapsing-remitting to secondary progressive. However, these immunomodulatory interventions do not address the underlying axonal injuries, and therefore do not impact the neurological damage resulting from acute demyelinating events, acute axonal transection and axonal loss.

Experimental autoimmune encephalomyelitis (EAE) is an animal model of MS induced by immunization with proteolipid protein (PLP). Animals mount an immune response resulting in inflammation, demyelination, and neuronal damage in the brain, spinal cord, and optic nerve, similar to MS patients. Assessment of clinical/neurological symptoms, and histological analysis of demyelination and axonal damage in the thoracic spinal cord are examined.

Chronic relapsing EAE is induced in 8-12 week old female SJL mice by subcutaneous (s.c.) injection with an emulsion containing PLP 139-151 peptide and complete Freund's adjuvant containing 150 μg of peptide and 200 μg of Mycobacterium tuberculosis in a total volume of 0.2 ml. In addition, mice are injected intraperitoneally (i.p.) with 200 ng pertussis toxin (List Biological, Campbell, Calif.) in 0.1 ml PBS on day 0 (day of immunization) and again on day 2. The animals are housed in standard conditions: constant temperature (22±1° C.), humidity (relative, 25%) and a 12-h light/12-h dark cycle, and are allowed free access to food and water. Animals are assessed daily for weight and clinical signs of EAE, beginning 11 days after immunization. Assessment continues until day 40 after the initial inoculation. During this time animals undergo an initial phase of EAE, followed by recovery. A relapse of EAE typically occurs 20-30 days post-immunization. Mice are considered to have had a relapse if they have an increase by 1 on the clinical scale for two or more days after a period of five or more days of stable or improved appearance.

Female SJL/J mice were immunized by a subcutaneous (s.c.) injection with proteolipid protein 139-151 peptide in complete Freund's adjuvant. Mice were treated with resveratrol (125 mM resveratrol in 40% Captisol, pH app. 6.0) or vehicle (40% Captisol) for 30 days by daily IP injection at a dose of either 200 mg/kg/day (low dose) or 400 mg/kg/day (high dose) beginning on day 11 (onset of paralysis) and perfused on day 40. As a positive control, the immunosuppressant FK506 (tacrolimus) was used at 5 mg/kg/day.

During the initial few weeks of treatment, most of the mice developed sores and scabbing. The mice receiving the high dose resveratrol were very irritated after injection, scratched their head area raw and, many developed a black hued skin color. Under direction from a veterinarian, antibiotic treatment was applied. Over the course of the next week, the skin lesions largely disappeared and the irritation following injection resolved.

Blood was collected at the time of perfusion, 1-1.25 hours after the last injection of resveratrol. The blood was centrifuged, serum collected, frozen prior to analysis.

Mice were examined for clinical signs of EAE daily beginning 11 days after immunization using the following scale: 0, no paralysis; 1, limp tail with minimal hind limb weakness (animal cannot be flipped easily onto its back); 2, mild hind limb weakness (animal can be easily flipped onto its back but rights itself easily); 3, moderate hind limb weakness; 4, moderately severe hind limb weakness; 5, severe hind limb weakness; 6, complete hind limb paralysis; 7, hind limb paralysis with mild fore limb weakness; 8, hind limb paralysis with moderate fore limb weakness; 9, hind limb paralysis with severe fore limb weakness. After initiation of treatment, mice were graded for EAE blinded to treatment status. To assess the severity of the initial clinical episode of EAE, a 10-Day Cumulative Disease Score (10 Day-CDS) was calculated for each animal by adding the daily disease score on 10 consecutive days commencing on the first day of disease. To assess severity throughout the course of chronic relapsing EAE, a Total Disease Score was determined by adding the daily disease score beginning on the first day of disease until the animals were sacrificed. Following recovery from the initial episode of EAE, mice were considered to have had a relapse if they had an increase in EAE score by 1 or more for >2 consecutive days after a period of >5 days of having stable or improving scores.

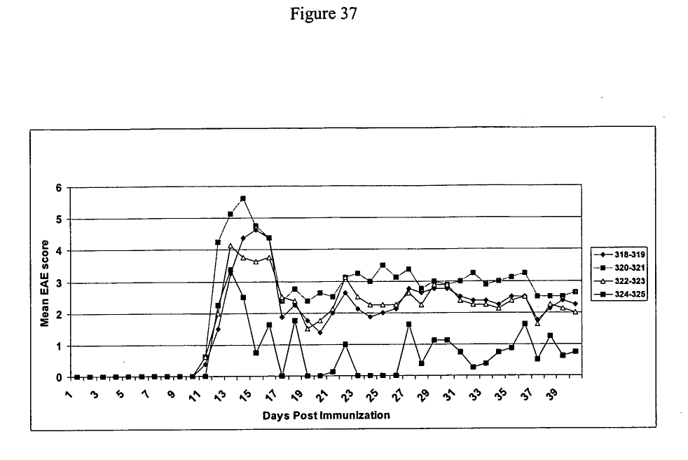

FIG. 37 are plots showing EAE scores over time of the four groups. The four groups are animals in the vehicle control group (labeled as 318-319); 200 mg/kg low dose resveratrol or Lo SRT (320-321); 400 mg/kg high dose resveratrol or Hi SRT (322-323); and 5 mg/kg FK506 (324-325).

The tables below show mean EAE scores for the entire study (Total EAE scores) and for the last 10 days. Each data set is derived from groups of 8 mice. In the low dose resveratrol group, the peak and overall disease level was slightly higher than controls. In the high dose resveratrol group, the clinical course was not different from controls. As previously reported, FK506 (5 mg/kg) reduced the severity of the initial disease and suppressed relapses.

Mean Total EAE Scores

| Treatment | Mean EAE Score | Std. Dev | p-value |
|---|---|---|---|
| Veh. Ctrl. | 72.38 | 28.44 | — |
| Lo SRT | 94.00 | 26.20 | 0.09 |
| Hi SRT | 73.00 | 40.61 | 0.67 |
| FK506 | 25.38 | 11.62 | 0.002 |

Mean EAE Scores - Last 10 Days

| Treatment | Mean EAE Score | Std. Dev | p-value |
|---|---|---|---|
| Veh. Ctrl. | 23.00 | 8.72 | — |
| Lo SRT | 28.63 | 6.67 | 0.08 |
| Hi SRT | 21.88 | 12.57 | >0.99 |
| FK506 | 7.75 | 6.16 | 0.003 |

At day 40 post-immunization, mice from each group are sacrificed with an overdose of ketamine/xylazine. Spinal cords are dissected, fixed in 10% buffered formalin, and embedded in paraffin. Five micron thick sections are stained with Hematoxylin and Eosin (H&E) and Luxol Fast Blue (LFB) to assess myelin loss. Bielshowesky's silver impregnation is used to evaluate axonal integrity. To asses the amount of axonal loss, paraffin sections are exposed to monoclonal antibodies against mouse non-phosphorylated neurofilament H (Clone SMI-32, Stemberger Monoclonals, Baltimore, USA) and monoclonal antibodies against APP (Clone 22C11, Chemicon). SMI-32 is detected with a Cy3-labeled antibody and visualized by fluorescence microscopy. Anti-APP antibodies are detected by incubation with ColonoPAP, and APP-positive axons are visualized with 3,3′-diaminobenzidine (DAB).

To evaluate the extent of axonal loss, images of slides are captured and the areas stained by immunohistochemistry are quantified blinded to treatment status. Axonal integrity and demyelination are assessed qualitatively.

The percentage of the spinal cord showing damage was determined in the cervical, thoracic and lumbar cord. At each level, regions in the 1) dorsal columns and 2) the lateral and ventral white matter tracts containing damaged fibers was circumscribed on photomontages (final magnification ×100) of the entire spinal cord. Damaged areas in each of the two regions were measured using a SummaSketch III (Summagraphics, Seymour, Conn.) digitizing tablet and BIOQUANT Classic 95 software (R&M Biometrics, Nashville, Tenn.). Measurements were also be made of the total area (damaged and nondamaged) of the 1) dorsal columns and 2) the lateral and ventral columns. For each section (one section per animal), the cumulative percent lesion areas were calculated for each region (dorsal column, lateral and ventral columns).

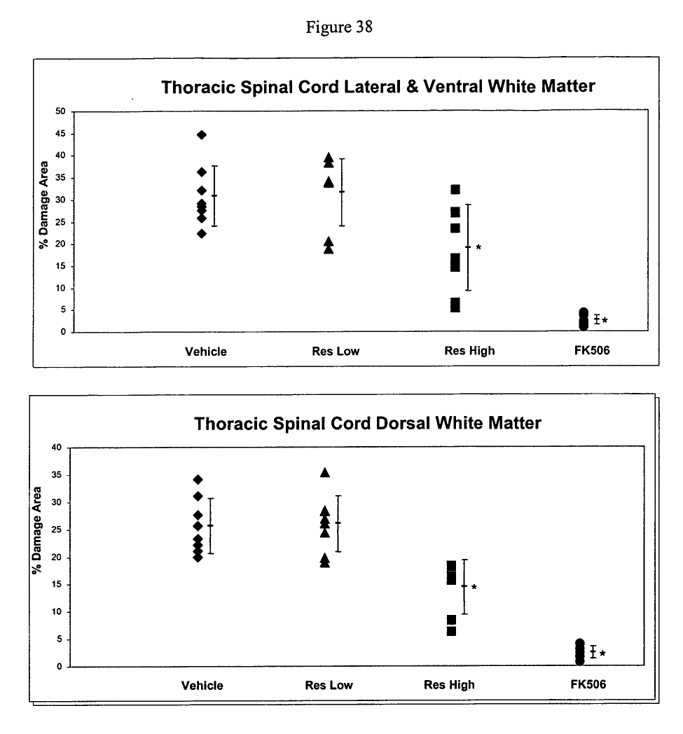

FIG. 38 are plots showing the degree of damage in the ventral/lateral (Top) and dorsal (Bottom) white matter of the thoracic spinal cords. The extent of damage is significantly reduced in 400 mg/kg high dose and FK506 groups. Each data set is derived from groups of 8 mice. One spinal cord section was analyzed from each animal. *p<0.005. In the thoracic spinal cord, resveratrol (low group) slightly increased, albeit not significantly, the degree of damage in the dorsal, lateral and ventral white matter, consistent with the clinical appearance. The resveratrol (high group) significantly reduced by approximately 40% the degree of damage in the dorsal, lateral and ventral white matter. In contrast, FK506 (5 mg/kg) significantly reduced the extent of damage by over 90% in the dorsal, lateral and ventral white matter, as previously reported.

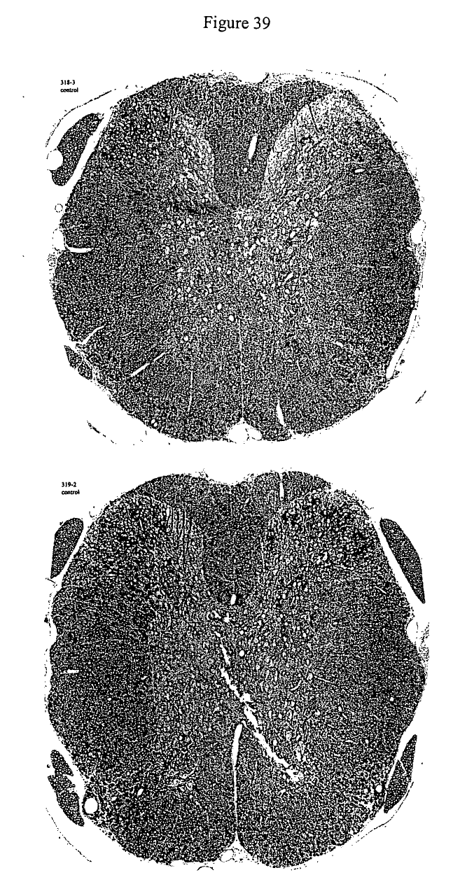

FIG. 39 shows representative sections from thoracic spinal cord from two mice treated with vehicle.

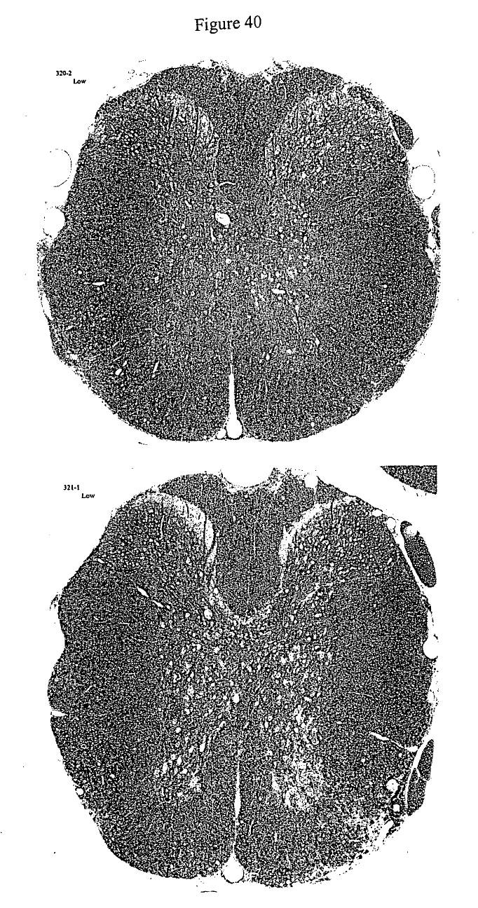

FIG. 40 shows representative sections from thoracic spinal cord from two mice treated with resveratrol (200 mg/kg).

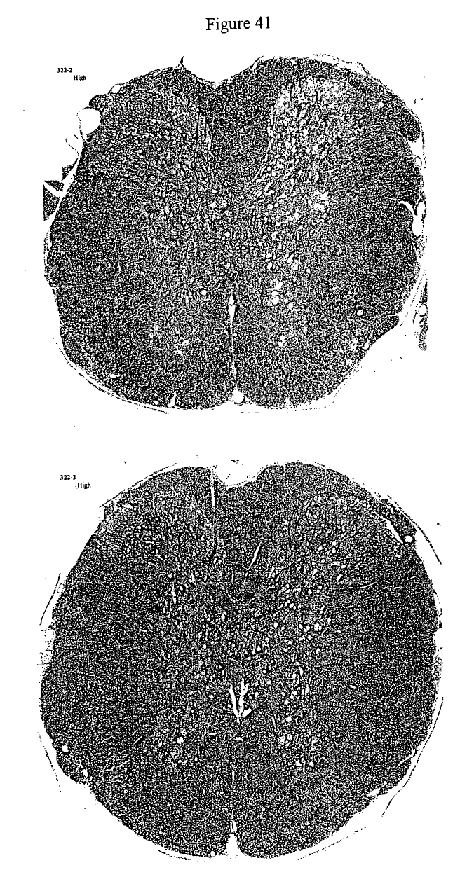

FIG. 41 shows representative sections from thoracic spinal cord from two mice treated with resveratrol (400 mg/kg).

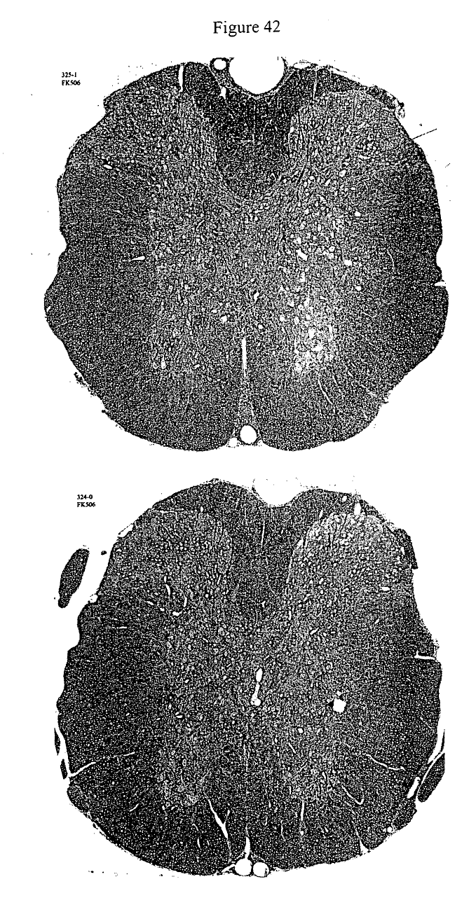

FIG. 42 shows representative sections from thoracic spinal cord from two mice treated with FK506 (5 mg/kg).

Taken together, the results demonstrate that high dose resveratrol is protective against both demyelination and axonal loss in a model of MS. The lack of effect on the clinical course indicates that the drug did not reduce T-cell infiltration (although this needs to be addressed by immunohistochemistry).

In order to assess the effect of sirtuin modulators on neurodegeneration, it is critical not to interfere with the lymphoid development of effector cells early in the disease process. Therefore, the sirtuin modulator is administered at the onset of clinical EAE. Even though immunosuppression is responsible for reducing the clinical severity of the initial phase of EAE, a recent study suggests that a combination of immnosuppression and neuroprotection may be critical to effectively inhibit relapses, demyelination and axonal injury, and that chronic immunosuppression in the absence of effective neuroprotection may worsen the clinical outcome in EAE and, perhaps, MS. This issue is addressed by evaluating the effect of immunosuppression (i.e. Copaxone (glatiramer acetate)) in combination with neuroprotection (by sirtuin modulators) in the PLP-induced EAE mouse model.

Chronic relapsing EAE is induced as described above. Mice are divided into three treatment groups: Group 1: vehicle control, daily i.p. injections of cyclodextrin (days 12-39); Group 2: Copaxone treatment, daily s.c. injection (days 0-9); and Group 3: Copaxone (days 0-9) and sirtuin modulator (days 12-39). As described above, disease progression is monitored, and mice from each group are sacrificed, the spinal cords harvested and analyzed for demyelination, axonal integrity and axonal damage.

### EXAMPLE 19

### Treatment of Huntington's Disease (Murine Model) using Sirtuin Modulators

The R_{6/2} mutant mouse model of Huntington's disease (HD) is used to test the efficacy of sirtuin modulating compounds to attenuate HD disease-related symptoms.

R_{6/2} mice are treated with a sirtuin modulating compound for at least 12 weeks. The mice are evaluated at 4, 6, 8 and 12 weeks of age (except for Grip Strength which will only be tested 12 weeks of age) using the Rotarod, grip strength, rearing/climbing, open field, and body weight/survival test.

During the course of the study, 12/12 light/dark cycles are maintained. The room temperature is maintained between 20 and 23° C. with a relative humidity maintained around 50%. Chow and water are provided ad libitum for the duration of the study. Each mouse is randomly assigned across the dose groups and balanced by cage numbers. The test is performed during the animal's light cycle phase unless otherwise specified.

Rotarod. Motor coordination and exercise capacity are assessed by rotarod at 4, 6, 8 and 12 weeks of age. Tests are performed on three separate days, with four trials per day. Animals are loaded on the continuous rotating rod (Accuscan, Columbus, Ohio) 8 animals at a time. They are given a 5-min training period at a slow speed of 4 rpm. If an animal falls off the rod it is placed back on the rod for the duration of the 5-min training period. Animals are then placed back into the home or test cage for at least one hour prior to actual testing. The mice are then placed on the rotarod and the speed is gradually and uniformly increased to a speed of 40 rpm by 300 s. The time that each mouse remains on the rotating rod before falling 20 cm onto a foam pad is recorded. Any abnormal behavior is also noted, i.e., looping behavior recording the number of rotation times per session trial, walking forward against the rod direction, and number of fecal boli. After rotarod testing animals are placed back into the test or home cage Grip-strength test. Grip strength is used to assess muscular strength in limb muscles and mice are tested at 12 weeks of age. Mice are held by the tail and lowered towards the mesh grip piece on the push-pull gauge (San Diego Instruments, San Diego, Calif.) until the animal grabs with both front paws. The animal is lowered toward the platform and gently pulled backwards with consistent force by the experimenter until it releases its grip. The forelimb grip force is recorded on the strain gauge. The experimenter continues to pull the animal backwards along the platform until the animal's hind paws grab the mesh grip piece on the push-pull gauge. The animal is gently pulled backwards with consistent force by the experimenter until it releases its grip. The hind limb grip force is recorded on the strain gauge. After testing animals are placed back into the test or home cage.

Rearing-Climbing. Rearing-climbing behavior is used to assess motor movement and coordination. The mouse is placed on a flat surface and a closed-top wire mesh cylinder 15 cm×20 cm tall is placed over the mouse. The animal's behavior is videotaped. The following parameters are then measured over a 5 min period: number of free rears, the number of times the animal rears in contact with the wall, number of times the animal lifts either 1, 2 or 3 paws from the floor, the number of climbing episodes (lifting 4 paws), the number of hanging episodes (from the mesh), and the time spent hanging and climbing. After the 5-min session animals are placed back into the home cage.

Open field—locomotor activity. Mice are acclimated to the test room at least 1 hour prior to the commencing the test. The open field test (OF) is used to assess both anxiety-like behavior and motor activity. The open field chambers are plexi-glass square chambers (27.3×27.3×20.3 cm; Med Associates Incs., St Albans, Vt.) surrounded by infrared photobeam sources (16×16×16). The enclosure is configured to split the open field into a center and periphery zone and the photocell beams are set to measure activity in the center and in the periphery of the OF chambers. Animals having higher levels of anxiety or lower levels of activity tend to stay in the corners of the OF enclosures. On the other hand, mice that have high levels of activity and low levels of anxiety tend to spend more time in the center of the enclosure. Horizontal activity (distance traveled) and vertical activity (rearing) are measured from consecutive beam breaks. Animals will be placed in the OF chambers for 30 minutes. Ambulatory distance in center and periphery; rearing in center and periphery; the number of zone entries and average velocity are measured.

Body Weight and Survival. Body weights are measured daily. The survival times of the mice tested as described above are determined. Fatalities are evaluated in the context of the other parameters measured. In previous studies in R_{6/2} Huntington's disease model mice, no differences were found between survival times in experimental versus non-experimental groups.

Statistical Analysis. Data are analyzed by a one-way or two-way analysis of variance (ANOVA) followed by post-hoc comparisons. An effect is considered significant if p<0.05. Data are represented as the mean and standard error to the mean (s.e.m.). Animals are removed from the group if the data is two standard deviations away from the mean.

### EXAMPLE 20

### Treatment of Chemotherapeutic-Induced Neuropathy (Rodent Model) using Sirtuin Modulators

The oncology drug Taxol (paclitaxel) is an effective treatment of ovarian, lung, breast and other cancers but its anti-microtubule activity can induce peripheral neuropathies. Taxol administration, either in a single large dose or several smaller doses, has been demonstrated to produce both sensory-motor deficits and histologically identified axonal abnormalitites in rodent models. These models are thought to be predictive of those neuropathies often seen in patients given Taxol for chemotherapy for various forms of cancer. Both sensory-motor behavioral testing and histological evaluation of nerve tissue in animals treated with Taxol and concomitantly treated with either vehicle or a sirtuin modulator are used to evaluate the effectiveness of sirtuin modulating compounds to attenuate the effects of Taxol on the peripheral nervous system.

Male Sprague-Dawley rats (Harlan Sprague Dawley Inc., Indianapolis, Ind., USA) are injected intra-peritoneally with Taxol at 20 mL/kg i.p. (32 mg/kg total dose) on Day 0 using a syringe and sterile needle. A first set of rats are treated with Normal Saline vehicle. The rats are dosed on Day 0 in combination with Taxol and are injected sub-cutaneously using a syringe and sterile needle. This dosing procedure is repeated at 24 and 48 hours post-Taxol injection. The volume of vehicle administered is 1 ml/kg bodyweight. A second set of rats are treated with a sirtuin modulating compound. The rats are treated with a sirtuin modulating compound commence on Day 0 in combination with Taxol.

Behavioral tests. Behavioral tests will include thermal paw stimulation for pain assessment test and the open field test for activity.

Thermal paw stimulation is a commonly-used method to assess hyper- and hypoalgesia in rodents. Using a thermal paw stimulator (UCSD), the latency for the rat to lift its paw is recorded in response to a heat source placed beneath the hindpaw. The rat is placed on a glass surface maintained at a constant temperature (30±1° C.) and then habituated to the apparatus for approximately 15 min prior to testing. Two measurements of paw lift latency are averaged for each animal if they are within 2 sec. of each other. If not, additional testing is performed until this criterion is met. Baseline testing is performed on Day-3. Further tests will be conducted on Days 4 and 7.

The open field test is performed as described above in Example 19.

Necropsy. On day 14 animals are euthanized by CO₂ asphyxiation and cervical dislocation. Following euthanasia the dorsal ganglia of the lumbar vertebra, sciatic nerve and hind paw dermis are harvested and fixed overnight in 10% neutral buffered formalin.

Histology. The harvested tissue is blocked, embedded in paraffin, sectioned and stained with H&E. The tissue is examined using light microscopy and scored by an evaluator blind to the treatment regimen. The tissue is ranked on a scale of 0 to 3 based on the degree and amount of axonal disruption observed in the section, with 0 being a normal appearance of the axon, 1 to 2 being a mild to moderate disruption of the axons and a 3 being a complete disruption and Wallerian degeneration of the axons.

Statistics. A two-way repeated measures ANOVA is performed on the thermal paw stimulation and open field measurements (group×time) to assess the effects of time and treatment on the behavioral performance in these rats. If there are any overall significant differences, a factorial ANOVA is performed at specific time points to determine where the difference occurred. The neuroanaotomical evaluation is assessed for statistical significance using a non-parametric analysis of the rating scores for axonal disruption.

### EXAMPLE 21

### Cell Based Assay for Examination of Amyloid Dependent Cell Toxicity

Primary neuronal cultures which contain a mixture of glia and cortical neurons are used to assess the neuroprotective effect of candidate compounds on microglia-dependent amyloid toxicity. Recent work has shown that SIRT1 overexpression or resveratrol treatment inhibits NF-κB activation and increases neuronal survival in this assay (Chen et al. 2005. SIRT1 protects against microglia-dependent beta amyloid toxicity through inhibiting NF-kappa B signaling. J Biol Chem. 280 (48) 40364).

To establish primary neuronal cultures, cortices are isolated from Sprague-Dawley rat pups on postnatal day 0. Cells are plated in culture medium (Dulbecco's modified Eagle's medium, DMEM, 10% fetal bovine serum (FBS), 0.5 mM Glutamax, 100 U/ml penicillin, and 100 μg/ml streptomycin). After 6 days, the medium is replaced with Neurobasal A medium supplemented with N2 (NBA/N2). Treatments are conducted on day 7.

Oligomeric preparations of Aβ-(1-42) are prepared as described previously (Dahlgren et al. 2002. Oligomeric and fibrillar species of amyloid-beta peptides differentially affect neuronal viability. J Biol Chem 277:32046-53; Stine et al. 2003. In vitro characterization of conditions for amyloid-beta peptide oligomerization and fibrillogenesis. J Biol Chem 278:11612-22). Briefly, Aβ-(1-42) lyophilized in hexafluoroisopropanol (HFIP) (obtainable from California Peptide, Napa, Calif.) is reconstituted in dry dimethylsulfoxide (final concentration 5 mM), and diluted in DMEM/F12 medium to a final concentration of 40-100 μM and allowed to oligomerize for 24 hours at 4° C. Oligomerization is assessed by electron microscopy.

Cells are pre-treated with candidate compounds for 30-60 min, followed by Aβ solution (final concentration 10 μM). Resveratrol is included as a positive control. Cis-resveratrol, which acts as an antioxidant, but does not activate SIRT1, is included in the assay as a negative control to distinguish SIRT1-dependent activity from potential antioxidant activity. Compounds will be added to the culture medium for the desired time at various low, medium, and high concentrations ranging from 1 to 100 μM.

To determine the effect of novel SIRT1 activators on cell viability under the assay conditions employed (without Aβ), cytotoxicity is measured by MTT test. Only compounds that exhibit a 100-fold window of dose that achieves efficacy versus cytotoxicity are considered for advancing into animal models.

The effect of novel SIRT1 activators on amyloid toxicity is also examined by immunohistochemistry (IHC). For IHC, cultures are fixed in 4% paraformaldehyde in phosphate-buffered saline (PBS), permeabilized with 0.1% Triton, and placed in blocking buffer (PBS with 10% FBS and 0.01% Triton). Neurons are immunostained with anti-MAP2 antibody and visualized with a fluorescently labeled secondary antibody. To determine neuronal loss, MAP2-positive neurons are counted in random fields under a fluorescence microscope.

NF-κB activation is assessed by electrophoretic mobility shift assay (EMSA) using nuclear extracts prepared from treated and untreated cultures. A double-stranded NF-κB consensus oligonucleotide (Sung et al. 2004. Modulation of nuclear factor-kappa B activity by indomethacin influences A beta levels but not A beta precursor protein metabolism in a model of Alzheimer's disease. Am J Pathol 165:2197-206), is used as a probe after 5′-end labeling and purification. For binding reactions, nuclear extracts (10 μg protein) are incubated with radiolabeled probes (2.5×10⁴ cpm). Competitor oligonucleotide is added to the reaction at 50-fold molar excess. The products of the binding reaction are separated by gel electrophoresis on 5% non-denaturing polyacrylamide gels. Gels are dried and analyzed by autoradiography.

Levels of endogenous acetylated NF-κB and total NF-κB in cortical cultures are determined by Western blot analysis using anti-ac-lys310 and anti-RelA/p65 antibodies. Cells are homogenized in lysis buffer (10% SDS, 62.5mM Tris pH 6.8, 5 mM EDTA). The sample is mixed with an equal volume of loading buffer (62.5 mM Tris pH 6.8, 20% glycerol, 200 mM DTT, 0.2% bromophenol blue) and run on a 12.5% polyacrylamide gel. Samples are transferred to Immobilon P (Millipore). The blot is blocked in 5% milk powder, 0.5% BSA in PBS-Tween for 1 hour and incubated for 1 hour with primary antibody followed by detection. The blots are then stripped and re-probed with a β-actin antibody to control for protein loading.

Equivalents

The present invention provides among other things sirtuin-activating compounds and methods of use thereof. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: PCT Publications WO 2005/002672; 2005/002555; and 2004/016726; and U.S. Patent Application Publication No. 2002/0049176.

1. A method for treating or preventing a neurodegenerative disorder in a subject, comprising administering daily to a subject in need thereof a sirtuin activating compound that has a sirtuin activating effect equal to or greater than 18 mg/kg resveratrol.

2. The method of claim 1, wherein the sirtuin-activating compound comprises a formula selected from the group consisting of formulas 1-25, 30, 32-65, and 69-88.

3. The method of claim 1, wherein the sirtuin-activating compound is resveratrol, fisetin, butein, piceatannol or quercetin.

4. The method claim 1, further comprising administering to the subject a therapeutically effective amount of an anti-neurodegeneration agent.

5. The method of claim 1, wherein the neurodegenerative disorder is selected from the group consisting of Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, Multiple Sclerosis (MS), and Friedreich's ataxia.

6. The method of claim 1, wherein the subject is a human.

7. The method of claim 1, wherein the subject,would benefit from increased mitochondrial activity.

8. The method of claim 7, wherein the sirtuin activating compound increases mitochondrial activity without increasing mitochondrial mass.

9. The method of claim 7, wherein the sirtuin activating compound increases mitochondrial mass.

10. A method for treating or preventing a neurodegenerative disorder in a subject, comprising administering to a subject in need thereof a therapeutically effective amount of a sirtuin activating compound and a PPAR agonist.

11. The method of claim 10, wherein the sirtuin-activating compound comprises a formula selected from the group consisting of formulas 1-25, 30, 32-65, and 69-88.

12. The method of claim 10, wherein the sirtuin-activating compound is resveratrol, fisetin, butein, piceatannol or quercetin.

13. The method of claim 10, wherein the PPAR agonist is a PPAR-alpha agonist, a PPAR-gamma agonist, or a PPAR-delta agonist.

14. The method of claim 10, wherein the neurodegenerative disorder is selected from the group consisting of Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, Multiple Sclerosis (MS), and Friedreich's ataxia.

15. The method of claim 10, wherein the subject is a human.

16. A method for treating or preventing a neurodegenerative disorder in a subject, comprising administering to a subject in need thereof a therapeutically effective amount of a sirtuin activating compound and an anti-inflammatory agent.

17. The method of claim 16, wherein the neurodegenerative disorder is Alzheimer's disease (AD), Huntington's Disease (HD) and other polyglutamine diseases, Parkinsons Disease (PD), amyotrophic lateral sclerosis (ALS; Lou Gehrig's disease), or Multiple Sclerosis (MS).

18. The method of claim 16, wherein the sirtuin-activating compound comprises a formula selected from the group consisting of formulas 1-25, 30, 32-65, and 69-88.

19. The method of claim 16, wherein the sirtuin-activating compound is resveratrol, fisetin, butein, piceatannol or quercetin.

20. The method of claim 16, wherein the anti-inflammatory agent is a steroidal anti-inflammatory agent, a non-steroidal anti-inflammatory agent, or a non-steroidal immunomodulatory agent.

21. The method of claim 16, wherein the subject is a human.

22. A method for treating or preventing a neurodegenerative disorder in a subject comprising administering to a subject in need thereof a therapeutically effective amount of a PPAR-delta agonist.

23. The method of claim 22, wherein the PPAR-delta agonist is GW0742 or GW501516.

24. The method of claim 22, wherein the subject is human.

25. The method of claim 22, wherein the neurodegenerative disorder is selected from the group consisting of Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, Multiple Sclerosis (MS), and Friedreich's ataxia.

26. A method for preventing or treating a traumatic injury to a neuronal cell, comprising contacting a neuronal cell with an agent that increases the activity or protein level of a sirtuin.

27. A method for treating or preventing chemotherapeutic induced neuropathy comprising administering to a subject in need thereof a therapeutically effective amount of an agent that increases the activity or protein level of a sirtuin in a cell.

28. The method of claim 27, wherein the chemotherapeutic comprises a vinka alkaloid or cisplatin.

29. The method of claim 28, wherein the vinka alkaloid is vinblastine, vincristine, or vindesine.

30. The method of claim 27, wherein the agent is a sirtuin activating compound, salt or prodrug thereof.

31. The method of claim 30, wherein the sirtuin activating compound comprises a formula selected from the group consisting of formulas 1-25, 30, 32-65, and 69-88.

32. The method of claim 30, wherein the sirtuin activating compound is resveratrol, fisetin, butein, piceatannol or quercetin.

33. The method of claim 30, wherein the therapeutically effective amount is an amount of the sirtuin activating compound that has a sirtuin activating effect equal to or greater than 18 mg/kg resveratrol.

34. The method of claim 27, wherein the subject is a human.

35. A method for treating or preventing neuropathy associated with an ischemic event or disease comprising administering to a subject in need thereof a therapeutically effective amount of an agent that increases the activity or protein level of a sirtuin in a cell.

36. The method of claim 35, wherein the ischemic event is a stroke, coronary heart disease, stroke, emphysema, hemorrhagic shock, arrhythmia (e.g. atrial fibrillation), peripheral vascular disease, or transplant related injuries.

37. The method of claim 36, wherein the ischemic event is congestive heart failure or a myocardial infarction.

38. The method of claim 35, wherein the agent is a sirtuin activating compound, salt or prodrug thereof.

39. The method of claim 38, wherein the sirtuin activating compound comprises a formula selected from the group consisting of formulas 1-25, 30, 32-65, and 69-88.

40. The method of claim 38, wherein the sirtuin activating compound is resveratrol, fisetin, butein, piceatannol or quercetin.

41. The method of claim 38, wherein the therapeutically effective amount is an amount of the sirtuin activating compound that has a sirtuin activating effect equal to or greater than 18 mg/kg resveratrol.

42. The method of claim 35, wherein the subject is a human.

43. A method for treating or preventing a polyglutamine disease comprising administering to a subject in need thereof a therapeutically effective amount of a sirtuin activating compound and an HDAC I/II inhibitor.

44. The method of claim 43, wherein the polyglutamine disease is spinobulbar muscular atrophy (Kennedy disease), Huntington's disease, dentatorubralpallidoluysian atrophy (Haw River syndrome), spinocerebellar ataxia type 1, spinocerebellar ataxia type 2, spinocerebellar ataxia type 3 (Machado-Joseph disease), spinocerebellar ataxia type 6, spinocerebellar ataxia type 7, or spinocerebellar ataxia type 17.

45. The method of claim 43, wherein the HDAC I/II inhibitor is a hydroxamic acid, a cyclic peptie, a benzamide, a short-chain fatty acid, or depudecin.

46. The method of claim 45, wherein the HDAC I/II inhibitor is at least one of the following: suberoylanilide hydroxamic acid (SAHA), butyrate, pyroxamide, depsipeptide, or MS-27-275.

47. The method of claim 43, wherein the agent is a sirtuin activating compound, salt or prodrug thereof.

48. The method of claim 47, wherein the sirtuin activating compound comprises a formula selected from the group consisting of formulas 1-25, 30, 32-65, and 69-88.

49. The method of claim 47, wherein the sirtuin activating compound is resveratrol, fisetin, butein, piceatannol or quercetin.

50. The method of claim 43, wherein the subject is a human.


SEQUENCE LISTING (14 sequences)

SEQ ID NO: 1 - protein, 8 aa, Artificial Sequence
  peptide substrate for sirtuins derived from human histone H3
  VARIANT 2, 8: Xaa = acetylated lysine
KXQTARKX

SEQ ID NO: 2 - protein, 8 aa, Artificial Sequence
  peptide substrate for sirtuins derived from human histone H3
  VARIANT 2, 8: Xaa = acetylated lysine
KXSTGGKX

SEQ ID NO: 3 - protein, 9 aa, Artificial Sequence
  peptide substrate for sirtuins derived from human histone H3
  VARIANT 2, 9: Xaa = acetylated lysine
  VARIANT 4: Xaa = phosphorylated serine
KXSXTGGKX

SEQ ID NO: 4 - protein, 7 aa, Artificial Sequence
  peptide substrate for sirtuins derived from human histone H3
  VARIANT 2, 7: Xaa = acetylated lysine
KXAPRKX

SEQ ID NO: 5 - protein, 6 aa, Artificial Sequence
  peptide substrate for sirtuins derived from human histone H4
  VARIANT 6: Xaa = acetylated lysine
SGRGKX

SEQ ID NO: 6 - protein, 6 aa, Artificial Sequence
  peptide substrate for sirtuins derived from human histone H4
  VARIANT 6: Xaa = acetylated lysine
KGGAKX

SEQ ID NO: 7 - protein, 7 aa, Artificial Sequence
  peptide substrate for sirtuins derived from human histone H4
  VARIANT 2, 7: Xaa = acetylated lysine
KXGGAKX

SEQ ID NO: 8 - protein, 5 aa, Artificial Sequence
  peptide substrate for sirtuins derived from human P53
  VARIANT 5: Xaa = acetylated lysine
QPKKX

SEQ ID NO: 9 - protein, 6 aa, Artificial Sequence
  peptide substrate for sirtuins derived from human P53
  VARIANT 2, 6: Xaa = acetylated lysine
KXSKKX

SEQ ID NO: 10 - protein, 5 aa, Artificial Sequence
  peptide substrate for sirtuins derived from human P53
  VARIANT 5: Xaa = acetylated lysine
RHKKX

SEQ ID NO: 11 - protein, 6 aa, Artificial Sequence
  peptide substrate for sirtuins derived from human P53
  VARIANT 4, 6: Xaa = acetylated lysine
RHKXKX

SEQ ID NO: 12 - protein, 369 aa, Homo sapiens [sequence omitted]

SEQ ID NO: 13 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 14 - protein, 562 aa, Saccharomyces cerevisiae
MTIPHMKYAVSKTSENKVSNTVSPTQDKDAIRKQPDDIINNDEPSHKKIKVAQPDSLRETNTTDPLGHTKAALGEVASMELKPTNDMDPLAVSAASVVSMSNDVLKPETPKGPIIISKNPSNGIFYGPSFTKRESLNARMFLKYYGAHKFLDTYLPEDLNSLYIYYLIKLLGFEVKDQALIGTINSIVHINSQERVQDLGSAISVTNVEDPLAKKQTVRLIKDLQRAINKVLCTRLRLSNFFTIDHFIQKLHTARKILVLTGAGVSTSLGIPDFRSSEGFYSKIKHLGLDDPQDVFNYNIFMHDPSVFYNIANMVLPPEKIYSPLHSFIKMLQMKGKLLRNYTQNIDNLESYAGISTDKLVQCHGSFATATCVTCHWNLPGERIFNKIRNLELPLCPYCYKKRREYFPEGYNNKVGVAASQGSMSERPPYILNSYGVLKPDITFFGEALPNKFHKSIREDILECDLLICIGTSLKVAPVSEIVNMVPSHVPQVLINRDPVKHAEFDLSLLGYCDDIAAMVAQKCGWTIPHKKWNDLKNKNFKCQEKDKGVYVVTSDEHPKTL